(19) United States
(12) Patent Application Publication
(10) Pub. No.: US 2014/0005188 A1
(43) Pub. Date: Jan. 2, 2014

(54) PYRIDO[3,4-B]PYRAZINE DERIVATIVES AS SYK INHIBITORS

(71) Applicants: Francis Louis Atkinson, Stevenage (GB); Stephen John Atkinson, Stevenage (GB); Michael David Barker, Stevenage (GB); Clement Douault, Stevenage (GB); Neil Stuart Garton, Stevenage (GB); John Liddle, Stevenage (GB); Vipulkumar Kantibhai Patel, Stevenage (GB); Alexander G. Preston, Stevenage (GB); Tracy Jane Shipley, Stevenage (GB); David Matthew Wilson, Stevenage (GB); Robert J. Watson, Stevenage (GB)

(72) Inventors: Francis Louis Atkinson, Stevenage (GB); Stephen John Atkinson, Stevenage (GB); Michael David Barker, Stevenage (GB); Clement Douault, Stevenage (GB); Neil Stuart Garton, Stevenage (GB); John Liddle, Stevenage (GB); Vipulkumar Kantibhai Patel, Stevenage (GB); Alexander G. Preston, Stevenage (GB); Tracy Jane Shipley, Stevenage (GB); David Matthew Wilson, Stevenage (GB); Robert J. Watson, Stevenage (GB)

(73) Assignee: GLAXO GROUP LIMITED, Brentford, Middlesex (GB)

(21) Appl. No.: 14/004,375

(22) PCT Filed: Mar. 8, 2012
(86) PCT No.: PCT/EP2012/053949

§ 371 Date: Sep. 10, 2013

(30) Foreign Application Priority Data
Mar. 11, 2011 (GB) 1104138.1
Jul. 5, 2011 (GB) 1111409.7

(51) Int. Cl. C07D 471/04; C07D 487/04

(52) U.S. Cl. CPC C07D 471/04; C07D 487/04
USPC 514/232.5; 544/350; 514/249; 514/234.2; 544/117; 544/331; 544/80

(57) ABSTRACT

A compound of formula (I):

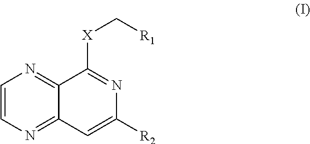

or a salt thereof;
which is an inhibitor of spleen tyrosine kinase (Syk) and therefore potentially of use in treating diseases resulting from inappropriate activation of mast cells, macrophages, and B-cells and related inflammatory responses and tissue damage, for instance inflammatory disease and/or allergic disorders, and in cancer therapy, specifically heme malignancies, and autoimmune conditions.

The present invention relates to novel chemical compounds which have activity against spleen tyrosine kinase (Syk), processes for their preparation, pharmaceutically acceptable formulations containing them and their use in therapy.

Syk is a non-receptor tyrosine kinase that is involved in coupling activated immunoreceptors to signal downstream events that mediate diverse cellular responses, including proliferation, differentiation, and phagocytosis. Syk is widely expressed in hematopoietic cells. Syk inhibitors have potential anti-inflammatory and immunomodulating activities. They inhibit Syk-mediated IgG Fc epsilon and gamma receptor and BCR receptor signalling, resulting in inhibition of the activation of mast cells, macrophages, and B-cells and related inflammatory responses and tissue damage. Accordingly, Syk inhibitors have attracted interest in a number of therapeutic areas, including the treatment of rheumatoid arthritis, B-cell lymphoma and asthma/rhinitis.

Rheumatoid arthritis (RA) is an auto-immune disease affecting approximately 1% of the population. It is characterised by inflammation of articular joints leading to debilitating destruction of bone and cartilage. Recent clinical studies with rituximab, which causes a reversible B cell depletion, (J. C. W. Edwards et al 2004, New Eng. J. Med. 350: 2572-2581), have shown that targeting B cell function is an appropriate therapeutic strategy in autoimmune diseases such as RA. Clinical benefit correlates with a reduction in auto-reactive antibodies (or rheumatoid factor) and these studies suggest that B cell function and indeed auto-antibody production are central to the ongoing pathology in the disease

Studies using cells from mice deficient in Syk have demonstrated a non-redundant role of this kinase in B cell function. The deficiency in Syk is characterised by a block in B cell development (M. Turner et al 1995 Nature 379: 298-302 and Cheng et al 1995, Nature 378: 303-306). These studies, along with studies on mature B cells deficient in Syk (Kurasaki et al 2000, Immunol. Rev. 176:19-29), demonstrate that Syk is required for the differentiation and activation of B cells. Hence, inhibition of Syk in RA patients is likely to block B cell function and hence reduce rheumatoid factor production. In addition to the role of Syk in B cell function, of relevance to the treatment of RA, is the requirement for Syk activity in Fc receptor (FcR) signalling. FcR activation by immune complexes in RA has been suggested to contribute to the release of multiple pro-inflammatory mediators.

The contribution of Syk dependent processes to the pathology of RA has been reviewed by Wong et al (2004, ibid).

The results of a 12 week proof of concept clinical trial for the Syk inhibitor R788 (fostamatinib disodium, Rigel) have been published: Treatment of rheumatoid arthritis with a Syk inhibitor: A twelve-week, randomized, placebo-controlled trial, Arthritis & Rheumatis, 58(11), 2008, 3309-3318.

Syk inhibitors may also be useful in cancer therapy, specifically heme malignancies, particularly Non-Hodgkin's Lymphomas including follicular (FL), mantle cell, Burkitt and diffuse large B cell (DLBCL) lymphomas.

Studies have shown that Syk is dysregulated by overexpression and/or constitutively activation in a variety of primary B-lymphoma tumours and also in B-lymphoma cell lines. Syk, through the PI3K/AKT pathway, the PLD pathway and AKT independent signalling, activates mTOR (mammalian target of rapamycin) which in turn increases B-cell survival and proliferation. Inhibition of Syk, in vitro, results in decreased mTOR activation and a reduction of clonicity in FL cells. Inhibition of Syk with curcumin in a murine model of B lymphoma (BKS-2) gave a significant reduction of tumour burden as measured by the total splenocyte number. (Leseux L. et al. Blood 15 Dec. 2006, Vol 108, No 13 pp 4156-4162 and Gururajan M. et al. Journal of Immunology, 2007, 178 pp 111-121).

Results of a Phase 2 clinical trial of R788 (fostamatinib disodium) in patients with relapsed or refractory B-Cell non-Hodgkin's lymphoma (NHL) show that the compound is well-tolerated by these patients, as well as a therapeutic benefit in patients suffering from diffuse large B-Cell lymphoma (DLBCL) and chronic lymphocytic leukemia/small lymphocytic lymphoma (CLL/SLL). Despite the fact that the patients enrolled in this trial had advanced disease and had failed treatment with marketed therapies, a significant number of them were particularly responsive to Syk inhibition with R788 (Chen et al Blood 2008 Vol 111 pp 2230-2237, www.Riqel.com)

Syk inhibitors may also be useful in the treatment of asthma and allergic rhinitis as they are important in transducing the downstream cellular signals associated with cross-linking FcεR1 and or FcγR1 receptors, and Syk is positioned early in the signalling cascade. In mast cells, for example, the early sequence of FcεR1 signalling following allergen cross-linking of receptor-IgE complexes involves first Lyn (a Src family tyrosine kinase) and then Syk.

Allergic rhinitis and asthma are diseases associated with hypersensitivity reactions and inflammatory events involving a multitude of cell types including mast cells, eosinophils, T cells and dendritic cells. Following exposure to allergen, high affinity immunoglobulin receptors for IgE (FcεRI) and IgG (FcγRI) become cross-linked and activate downstream processes in mast cells and other cell types leading to the release of pro-inflammatory mediators and airway spasmogens. In the mast cell, for example, IgE receptor cross-linking by allergen leads to release of mediators including histamine from pre-formed granules, as well as the synthesis and release of newly synthesised lipid mediators including prostaglandins and leukotrienes.

The Syk inhibitor R112 (Rigel), dosed intranasally in a phase I/II study for the treatment of allergic rhinitis, was shown to give a statistically significant decrease in PGD₂, a key immune mediator that is highly correlated with improvements in allergic rhinorrhea, as well as being safe across a range of indicators, thus providing the first evidence for the clinical safety and efficacy of a topical Syk inhibitor (see Meltzer, Eli O.; Berkowitz, Robert B.; Grossbard, Elliott B. An intranasal Syk inhibitor (R112) improves the symptoms of seasonal allergic rhinitis in a park environment. Journal of Allergy and Clinical Immunology (2005), 115(4), 791-796). In a further phase II clinical trial, for allergic rhinitis, R112 was however shown as having a lack of efficacy versus placebo (Clinical Trials.gov Identifier NCT0015089).

WO 03/057695 (Boehringer Ingelheim Pharmaceuticals, Inc) describes 1,6 Naphthyridines that have Syk inhibitory activity. These are further described in “Discovery and SAR of Novel [1,6] Naphthyridines as Potent Inhibitors of Spleen Tyrosine Kinase (SYK) (Bioorganic & Medicinal Chemistry Letters 13 (2003) 1415-1418). This has been followed with two more recent patent applications, WO 2010/015518 and WO 2010/015529 (Boehringer Ingelheim Pharmaceuticals, Inc), describing 4-dimethylamino-phenyl-substituted naphthyridines and substituted naphthyridines, respectively.

WO 04/035604 discloses the structural co-ordinates of the human Syk protein.

There remains however the need to identify further compounds which are inhibitors of spleen tyrosine kinase (Syk).

Thus, in one embodiment, the present invention provides a compound of formula (I):

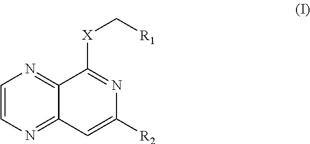

wherein:

### X is O, CH₂ or NH;

R₁ is a 5- or 6-membered heterocyclyl or —(CH₂)ₙR₅;
wherein the heterocyclyl is optionally substituted by one or two groups each independently selected from fluoro, methyl, ethyl and trifluoroethyl;
R₂ is a 5- or 6-membered heteroaryl, heterocyclyl or phenyl, or a 9- or 10-membered fused heteroaryl;
wherein the heteroaryl, heterocyclyl, phenyl or fused heteroaryl is optionally substituted by one or two groups each independently selected from C₁₋₆alkyl, OH, C₁₋₆alkoxy, —NR₃R₄, C₁₋₆-fluoroalkyl, benzyl, C₃₋₆cycloalkyl, oxo (═O), OC₁₋₆-fluoroalkyl and halogen;
R₃ and R₄ are each independently selected from hydrogen and methyl, or R₃ and R₄ together with the nitrogen to which they are attached form a 5- or 6-membered heterocyclyl;

### R₅ is —NH₂, —CF₃, —C(O)NH₂ or OH; and

n is an integer selected from 0, 1, 2 and 3; or
a salt thereof.

In another embodiment, the present invention provides a compound of formula (I):

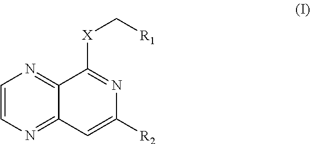

wherein:

### X is O, CH₂ or NH;

R₁ is a 5- or 6-membered heterocyclyl or —(CH₂)ₙR₅;
wherein the heterocyclyl is optionally substituted by one or two groups each independently selected from fluoro and methyl;
R₂ is a 5- or 6-membered heteroaryl, heterocyclyl or phenyl, or a 9- or 10-membered fused heteroaryl;
wherein the heteroaryl, heterocyclyl, phenyl or fused heteroaryl is optionally substituted by one or two groups each independently selected from C₁₋₆alkyl, OH, C₁₋₆alkoxy, —NR₃R₄, C₁₋₆-fluoroalkyl, benzyl, C₃₋₆cycloalkyl, and oxo (═O);
R₃ and R₄ are each independently selected from hydrogen and methyl, or R₃ and R₄ together with the nitrogen to which they are attached form a 5- or 6-membered heterocyclyl;

### R₅ is —NH₂, —CF₃, —C(O)NH₂ or OH; and

n is an integer selected from 0, 1, 2 and 3; or
a salt thereof.

In another embodiment, the present invention provides a compound of formula (I):

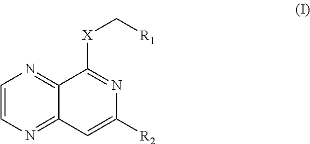

wherein:

### X is O, CH₂ or NH;

R₁ is a 5- or 6-membered heterocyclyl or —(CH₂)ₙR₅;
wherein the heterocyclyl is optionally substituted by one or two groups each independently selected from fluoro and methyl;
R₂ is a 5- or 6-membered heteroaryl or phenyl;
wherein the heteroaryl or phenyl is optionally substituted by one or two groups each independently selected from C₁₋₆alkyl, C₁₋₆alkoxy, —NR₃R₄, C₁₋₆-fluoroalkyl, benzyl and C₃₋₆cycloalkyl;
R₃ and R₄ are each independently selected from hydrogen and methyl;

### R₅ is —NH₂, —CF₃, —C(O)NH₂ or OH; and

n is an integer selected from 1, 2 and 3; or
a salt thereof.

In one embodiment X is O, CH₂ or NH. In another embodiment X is O or NH. In another embodiment X is O. In a further embodiment X is NH.

In one embodiment R₁ is a 5- or 6-membered heterocyclyl optionally substituted by one or two groups each independently selected from fluoro, methyl, ethyl and trifluoroethyl. In another embodiment R₁ is a 6-membered heterocyclyl optionally substituted by one or two groups each independently selected from fluoro, methyl, ethyl and trifluoroethyl. In another embodiment R₁ is a 6-membered heterocyclyl selected from piperidine, piperazine and morpholine optionally substituted by one or two groups each independently selected from fluoro, methyl, ethyl and trifluoroethyl. In another embodiment R₁ is a 6-membered heterocyclyl and substituents selected from:

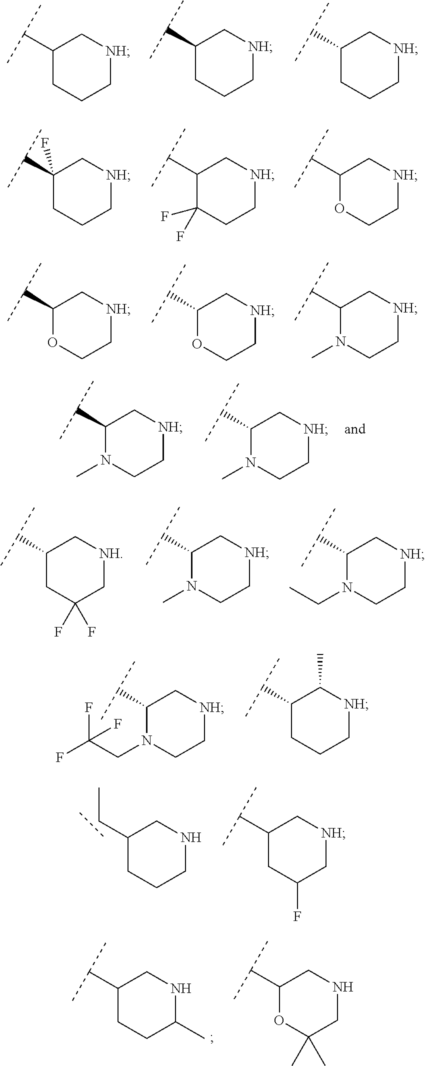

In another embodiment R₁ is a 6-membered heterocyclyl and substituents selected from:

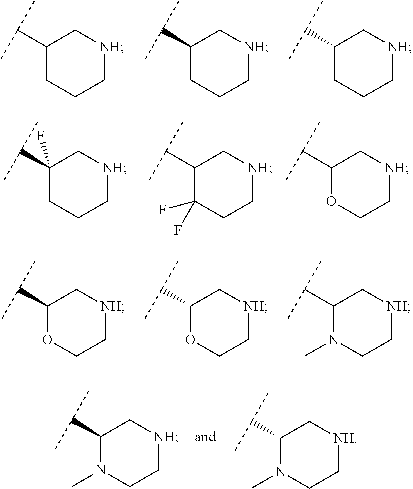

In a further embodiment R₁ is a 6-membered heterocyclyl and substituents selected from:

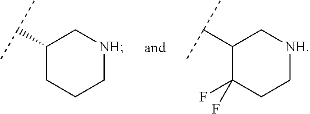

In one embodiment R₁ is —(CH₂)ₙR₅. In another embodiment R₁ is —(CH₂)ₙR₅ and R₅ is —NH₂, —CF₃, —C(O)NH₂ or OH. In a further embodiment R₅ is —NH₂.

In one embodiment n is selected from 0, 1, 2 and 3. In another embodiment n is selected from 1, 2 and 3. In a further embodiment n is 3.

In one embodiment R₂ is a 5- or 6-membered heteroaryl, heterocyclyl or phenyl; or a 9- or 10-membered fused heteroaryl wherein the heteroaryl, heterocyclyl, phenyl or fused heteroaryl is

optionally substituted by one or two groups each independently selected from C₁₋₆alkyl, OH, C₁₋₆alkoxy, —NR₃R₄, C₁₋₆-fluoroalkyl, benzyl, C₃₋₆cycloalkyl, oxo (═O), OC₁₋₆-fluoroalkyl and halogen;

In another embodiment R₂ is a 5- or 6-membered heteroaryl, heterocyclyl or phenyl, or a 9- or 10-membered fused heteroaryl wherein the heteroaryl, heterocyclyl, phenyl or fused heteroaryl is optionally substituted by one or two groups each independently selected from methyl, methoxy, —NH₂, —CH₂CF₃, benzyl, cyclopentyl, oxo, pyrrolidine and piperazine.

In one embodiment R₂ is selected from pyrazole, pyridine, phenyl, piperazine, pyrimidine and pyrrolopyridine optionally substituted by one or two groups each independently selected from C₁₋₆alkyl, OH, C₁₋₆alkoxy, —NR₃R₄, C₁₋₆-fluoroalkyl, benzyl, C₃₋₆cycloalkyl, oxo (═O), OC₁₋₆-fluoroalkyl and halogen;

In another embodiment R₂ is selected from:

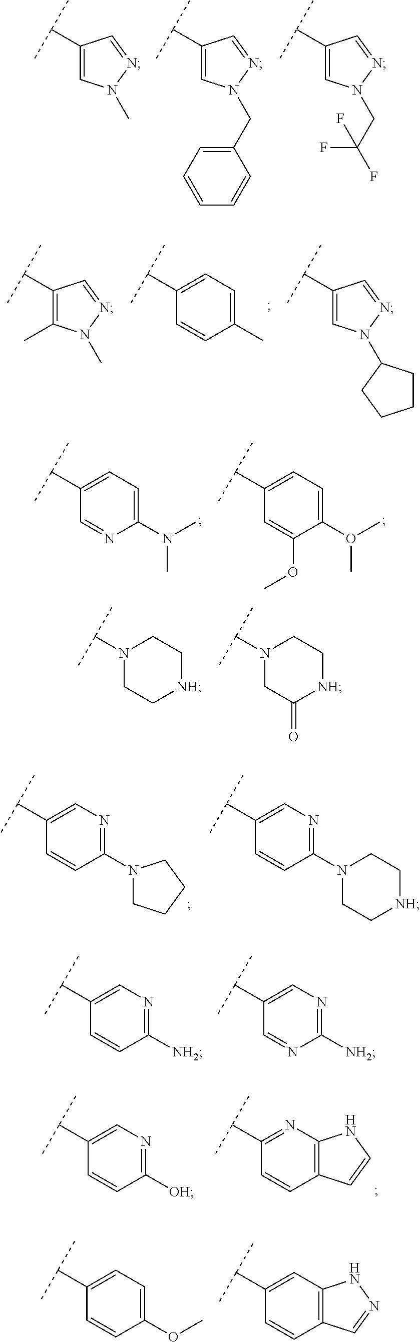

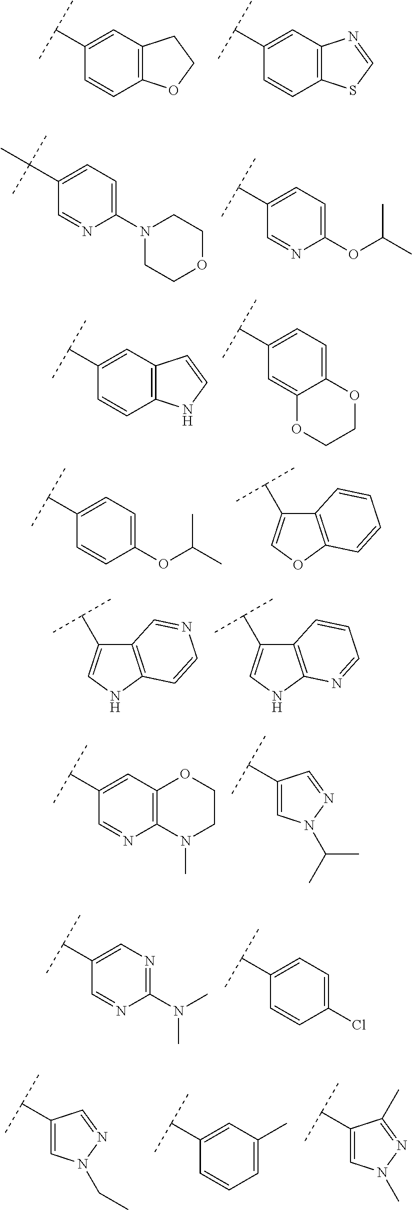

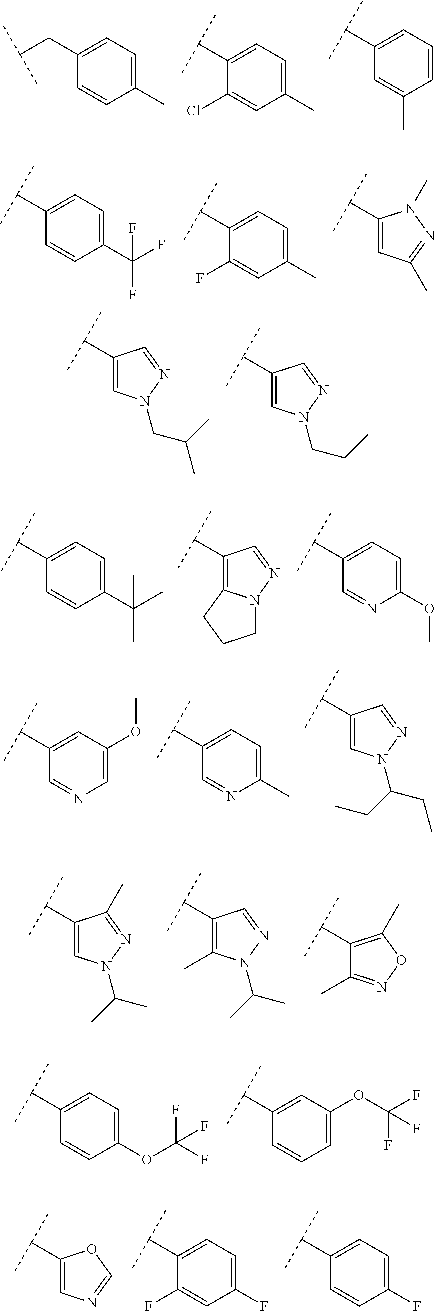

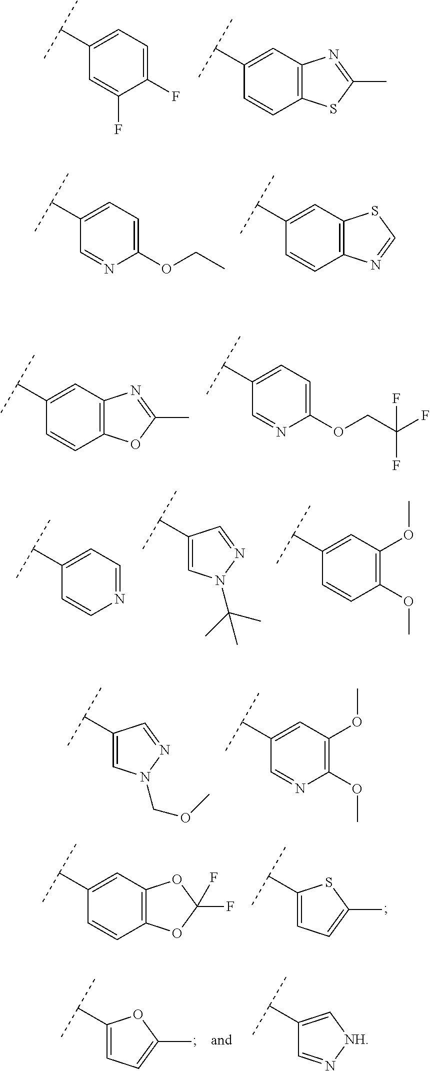

In one embodiment R₂ is a 5- or 6-membered heteroaryl or phenyl; wherein the heteroaryl or phenyl is optionally substituted by one or two groups each independently selected from C₁₋₆alkyl, C₁₋₆alkoxy, —NR₃R₄, C₁₋₆-fluoroalkyl, benzyl and C₃₋₆cycloalkyl. In another embodiment R₂ is a 5- or 6-membered heteroaryl or phenyl; wherein the heteroaryl or phenyl is optionally substituted by one or two groups each independently selected from methyl, methoxy, —NR₃R₄, —CH₂CF₃, benzyl and cyclopentyl.

In one embodiment R₂ is selected from pyrazole, pyridine and phenyl. In another embodiment R₂ is selected from:

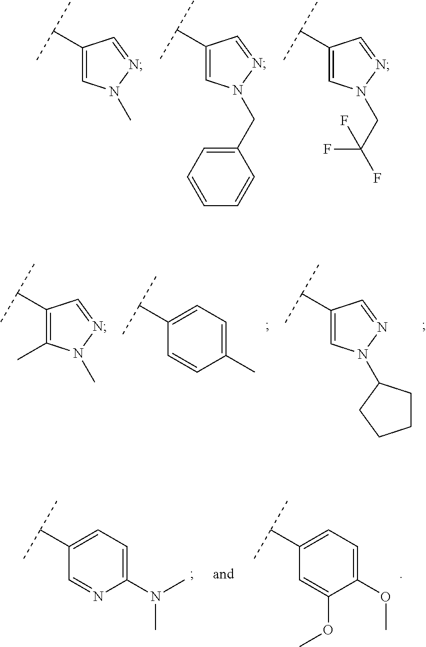

In a further embodiment R₂ is selected from:

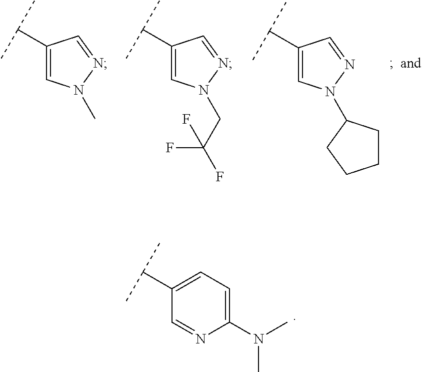

In one embodiment R₃ and R₄ are each independently selected from hydrogen and methyl. In another embodiment R₃ and R₄ are both methyl. In a further embodiment, R₃ and R₄ together with the nitrogen to which they are attached form a 5- or 6-membered heterocycyl.

In one embodiment, representative compounds of the invention include:

Examples 1-42 (ACD names):
7-[1-(Phenylmethyl)-1H-pyrazol-4-yl]-N-[(3S)-3-piperidinylmethyl]pyrido[3,4-b]pyrazin-5-amine
7-(1-Cyclopentyl-1H-pyrazol-4-yl)-N-[(3S)-3-piperidinylmethyl]pyrido[3,4-b]pyrazin-5-amine
N-{[(3S)-3-fluoro-3-piperidinyl]methyl}-7-[1-(2,2,2-trifluoroethyl)-1H-pyrazol-4-yl]pyrido[3,4-b]pyrazin-5-amine
7-[3,4-bis(Methyloxy)phenyl]-N-{[(3S)-3-fluoro-3-piperidinyl]methyl}pyrido[3,4-b]pyrazin-5-amine hydrochloride
N-{[(3S)-3-Fluoro-3-piperidinyl]methyl}-7-(1-methyl-1H-pyrazol-4-yl)pyrido[3,4-b]pyrazin-5-amine hydrochloride
5-{[(3S)-3-piperidinylmethyl]oxy}-7-[1-(2,2,2-trifluoroethyl)-1H-pyrazol-4-yl]pyrido[3,4-b]pyrazine hydrochloride
7-(1,5-Dimethyl-1H-pyrazol-4-yl)-5-{[(3S)-3-piperidinylmethyl]oxy}pyrido[3,4-b]pyrazine, hydrochloride
N,N-Dimethyl-5-(5-{[(3S)-3-piperidinylmethyl]oxy}pyrido[3,4-b]pyrazin-7-yl)-2-pyridinamine hydrochloride
7-(1-Methyl-1H-pyrazol-4-yl)-5-{[(3S)-3-piperidinylmethyl]oxy}pyrido[3,4-b]pyrazine hydrochloride
7-(1,5-Dimethyl-1H-pyrazol-4-yl)-5-{[(2S)-2-morpholinylmethyl]oxy}pyrido[3,4-b]pyrazine, hydrochloride
7-(1-Methyl-1H-pyrazol-4-yl)-N-[(2S)-2-morpholinylmethyl]pyrido[3,4-b]pyrazin-5-amine, hydrochloride
N-{[(2S)-1-methyl-2-piperazinyl]methyl}-7-(1-methyl-1H-pyrazol-4-yl)pyrido[3,4-b]pyrazin-5-amine hydrochloride
5-{[(4,4-Difluoro-3-piperidinyl)methyl]oxy}-7-(1-methyl-1H-pyrazol-4-yl)pyrido[3,4-b]pyrazine
7-(1-Methyl-1H-pyrazol-4-yl)-5-[2-(3-piperidinyl)ethyl]pyrido[3,4-b]pyrazine hydrochloride
N-{7-[6-(Dimethylamino)-3-pyridinyl]pyrido[3,4-b]pyrazin-5-yl}-1,4-butanediamine hydrochloride
7-[6-(dimethylamino)-3-pyridinyl]-N-[(2)-2-morpholinylmethyl]pyrido[3,4-b]pyrazin-5-amine, hydrochloride (Isomer 1)
7-[6-(dimethylamino)-3-pyridinyl]-N-[2-morpholinylmethyl]pyrido[3,4-b]pyrazin-5-amine, hydrochloride (Isomer 2)
N-[(4,4-difluoro-3-piperidinyl)methyl]-7-[6-(dimethylamino)-3-pyridinyl]pyrido[3,4-b]pyrazin-5-amine (Isomer 2)
N-[(4,4-difluoro-3-piperidinyl)methyl]-7-[6-(dimethylamino)-3-pyridinyl]pyrido[3,4-b]pyrazin-5-amine (Isomer 1)
N-((5,5-difluoropiperidin-3-yl)methyl)-7-(6-(dimethylamino)pyridin-3-yl)pyrido[3,4-b]pyrazin-5-amine
4-(5-{[(3S)-3-piperidinylmethyl]amino}pyrido[3,4-b]pyrazin-7-yl)-2-piperazinone, hydrochloride
7-(1-piperazinyl)-N-[(3S)-3-piperidinylmethyl]pyrido[3,4-b]pyrazin-5-amine, hydrochloride
N,N-dimethyl-5-(5-{[(3R)-3-piperidinylmethyl]oxy}pyrido[3,4-b]pyrazin-7-yl)-2-pyridinamine
7-[6-(dimethylamino)-3-pyridinyl]-N-(2,2,2-trifluoroethyl)pyrido[3,4-b]pyrazin-5-amine
4-({7-[6-(dimethylamino)-3-pyridinyl]pyrido[3,4-b]pyrazin-5-yl}amino)-1-butanol
N³-{7-[6-(dimethylamino)-3-pyridinyl]pyrido[3,4-b]pyrazin-5-yl}-β-alaninamide
7-[6-(dimethylamino)-3-pyridinyl]-N-[(3S)-3-piperidinylmethyl]pyrido[3,4-b]pyrazin-5-amine
N,N-dimethyl-5-{5-[2-(3-piperidinyl)ethyl]pyrido[3,4-b]pyrazin-7-yl}-2-pyridinamine
7-(1-methyl-1H-pyrazol-4-yl)-N-[(3S)-3-piperidinylmethyl]pyrido[3,4-b]pyrazin-5-amine
N-[(5,5-difluoro-3-piperidinyl)methyl]-7-(1-methyl-1H-pyrazol-4-yl)pyrido[3,4-b]pyrazin-5-amine
7-[6-(dimethylamino)-3-pyridinyl]-N-[(3R)-3-piperidinylmethyl]pyrido[3,4-b]pyrazin-5-amine
N-[(3S)-3-piperidinylmethyl]-7-[6-(1-pyrrolidinyl)-3-pyridinyl]pyrido[3,4-b]pyrazin-5-amine
7-[6-(1-piperazinyl)-3-pyridinyl]-N-[(3S)-3-piperidinylmethyl]pyrido[3,4-b]pyrazin-5-amine
7-(6-amino-3-pyridinyl)-N-[(3S)-3-piperidinylmethyl]pyrido[3,4-b]pyrazin-5-amine
7-(2-amino-5-pyrimidinyl)-N-[(3S)-3-piperidinylmethyl]pyrido[3,4-b]pyrazin-5-amine
5-(5-{[(3S)-3-piperidinylmethyl]amino}pyrido[3,4-b]pyrazin-7-yl)-2(1H)-pyridinone
N-[(3S)-3-piperidinylmethyl]-7-(1H-pyrrolo[2,3-b]pyridin-5-yl)pyrido[3,4-b]pyrazin-5-amine
7-(5-methyl-2-thienyl)-N-[(3S)-3-piperidinylmethyl]pyrido[3,4-b]pyrazin-5-amine
7-(5-methyl-2-furanyl)-N-[(3S)-3-piperidinylmethyl]pyrido[3,4-b]pyrazin-5-amine
N-[(3S)-3-piperidinylmethyl]-7-(1H-pyrazol-3-yl)pyrido[3,4-b]pyrazin-5-amine
N-[(3S)-3-piperidinylmethyl]-7-(1H-pyrazol-4-yl)pyrido[3,4-b]pyrazin-5-amineN-[7-(4-methylphenyl)pyrido[3,4-b]pyrazin-5-yl]-1,4-butanediamine

Examples 43-203 (IUPAC names):
7-(4-methylphenyl)-N-(morpholin-2-ylmethyl)pyrido[3,4-b]pyrazin-5-amine hydrochloride, single unknown enantiomer
7-(4-methylphenyl)-N-(morpholin-2-ylmethyl)pyrido[3,4-b]pyrazin-5-amine hydrochloride, single unknown enantiomer
7-(4-methoxyphenyl)-N-[(3S)-piperidin-3-ylmethyl]pyrido[3,4-b]pyrazin-5-amine hydrochloride
(3S)-3-({[7-(4-methylphenyl)pyrido[3,4-b]pyrazin-5-yl]oxy}methyl)piperidine hydrochloride
7-(2,3-dihydro-1-benzofuran-5-yl)-N-[(3S)-piperidin-3-ylmethyl]pyrido[3,4-b]pyrazin-5-amine hydrochloride
7-(1,3-benzothiazol-5-yl)-N-[(3S)-piperidin-3-ylmethyl]pyrido[3,4-b]pyrazin-5-amine hydrochloride
7-(1H-indol-5-yl)-N-[(3S)-piperidin-3-ylmethyl]pyrido[3,4-b]pyrazin-5-amine hydrochloride
7-(2,3-dihydro-1,4-benzodioxin-6-yl)-N-[(3S)-piperidin-3-ylmethyl]pyrido[3,4-b]pyrazin-5-amine hydrochloride
7-[6-(morpholin-4-yl)pyridin-3-yl]-N-[(3S)-piperidin-3-ylmethyl]pyrido[3,4-b]pyrazin-5-amine hydrochloride
N-[(3S)-piperidin-3-ylmethyl]-7-[6-(propan-2-yloxy)pyridin-3-yl]pyrido[3,4-b]pyrazin-5-amine hydrochloride
(3S)-3-({[7-(4-methoxyphenyl)pyrido[3,4-b]pyrazin-5-yl]oxy}methyl)piperidine hydrochloride
(3S)-3-({[7-(1H-pyrazol-4-yl)pyrido[3,4-b]pyrazin-5-yl]oxy}methyl)piperidine hydrochloride
(3S)-3-({[7-(1-benzofuran-3-yl)pyrido[3,4-b]pyrazin-5-yl]oxy}methyl)piperidine hydrochloride
(3S)-3-{[(7-{1H-pyrrolo[3,2-c]pyridin-3-yl}pyrido[3,4-b]pyrazin-5-yl)oxy]methyl}piperidine hydrochloride
(3S)-3-{[(7-{1H-pyrrolo[2,3-b]pyridin-3-yl}pyrido[3,4-b]pyrazin-5-yl)oxy]methyl}piperidine hydrochloride
(2R)-2-({[7-(1H-pyrazol-4-yl)pyrido[3,4-b]pyrazin-5-yl]oxy}methyl)morpholine hydrochloride
(2R)-2-({[7-(1-methyl-1H-pyrazol-4-yl)pyrido[3,4-b]pyrazin-5-yl]oxy}methyl)morpholine hydrochloride
N,N-dimethyl-5-{5-[(2R)-morpholin-2-ylmethoxy]pyrido[3,4-b]pyrazin-7-yl}pyridin-2-amine hydrochloride
N-(morpholin-2-ylmethyl)-7-(1H-pyrazol-4-yl)pyrido[3,4-b]pyrazin-5-amine hydrochloride, single unknown enantiomer
(3S)-3-{[(7-{4-methyl-2H,3H,4H-pyrido[3,2-b][1,4]oxazin-7-yl}pyrido[3,4-b]pyrazin-5-yl)oxy]methyl}piperidine hydrochloride
(3S)-3-[({7-[1-(propan-2-yl)-1H-pyrazol-4-yl]pyrido[3,4-b]pyrazin-5-yl}oxy)methyl]piperidine hydrochloride
(3S)-3-[({7-[1-(propan-2-yl)-1H-pyrazol-4-yl]pyrido[3,4-b]pyrazin-5-yl}oxy)methyl]piperidine
N,N-dimethyl-5-{5-[(3S)-piperidin-3-ylmethoxy]pyrido[3,4-b]pyrazin-7-yl}pyrimidin-2-amine hydrochloride
(3S)-3-({[7-(2,3-dihydro-1,4-benzodioxin-6-yl)pyrido[3,4-b]pyrazin-5-yl]oxy}methyl)piperidine hydrochloride
(3S)-3-({[7-(4-chlorophenyl)pyrido[3,4-b]pyrazin-5-yl]oxy}methyl)piperidine
(3S)-3-[({7-[4-(propan-2-yloxy)phenyl]pyrido[3,4-b]pyrazin-5-yl}oxy)methyl]piperidine
(3S)-3-({[7-(1-ethyl-1H-pyrazol-4-yl)pyrido[3,4-b]pyrazin-5-yl]oxy}methyl)piperidine hydrochloride
N,N-dimethyl-5-[5-({[(2S)-1-methylpiperazin-2-yl]methyl}amino)pyrido[3,4-b]pyrazin-7-yl]pyridin-2-amine hydrochloride
4-(5-{5-[(3S)-piperidin-3-ylmethoxy]pyrido[3,4-b]pyrazin-7-yl}pyridin-2-yl)morpholine
(3S)-3-({[7-(3-methylphenyl)pyrido[3,4-b]pyrazin-5-yl]oxy}methyl)piperidine dihydrochloride
N-(morpholin-2-ylmethyl)-7-[6-(morpholin-4-yl)pyridin-3-yl]pyrido[3,4-b]pyrazin-5-amine hydrochloride, single unknown enantiomer
N-{[(2S)-1-methylpiperazin-2-yl]methyl}-7-(1H-pyrazol-4-yl)pyrido[3,4-b]pyrazin-5-amine hydrochloride
N-{[(2S)-1-methylpiperazin-2-yl]methyl}-7-[6-(morpholin-4-yl)pyridin-3-yl]pyrido[3,4-b]pyrazin-5-amine hydrochloride
(3S)-3-({[7-(1,3-dimethyl-1H-pyrazol-4-yl)pyrido[3,4-b]pyrazin-5-yl]oxy}methyl)piperidine
(3S)-3-({[7-(1,3-dimethyl-1H-pyrazol-4-yl)pyrido[3,4-b]pyrazin-5-yl]oxy}methyl)piperidine hydrochloride
7-(1-ethyl-1H-pyrazol-4-yl)-N-[(2R)-morpholin-2-ylmethyl]pyrido[3,4-b]pyrazin-5-amine
N-[(2R)-morpholin-2-ylmethyl]-7-[1-(propan-2-yl)-1H-pyrazol-4-yl]pyrido[3,4-b]pyrazin-5-amine
(3S)-3-({[7-(2-chloro-4-methylphenyl)pyrido[3,4-b]pyrazin-5-yl]oxy}methyl)piperidine hydrochloride
(3S)-3-({[7-(3-chlorophenyl)pyrido[3,4-b]pyrazin-5-yl]oxy}methyl)piperidine hydrochloride
N-{[(2S)-1-methylpiperazin-2-yl]methyl}-7-[1-(propan-2-yl)-1H-pyrazol-4-yl]pyrido[3,4-b]pyrazin-5-amine hydrochloride
(3S)-3-[({7-[4-(trifluoromethyl)phenyl]pyrido[3,4-b]pyrazin-5-yl}oxy)methyl]piperidine hydrochloride
(3S)-3-[({7-[4-(trifluoromethyl)phenyl]pyrido[3,4-b]pyrazin-5-yl}oxy)methyl]piperidine
(3S)-3-({[7-(2-fluoro-4-methylphenyl)pyrido[3,4-b]pyrazin-5-yl]oxy}methyl)piperidine
N,N-dimethyl-5-(5-{[(2S)-1-methylpiperazin-2-yl]methoxy}pyrido[3,4-b]pyrazin-7-yl)pyridin-2-amine
(2S)-1-methyl-2-({[7-(1-methyl-1H-pyrazol-4-yl)pyrido[3,4-b]pyrazin-5-yl]oxy}methyl)piperazine
7-(1,3-dimethyl-1H-pyrazol-4-yl)-N-[(2S)-morpholin-2-ylmethyl]pyrido[3,4-b]pyrazin-5-amine hydrochloride
N,N-dimethyl-5-[5-({[(2R)-1-methylpiperazin-2-yl]methyl}amino)pyrido[3,4-b]pyrazin-7-yl]pyridin-2-amine hydrochloride
7-(1,5-dimethyl-1H-pyrazol-4-yl)-N-[(2S)-morpholin-2-ylmethyl]pyrido[3,4-b]pyrazin-5-amine hydrochloride
(2S)-2-({[7-(4-methylphenyl)pyrido[3,4-b]pyrazin-5-yl]oxy}methyl)morpholine hydrochloride
7-(1-methyl-1H-pyrazol-5-yl)-N-[(2S)-morpholin-2-ylmethyl]pyrido[3,4-b]pyrazin-5-amine hydrochloride
7-(1,3-dimethyl-1H-pyrazol-5-yl)-N-[(2S)-morpholin-2-ylmethyl]pyrido[3,4-b]pyrazin-5-amine
7-[1-(2-methylpropyl)-1H-pyrazol-4-yl]-N-[(2S)-morpholin-2-ylmethyl]pyrido[3,4-b]pyrazin-5-amine
N-[(2S)-morpholin-2-ylmethyl]-7-[6-(morpholin-4-yl)pyridin-3-yl]pyrido[3,4-b]pyrazin-5-amine hydrochloride
7-(1-ethyl-1H-pyrazol-4-yl)-N-[(2S)-morpholin-2-ylmethyl]pyrido[3,4-b]pyrazin-5-amine hydrochloride
N-[(2S)-morpholin-2-ylmethyl]-7-[1-(propan-2-yl)-1H-pyrazol-4-yl]pyrido[3,4-b]pyrazin-5-amine
(2S)-2-({[7-(1,5-dimethyl-1H-pyrazol-4-yl)pyrido[3,4-b]pyrazin-5-yl]oxy}methyl)-1-methylpiperazine
(2S)-2-({[7-(1-methyl-1H-pyrazol-4-yl)pyrido[3,4-b]pyrazin-5-yl]oxy}methyl)morpholine hydrochloride
N-[(2S)-morpholin-2-ylmethyl]-7-[1-(2,2,2-trifluoroethyl)-1H-pyrazol-4-yl]pyrido[3,4-b]pyrazin-5-amine hydrochloride
N-[(2S)-morpholin-2-ylmethyl]-7-[1-(2,2,2-trifluoroethyl)-1H-pyrazol-4-yl]pyrido[3,4-b]pyrazin-5-amine
N-[(2S)-morpholin-2-ylmethyl]-7-(1-propyl-1H-pyrazol-4-yl)pyrido[3,4-b]pyrazin-5-amine
N-[(2S)-morpholin-2-ylmethyl]-7-(1-propyl-1H-pyrazol-4-yl)pyrido[3,4-b]pyrazin-5-amine hydrochloride
7-(4-tert-butylphenyl)-N-[(2S)-morpholin-2-ylmethyl]pyrido[3,4-b]pyrazin-5-amine
(2S)-1-ethyl-2-({[7-(1-methyl-1H-pyrazol-4-yl)pyrido[3,4-b]pyrazin-5-yl]oxy}methyl)piperazine hydrochloride
5-{5-[(3-fluoropiperidin-3-yl)methoxy]pyrido[3,4-b]pyrazin-7-yl}-N,N-dimethylpyridin-2-amine
5-{5-[(3-fluoropiperidin-3-yl)methoxy]pyrido[3,4-b]pyrazin-7-yl}-N,N-dimethylpyridin-2-amine, single unknown enantiomer
5-{5-[(3-fluoropiperidin-3-yl)methoxy]pyrido[3,4-b]pyrazin-7-yl}-N,N-dimethylpyridin-2-amine, single unknown enantiomer
5-(5-{[(2S)-1-ethylpiperazin-2-yl]methoxy}pyrido[3,4-b]pyrazin-7-yl)-N,N-dimethylpyridin-2-amine hydrochloride
(3S)-3-{[(7-{4H,5H,6H-pyrrolo[1,2-b]pyrazol-3-yl}pyrido[3,4-b]pyrazin-5-yl)oxy]methyl}piperidine
(3R)-3-({[7-(1-methyl-1H-pyrazol-4-yl)pyrido[3,4-b]pyrazin-5-yl]oxy}methyl)piperidine
N,N-dimethyl-5-(5-{[(2S)-1-(2,2,2-trifluoroethyl)piperazin-2-yl]methoxy}pyrido[3,4-b]pyrazin-7-yl)pyridin-2-amine
(2S)-2-({[7-(1-methyl-1H-pyrazol-4-yl)pyrido[3,4-b]pyrazin-5-yl]oxy}methyl)-1-(2,2,2-trifluoroethyl)piperazine
(6S)-2,2-dimethyl-6-({[7-(1-methyl-1H-pyrazol-4-yl)pyrido[3,4-b]pyrazin-5-yl]oxy}methyl)morpholine
N-[(4,4-difluoropiperidin-3-yl)methyl]-7-(1-methyl-1H-pyrazol-4-yl)pyrido[3,4-b]pyrazin-5-amine
3-fluoro-3-({[7-(1-methyl-1H-pyrazol-4-yl)pyrido[3,4-b]pyrazin-5-yl]oxy}methyl)piperidine, single unknown enantiomer
3-fluoro-3-({[7-(1-methyl-1H-pyrazol-4-yl)pyrido[3,4-b]pyrazin-5-yl]oxy}methyl)piperidine, single unknown enantiomer
5-[5-({[(3R)-3-fluoropiperidin-3-yl]methyl}amino)pyrido[3,4-b]pyrazin-7-yl]-N,N-dimethylpyridin-2-amine hydrochloride
N-{[(3S)-3-fluoropiperidin-3-yl]methyl}-7-(1-methyl-1H-pyrazol-4-yl)pyrido[3,4-b]pyrazin-5-amine hydrochloride
N-[(4,4-difluoropiperidin-3-yl)methyl]-7-(1-methyl-1H-pyrazol-4-yl)pyrido[3,4-b]pyrazin-5-amine, single unknown enantiomer
N-[(4,4-difluoropiperidin-3-yl)methyl]-7-(1-methyl-1H-pyrazol-4-yl)pyrido[3,4-b]pyrazin-5-amine, single unknown enantiomer
5-[5-({[(3S)-3-fluoropiperidin-3-yl]methyl}amino)pyrido[3,4-b]pyrazin-7-yl]-N,N-dimethylpyridin-2-amine hydrochloride
(2S,3S)-2-methyl-3-({[7-(1-methyl-1H-pyrazol-4-yl)pyrido[3,4-b]pyrazin-5-yl]oxy}methyl)piperidine
7-(1-methyl-1H-pyrazol-4-yl)-N-[(2R)-morpholin-2-ylmethyl]pyrido[3,4-b]pyrazin-5-amine
3,3-difluoro-5-({[7-(1-methyl-1H-pyrazol-4-yl)pyrido[3,4-b]pyrazin-5-yl]oxy}methyl)piperidine hydrochloride
N-[(3-fluoropiperidin-3-yl)methyl]-7-(4-methylphenyl)pyrido[3,4-b]pyrazin-5-amine
N-[(3-fluoropiperidin-3-yl)methyl]-7-(6-methoxypyridin-3-yl)pyrido[3,4-b]pyrazin-5-amine
N-[(3-fluoropiperidin-3-yl)methyl]-7-(5-methoxypyridin-3-yl)pyrido[3,4-b]pyrazin-5-amine
N-[(3-fluoropiperidin-3-yl)methyl]-7-(6-methylpyridin-3-yl)pyrido[3,4-b]pyrazin-5-amine
N-[(3-fluoropiperidin-3-yl)methyl]-7-(4-methoxyphenyl)pyrido[3,4-b]pyrazin-5-amine
N-[(4,4-difluoropiperidin-3-yl)methyl]-7-(4-methylphenyl)pyrido[3,4-b]pyrazin-5-amine
N-[(4,4-difluoropiperidin-3-yl)methyl]-7-(4-methoxyphenyl)pyrido[3,4-b]pyrazin-5-amine
5-(5-{[(3-fluoropiperidin-3-yl)methyl]amino}pyrido[3,4-b]pyrazin-7-yl)-N,N-dimethylpyrimidin-2-amine
N-(4-methylphenyl)-5-[(3S)-piperidin-3-ylmethoxy]pyrido[3,4-b]pyrazin-7-amine hydrochloride
7-(1-cyclopentyl-1H-pyrazol-4-yl)-N-[(3-fluoropiperidin-3-yl)methyl]pyrido[3,4-b]pyrazin-5-amine hydrochloride
7-(1-cyclopentyl-1H-pyrazol-4-yl)-N-[(3-fluoropiperidin-3-yl)methyl]pyrido[3,4-b]pyrazin-5-amine
N-[(3-fluoropiperidin-3-yl)methyl]-7-[1-(propan-2-yl)-1H-pyrazol-4-yl]pyrido[3,4-b]pyrazin-5-amine hydrochloride
N-[(3-fluoropiperidin-3-yl)methyl]-7-[1-(pentan-3-yl)-1H-pyrazol-4-yl]pyrido[3,4-b]pyrazin-5-amine hydrochloride
7-(1-benzyl-1H-pyrazol-4-yl)-N-[(3-fluoropiperidin-3-yl)methyl]pyrido[3,4-b]pyrazin-5-amine hydrochloride
7-(1-ethyl-1H-pyrazol-4-yl)-N-[(3-fluoropiperidin-3-yl)methyl]pyrido[3,4-b]pyrazin-5-amine hydrochloride
7-(1,5-dimethyl-1H-pyrazol-4-yl)-N-[(3-fluoropiperidin-3-yl)methyl]pyrido[3,4-b]pyrazin-5-amine hydrochloride
N-[(3-fluoropiperidin-3-yl)methyl]-7-[1-(2-methylpropyl)-1H-pyrazol-4-yl]pyrido[3,4-b]pyrazin-5-amine hydrochloride
N-[(3-fluoropiperidin-3-yl)methyl]-7-(1-propyl-1H-pyrazol-4-yl)pyrido[3,4-b]pyrazin-5-amine hydrochloride
7-[1-(2-methylpropyl)-1H-pyrazol-4-yl]-N-[(3S)-piperidin-3-ylmethyl]pyrido[3,4-b]pyrazin-5-amine
7-(1,5-dimethyl-1H-pyrazol-4-yl)-N-[(3S)-piperidin-3-ylmethyl]pyrido[3,4-b]pyrazin-5-amine hydrochloride
(3S)-3-[({7-[(4-methylphenyl)methyl]pyrido[3,4-b]pyrazin-5-yl}oxy)methyl]piperidine hydrochloride
7-(1,3-dimethyl-1H-pyrazol-4-yl)-N-[(3S)-piperidin-3-ylmethyl]pyrido[3,4-b]pyrazin-5-amine hydrochloride
N-[(3S)-piperidin-3-ylmethyl]-7-(1-propyl-1H-pyrazol-4-yl)pyrido[3,4-b]pyrazin-5-amine hydrochloride
7-(1,3-dimethyl-1H-pyrazol-4-yl)-N-[(3-fluoropiperidin-3-yl)methyl]pyrido[3,4-b]pyrazin-5-amine hydrochloride
7-(1-ethyl-1H-pyrazol-4-yl)-N-[(3S)-piperidin-3-ylmethyl]pyrido[3,4-b]pyrazin-5-amine
7-[1-(pentan-3-yl)-1H-pyrazol-4-yl]-N-[(3S)-piperidin-3-ylmethyl]pyrido[3,4-b]pyrazin-5-amine
N-[(3S)-piperidin-3-ylmethyl]-7-[1-(propan-2-yl)-1H-pyrazol-4-yl]pyrido[3,4-b]pyrazin-5-amine
N-[(3S)-piperidin-3-ylmethyl]-7-[1-(propan-2-yl)-1H-pyrazol-4-yl]pyrido[3,4-b]pyrazin-5-amine hydrochloride
7-[3-methyl-1-(propan-2-yl)-1H-pyrazol-4-yl]-N-[(3S)-piperidin-3-ylmethyl]pyrido[3,4-b]pyrazin-5-amine hydrochloride
N-[(3-fluoropiperidin-3-yl)methyl]-7-[3-methyl-1-(propan-2-yl)-1H-pyrazol-4-yl]pyrido[3,4-b]pyrazin-5-amine hydrochloride
N-[(3S)-piperidin-3-ylmethyl]-7-[1-(2,2,2-trifluoroethyl)-1H-pyrazol-4-yl]pyrido[3,4-b]pyrazin-5-amine
7-(dimethyl-1,2-oxazol-4-yl)-N-[(3-fluoropiperidin-3-yl)methyl]pyrido[3,4-b]pyrazin-5-amine hydrochloride
N-[(3-fluoropiperidin-3-yl)methyl]-7-[1-(2,2,2-trifluoroethyl)-1H-pyrazol-4-yl]pyrido[3,4-b]pyrazin-5-amine hydrochloride
3-(2-{7-[1-(propan-2-yl)-1H-pyrazol-4-yl]pyrido[3,4-b]pyrazin-5-yl}ethyl)piperidine
7-[5-methyl-1-(propan-2-yl)-1H-pyrazol-4-yl]-N-[(3S)-piperidin-3-ylmethyl]pyrido[3,4-b]pyrazin-5-amine hydrochloride
(+/−)(3S,5R)-3-fluoro-5-({[7-(1-methyl-1H-pyrazol-4-yl)pyrido[3,4-b]pyrazin-5-yl]oxy}methyl)piperidine
(+/−)(3R,5R)-3-fluoro-5-({[7-(1-methyl-1H-pyrazol-4-yl)pyrido[3,4-b]pyrazin-5-yl]oxy}methyl)piperidine
N-[(3-fluoropiperidin-3-yl)methyl]-7-[3-(trifluoromethoxy)phenyl]pyrido[3,4-b]pyrazin-5-amine hydrochloride
N-[(3-fluoropiperidin-3-yl)methyl]-7-[4-(trifluoromethoxy)phenyl]pyrido[3,4-b]pyrazin-5-amine hydrochloride
N-[(3-fluoropiperidin-3-yl)methyl]-7-(1,3-oxazol-5-yl)pyrido[3,4-b]pyrazin-5-amine
7-(2,4-difluorophenyl)-N-[(3-fluoropiperidin-3-yl)methyl]pyrido[3,4-b]pyrazin-5-amine
7-(4-fluorophenyl)-N-[(3-fluoropiperidin-3-yl)methyl]pyrido[3,4-b]pyrazin-5-amine
7-(3,4-difluorophenyl)-N-[(3-fluoropiperidin-3-yl)methyl]pyrido[3,4-b]pyrazin-5-amine
3-fluoro-3-(2-{7-[1-(propan-2-yl)-1H-pyrazol-4-yl]pyrido[3,4-b]pyrazin-5-yl}ethyl)piperidine
N-[(3-fluoropiperidin-3-yl)methyl]-7-(2-methyl-1,3-benzothiazol-5-yl)pyrido[3,4-b]pyrazin-5-amine hydrochloride
7-(1-methyl-1H-pyrazol-4-yl)-N-[(3R)-piperidin-3-ylmethyl]pyrido[3,4-b]pyrazin-5-amine hydrochloride
7-(1,3-dimethyl-1H-pyrazol-4-yl)-N-{[(3S)-3-fluoropiperidin-3-yl]methyl}pyrido[3,4-b]pyrazin-5-amine hydrochloride
7-(6-ethoxypyridin-3-yl)-N-[(3-fluoropiperidin-3-yl)methyl]pyrido[3,4-b]pyrazin-5-amine hydrochloride
7-(1,3-benzothiazol-6-yl)-N-[(3-fluoropiperidin-3-yl)methyl]pyrido[3,4-b]pyrazin-5-amine hydrochloride
N-[(3-fluoropiperidin-3-yl)methyl]-7-(2-methyl-1,3-benzoxazol-5-yl)pyrido[3,4-b]pyrazin-5-amine hydrochloride
7-(1-methyl-1H-pyrazol-4-yl)-N-[(6-methylpiperidin-3-yl)methyl]pyrido[3,4-b]pyrazin-5-amine hydrochloride, single unknown enantiomer
7-(2,3-dihydro-1,4-benzodioxin-6-yl)-N-[(3-fluoropiperidin-3-yl)methyl]pyrido[3,4-b]pyrazin-5-amine hydrochloride
N-[(3-fluoropiperidin-3-yl)methyl]-7-[6-(2,2,2-trifluoroethoxy)pyridin-3-yl]pyrido[3,4-b]pyrazin-5-amine
N-[(3-fluoropiperidin-3-yl)methyl]-7-(pyridin-4-yl)pyrido[3,4-b]pyrazin-5-amine hydrochloride
3-({[7-(1-ethyl-1H-pyrazol-4-yl)pyrido[3,4-b]pyrazin-5-yl]oxy}methyl)-3-fluoropiperidine hydrochloride
7-(1,3-benzothiazol-5-yl)-N-[(3-fluoropiperidin-3-yl)methyl]pyrido[3,4-b]pyrazin-5-amine hydrochloride
7-(1-methyl-1H-pyrazol-4-yl)-N-[(6-methylpiperidin-3-yl)methyl]pyrido[3,4-b]pyrazin-5-amine hydrochloride, single unknown enantiomer
N-[(3-fluoropiperidin-3-yl)methyl]-7-(2-methyl-1,3-benzoxazol-6-yl)pyrido[3,4-b]pyrazin-5-amine hydrochloride
7-(1-methyl-1H-pyrazol-4-yl)-N-[(6-methylpiperidin-3-yl)methyl]pyrido[3,4-b]pyrazin-5-amine hydrochloride, single unknown enantiomer
7-(1-methyl-1H-pyrazol-4-yl)-N-[(6-methylpiperidin-3-yl)methyl]pyrido[3,4-b]pyrazin-5-amine hydrochloride, single unknown enantiomer
5-{5-[(3-fluoropiperidin-3-yl)methoxy]pyrido[3,4-b]pyrazin-7-yl}-N,N-dimethylpyrimidin-2-amine hydrochloride
N-[(3-fluoropiperidin-3-yl)methyl]-7-[1-(2,2,2-trifluoroethyl)-1H-pyrazol-4-yl]pyrido[3,4-b]pyrazin-5-amine, single unknown enantiomer
N-[(3-fluoropiperidin-3-yl)methyl]-7-[1-(2,2,2-trifluoroethyl)-1H-pyrazol-4-yl]pyrido[3,4-b]pyrazin-5-amine hydrochloride, single unknown enantiomer
3-fluoro-3-[({7-[1-(propan-2-yl)-1H-pyrazol-4-yl]pyrido[3,4-b]pyrazin-5-yl}oxy)methyl]piperidine hydrochloride
7-(1-ethyl-1H-pyrazol-4-yl)-N-[(3R)-piperidin-3-ylmethyl]pyrido[3,4-b]pyrazin-5-amine hydrochloride
7-(1-ethyl-1H-pyrazol-4-yl)-N-[(3R)-piperidin-3-ylmethyl]pyrido[3,4-b]pyrazin-5-amine
N-[(3R)-piperidin-3-ylmethyl]-7-[1-(propan-2-yl)-1H-pyrazol-4-yl]pyrido[3,4-b]pyrazin-5-amine
7-(1-tert-butyl-1H-pyrazol-4-yl)-N-[(3-fluoropiperidin-3-yl)methyl]pyrido[3,4-b]pyrazin-5-amine hydrochloride
7-(3,4-dimethoxyphenyl)-N-[(3-fluoropiperidin-3-yl)methyl]pyrido[3,4-b]pyrazin-5-amine
(3R)-3-({[7-(1-ethyl-1H-pyrazol-4-yl)pyrido[3,4-b]pyrazin-5-yl]oxy}methyl)piperidine
N-[(3-fluoropiperidin-3-yl)methyl]-7-[5-methyl-1-(propan-2-yl)-1H-pyrazol-4-yl]pyrido[3,4-b]pyrazin-5-amine
N-[(3-fluoropiperidin-3-yl)methyl]-7-[1-(methoxymethyl)-1H-pyrazol-4-yl]pyrido[3,4-b]pyrazin-5-amine
7-(1-tert-butyl-1H-pyrazol-4-yl)-N-{[(3S)-3-fluoropiperidin-3-yl]methyl}pyrido[3,4-b]pyrazin-5-amine
7-(1-tert-butyl-1H-pyrazol-4-yl)-N-{[(3S)-3-fluoropiperidin-3-yl]methyl}pyrido[3,4-b]pyrazin-5-amine hydrochloride
7-(3,4-dimethoxyphenyl)-N-[(3R)-piperidin-3-ylmethyl]pyrido[3,4-b]pyrazin-5-amine
7-(3,4-dimethoxyphenyl)-N-[(3R)-piperidin-3-ylmethyl]pyrido[3,4-b]pyrazin-5-amine hydrochloride
7-(5,6-dimethoxypyridin-3-yl)-N-{[(3S)-3-fluoropiperidin-3-yl]methyl}pyrido[3,4-b]pyrazin-5-amine
7-(2,2-difluoro-2H-1,3-benzodioxol-5-yl)-N-{[(3S)-3-fluoropiperidin-3-yl]methyl}pyrido[3,4-b]pyrazin-5-amine
   or a salt thereof.

In another embodiment, representative compounds of the invention include:
7-[1-(phenylmethyl)-1H-pyrazol-4-yl]-N-[(3S)-3-piperidinylmethyl]pyrido[3,4-b]pyrazin-5-amine;
7-(1-cyclopentyl-1H-pyrazol-4-yl)-N-[(3S)-3-piperidinylmethyl]pyrido[3,4-b]pyrazin-5-amine;
N-{[(3S)-3-fluoro-3-piperidinyl]methyl}-7-[1-(2,2,2-trifluoroethyl)-1H-pyrazol-4-yl]pyrido[3,4-b]pyrazin-5-amine;
7-[3,4-bis(methyloxy)phenyl]-N-{[(3S)-3-fluoro-3-piperidinyl]methyl}pyrido[3,4-b]pyrazin-5-amine;
N-{[(3S)-3-fluoro-3-piperidinyl]methyl}-7-(1-methyl-1H-pyrazol-4-yl)pyrido[3,4-b]pyrazin-5-amine;
5-{[(3S)-3-piperidinylmethyl]oxy}-7-[1-(2,2,2-trifluoroethyl)-1H-pyrazol-4-yl]pyrido[3,4-b]pyrazine;
7-(1,5-dimethyl-1H-pyrazol-4-yl)-5-{[(3S)-3-piperidinylmethyl]oxy}pyrido[3,4-b]pyrazine;
N,N-dimethyl-5-(5-{[(3S)-3-piperidinylmethyl]oxy}pyrido[3,4-b]pyrazin-7-yl)-2-pyridinamine;
7-(1-methyl-1H-pyrazol-4-yl)-5-{[(3S)-3-piperidinylmethyl]oxy}pyrido[3,4-b]pyrazine;
7-(1,5-dimethyl-1H-pyrazol-4-yl)-5-{[(2S)-2-morpholinylmethyl]oxy}pyrido[3,4-b]pyrazine;
7-(1-methyl-1H-pyrazol-4-yl)-N-[(2S)-2-morpholinylmethyl]pyrido[3,4-b]pyrazin-5-amine;
N-{[(2S)-1-methyl-2-piperazinyl]methyl}-7-(1-methyl-1H-pyrazol-4-yl)pyrido[3,4-b]pyrazin-5-amine;
5-{[(4,4-difluoro-3-piperidinyl)methyl]oxy}-7-(1-methyl-1H-pyrazol-4-yl)pyrido[3,4-b]pyrazine;
7-(1-methyl-1H-pyrazol-4-yl)-5-[2-(3-piperidinyl)ethyl]pyrido[3,4-b]pyrazine;
N-{7-[6-(dimethylamino)-3-pyridinyl]pyrido[3,4-b]pyrazin-5-yl}-1,4-butanediamine;
N-[7-(4-methylphenyl)pyrido[3,4-b]pyrazin-5-yl]-1,4-butanediamine;
7-[6-(dimethylamino)-3-pyridinyl]-N-(2,2,2-trifluoroethyl)pyrido[3,4-b]pyrazin-5-amine;
4-({7-[6-(dimethylamino)-3-pyridinyl]pyrido[3,4-b]pyrazin-5-yl}amino)-1-butanol;
N³-{7-[6-(dimethylamino)-3-pyridinyl]pyrido[3,4-b]pyrazin-5-yl}-β-alaninamide;
7-[6-(dimethylamino)-3-pyridinyl]-N-[(3S)-3-piperidinylmethyl]pyrido[3,4-b]pyrazin-5-amine;
N,N-dimethyl-5-{5-[2-(3-piperidinyl)ethyl]pyrido[3,4-b]pyrazin-7-yl}-2-pyridinamine;
7-(1-methyl-1H-pyrazol-4-yl)-N-[(3S)-3-piperidinylmethyl]pyrido[3,4-b]pyrazin-5-amine;
N-[(5,5-difluoro-3-piperidinyl)methyl]-7-(1-methyl-1H-pyrazol-4-yl)pyrido[3,4-b]pyrazin-5-amine; and
7-[6-(dimethylamino)-3-pyridinyl]-N-[(3R)-3-piperidinylmethyl]pyrido[3,4-b]pyrazin-5-amine;
   or a salt thereof.

In another embodiment, representative compounds of the invention include:
7-[1-(phenylmethyl)-1H-pyrazol-4-yl]-N-[(3S)-3-piperidinylmethyl]pyrido[3,4-b]pyrazin-5-amine;
7-(1-cyclopentyl-1H-pyrazol-4-yl)-N-[(3S)-3-piperidinylmethyl]pyrido[3,4-b]pyrazin-5-amine;
N-{[(3S)-3-fluoro-3-piperidinyl]methyl}-7-[1-(2,2,2-trifluoroethyl)-1H-pyrazol-4-yl]pyrido[3,4-b]pyrazin-5-amine;
7-[3,4-bis(methyloxy)phenyl]-N-{[(3S)-3-fluoro-3-piperidinyl]methyl}pyrido[3,4-b]pyrazin-5-amine hydrochloride;
N-{[(3S)-3-fluoro-3-piperidinyl]methyl}-7-(1-methyl-1H-pyrazol-4-yl)pyrido[3,4-b]pyrazin-5-amine hydrochloride;
5-{[(3S)-3-piperidinylmethyl]oxy}-7-[1-(2,2,2-trifluoroethyl)-1H-pyrazol-4-yl]pyrido[3,4-b]pyrazine hydrochloride;
7-(1,5-dimethyl-1H-pyrazol-4-yl)-5-{[(3S)-3-piperidinylmethyl]oxy}pyrido[3,4-b]pyrazine, hydrochloride;
N,N-dimethyl-5-(5-{[(3S)-3-piperidinylmethyl]oxy}pyrido[3,4-b]pyrazin-7-yl)-2-pyridinamine hydrochloride;
7-(1-methyl-1H-pyrazol-4-yl)-5-{[(3S)-3-piperidinylmethyl]oxy}pyrido[3,4-b]pyrazine hydrochloride;
7-(1,5-dimethyl-1H-pyrazol-4-yl)-5-{[(2S)-2-morpholinylmethyl]oxy}pyrido[3,4-b]pyrazine, hydrochloride;
7-(1-methyl-1H-pyrazol-4-yl)-N-[(2S)-2-morpholinylmethyl]pyrido[3,4-b]pyrazin-5-amine, hydrochloride;
N-{[(2S)-1-methyl-2-piperazinyl]methyl}-7-(1-methyl-1H-pyrazol-4-yl)pyrido[3,4-b]pyrazin-5-amine hydrochloride;
5-{[(4,4-difluoro-3-piperidinyl)methyl]oxy}-7-(1-methyl-1H-pyrazol-4-yl)pyrido[3,4-b]pyrazine;
7-(1-methyl-1H-pyrazol-4-yl)-5-[2-(3-piperidinyl)ethyl]pyrido[3,4-b]pyrazine hydrochloride; and
N-{7-[6-(dimethylamino)-3-pyridinyl]pyrido[3,4-b]pyrazin-5-yl}-1,4-butanediamine.

In another embodiment, representative compounds of the invention include:
7-(1-cyclopentyl-1H-pyrazol-4-yl)-N-[(3S)-3-piperidinylmethyl]pyrido[3,4-b]pyrazin-5-amine;
5-{[(3S)-3-piperidinylmethyl]oxy}-7-[1-(2,2,2-trifluoroethyl)-1H-pyrazol-4-yl]pyrido[3,4-b]pyrazine;
N,N-dimethyl-5-(5-{[(3S)-3-piperidinylmethyl]oxy}pyrido[3,4-b]pyrazin-7-yl)-2-pyridinamine; and
5-{[(4,4-difluoro-3-piperidinyl)methyl]oxy}-7-(1-methyl-1H-pyrazol-4-yl)pyrido[3,4-b]pyrazine;
   or a salt thereof.

It will be appreciated that compounds of formula (I) and salts thereof may exist in solvated forms. In another embodiment, the present invention provides compounds of formula (I) and salts thereof. In another embodiment, the present invention provides compounds of formula (I) and pharmaceutically acceptable salts thereof. In another embodiment, the present invention provides compounds of formula (I) and solvates thereof. In a further embodiment, the present invention provides compounds of formula (I) as the free base.

Compounds of formula (I) are useful as inhibitors of Syk.

As used herein, the term “alkyl” refers to a straight or branched saturated hydrocarbon chain containing the specified number of carbon atoms. For example, C₁₋₆alkyl means a straight or branched alkyl group containing at least 1, and at most 6, carbon atoms. Examples of “alkyl” as used herein include, but are not limited to, methyl, ethyl, n-propyl, n-butyl, n-pentyl, n-hexyl, isobutyl, isopropyl, t-butyl and 1,1-dimethylpropyl.

As used herein, the term “alkoxy” refers to a straight or branched saturated alkoxy chain containing the specified number of carbon atoms. For example, C₁₋₆alkoxy means a straight or branched alkoxy group containing at least 1, and at most 6, carbon atoms. Examples of “alkoxy” as used herein include, but are not limited to, methoxy, ethoxy, propoxy, prop-2-oxy, butoxy, but-2-oxy, 2-methylprop-1-oxy, 2-methylprop-2-oxy, pentoxy or hexyloxy.

As used herein the term “cycloalkyl” refers to carbocyclic rings having from three to seven ring carbon atoms, for example from three to six ring carbon atoms. Examples of “cycloalkyl” as used herein include, but are not limited to, cyclopropyl, cyclobutyl, cyclopentyl, cyclohexyl and cycloheptyl. In one embodiment the cycloalkyl ring comprises five or six ring carbon atoms.

As used herein the term “halo” or, alternatively, “halogen” refers to fluoro, chloro or bromo.

As used herein the term “haloalkyl” refers to an alkyl group substituted with one to three halo groups or with combinations thereof. Examples of “haloalkyl” as used herein include, but are not limited to, 1,1,1-trifluoroethyl, 1,1-difluoroethyl and fluoroethyl.

As used herein the term “fluoroalkyl” refers to a haloalkyl group wherein the one to three halo groups are fluorine. Examples of “fluoroalkyl” as used herein include, but are not limited to, 1,1,1-trifluoroethyl, 1,1-difluoroethyl and fluoroethyl.

As used herein the term “heterocyclyl” refers to saturated heterocyclic rings containing 5 or 6 ring-atoms up to 2 of which may be hetero-atoms such as nitrogen, oxygen and sulfur. Examples of “heterocyclyl” as used herein include, but are not limited to, pyrrolidine, tetrahydrofuran, tetrahydrothiophene, pyrazolidine, imidazolidine, 3-dioxolane, thiazolidine, isoxazolidine, piperidine, piperazine, morpholine, 1,4-dioxane, thiomorpholine and 1,4-oxathiane.

As used herein the term “heteroaryl” refers to unsaturated, aromatic, heterocyclic rings containing 5 or 6 ring-atoms up to 2 of which may be hetero-atoms such as nitrogen, oxygen and sulfur. Examples of heteroaryl groups include pyrrole, furan, thiophene, pyrazole, imidazole, oxazole, isoxazole, thiazole, isothiazole, pyridine, pyran, pyridazine, pyrimidine, pyrazine, oxazine and dioxine.

As used herein the term “fused heteroaryl” refers to unsaturated, aromatic, heterocyclic rings containing 9 or 10 ring-atoms up to 3 of which may be hetero-atoms such as nitrogen, oxygen and sulfur. Examples of fused heteroaryl groups include indole, benzofuran, benzothiophene, isoindole, isobenzofuran, isobenzothiophene, indazole, benzimidazole, benzthiazole, pyrrolopyridine, quinoline and isoquinoline.

As used herein, the term “pharmaceutically acceptable” refers to those compounds, materials, compositions, and dosage forms which are, within the scope of sound medical judgment, suitable for use in contact with the tissues of human beings and animals without excessive toxicity, irritation, or other problems or complications, commensurate with a reasonable benefit/risk ratio. The skilled artisan will appreciate that pharmaceutically acceptable salts of the compound of the present invention may be prepared.

As used herein, the term “pharmaceutically acceptable salts” refers to salts that retain the desired biological activity of the subject compound and exhibit minimal undesired toxicological effects. These pharmaceutically acceptable salts may be prepared in situ during the final isolation and purification of the compound, or by separately reacting the purified compound in its free acid or free base form with a suitable base or acid, respectively. Indeed, in certain embodiments of the invention, pharmaceutically acceptable salts may be preferred over the respective free base or free acid because such salts impart greater stability or solubility to the molecule thereby facilitating formulation into a dosage form. In one embodiment the pharmaceutically acceptable salt is the hydrochloride salt.

The compounds of formula (I) are basic and accordingly generally capable of forming pharmaceutically acceptable acid addition salts by treatment with a suitable acid. Suitable acids include pharmaceutically acceptable inorganic acids and pharmaceutically acceptable organic acids. Representative pharmaceutically acceptable acid addition salts include hydrochloride, hydrobromide, nitrate, methylnitrate, sulfate, bisulfate, sulfamate, phosphate, acetate, hydroxyacetate, phenylacetate, propionate, butyrate, isobutyrate, valerate, maleate, hydroxymaleate, acrylate, fumarate, malate, tartrate, citrate, salicylate, p-aminosalicyclate, glycollate, lactate, heptanoate, phthalate, oxalate, succinate, benzoate, o-acetoxybenzoate, chlorobenzoate, methylbenzoate, dinitrobenzoate, hydroxybenzoate, methoxybenzoate, mandelate, tannate, formate, stearate, ascorbate, palmitate, oleate, pyruvate, pamoate, malonate, laurate, glutarate, glutamate, estolate, methanesulfonate (mesylate), ethanesulfonate (esylate), 2-hydroxyethanesulfonate, benzenesulfonate (besylate), p-aminobenzenesulfonate, p-toluenesulfonate (tosylate), and napthalene-2-sulfonate. In one embodiment, the present invention provides a pharmaceutically acceptable salt of a compound of formula (I) which is the hydrochloride salt.

The compounds of formula (I) may contain a chiral centre in the R₁ position and, therefore, may exist as individual enantiomers, or as mixtures thereof. Where the stereochemistry of the chiral centre is not specified the structure is intended to encompass each enantiomer and all mixtures thereof. Thus, the compounds of formula (I) may be used as racemic mixtures, enantiomerically enriched mixtures, or as enantiomerically pure individual stereoisomers. The present invention includes all such mixtures as well as pure individual enantiomers. Generally it is preferred to use a compound of formula (I) in the form of a purified single enantiomer. It will be appreciated by those skilled in the art that at least one enantiomer of the racemate has the described activity. The other enantiomer may have similar activity, less activity, no activity or may have some antagonist activity in a functional assay.

A mixture of enantiomers, such as a racemic mixture, may be preferred. Thus, in one embodiment of the invention the compound of formula (I) is the racemic mixture (the racemate).

Alternatively, a single enantiomer may be preferred, for example the S-enantiomer. Thus, in one embodiment of the invention the compound of formula (I) is the S-enantiomer. In a further embodiment of the invention the compound of formula (I) is the R-enantiomer.

The individual enantiomers of a compound of formula (I) may be resolved by methods known to those skilled in the art. For example, such resolution may be carried out (1) by formation of diastereoisomeric salts, complexes or other derivatives; (2) by selective reaction with a stereoisomer-specific reagent, for example by enzymatic oxidation or reduction; or (3) by gas-liquid or liquid chromatography in a chiral environment, for example, on a chiral support such as silica with a bound chiral ligand or in the presence of a chiral solvent. The skilled person will appreciate that where the desired stereoisomer is converted into another chemical entity by one of the separation procedures described above, a further step is required to liberate the desired form. Alternatively, specific enantiomers may be synthesized by asymmetric synthesis using optically active reagents, substrates, catalysts or solvents, or by converting one enantiomer to the other by asymmetric transformation.

A compound of the present invention may exist in solid or liquid form. In the solid state, the compound of the present invention may exist in crystalline or non-crystalline (amorphous) form, or as a mixture thereof. For a compound of the present invention that is in crystalline form, the skilled artisan will appreciate that pharmaceutically acceptable solvates may be formed wherein solvent molecules are incorporated into the crystalline lattice during crystallization. Solvates may involve non-aqueous solvents such as, but not limited to, ethanol, isopropanol, n-butanol, i-butanol, acetone, tetrahydrofuran, dioxane, DMSO, acetic acid, ethanolamine, and ethyl acetate, or they may involve water as the solvent that is incorporated into the crystalline lattice. Solvates wherein water is the solvent incorporated into the crystalline lattice are typically referred to as “hydrates”. Hydrates include stoichiometric hydrates as well as compositions containing variable amounts of water.

The skilled artisan will further appreciate that a compound of the present invention that exists in crystalline form, including the various solvates thereof, may exhibit polymorphism (i.e. the capacity to occur in different crystalline structures). These different crystalline forms are typically known as “polymorphs.” The invention includes all such polymorphs. Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties. Polymorphs typically exhibit different melting points, IR spectra, and X-ray powder diffraction patterns, which may be used for identification. The skilled artisan will appreciate that different polymorphs may be produced, for example, by changing or adjusting the reaction conditions or reagents, used in making the compound. For example, changes in temperature, pressure, or solvent may result in polymorphs. In addition, one polymorph may spontaneously convert to another polymorph under certain conditions.

A compound of formula (I) may be prepared by the general synthetic schemes described hereinafter.

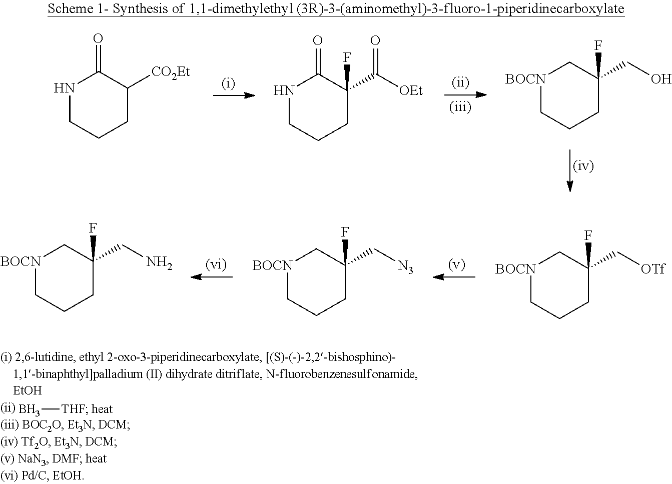

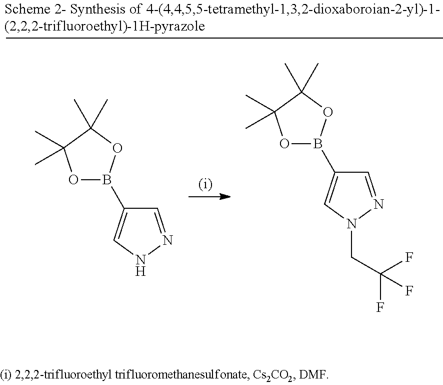 [SMILES: CC1(C)OB(c2cn[nH]c2)OC1(C)C ; CC1(C)OB(c2cnn(CC(F)(F)F)c2)OC1(C)C]

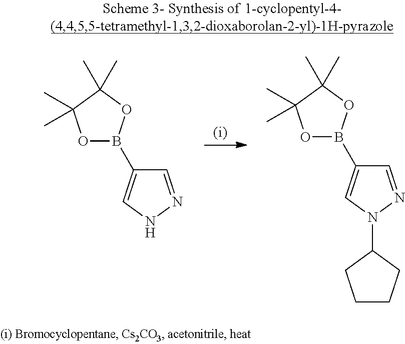 [SMILES: CC1(C)OB(c2cn[nH]c2)OC1(C)C ; CC1(C)OB(c2cnn(C3CCCC3)c2)OC1(C)C]

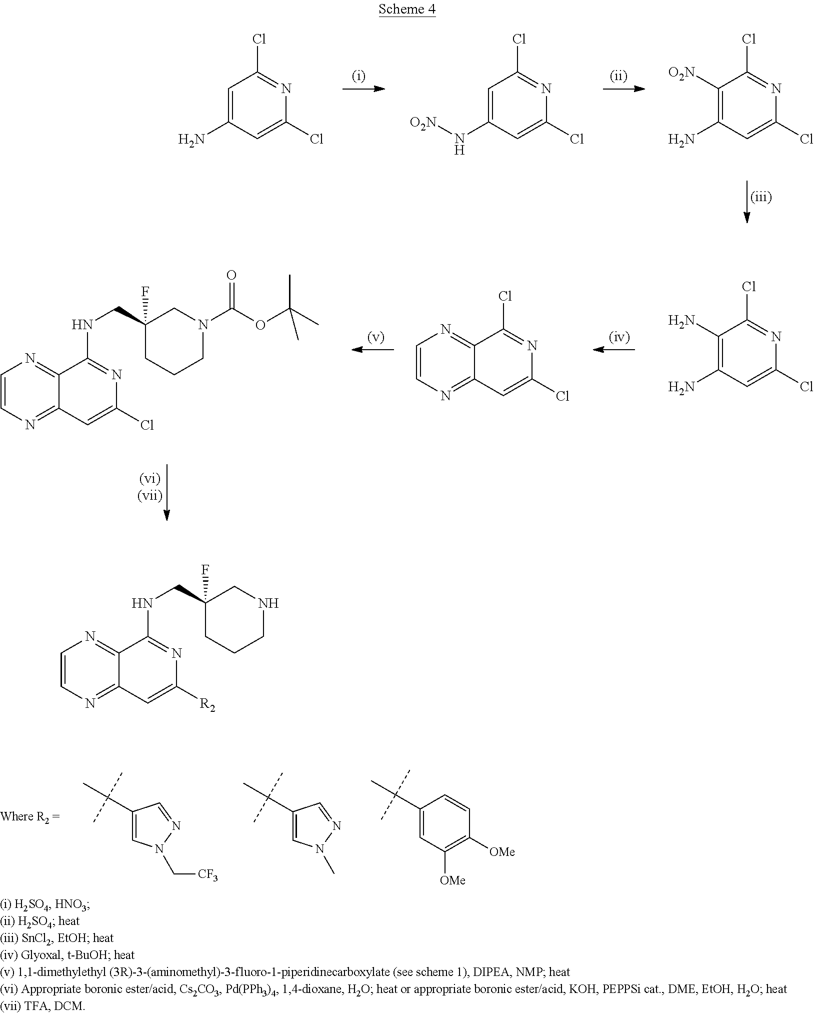

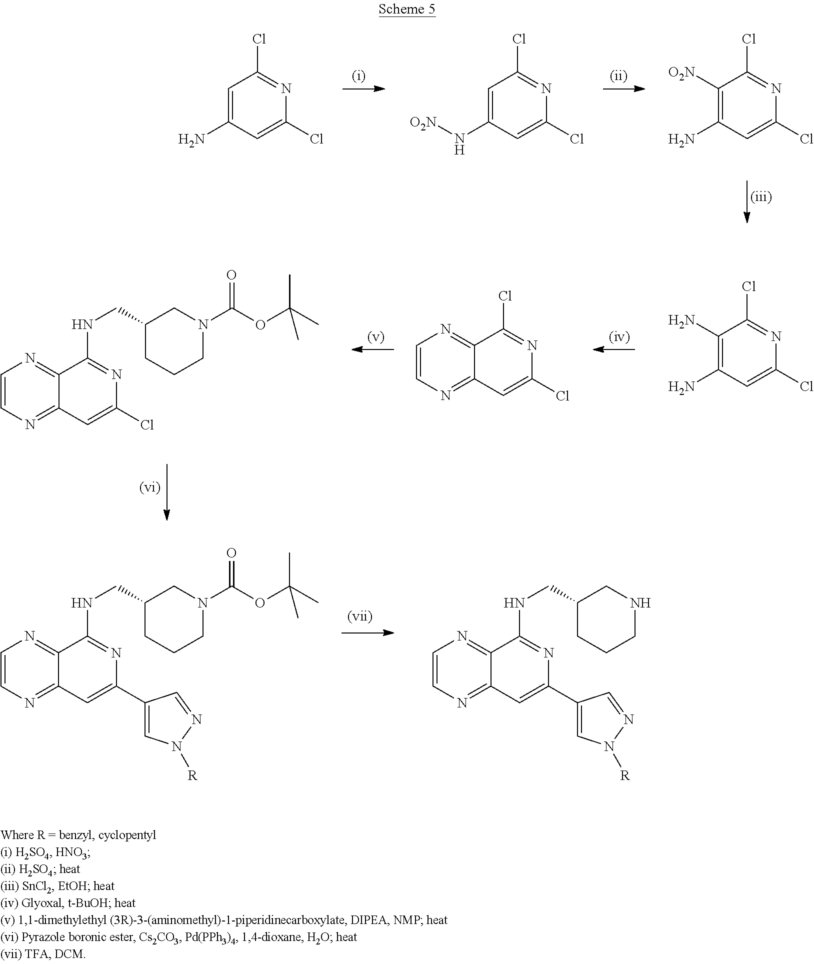

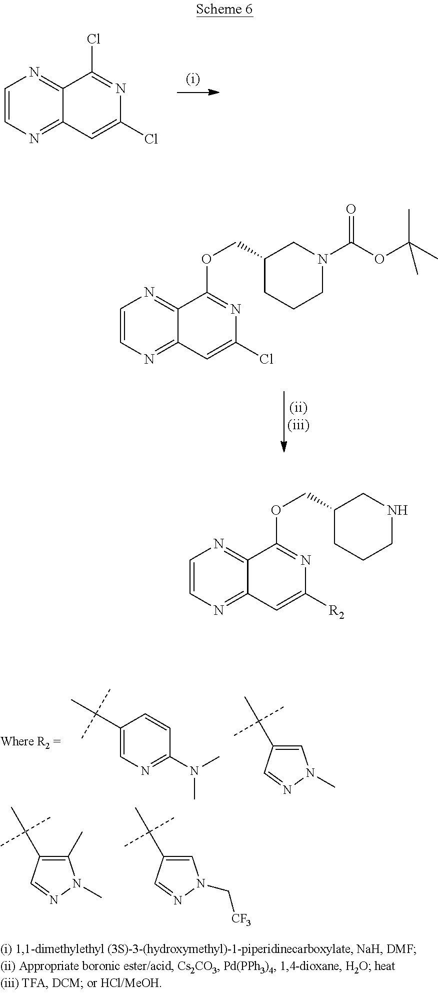

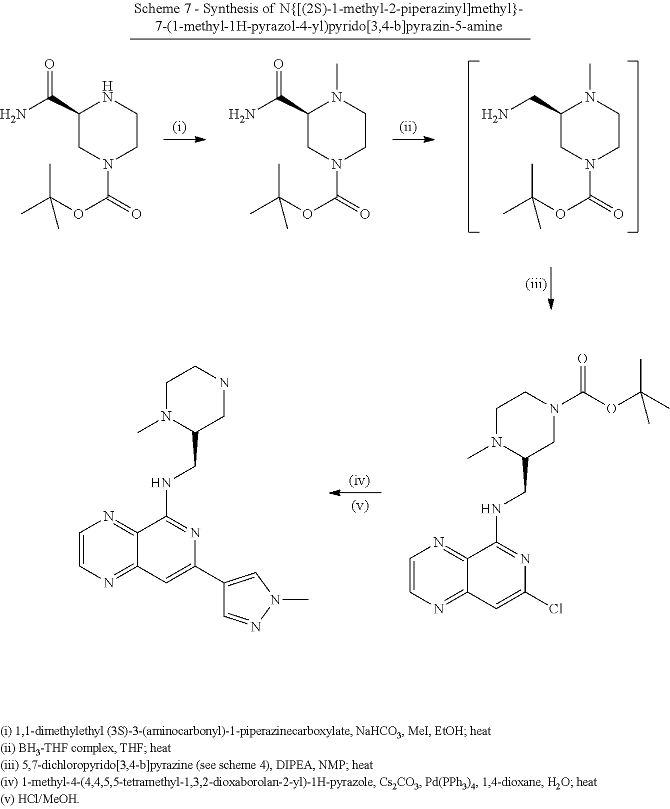

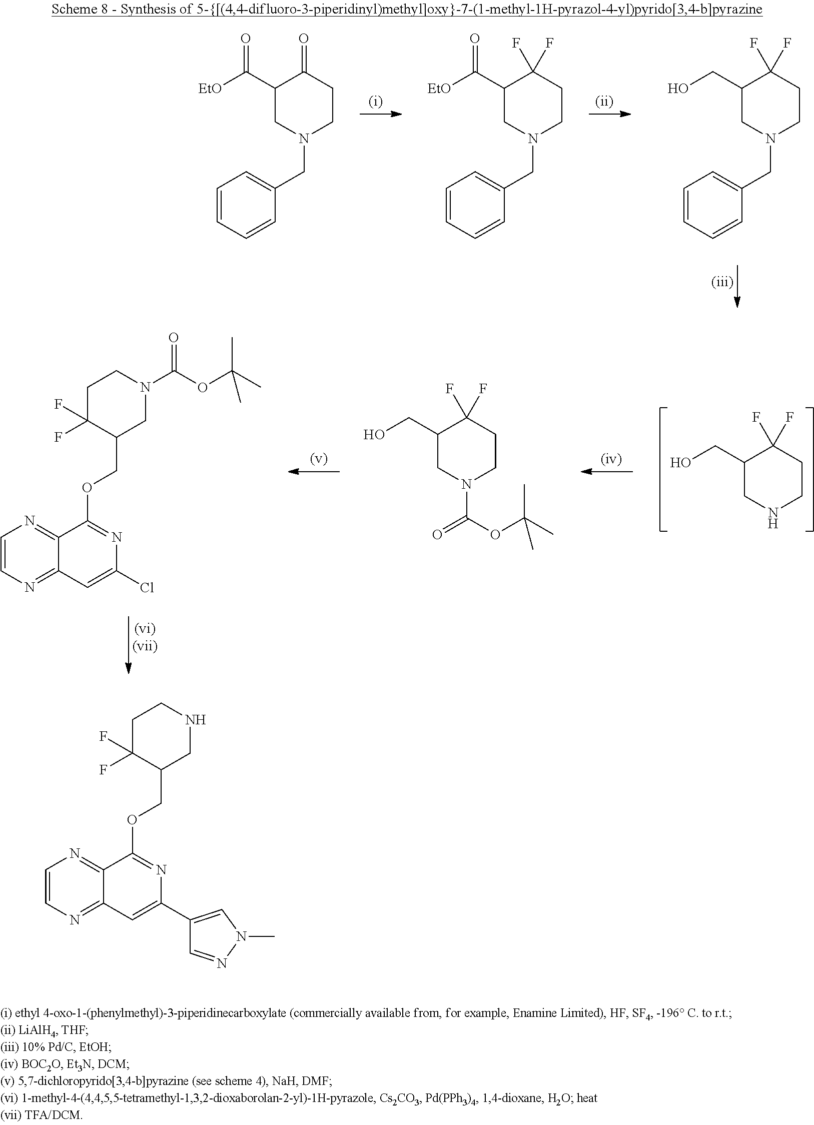

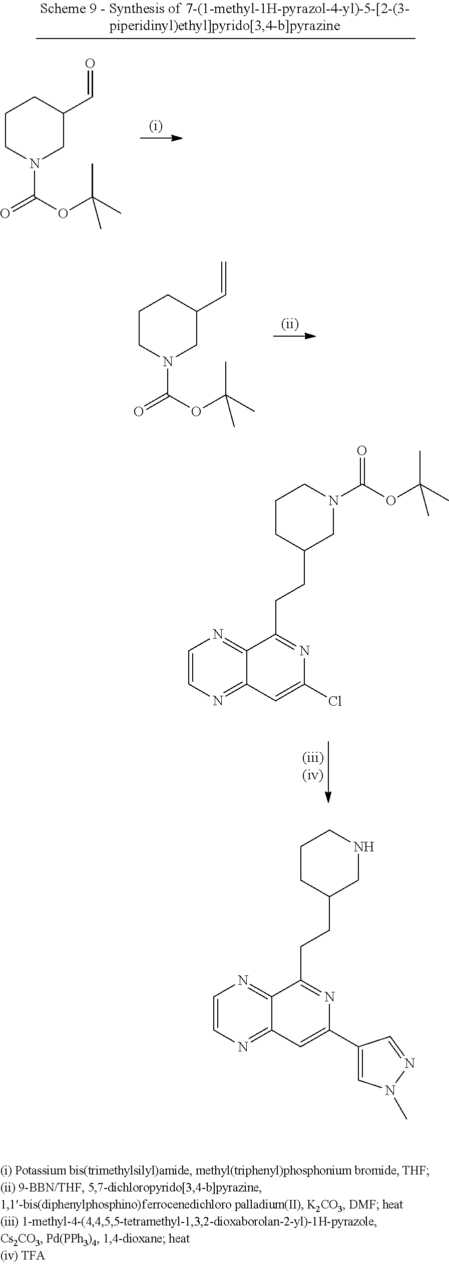 [SMILES: CC(C)(C)OC(=O)N1CCCC(C=O)C1 ; C=CC1CCCN(C(=O)OC(C)(C)C)C1 ; CC(C)(C)OC(=O)N1CCCC(CCc2nc(Cl)cc3nccnc23)C1 ; Cn1cc(-c2cc3nccnc3c(CCC3CCCNC3)n2)cn1]

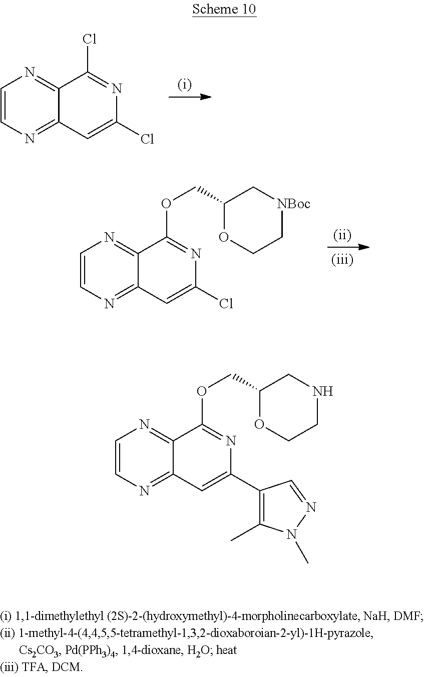 [SMILES: Clc1cc2nccnc2c(Cl)n1 ; CC(C)(C)OC(=O)N1CCO[C@H](COc2nc(Cl)cc3nccnc23)C1 ; Cc1c(-c2cc3nccnc3c(OC[C@@H]3CNCCO3)n2)cnn1C]

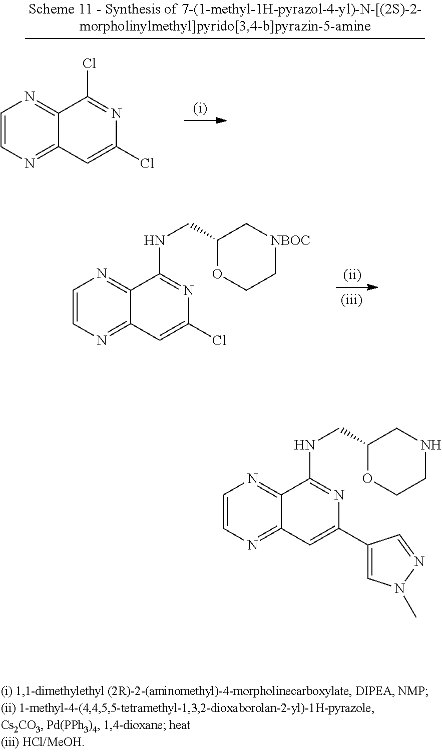

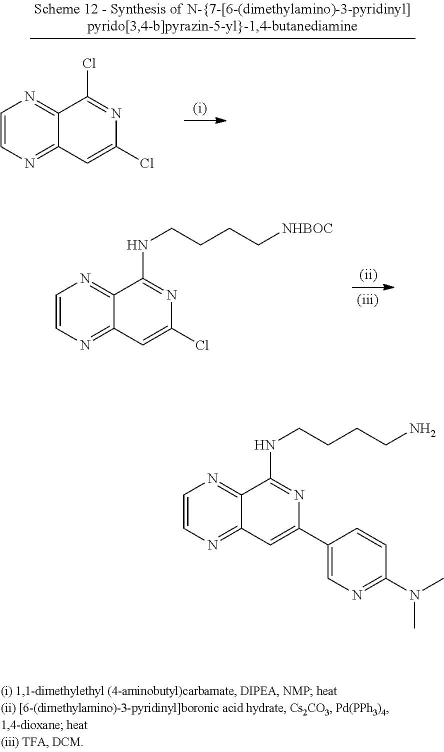

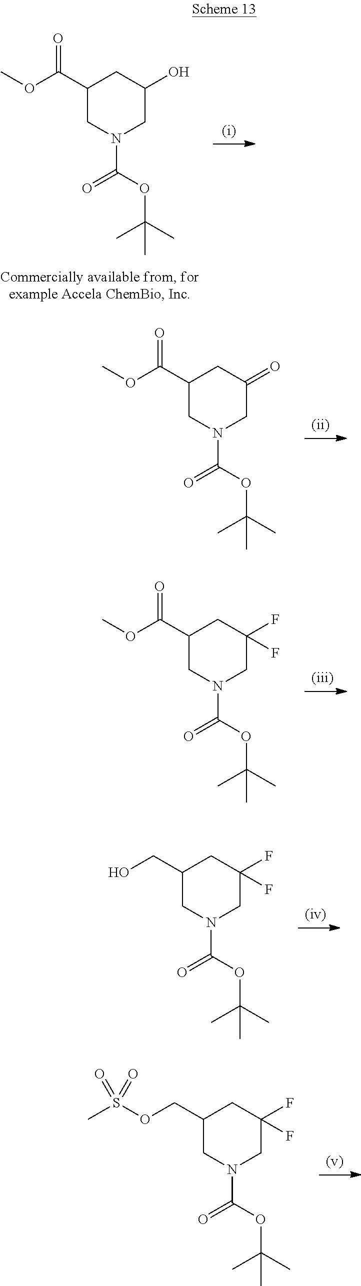 [SMILES: COC(=O)C1CC(O)CN(C(=O)OC(C)(C)C)C1 ; COC(=O)C1CC(=O)CN(C(=O)OC(C)(C)C)C1 ; COC(=O)C1CN(C(=O)OC(C)(C)C)CC(F)(F)C1 ; CC(C)(C)OC(=O)N1CC(CO)CC(F)(F)C1 ; CC(C)(C)OC(=O)N1CC(COS(C)(=O)=O)CC(F)(F)C1]

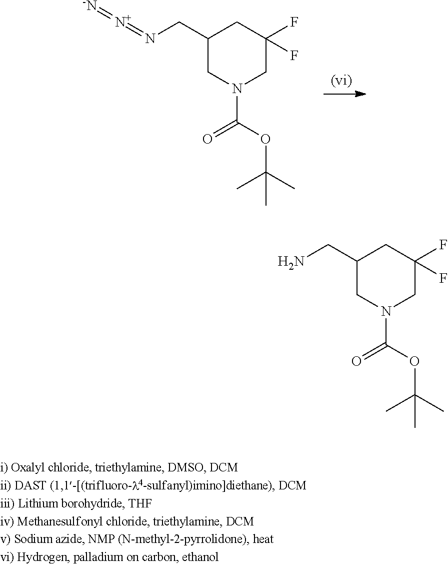

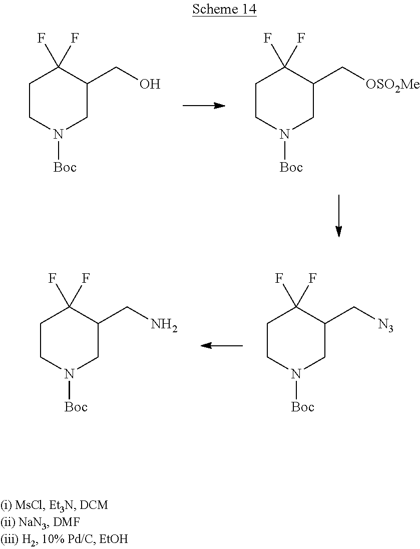

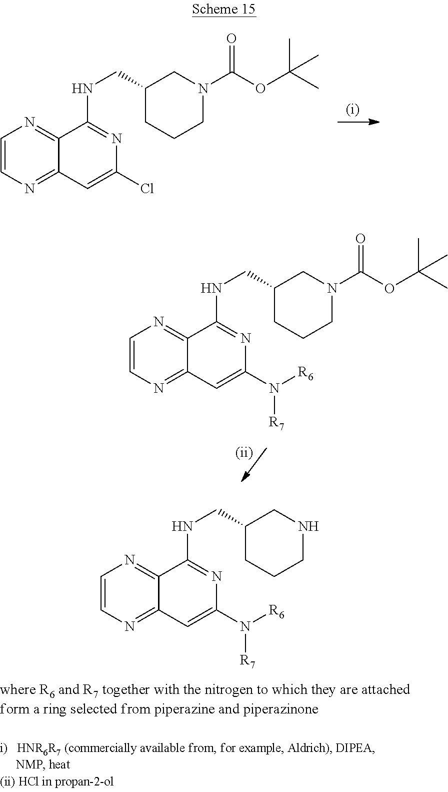

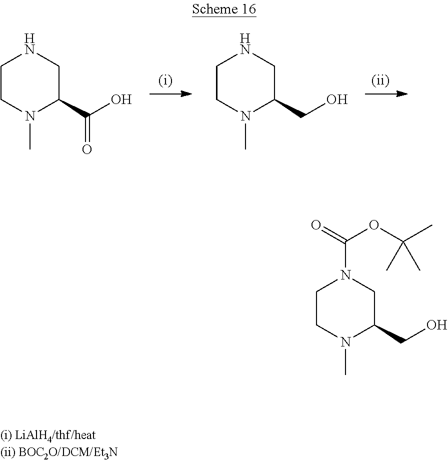

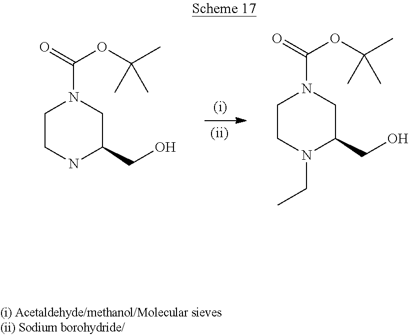 [SMILES: CC(C)(C)OC(=O)N1CCN[C@H](CO)C1 ; CCN1CCN(C(=O)OC(C)(C)C)C[C@H]1CO]

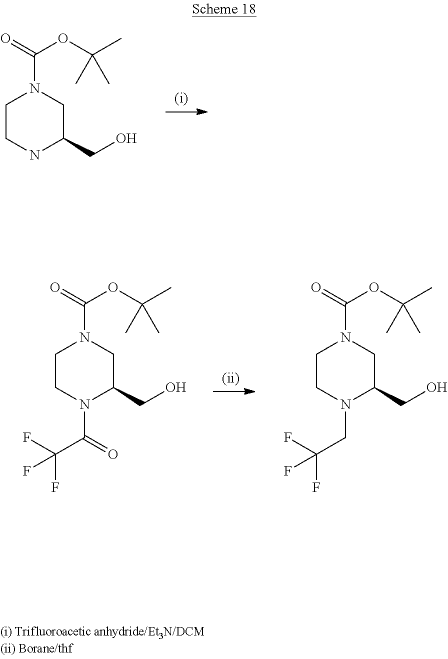 [SMILES: CC(C)(C)OC(=O)N1CCN[C@H](CO)C1 ; CC(C)(C)OC(=O)N1CCN(C(=O)C(F)(F)F)[C@H](CO)C1 ; CC(C)(C)OC(=O)N1CCN(CC(F)(F)F)[C@H](CO)C1]

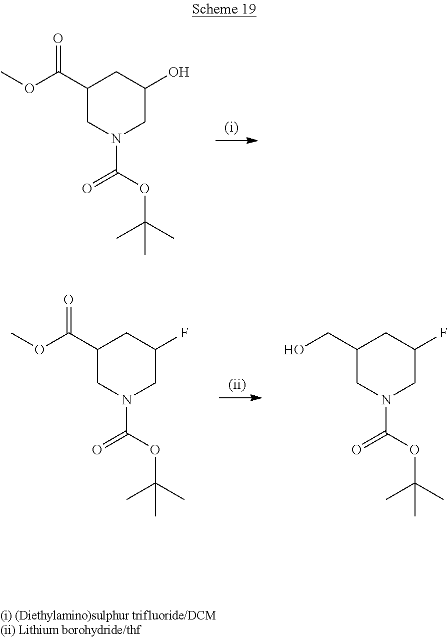 [SMILES: COC(=O)C1CC(O)CN(C(=O)OC(C)(C)C)C1 ; COC(=O)C1CC(F)CN(C(=O)OC(C)(C)C)C1 ; CC(C)(C)OC(=O)N1CC(F)CC(CO)C1]

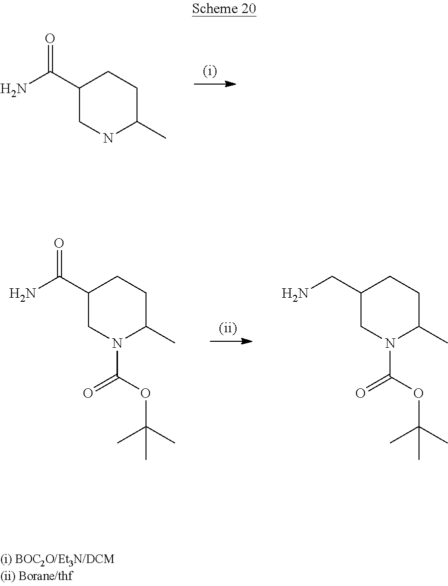

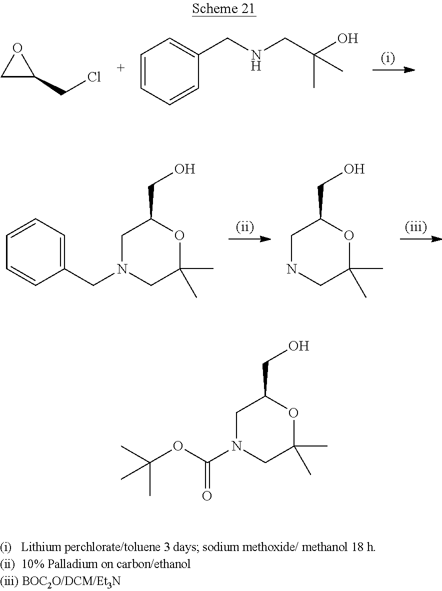

Thus, in a further aspect, the present invention provides a process for preparing a compound of formula (I) which process comprises reacting a pyrido[3,4-b]pyrazine compound of formula (II):

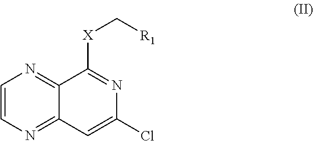

wherein X and R₁ are as hereinbefore defined;
and any nitrogen atoms in the R₁ substituent are protected from substitution by a protecting group;
with a pyrazole boronic ester or acid of formula (III):

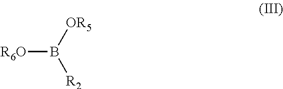

wherein R₅ and R₆ which may be the same or different are each hydrogen, C₁₋₆alkyl or R₅ and R₆ may be joined to form a C₁₋₃alkylene group optionally substituted by up to four methyl groups, for instance —C(Me)₂C(Me)₂-; and
R₂ is as hereinbefore defined;
in the presence of a catalyst, under conditions typically used for a boronic ester/acid coupling; and
thereafter, removing any protecting group.

Conditions typically used for a boronic ester/acid coupling includes the use of the Pd(PPh₃)⁴ as catalyst, with caesium carbonate in a solvent such as aqueous 1,4-dioxane. Alternatively conditions that could be used include the use of PEPPSI™ as catalyst, with potassium hydroxide in a solvent such as aqueous dimethoxyethane (DME) with ethanol.

Examples of protecting groups and the means for their removal can be found in T. W. Greene ‘Protective Groups in Organic Synthesis’ (J. Wiley and Sons, 1991). Suitable amine protecting groups include, but are not restricted to, sulphonyl (such as tosyl), acyl (such as benzyloxycarbonyl or t-butoxycarbonyl) and arylalkyl (such as benzyl), which may be removed by hydrolysis or hydrogenolysis as appropriate. Other suitable amine protecting groups include trifluoroacetyl (—C(O)CF₃), which may be removed by base catalysed hydrolysis, or a solid phase resin bound benzyl group, such as a Merrifield resin bound 2,6-dimethoxybenzyl group (Ellman linker) which may be removed by acid catalysed hydrolysis (using, for example, trifluoroacetic acid).

In one embodiment of the present invention the protecting group (P) is selected from tert-butyloxycarbonyl “BOC” and 9-fluorenylmethyloxycarbonyl “FmoC”.

Compounds of formula (I) are useful as inhibitors of Syk and thus potentially of use in treating some cancer therapies, in particular heme malignancies, as well as inflammatory conditions which involve B cells, and also diseases resulting from inappropriate mast cell activation, for instance allergic and inflammatory diseases such as cutaneous mast cell mediated diseases including acute and chronic urticaria, mastocytosis, atopic dermatitis and autoimmune diseases such as cutaneous lupus and autoimmune bullous conditions including pemphigus and pemphigoid.

In one aspect, the present invention provides a compound of formula (I) or a pharmaceutically acceptable salt thereof, for use in therapy.

In another aspect, the present invention provides a compound of formula (I) or a pharmaceutically acceptable salt thereof, for use in inhibiting spleen tyrosine kinase (Syk).

In a further aspect, the present invention provides a method comprising administering to a patient in need thereof an effective amount of a compound of formula (I) or a pharmaceutically acceptable salt thereof, to inhibit spleen tyrosine kinase (Syk).

Syk inhibitors may be useful in cancer therapy, specifically heme malignancies, particularly Non-Hodgkin's Lymphomas including follicular (FL), mantle cell, small lymphocytic lymphoma/chronic lymphocytic lymphoma (SLL/CLL), Burkitt and diffuse large B cell (DLBCL) lymphomas.

In one aspect, the present invention provides a compound of formula (I) or a pharmaceutically acceptable salt thereof, for use in the treatment of cancer, for example heme malignancies, particularly Non-Hodgkin's lymphomas including follicular (FL), mantle cell, small lymphocytic lymphoma/chronic lymphocytic lymphoma (SLL/CLL), Burkitt and diffuse large B cell (DLBCL) lymphomas.

In another aspect, the present invention provides a method of treating cancer, for example Acute myeloid leukaemia, retinoblastoma, heme malignancies, particularly Non-Hodgkin's Lymphomas including follicular (FL), mantle cell, small lymphocytic lymphoma/chronic lymphocytic lymphoma (SLL/CLL), Burkitt and diffuse large B cell (DLBCL) lymphomas, which method comprises administering to a patient in need thereof a therapeutically effective amount of a compound of formula (I) or a pharmaceutically acceptable salt thereof.

In a further aspect, the present invention provides the use of a compound of formula (I) or a pharmaceutically acceptable salt thereof, for the manufacture of a medicament for the treatment of cancer, for example, Acute myeloid leukaemia, retinoblastoma, heme malignancies, particularly Non-Hodgkin's lymphomas including follicular (FL), mantle cell, small lymphocytic lymphoma/chronic lymphocytic lymphoma (SLL/CLL), Burkitt and diffuse large B cell (DLBCL) lymphomas.

Compounds of formula (I) may also be used in cancer chemotherapy in combination with other classes of cancer chemotherapy agents which are known in the art.

Representative classes of agents for use in such combinations for Non-Hodgkin's Lymphomas include rituximab, BEXXAR (tositumomab and Iodine I 131 tositumomab) and pixantrone. Compounds of formula (I) may also be used in combination with the CHOP drug regime (cyclophosphamide, adriamycin, vincristine, prednisone) or CHOP plus rituximab (CHOP+R).

Compounds of formula (I) are potentially of use in treating autoimmune conditions which involve B cells and/or macrophage activation, for example systemic lupus erythematosus (SLE), discoid (cutaneous) lupus, Sjorgens syndrome, Wegners granulomatosis and other vasculitides, bullous pemphigoid and pemphigus, idiopathic thrombocytopenic purpura (ITP), giant cell arteriosis, chronic idiopathic urticaria with and without auto-antibody status (chronic autoimmune urticaria (New concepts in chronic urticaria, Current Opinions in Immunology 2008 20:709-716)), glomerulonephritis, chronic transplant rejection, and rheumatoid arthritis.

In one aspect, the present invention provides a compound of formula (I) or a pharmaceutically acceptable salt thereof, for use in the treatment of an autoimmune condition, for example systemic lupus erythematosus (SLE), discoid (cutaneous) lupus, Sjorgens syndrome, Wegners granulomatosis and other vasculitides, bullous pemphigoid and pemphigus, idiopathic thrombocytopenic purpura (ITP), giant cell arteriosis, chronic idiopathic urticaria with and without auto-antibody status (chronic autoimmune urticaria (New concepts in chronic urticaria, Current Opinions in Immunology 2008 20:709-716)), glomerulonephritis, chronic transplant rejection, and rheumatoid arthritis. In one embodiment, the present invention provides a compound of formula (I) or a pharmaceutically acceptable salt thereof for use in the treatment of an autoimmune condition which is chronic idiopathic urticaria with and without auto-antibody status. In another embodiment, the present invention provides a compound of formula (I) or a pharmaceutically acceptable salt thereof for use in the treatment of an autoimmune condition which is discoid (cutaneous) lupus.

In another aspect, the present invention provides a method of treating an autoimmune condition, for example systemic lupus erythematosus (SLE), discoid (cutaneous) lupus, Sjorgens syndrome, Wegners granulomatosis and other vasculitides, bullous pemphigoid and pemphigus, idiopathic thrombocytopenic purpura (ITP), giant cell arteriosis, chronic idiopathic urticaria with and without auto-antibody status, glomerulonephritis, chronic transplant rejection and rheumatoid arthritis, which method comprises administering to a patient in need thereof a therapeutically effective amount of a compound of formula (I) or a pharmaceutically acceptable salt thereof. In one embodiment, the present invention provides a method of treating an autoimmune disease which is chronic idiopathic urticaria with and without auto-antibody status, which method comprises administering to a patient in need thereof a therapeutically effective amount of a compound of formula (I) or a pharmaceutically acceptable salt thereof. In another embodiment, the present invention provides a method of treating an autoimmune disease which is discoid (cutaneous) lupus, which method comprises administering to a patient in need thereof a therapeutically effective amount of a compound of formula (I) or a pharmaceutically acceptable salt thereof.

In a further aspect, the present invention provides the use of a compound of formula (I) or a pharmaceutically acceptable salt thereof, for the manufacture of a medicament for the treatment of an autoimmune condition, for example systemic lupus erythematosus (SLE), discoid (cutaneous) lupus, Sjorgens syndrome, Wegners granulomatosis and other vasculitides, bullous pemphigoid and pemphigus, idiopathic thrombocytopenic purpura (ITP), giant cell arteriosis, chronic idiopathic urticaria with and without auto-antibody status, glomerulonephritis, chronic transplant rejection and rheumatoid arthritis. In one embodiment, the present invention provides the use of a compound of formula (I) or a pharmaceutically acceptable salt thereof for the manufacture of a medicament for the treatment of an autoimmune condition which is chronic idiopathic urticaria with and without auto-antibody status. In another embodiment, the present invention provides the use of a compound of formula (I) or a pharmaceutically acceptable salt thereof for the manufacture of a medicament for the treatment of an autoimmune condition which is discoid (cutaneous) lupus.

In one aspect, the present invention provides a compound of formula (I) or a pharmaceutically acceptable salt thereof, for use in the treatment of an inflammatory disease which involves B cells.

In another aspect, the present invention provides a method of treating an inflammatory disease which involves B cells which method comprises administering to a patient in need thereof a therapeutically effective amount of a compound of formula (I) or a pharmaceutically acceptable salt thereof.

In a further aspect, the present invention provides the use of a compound of formula (I) or a pharmaceutically acceptable salt thereof, for the manufacture of a medicament for the treatment of an inflammatory disease which involves B cells.

Compounds of formula (I) are potentially of use in treating diseases resulting from inappropriate mast cell activation, for instance allergic and inflammatory diseases.

In one aspect, the present invention provides a compound of formula (I) or a pharmaceutically acceptable salt thereof, for use in the treatment of a disease associated with inappropriate mast cell activation including those diseases with skin manifestations

In another aspect, the present invention provides a method of treating a disease associated with inappropriate mast cell activation which method comprises administering to a patient in need thereof a therapeutically effective amount of a compound of formula (I) or a pharmaceutically acceptable salt thereof.

In a further aspect, the present invention provides the use of a compound of formula (I) or a pharmaceutically acceptable salt thereof, for the manufacture of a medicament for the treatment of a disease associated with inappropriate mast cell activation.

In one aspect, the present invention provides a compound of formula (I) or a pharmaceutically acceptable salt thereof, for use in the treatment of an inflammatory disease and/or allergic disorder for example, chronic obstructive pulmonary disease (COPD), adult respiratory distress syndrome (ARDS), asthma, severe asthma, ulcerative colitis, Crohn's disease, bronchitis, conjunctivitis, psoriasis, scleroderma, dermatitis, allergy, rhinitis, cutaneous lupus, autoimmune bullous conditions including pemphigus and pemphigoid, mastocytosis and anaphylaxis.

In another aspect, the present invention provides a method of treating an inflammatory disease and/or allergic disorder for example, chronic obstructive pulmonary disease (COPD), adult respiratory distress syndrome (ARDS), asthma, severe asthma, ulcerative colitis, Crohn's disease, bronchitis, conjunctivitis, psoriasis, scleroderma, dermatitis, allergy, rhinitis, cutaneous lupus, autoimmune bullous conditions including pemphigus and pemphigoid, mastocytosis and anaphylaxis, which method comprises administering to a patient in need thereof a therapeutically effective amount of a compound of formula (I) or a pharmaceutically acceptable salt thereof.

In a further aspect, the present invention provides the use of a compound of formula (I) or a pharmaceutically acceptable salt thereof, for the manufacture of a medicament for the treatment of an inflammatory disease and/or allergic disorder for example, chronic obstructive pulmonary disease (COPD), adult respiratory distress syndrome (ARDS), asthma, severe asthma, ulcerative colitis, Crohn's disease, bronchitis, conjunctivitis, psoriasis, scleroderma, dermatitis, allergy, rhinitis, cutaneous lupus, autoimmune bullous conditions including pemphigus and pemphigoid, mastocytosis and anaphylaxis.

Compounds of formula (I) may also be used in combination with other classes of therapeutic agents, for example selected from anti-inflammatory agents, anticholinergic agents (particularly an M₁/M₂/M₃ receptor antagonist), β₂-adrenoreceptor agonists, antiinfective agents such as antibiotics or antivirals, or antihistamines.

In another embodiment, compounds of formula (I) may be used in combination with other classes of therapeutic agents which are known in the art for treating autoimmune diseases, for instance disease modifying anti-rheumatic drugs including cyclosporine, methotrexate, sulphasalazine, prednisone, leflunomide, and chloroquine/hydrochloroquine and also biopharmaceutical agents such as humanised monoclonal antibodies (mabs), for example including anti-TNF alpha blockers such as remicade, enbrel and humira, B cell depleting therapies such as rituximab and ofatumumab, and anti-Blys mabs such as belilumab.

The invention thus provides, a combination comprising a compound of formula (I) or a pharmaceutically acceptable salt thereof together with one or more other therapeutically active agents, for example selected from an anti-inflammatory agent such as a corticosteroid or an NSAID, an anticholinergic agent, a β₂-adrenoreceptor agonist, an antiinfective agent such as an antibiotic or an antiviral, an antihistamine, a disease modifying anti-rheumatic drug, and a biopharmaceutical agent such as humanised monoclonal antibodies (mabs), B cell depleting therapies and anti-Blys mabs. One embodiment of the invention encompasses combinations comprising a compound of formula (I) or a pharmaceutically acceptable salt thereof together with a β₂-adrenoreceptor agonist, and/or an anticholinergic, and/or a PDE-4 inhibitor, and/or an antihistamine, and/or a disease modifying anti-rheumatic drug, and/or a biopharmaceutical agent.

One embodiment of the invention encompasses combinations comprising one or two other therapeutic agents.

It will be clear to a person skilled in the art that, where appropriate, the other therapeutic ingredient(s) may be used in the form of salts, for example as alkali metal or amine salts or as acid addition salts, or prodrugs, or as esters, for example lower alkyl esters, or as solvates, for example hydrates to optimise the activity and/or stability and/or physical characteristics, such as solubility, of the therapeutic ingredient. It will be clear also that, where appropriate, the therapeutic ingredients may be used in optically pure form.

Examples of β₂-adrenoreceptor agonists include salmeterol (which may be a racemate or a single enantiomer such as the R-enantiomer), salbutamol (which may be a racemate or a single enantiomer such as the R-enantiomer), formoterol (which may be a racemate or a single diastereomer such as the R,R-diastereomer), salmefamol, fenoterol, carmoterol, etanterol, naminterol, clenbuterol, pirbuterol, flerbuterol, reproterol, bambuterol, indacaterol, terbutaline and salts thereof, for example the xinafoate (1-hydroxy-2-naphthalenecarboxylate) salt of salmeterol, the sulphate salt or free base of salbutamol or the fumarate salt of formoterol. In one embodiment the β₂-adrenoreceptor agonists are long-acting β₂-adrenoreceptor agonists, for example, compounds which provide effective bronchodilation for about 12 hours or longer.

Other β₂-adrenoreceptor agonists include those described in WO02/066422, WO02/070490, WO02/076933, WO03/024439, WO03/072539, WO03/091204, WO04/016578, WO04/022547, WO04/037807, WO04/037773, WO04/037768, WO04/039762, WO04/039766, WO01/42193 and WO03/042160.

Examples of β₂-adrenoreceptor agonists include:
3-(4-{[6-({(2R)-2-hydroxy-2-[4-hydroxy-3-(hydroxymethyl)phenyl]ethyl}amino)hexyl]oxy}butyl)benzenesulfonamide;
3-(3-{[7-({(2R)-2-hydroxy-2-[4-hydroxy-3-hydroxymethyl)phenyl]ethyl}-amino)heptyl]oxy}propyl)benzenesulfonamide;
4-{(1R)-2-[(6-{2-[(2,6-dichlorobenzyl)oxy]ethoxy}hexyl)amino]-1-hydroxyethyl}-2-(hydroxymethyl)phenol;
4-{(1R)-2-[(6-{4-[3-(cyclopentylsulfonyl)phenyl]butoxy}hexyl)amino]-1-hydroxyethyl}-2-(hydroxymethyl)phenol;
N-[2-hydroxyl-5-[(1R)-1-hydroxy-2-[[2-4-[[(2R)-2-hydroxy-2-phenylethyl]amino]phenyl]ethyl]amino]ethyl]phenyl]formamide;
N-2{2-[4-(3-phenyl-4-methoxyphenyl)aminophenyl]ethyl}-2-hydroxy-2-(8-hydroxy-2(1H)-quinolinon-5-yl)ethylamine; and
5-[(R)-2-(2-{4-[4-(2-amino-2-methyl-propoxy)-phenylamino]-phenyl}-ethylamino)-1-hydroxy-ethyl]-8-hydroxy-1H-quinolin-2-one.

The β₂-adrenoreceptor agonist may be in the form of a salt formed with a pharmaceutically acceptable acid selected from sulphuric, hydrochloric, fumaric, hydroxynaphthoic (for example 1- or 3-hydroxy-2-naphthoic), cinnamic, substituted cinnamic, triphenylacetic, sulphamic, sulphanilic, naphthaleneacrylic, benzoic, 4-methoxybenzoic, 2- or 4-hydroxybenzoic, 4-chlorobenzoic and 4-phenylbenzoic acid.

Examples of corticosteroids may include those described in WO02/088167, WO02/100879, WO02/12265, WO02/12266, WO05/005451, WO05/005452, WO06/072599 and WO06/072600.

Anti-inflammatory corticosteroids are well known in the art. Representative examples include fluticasone propionate (e.g. see U.S. Pat. No. 4,335,121), fluticasone furoate (e.g. see U.S. Pat. No. 7,101,866), beclomethasone 17-propionate ester, beclomethasone 17,21-dipropionate ester, dexamethasone or an ester thereof, mometasone or an ester thereof (e.g. mometasone furoate), ciclesonide, budesonide, flunisolide, methyl prednisolone, prednisolone, dexamethasone and 6α,9α-difluoro-11β-hydroxy-16α-methyl-3-oxo-17α-(2,2,3,3-tetramethycyclopropylcarbonyl)oxy-androsta-1,4-diene-17β-carbothioic acid S-cyanomethyl ester. Further examples of anti-inflammatory corticosteroids are described in WO02/088167, WO02/100879, WO02/12265, WO02/12266, WO05/005451, WO05/005452, WO06/072599 and WO06/072600.

Non-steroidal compounds having glucocorticoid agonism that may possess selectivity for transrepression over transactivation and that may be useful in combination therapy include those covered in the following published patent applications and patents: WO03/082827, WO98/54159, WO04/005229, WO04/009017, WO04/018429, WO03/104195, WO03/082787, WO03/082280, WO03/059899, WO03/101932, WO02/02565, WO01/16128, WO00/66590, WO03/086294, WO04/026248, WO03/061651, WO03/08277, WO06/000401, WO06/000398, WO06/015870, WO06/108699, WO07/000334 and WO07/054294.

Examples of anti-inflammatory agents include non-steroidal anti-inflammatory drugs (NSAID's).

Examples of NSAID's include sodium cromoglycate, nedocromil sodium, phosphodiesterase (PDE) inhibitors (for example, theophylline, PDE4 inhibitors or mixed PDE3/PDE4 inhibitors), leukotriene antagonists, inhibitors of leukotriene synthesis (for example montelukast), iNOS inhibitors, tryptase and elastase inhibitors, beta-2 integrin antagonists and adenosine receptor agonists or antagonists (e.g. adenosine 2a agonists), cytokine antagonists (for example chemokine antagonists, such as a CCR3 antagonist) or inhibitors of cytokine synthesis, or 5-lipoxygenase inhibitors. An iNOS (inducible nitric oxide synthase inhibitor) is preferably for oral administration. Examples of iNOS inhibitors include those disclosed in WO93/13055, WO98/30537, WO02/50021, WO95/34534 and WO99/62875. Examples of CCR3 inhibitors include those disclosed in WO02/26722.

Examples of PDE4 inhibitors include cis-4-cyano-4-(3-cyclopentyloxy-4-methoxyphenyl)cyclohexan-1-carboxylic acid, 2-carbomethoxy-4-cyano-4-(3-cyclopropylmethoxy-4-difluoromethoxyphenyl)cyclohexan-1-one and cis-[4-cyano-4-(3-cyclopropylmethoxy-4-difluoromethoxyphenyl)cyclohexan-1-ol]. Also, cis-4-cyano-4-[3-(cyclopentyloxy)-4-methoxyphenyl]cyclohexane-1-carboxylic acid (also known as cilomilast) and its salts, esters, pro-drugs or physical forms (e.g. see U.S. Pat. No. 5,552,438).

Other compounds include AWD-12-281 from Elbion (Hofgen, N. et al. 15th EFMC Int Symp Med Chem (September 6-10, Edinburgh) 1998, Abst P. 98; CAS reference No. 247584020-9); a 9-benzyladenine derivative nominated NCS-613 (INSERM); D-4418 from Chiroscience and Schering-Plough; a benzodiazepine PDE4 inhibitor identified as CI-1018 (PD-168787) and attributed to Pfizer; a benzodioxole derivative disclosed by Kyowa Hakko in WO99/16766; K-34 from Kyowa Hakko; V-11294A from Napp (Landells, L. J. et al. Eur Resp J [Annu Cong Eur Resp Soc (September 19-23, Geneva) 1998] 1998, 12 (Suppl. 28): Abst P2393); roflumilast (CAS reference No 162401-32-3) and a pthalazinone (e.g. see WO99/47505) from Byk-Gulden; Pumafentrine, (−)-p-[(4aR*,10bS*)-9-ethoxy-1,2,3,4,4a,10b-hexahydro-8-methoxy-2-methylbenzo[c][1,6]naphthyridin-6-yl]-N,N-diisopropylbenzamide which is a mixed PDE3/PDE4 inhibitor which has been prepared and published on by Byk-Gulden, now Altana; arofylline under development by Almirall-Prodesfarma; VM554/UM565 from Vernalis; or T-440 (Tanabe Seiyaku; Fuji, K. et al. J Pharmacol Exp Ther, 1998, 284(1): 162), and T2585.

Further compounds are disclosed in the published international patent application WO04/024728 (Glaxo Group Ltd), WO04/056823 (Glaxo Group Ltd) and WO04/103998 (Glaxo Group Ltd).

Examples of anticholinergic agents are those compounds that act as antagonists at the muscarinic receptors, in particular those compounds which are antagonists of the M₁ or M₃ receptors, dual antagonists of the M₁/M₃ or M₂/M₃, receptors or pan-antagonists of the M₁/M₂/M₃ receptors. Exemplary compounds for administration via inhalation include ipratropium (for example, as the bromide, CAS 22254-24-6, sold under the name Atrovent), oxitropium (for example, as the bromide, CAS 30286-75-0) and tiotropium (for example, as the bromide, CAS 136310-93-5, sold under the name Spiriva). Also of interest are revatropate (for example, as the hydrobromide, CAS 262586-79-8) and LAS-34273 which is disclosed in WO01/04118. Exemplary compounds for oral administration include pirenzepine (CAS 28797-61-7), darifenacin (CAS 133099-04-4, or CAS 133099-07-7 for the hydrobromide sold under the name Enablex), oxybutynin (CAS 5633-20-5, sold under the name Ditropan), terodiline (CAS 15793-40-5), tolterodine (CAS 124937-51-5, or CAS 124937-52-6 for the tartrate, sold under the name Detrol), otilonium (for example, as the bromide, CAS 26095-59-0, sold under the name Spasmomen), trospium chloride (CAS 10405-02-4) and solifenacin (CAS 242478-37-1, or CAS 242478-38-2 for the succinate also known as YM-905 and sold under the name Vesicare).

Other anticholinergic agents include compounds which are disclosed in U.S. patent application 60/487,981 including, for example:
(3-endo)-3-(2,2-di-2-thienylethenyl)-8,8-dimethyl-8-azoniabicyclo[3.2.1]octane bromide;
(3-endo)-3-(2,2-diphenylethenyl)-8,8-dimethyl-8-azoniabicyclo[3.2.1]octane bromide;
(3-endo)-3-(2,2-diphenylethenyl)-8,8-dimethyl-8-azoniabicyclo[3.2.1]octane 4-methylbenzenesulfonate;
(3-endo)-8,8-dimethyl-3-[2-phenyl-2-(2-thienyl)ethenyl]-8-azoniabicyclo[3.2.1]octane bromide; and
(3-endo)-8,8-dimethyl-3-[2-phenyl-2-(2-pyridinyl)ethenyl]-8-azoniabicyclo[3.2.1]octane bromide.

Further anticholinergic agents include compounds which are disclosed in U.S. patent application 60/511,009 including, for example:
(endo)-3-(2-methoxy-2,2-di-thiophen-2-yl-ethyl)-8,8-dimethyl-8-azonia-bicyclo[3.2.1]octane iodide;
3-((endo)-8-methyl-8-aza-bicyclo[3.2.1]oct-3-yl)-2,2-diphenyl-propionitrile;
(endo)-8-methyl-3-(2,2,2-triphenyl-ethyl)-8-aza-bicyclo[3.2.1]octane;
3-((endo)-8-methyl-8-aza-bicyclo[3.2.1]oct-3-yl)-2,2-diphenyl-propionamide;
3-((endo)-8-methyl-8-aza-bicyclo[3.2.1]oct-3-yl)-2,2-diphenyl-propionic acid;
(endo)-3-(2-cyano-2,2-diphenyl-ethyl)-8,8-dimethyl-8-azonia-bicyclo[3.2.1]octane iodide;
(endo)-3-(2-cyano-2,2-diphenyl-ethyl)-8,8-dimethyl-8-azonia-bicyclo[3.2.1]octane bromide;
3-((endo)-8-methyl-8-aza-bicyclo[3.2.1]oct-3-yl)-2,2-diphenyl-propan-1-ol;
N-benzyl-3-((endo)-8-methyl-8-aza-bicyclo[3.2.1]oct-3-yl)-2,2-diphenyl-propionamide;
(endo)-3-(2-carbamoyl-2,2-diphenyl-ethyl)-8,8-dimethyl-8-azonia-bicyclo[3.2.1]octane iodide;
1-benzyl-3-[3-((endo)-8-methyl-8-aza-bicyclo[3.2.1]oct-3-yl)-2,2-diphenyl-propyl]-urea;
1-ethyl-3-[3-((endo)-8-methyl-8-aza-bicyclo[3.2.1]oct-3-yl)-2,2-diphenyl-propyl]-urea;
N-[3-((endo)-8-methyl-8-aza-bicyclo[3.2.1]oct-3-yl)-2,2-diphenyl-propyl]-acetamide;
N-[3-((endo)-8-methyl-8-aza-bicyclo[3.2.1]oct-3-yl)-2,2-diphenyl-propyl]-benzamide;
3-((endo)-8-methyl-8-aza-bicyclo[3.2.1]oct-3-yl)-2,2-di-thiophen-2-yl-propionitrile;
(endo)-3-(2-cyano-2,2-di-thiophen-2-yl-ethyl)-8,8-dimethyl-8-azonia-bicyclo[3.2.1]octane iodide;
N-[3-((endo)-8-methyl-8-aza-bicyclo[3.2.1]oct-3-yl)-2,2-diphenyl-propyl]-benzenesulfonamide;
[3-((endo)-8-methyl-8-aza-bicyclo[3.2.1]oct-3-yl)-2,2-diphenyl-propyl]-urea;
N-[3-((endo)-8-methyl-8-aza-bicyclo[3.2.1]oct-3-yl)-2,2-diphenyl-propyl]-methanesulfonamide; and
(endo)-3-{2,2-diphenyl-3-[(1-phenyl-methanoyl)-amino]-propyl}-8,8-dimethyl-8-azonia-bicyclo[3.2.1]octane bromide.

Further compounds include:
(endo)-3-(2-methoxy-2,2-di-thiophen-2-yl-ethyl)-8,8-dimethyl-8-azonia-bicyclo[3.2.1]octane iodide;
(endo)-3-(2-cyano-2,2-diphenyl-ethyl)-8,8-dimethyl-8-azonia-bicyclo[3.2.1]octane iodide;
(endo)-3-(2-cyano-2,2-diphenyl-ethyl)-8,8-dimethyl-8-azonia-bicyclo[3.2.1]octane bromide;
(endo)-3-(2-carbamoyl-2,2-diphenyl-ethyl)-8,8-dimethyl-8-azonia-bicyclo[3.2.1]octane iodide;
(endo)-3-(2-cyano-2,2-di-thiophen-2-yl-ethyl)-8,8-dimethyl-8-azonia-bicyclo[3.2.1]octane iodide; and
(endo)-3-{2,2-diphenyl-3-[(1-phenyl-methanoyl)-amino]-propyl}-8,8-dimethyl-8-azonia-bicyclo[3.2.1]octane bromide.

In one embodiment the invention provides a combination comprising a compound of formula (I), or a pharmaceutically acceptable salt thereof, together with an H1 antagonist. Examples of H1 antagonists include, without limitation, methapyrilene, desloratadine, amelexanox, astemizole, azatadine, azelastine, acrivastine, brompheniramine, cetirizine, levocetirizine, efletirizine, chlorpheniramine, clemastine, cyclizine, carebastine, cyproheptadine, carbinoxamine, descarboethoxyloratadine, doxylamine, dimethindene, ebastine, epinastine, efletirizine, fexofenadine, hydroxyzine, ketotifen, loratadine, levocabastine, mizolastine, mequitazine, mianserin, noberastine, meclizine, norastemizole, olopatadine, picumast, pyrilamine, promethazine, terfenadine, tripelennamine, temelastine, trimeprazine and triprolidine, particularly cetirizine, levocetirizine, efletirizine and fexofenadine. In a further embodiment the invention provides a combination comprising a compound of formula (I), or a pharmaceutically acceptable salt thereof, together with an H3 antagonist (and/or inverse agonist). Examples of H3 antagonists include, for example, those compounds disclosed in WO2004/035556 and in WO2006/045416. Other histamine receptor antagonists which may be used in combination with the compounds of formula (I), or a pharmaceutically acceptable salt thereof, include antagonists (and/or inverse agonists) of the H4 receptor, for example, the compounds disclosed in Jablonowski et al., J. Med. Chem. 46:3957-3960 (2003).

In one embodiment there is provided, a combination comprising a compound of formula (I) or a pharmaceutically acceptable salt thereof together with a corticosteroid. In another embodiment there is provided, a combination comprising a compound of formula (I) or a pharmaceutically acceptable salt thereof together with an NSAID. In another embodiment there is provided, a combination comprising a compound of formula (I) or a pharmaceutically acceptable salt thereof together with an anticholinergic. In another embodiment there is provided, a combination comprising a compound of formula (I) or a pharmaceutically acceptable salt thereof together with a β₂-adrenoreceptor agonist. In another embodiment there is provided, a combination comprising a compound of formula (I) or a pharmaceutically acceptable salt thereof together with an antiinfective. In another embodiment there is provided, a combination comprising a compound of formula (I) or a pharmaceutically acceptable salt thereof together with an antihistamine. In another embodiment there is provided, a combination comprising a compound of formula (I) or a pharmaceutically acceptable salt thereof together with a disease modifying anti-rheumatic drug. In a further embodiment there is provided, a combination comprising a compound of formula (I) or a pharmaceutically acceptable salt thereof together with a biopharmaceutical agent.

A compound of the present invention will normally, but not necessarily, be formulated into pharmaceutical compositions prior to administration to a patient. Accordingly, in another aspect the invention is directed to pharmaceutical compositions comprising a compound of formula (I), or a pharmaceutically acceptable salt thereof, and one or more pharmaceutically acceptable carriers, diluents or excipients.

The pharmaceutical compositions of the invention may be prepared and packaged in bulk form wherein a safe and effective amount of a compound of the invention can be extracted and then given to the patient, such as with powders or syrups. Alternatively, the pharmaceutical compositions of the invention may be prepared and packaged in unit dosage form wherein each physically discrete unit contains a safe and effective amount of a compound of the invention. The pharmaceutical compositions of the invention may also be prepared and packaged in a sub-unit dosage form wherein two or more sub-unit dosage forms provide the unit dosage form. When prepared in unit dosage form, the pharmaceutical compositions of the invention typically contain from about 0.1 to 99.9 wt. %, of the compound of the invention, depending on the nature of the formulation.

In addition, the pharmaceutical compositions of the invention may optionally further comprise one or more additional pharmaceutically active compounds.

As used herein, “pharmaceutically acceptable excipient” means a pharmaceutically acceptable material, composition or vehicle involved in giving form or consistency to the pharmaceutical composition. Each excipient must be compatible with the other ingredients of the pharmaceutical composition when commingled, such that interactions which would substantially reduce the efficacy of the compound of the invention when administered to a patient and would result in pharmaceutically unacceptable compositions are avoided. In addition, each excipient must of course be of sufficiently high purity to render it pharmaceutically acceptable.

Compositions of the present invention comprising a compound of formula (I), or a pharmaceutically acceptable salt thereof and one or more pharmaceutically acceptable carriers, diluents or excipients will typically be provided as a dosage form adapted for administration to the patient by the desired route of administration. For example, dosage forms include those adapted for: (1) oral administration, such as tablets, capsules, caplets, pills, troches, powders, syrups, elixers, suspensions, solutions, emulsions, sachets, and cachets; (2) topical dermal administration, such as creams, ointments, lotions, solutions, pastes, sprays, foams, and gels, (3) inhalation, such as aerosols and solutions; (4) intranasal administration, such as solutions or sprays; (5) parenteral administration, such as sterile solutions, suspensions, and powders for reconstitution and (6) intravitreal administration.

It will be appreciated that dosage forms adapted for oral administration are commonly used for treating autoimmune disease including rheumatoid arthritis and systemic lupus erythematosus, chronic idiopathic urticarias and heme malignancies. Dosage forms adapted for topical administration to the skin are commonly used for treating atopic dermatitis, psoriasis and chronic and acute urticaria conditions, and autoimmune bullous conditions including pemphigus and pemphigoid. Dosage forms adapted for inhalation or oral administration are commonly used for treating COPD; whilst dosage forms adapted for intranasal administration are commonly used for treating allergic rhinitis.

Suitable pharmaceutically acceptable excipients will vary depending upon the particular dosage form chosen. In addition, suitable pharmaceutically acceptable excipients may be chosen for a particular function that they may serve in the composition. For example, certain pharmaceutically acceptable excipients may be chosen for their ability to facilitate the production of uniform dosage forms. Certain pharmaceutically acceptable excipients may be chosen for their ability to facilitate the production of stable dosage forms. Certain pharmaceutically acceptable excipients may be chosen for their ability to facilitate the carrying or transporting the compound of the present invention once administered to the patient from one organ, or portion of the body, to another organ, or portion of the body. Certain pharmaceutically acceptable excipients may be chosen for their ability to enhance patient compliance.

Suitable pharmaceutically acceptable excipients include the following types of excipients: Diluents, fillers, binders, disintegrants, lubricants, glidants, granulating agents, coating agents, wetting agents, solvents, co-solvents, suspending agents, emulsifiers, sweetners, flavouring agents, flavour masking agents, colouring agents, anticaking agents, humectants, chelating agents, plasticizers, viscosity increasing agents, antioxidants, preservatives, stabilizers, surfactants, and buffering agents. The skilled artisan will appreciate that certain pharmaceutically acceptable excipients may serve more than one function and may serve alternative functions depending on how much of the excipient is present in the formulation and what other ingredients are present in the formulation.

Skilled artisans possess the knowledge and skill in the art to enable them to select suitable pharmaceutically acceptable excipients in appropriate amounts for use in the invention. In addition, there are a number of resources that are available to the skilled artisan which describe pharmaceutically acceptable excipients and may be useful in selecting suitable pharmaceutically acceptable excipients. Examples include Remington's Pharmaceutical Sciences (Mack Publishing Company), Remington: The Science and Practice of Pharmacy, (Lippincott Williams & Wilkins), The Handbook of Pharmaceutical Additives (Gower Publishing Limited), and The Handbook of Pharmaceutical Excipients (the American Pharmaceutical Association and the Pharmaceutical Press).

The pharmaceutical compositions of the invention are prepared using techniques and methods known to those skilled in the art. Some of the methods commonly used in the art are described in Remington's Pharmaceutical Sciences (Mack Publishing Company). Oral solid dosage forms such as tablets will typically comprise one or more pharmaceutically acceptable excipients, which may for example help impart satisfactory processing and compression characteristics, or provide additional desirable physical characteristics to the tablet. Such pharmaceutically acceptable excipients may be selected from diluents, binders, glidants, lubricants, disintegrants, colorants, flavorants, sweetening agents, polymers, waxes or other solubility-modulating materials.

Dosage forms for topical administration to the skin may, for example, be in the form of ointments, creams, lotions, eye ointments, eye drops, ear drops, impregnated dressings, and aerosols, and may contain appropriate conventional additives, including, for example, preservatives, solvents to assist drug penetration, and emollients in ointments and creams. Such topical formulations may also contain compatible conventional carriers, for example cream or ointment bases, and ethanol or oleyl alcohol for lotions. Such carriers may constitute from about 1% to about 98% by weight of the formulation; more usually they will constitute up to about 80% by weight of the formulation.

Dosage forms for parenteral administration will generally comprise fluids, particularly intravenous fluids, i.e., sterile solutions of simple chemicals such as sugars, amino acids or electrolytes, which can be easily carried by the circulatory system and assimilated. Such fluids are typically prepared with water for injection USP. Fluids used commonly for intravenous (IV) use are disclosed in Remington, The Science and Practice of Pharmacy [ibid]. The pH of such IV fluids may vary, and will typically be from 3.5 to 8, as known in the art.

Dosage forms for nasal or inhaled administration may conveniently be formulated as aerosols, solutions, drops, gels or dry powders.

Dosage forms for topical administration to the nasal cavity (nasal administration) include pressurised aerosol formulations and aqueous formulations administered to the nose by pressurised pump. Formulations which are non-pressurised and adapted for nasal administration are of particular interest. Suitable formulations contain water as the diluent or carrier for this purpose. Aqueous formulations for administration to the nose may be provided with conventional excipients such as buffering agents, tonicity modifying agents and the like. Aqueous formulations may also be administered to the nose by nebulisation.

Dosage forms for nasal administration are provided in a metered dose device. The dosage form may be provided as a fluid formulation for delivery from a fluid dispenser having a dispensing nozzle or dispensing orifice through which a metered dose of the fluid formulation is dispensed upon the application of a user-applied force to a pump mechanism of the fluid dispenser. Such fluid dispensers are generally provided with a reservoir of multiple metered doses of the fluid formulation, the doses being dispensable upon sequential pump actuations. The dispensing nozzle or orifice may be configured for insertion into the nostrils of the user for spray dispensing of the fluid formulation into the nasal cavity. In one embodiment, the fluid dispenser is of the general type described and illustrated in WO2005/044354A1. The dispenser has a housing which houses a fluid discharge device having a compression pump mounted on a container for containing a fluid formulation. The housing has at least one finger-operable side lever which is movable inwardly with respect to the housing to cam the container upwardly in the housing to cause the pump to compress and pump a metered dose of the formulation out of a pump stem through a nasal nozzle of the housing. A particularly preferred fluid dispenser is of the general type illustrated in FIGS. 30-40 of WO2005/044354A1.

Aerosol compositions, e.g. for inhaled administration, can comprise a solution or fine suspension of the active substance in a pharmaceutically acceptable aqueous or non-aqueous solvent. Aerosol formulations can be presented in single or multidose quantities in sterile form in a sealed container, which can take the form of a cartridge or refill for use with an atomising device or inhaler. Alternatively the sealed container may be a unitary dispensing device such as a single dose nasal inhaler or an aerosol dispenser fitted with a metering valve (metered dose inhaler) which is intended for disposal once the contents of the container have been exhausted.

Where the dosage form comprises an aerosol dispenser, it preferably contains a suitable propellant under pressure such as compressed air, carbon dioxide or an organic propellant such as a hydrofluorocarbon (HFC). Suitable HFC propellants include 1,1,1,2,3,3,3-heptafluoropropane and 1,1,1,2-tetrafluoroethane. The aerosol dosage forms can also take the form of a pump-atomiser. The pressurised aerosol may contain a solution or a suspension of the active compound. This may require the incorporation of additional excipients e.g. co-solvents and/or surfactants to improve the dispersion characteristics and homogeneity of suspension formulations. Solution formulations may also require the addition of co-solvents such as ethanol. Other excipient modifiers may also be incorporated to improve, for example, the stability and/or taste and/or fine particle mass characteristics (amount and/or profile) of the formulation.

For pharmaceutical compositions suitable and/or adapted for inhaled administration, it is preferred that the pharmaceutical composition is a dry powder inhalable composition. Such a composition can comprise a powder base such as lactose, glucose, trehalose, mannitol or starch, a compound of the invention (preferably in particle-size-reduced form, e.g. in micronised form), and optionally a performance modifier such as L-leucine or another amino acid, cellobiose octaacetate and/or metals salts of stearic acid such as magnesium or calcium stearate. Preferably, the dry powder inhalable composition comprises a dry powder blend of lactose and the compound of the invention. The lactose is preferably lactose hydrate e.g. lactose monohydrate and/or is preferably inhalation-grade and/or fine-grade lactose. Preferably, the particle size of the lactose is defined by 90% or more (by weight or by volume) of the lactose particles being less than 1000 microns (micrometres) (e.g. 10-1000 microns e.g. 30-1000 microns) in diameter, and/or 50% or more of the lactose particles being less than 500 microns (e.g. 10-500 microns) in diameter. More preferably, the particle size of the lactose is defined by 90% or more of the lactose particles being less than 300 microns (e.g. 10-300 microns e.g. 50-300 microns) in diameter, and/or 50% or more of the lactose particles being less than 100 microns in diameter. Optionally, the particle size of the lactose is defined by 90% or more of the lactose particles being less than 100-200 microns in diameter, and/or 50% or more of the lactose particles being less than 40-70 microns in diameter. Most importantly, it is preferable that about 3 to about 30% (e.g. about 10%) (by weight or by volume) of the particles are less than 50 microns or less than 20 microns in diameter. For example, without limitation, a suitable inhalation-grade lactose is E9334 lactose (10% fines) (Borculo Domo Ingredients, Hanzeplein 25, 8017 JD Zwolle, Netherlands).

Optionally, in particular for dry powder inhalable compositions, a pharmaceutical composition for inhaled administration can be incorporated into a plurality of sealed dose containers (e.g. containing the dry powder composition) mounted longitudinally in a strip or ribbon inside a suitable inhalation device. The container is rupturable or peel-openable on demand and the dose of e.g. the dry powder composition can be administered by inhalation via the device such as the DISKUS® device, marketed by GlaxoSmithKline. The DISKUS® inhalation device is for example described in GB 2242134A, and in such a device at least one container for the pharmaceutical composition in powder form (the container or containers preferably being a plurality of sealed dose containers mounted longitudinally in a strip or ribbon) is defined between two members peelably secured to one another; the device comprises: a means of defining an opening station for the said container or containers; a means for peeling the members apart at the opening station to open the container; and an outlet, communicating with the opened container, through which a user can inhale the pharmaceutical composition in powder form from the opened container.

A composition of the present invention, for intranasal administration, may also be adapted for dosing by insufflation, as a dry powder formulation.

For dosage forms for inhaled administration, where the compound of the invention is present as a dry powder or in suspension, then it is preferred that it is in a particle-size-reduced form. Preferably the size-reduced form is obtained or obtainable by micronisation. The preferable particle size of the size-reduced (e.g. micronised) compound or salt is defined by a D50 value of about 0.5 to about 10 microns (for example as measured using laser diffraction).

It will be appreciated that when the compounds of formula (I) are administered in combination with other therapeutic agents normally administered by the inhaled, intravenous, oral, topical or intranasal route, that the resultant pharmaceutical composition may be administered by the same routes.

The compounds of formula (I) may conveniently be administered in amounts of, for example, 1 μg to 2 g. The precise dose will of course depend on the age and condition of the patient and the particular route of administration chosen.

### Biological Test Methods

Compounds of the invention may be tested for in vitro activity in accordance with the following assays:

### 1. Basic SYK Enzyme Activity

3 μl of SYK lysate diluted 16-fold in assay buffer (20 mM TRIS pH 7.4, 0.01% BSA, 0.1% Pluronic F-68) was added to wells containing 0.1 μl of various concentrations of compound or DMSO vehicle (1.7% final) in a Greiner low volume 384 well black plate. Following 15 minutes pre-incubation at room temperature, the reaction was initiated by the addition of 3 μl of substrate reagent containing Y7 Sox peptide, (Invitrogen Cat. #KNZ3071, 5 μM final), ATP (35 μM final) and MgCl₂ (10 mM final) in assay buffer. The reaction was incubated at room temperature before measuring fluorescence intensity (λₑₓ 360/λₑₘ 485) on an Envision plate reader (Perkin Elmer Life Sciences, Waltham, Mass., USA) at 15 minutes and 55 minutes post-substrate addition.

The compounds of Examples 1 to 29 and 32 to 42, 43 to 91, 93 to 127, 129 to 156, 158 to 165, 167 to 175 and 177 to 203 were tested essentially as described above and were found to have an average plC₅₀ value in this assay of ≧6.0. The compounds of Examples 30, 92, 128, 157, 166, and 176 were tested essentially as described above and were found to have an average plC₅₀ value in this assay of ≧5.0. The compound of Example 31 was tested essentially as described above and was found to have an average plC50 of <4.56.

Those of skill in the art will recognize that in vitro binding assays and cell-based assays for functional activity are subject to variability. Accordingly, it is to be understood that the values for the plC₅₀s recited above are exemplary only.

### Preparation of SYK Lysate

i. Preparation of Ramos Cell Lysates

Ramos B Cells (human B cells of Burkitt's lymphoma, clone 296.4C10, ATCC) were cultured in suspension in growth medium (RPMI-1640, Sigma; supplemented with 2 mM L-glutamine, Gibco; 10 mM Hepes, Sigma; 1 mM sodium pyruvate, Sigma; 10% v/v heat-inactivated FCS, Gibco). Cells were grown in Corning Cellstacks (6360 cm²) in 1 litre volume and viability and cell density were monitored daily. Cells were maintained at <1.5×10e6/ml and >92% viability

Large scale production runs were generated from Large Scale Intermediate Aliquots (LSIA's) of frozen Ramos cells as this was found to give greater reproducibility than production from a continuously growing culture of Ramos cells.

The large scale production run cells were generated in four steps:

1. Thaw LSIA into 1× Cellstack;
2. Expand culture into 4× Cellstack;
3. Expand from 4 to 12× Cellstacks;

### 4. Harvest all 12 Cellstacks

Cellstacks were harvested in 2 L centrifuge bottles using a Sorvall Mistral centrifuge, 2000 rpm, 10 minutes, 4° C. (2 L×2×10⁶ cells/ml=4×10⁹ cells total)

(Notes for cell scale-up: If the cell density exceeded 1.8×10e6/ml or viability dropped below 90% the Syk prep obtained post-stimulation was likely to be of lower activity).

Also, repeated passage of the Ramos cells seemed to have a detrimental effect on Syk activity when cell growth is done at scale (this did not seem to be the case in small scale cultures)—it is recommended always to use LSIA's and modular scale-up for large scale preps.

ii. Stimulation of Ramos Cells with Anti-IgM Ab to Produce Syk & Preparation of Lysates

Cells were stimulated at 20×10⁶ cells/ml using 15 ug/ml (final concentration) anti-IgM antibody. Following harvest (as described above), a total of 4×10⁹ cells were resuspended in 180 mls pre-warmed (37° C.) DPBS in a Corning 500 ml centrifuge bottle. 20 mls anti-IgM antibody at 150 ug/ml were added to each 500 ml centrifuge bottle. (working stock made up in DPBS pre-warmed to 37° C.). Cells were incubated for exactly 5 minutes at 37° C. following the addition of anti IgM antibody. Following 5 minutes stimulation, 300 mls ice-cold DPBS were added to each bottle to stop the stimulation (temperature drops to ˜12 deg C.) then cells were centrifuged at 2000 rpm (Sorvall Legend RT+centrifuge—pre-chilled to 4 deg C.). Cells were washed by resuspension in ice-cold DPBS and centrifugation as above. The cell pellet was then lysed in ice-cold lysis buffer containing 1% triton-x-100 at a ratio of 150 ul/1×10⁷ cells (i.e. 48 mls lysis buffer). Following the addition of lysis buffer, the cells were pipetted up & down & kept on ice for 15 minutes. The clarified lysate was then obtained by centrifugation (Sorvall Evolution RC (SLA-1500 rotor, ˜20,000 g (˜14,500 rpm), 45 min, 4° C.).

Lysate was aliquoted, snap-frozen on dry-ice & stored at −80° C. prior to assay.

### Materials

Ramos Cells Human B cells of Burkitts lymphoma, clone 296.4C10 (ATCC).

Growth Media: 500 ml RPMI, 10% heat inactivated FCS, 2 mM L-Glutamine, 2 mM

HEPES, 1 mM sodium pyruvate.

RPMI: Sigma R0883, stores CT5652

Foetal Calf Serum: Gibco 10099-141, stores CT2509

L-Glutamine: 200 mM, Gibco 25030, stores CT3005

HEPES: 1M, Sigma H0887, stores CT5637

Sodium Pyruvate: 100 mM, Sigma S8636, stores CT7741

Anti-IgM Ab: Goat anti-human IgM ((Fab′)₂ fragments) in PBS. Invitrogen, custom-made preparation (azide free and low endotoxin levels). Catalogue no. NON0687, Lot 1411913. 2.74 mg/ml.

D-PBS: Dulbeccos phosphate buffered saline, Sigma D8537

Lysis Buffer: 50 mM TRIS pH7.5+150 mM NaCl+1% Triton-X-100+2 mM EGTA+1:100 dilution inhibitor cocktails (Phosphatase inhibitor cocktail set II, Calbiochem cat no. 524625 & Protease inhibitor cocktail set V, Calbiochem cat no. 539137)

Triton-X-100: Roche 10 789 704 001 (GI 198233×, SC/159824). Made up as a 20% stock in water.

EGTA: Sigma E4378. Added solid directly to buffer.

### INTERMEDIATES AND EXAMPLES

### General

All temperatures are in ° C.

BH₃-THF refers to borane tetrahydrofuran complex

BOC/Boc refers to tert-butoxycarbonyl

BOC₂O refers to Di-tert-butyl dicarbonate

BuOH refers to butanol

Cs₂CO₃ refers to caesium carbonate

CV refers to column volume

DCM/CH₂Cl₂ refers to dichloromethane

Dioxane refers to 1,4-dioxane

DIPEA refers to N,N-diisopropylethylamine

DMSO refers to dimethylsulfoxide

DME refers to dimethoxy ethane

DMF refers to N,N-dimethylformamide

Dppf refers to 1,1′-Bis(diphenylphosphino)ferrocene

Et₃N refers to triethylamine

Ether refers to diethyl ether

EtOAc refers to ethyl acetate

h refers to hours

HF refers to hydrogen fluoride

HNO₃ refers to nitric acid

H₂SO₄ refers to sulfuric acid

HPLC refers to high performance liquid chromatography

K₂CO₃ refers to potassium carbonate

KMnO₄ refers to potassium permanganate

KOH refers to potassium hydroxide

LCMS refers to liquid chromatography-mass spectroscopy

LiAlH₄ refers to lithium aluminium hydride

MDAP refers to mass directed automated preparative chromatography

MsCl refers to methanesulfonyl chloride

min refers to minutes

NaHCO₃ refers to sodium bicarbonate

NaN₃ refers to sodium azide

NH₄Cl refers to ammonium chloride

NMP refers to N-methylpyrrolidone

PEPPSI refers to Pyridine-Enhanced Precatalyst Preparation Stabilization and

Initiation

Pd/C refers to palladium on carbon

PdCl₂.dppf refers to [1,1′-bis(diphenylphosphino)ferrocene]dichloropalladium

Pd(PPh₃)₄ or Tetrakis refers to tetrakis(triphenylphosphine) palladium (0)

20 r.t. refers to room temperature

Rt refers to retention time

SF₄ refers to sulfur tetrafluoride

SiO₂ refers to silicon dioxide

SnCl₂ refers to tin (II) chloride

Tf refers to trifluoromethanesulfonyl

Tf₂O refers to trifluoromethylsulfonic anhydride

TFA refers to trifluoroacetic acid

THF refers to tetrahydrofuran

TLC/tlc refers to thin layer chromatography

¹H NMR spectra were recorded using a Bruker DPX 400 MHz, referenced to tetramethylsilane.

GC is Agilent 6850

Column is DB5, 30 m, 0.25 microM×250 microM

Conditions: 100° C. for 2 min then 15° C./min to final temp of 200° C. held at this temp for 11 min

Dectector Temp 300° C.

Injector Temp 200° C.

LC/MS (Method A) was conducted on an Acquity UPLC BEH C18 column (50 mm×2.1 mm i.d. 1.7 μm packing diameter) at 40 degrees centigrade, eluting with 10 mM ammonium bicarbonate in water adjusted to pH 10 with ammonia solution (Solvent A) and acetonitrile (Solvent B) using the following elution gradient 0-1.5 min 1-97% B, 1.5-1.9 min 97% B, 1.9-2.0 min 100% B at a flow rate of 1 ml/min. The UV detection was a summed signal from wavelength of 210 nm to 350 nm. The mass spectra were recorded on a Waters ZQ Mass Spectrometer using Alternate-scan Positive and Negative Electrospray. Ionisation data was rounded to the nearest integer.

LC/MS (Method B) was conducted on an Acquity UPLC BEH C18 column (50 mm×2.1 mm i.d. 1.7 μm packing diameter) at 40 degrees centigrade, eluting with 0.1% v/v solution of formic acid in water (Solvent A) and 0.1% v/v solution of formic acid in acetonitrile (Solvent B) using the following elution gradient 0-1.5 min 3-100% B, 1.5-1.9 min 100% B, 1.9-2.0 min 3% B at a flow rate of 1 ml/min. The UV detection was a summed signal from wavelength of 210 nm to 350 nm. The mass spectra were recorded on a Waters ZQ Mass Spectrometer using Alternate-scan Positive and Negative Electrospray. Ionisation data was rounded to the nearest integer.

LC/MS (Method C) was conducted on an Acquity UPLC BEH C18 column (50 mm×2.1 mm i.d. 1.7 μm packing diameter) at 40 degrees centigrade, eluting with 0.1% v/v solution of trifluoroacetic acid in water (Solvent A) and 0.1% v/v solution of trifluoroacetic acid in acetonitrile (Solvent B) using the following elution gradient 0-1.5 min 3-100% B, 1.5-1.9 min 100% B, 1.9-2.0 min 3% B at a flow rate of 1 ml/min. The UV detection was a summed signal from wavelength of 210 nm to 350 nm. The mass spectra were recorded on a Waters ZQ Mass Spectrometer using Alternate-scan Positive and Negative Electrospray. Ionisation data was rounded to the nearest integer.

LC/MS (Method D) was conducted on a Sunfire C18 column (30 mm×4.6 mm i.d. 3.5 μm packing diameter) at 30 degrees centigrade. The solvents employed were: 0.1% v/v solution of formic acid in water (Solvent A) and 0.1% v/v solution of formic acid in acetonitrile (Solvent B).

The gradient employed was:

| Time (min) | Flow Rate (ml/min) | % A | % B |
|---|---|---|---|
| 0 | 3 | 97 | 3 |
| 0.1 | 3 | 97 | 3 |
| 4.2 | 3 | 0 | 100 |
| 4.8 | 3 | 0 | 100 |
| 4.9 | 3 | 97 | 3 |
| 5.0 | 3 | 97 | 3 |

The UV detection was a summed signal from wavelength of 210 nm to 350 nm.

MS Conditions

MS: Waters ZQ

Ionisation mode: Alternate-scan Positive and Negative Electrospray

Scan Range: 100 to 1000 AMU

Scan Time: 0.50 seconds

Inter scan Delay: 0.20 seconds

LC/MS (Method E)

The HPLC analysis was conducted on an XBridge C18 column (50 mm×4.6 mm i.d. 3.5 μm packing diameter) at 30 degrees centigrade.

The solvents employed were:

A=10 mM Ammonium Bicarbonate in water adjusted to pH 10 with Ammonia solution.

B=Acetonitrile.

The gradient employed was:

| Time (min) | Flow Rate (ml/min) | % A | % B |
|---|---|---|---|
| 0 | 3 | 99 | 1 |
| 0.1 | 3 | 99 | 1 |
| 4.0 | 3 | 3 | 97 |
| 5.0 | 3 | 3 | 97 |

The UV detection was a summed signal from wavelength of 210 nm to 350 nm.

### MS Conditions

MS: Waters ZQ

Ionisation mode: Alternate-scan Positive and Negative Electrospray

Scan Range: 100 to 1000 AMU

Scan Time: 0.50 seconds

Inter scan Delay: 0.20 seconds

MDAP (Method A). The HPLC analysis was conducted on an XBridge C18 column (100 mm×30 mm i.d. 5 μm packing diameter) at ambient temperature, eluting with 10 mM ammonium bicarbonate in water adjusted to pH 10 with ammonia solution (Solvent A) and acetonitrile (Solvent B) using the following elution gradient:

| Time (min) | Flow Rate (ml/min) | % A | % B |
|---|---|---|---|
| 0 | 40 | 85 | 15 |
| 1 | 40 | 85 | 15 |
| 10 | 40 | 45 | 55 |
| 11 | 40 | 1 | 99 |
| 15 | 40 | 1 | 99 |

The UV detection was an averaged signal from wavelength of 210 nm to 350 nm. The mass spectra were recorded on a Waters ZQ Mass Spectrometer using Alternate-scan Positive and Negative Electrospray. Ionisation data was rounded to the nearest integer.

MDAP (Method B). The HPLC analysis was conducted on an XBridge C18 column (100 mm×30 mm i.d. 5 μm packing diameter) at ambient temperature, eluting with 10 mM ammonium bicarbonate in water adjusted to pH 10 with ammonia solution (Solvent A) and acetonitrile (Solvent B) using the following elution gradient:

| Time (min) | Flow Rate (ml/min) | % A | % B |
|---|---|---|---|
| 0 | 40 | 85 | 15 |
| 1 | 40 | 85 | 15 |
| 20 | 40 | 45 | 55 |
| 21 | 40 | 1 | 99 |
| 25 | 40 | 1 | 99 |

The UV detection was an averaged signal from wavelength of 210 nm to 350 nm. The mass spectra were recorded on a Waters ZQ Mass Spectrometer using Alternate-scan Positive and Negative Electrospray. Ionisation data was rounded to the nearest integer.

MDAP (Method C). The HPLC analysis was conducted on a Sunfire C18 column (150 mm×30 mm i.d. 5 μm packing diameter) at ambient temperature, eluting with 0.1% v/v solution of trifluoroacetic Acid in Water (Solvent A) and 0.1% v/v solution of trifluoroacetic acid in acetonitrile (Solvent B) using the following elution gradient:

| Time (min) | Flow Rate (ml/min) | % A | % B |
|---|---|---|---|
| 0 | 40 | 100 | 0 |
| 3 | 40 | 100 | 0 |
| 3.5 | 30 | 100 | 0 |
| 24.5 | 30 | 70 | 30 |
| 25 | 30 | 1 | 99 |
| 32 | 30 | 1 | 99 |

The UV detection was an averaged signal from wavelength of 210 nm to 350 nm. The mass spectra were recorded on a Waters ZQ Mass Spectrometer using Alternate-scan Positive and Negative Electrospray. Ionisation data was rounded to the nearest integer.

MDAP (Method D). The HPLC analysis was conducted on a Sunfire C18 column (150 mm×30 mm i.d. 5 μm packing diameter) at ambient temperature, eluting with 10 mM Ammonium Bicarbonate in water adjusted to pH 10 with Ammonia solution (Solvent A) and Acetonitrile (Solvent B) using the following elution gradient:

| Time (min) | Flow Rate (ml/min) | % A | % B |
|---|---|---|---|
| 0 | 40 | 100 | 0 |
| 3 | 40 | 100 | 0 |
| 3.5 | 30 | 100 | 0 |
| 24.5 | 30 | 70 | 30 |
| 25 | 30 | 1 | 99 |
| 32 | 30 | 1 | 99 |

The UV detection was an averaged signal from wavelength of 210 nm to 350 nm. The mass spectra were recorded on a Waters ZQ Mass Spectrometer using Alternate-scan Positive and Negative Electrospray. Ionisation data was rounded to the nearest integer.

MDAP (Method E). The HPLC analysis was conducted on a Sunfire C18 column (150 mm×30 mm i.d. 5 μm packing diameter) at ambient temperature.

The solvents employed were:

A=0.1% v/v solution of formic acid in water.

B=0.1% v/v solution of formic acid in acetonitrile.

The gradient employed was:

| Time (min) | Flow Rate (ml/min) | % A | % B |
|---|---|---|---|
| 0 | 40 | 95 | 5 |
| 1 | 40 | 95 | 5 |
| 20 | 40 | 70 | 30 |
| 20.5 | 40 | 1 | 99 |
| 25 | 40 | 1 | 99 |

The UV detection was an averaged signal from wavelength of 210 nm to 350 nm.

MS Conditions

MS: Waters ZQ

Ionisation mode: Alternate-scan Positive and Negative Electrospray

Scan Range: 100 to 1000 AMU

Scan Time: 0.50 seconds

Inter scan Delay: 0.20 seconds

Silica chromatography techniques include either automated (Flashmaster, Biotage SP4) techniques or manual chromatography on pre-packed cartridges (SPE) or manually-packed flash columns.

When the name of a commercial supplier is given after the name of a compound or a reagent, for instance “compound X (Aldrich)” or “compound X/Aldrich”, this means that compound X is obtainable from a commercial supplier, such as the commercial supplier named.

Similarly, when a literature or a patent reference is given after the name of a compound, for instance compound Y (EP 0 123 456), this means that the preparation of the compound is described in the named reference.

The names of the above mentioned Examples have been obtained using the compound naming programme “ACD Name Pro 6.02” (IUPAC names are given for examples 43-203)

When mono hydrochloride salts were made, this was typically done by dissolving the compound in minimum DCM, adding a solution of hydrogen chloride in diethyl ether (1 mole equivalent) and blowing down under nitrogen and drying in vacuo.

### Intermediate 1: 2,6-Dichloro-N-nitro-4-pyridinamine

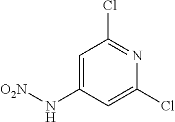 [SMILES: O=[N+]([O-])Nc1cc(Cl)nc(Cl)c1]

2,6-dichloro-4-pyridinamine (1 g, 6.13 mmol) (Peakdale Technical Molecular) was suspended in sulfuric acid (10 ml, 188 mmol) and allowed to stir at 0° C. under N₂ for 5 min. Nitric acid (0.548 ml, 12.27 mmol) was then added drop wise over 20 min maintaining the internal temp around 0° C. After addition the reaction was allowed to stir at 0° C. for 15 min and then to warm to room temperature over 30 min. The reaction was carefully poured onto ice, a precipitate formed which was removed by filtration and dried to give the title compound as a cream solid (1.310 g)

LCMS (Method B): Rt=0.85 min, MH⁺=207.87

### Intermediate 2: 2,6-Dichloro-3-nitro-4-pyridinamine

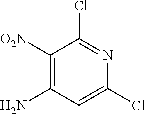 [SMILES: Nc1cc(Cl)nc(Cl)c1[N+](=O)[O-]]

2,6-dichloro-N-nitro-4-pyridinamine (1.310 g, 6.30 mmol) was suspended in sulfuric acid (10 ml, 188 mmol), a slight exotherm was observed. The resulting orange solution was allowed to stir at 50° C. under N₂ for 2 h. The reaction was poured onto ice and the resulting precipitate removed by filtration and dried. The resulting solid was suspended in water and made pH10 with aqueous ammonia. The resulting solid was removed by filtration and dried to give the title compound as a yellow solid (0.88 g).

LCMS (Method B): Rt=0.91 min, MH⁺=207.96

### Intermediate 3: 2,6-Dichloro-3,4-pyridinediamine

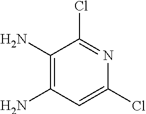

2,6-dichloro-3-nitro-4-pyridinamine (881 mg, 4.24 mmol) was taken up in ethanol (15 ml) and tin(II) chloride (3212 mg, 16.94 mmol) was added portion wise over 5 min. The resulting pale yellow solution was allowed to stir at 50° C. under N₂ for 3 h, LCMS showed approx 60% conversion, the reaction was left for a further 3 h, LCMS showed almost complete conversion. The reaction was allowed to cool to room temperature and was partitioned between NaHCO₃ (aq) (50 ml) and EtOAc (50 ml). The organic layer was dried using a hydrophobic frit, concentrated and dried in vacuo overnight to give the title compound as a yellow solid (734 mg).

LCMS (Method B): Rt=0.57 min, MH⁺=178

### Intermediate 4: 5,7-Dichloropyrido[3,4-b]pyrazine

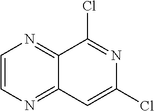 [SMILES: Clc1cc2nccnc2c(Cl)n1]

2,6-dichloro-3,4-pyridinediamine (10 g, 56.2 mmol) was suspended in tert-butanol (50 ml) and treated with glyoxal (10.27 mL, 225 mmol). The resulting solution was allowed to stir at reflux for 1 h. The hot solution was poured onto water (200 ml) and allowed to stir for 20 min. The resulting precipitate was removed by filtration and washed with water (100 ml). The resulting brown solid was taken up in DCM, filtered and loaded onto a 2 inch silica plug on a sinter funnel and eluted with EtOAc (2×100 ml). The combined eluents were concentrated to give the title compound as a deep grey solid (8.17 g).

LCMS (Method B): Rt=0.81 min, MH⁺=199.86, 201.42

### Intermediate 5: 1,1-Dimethylethyl (3R)-3-{[(7-chloropyrido[3,4-b]pyrazin-5-yl)amino]methyl}-1-piperidinecarboxylate

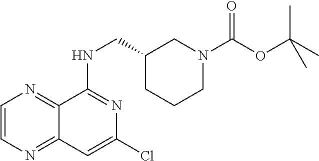 [SMILES: CC(C)(C)OC(=O)N1CCC[C@H](CNc2nc(Cl)cc3nccnc23)C1]

5,7-dichloropyrido[3,4-b]pyrazine (1 g, 5.00 mmol) was taken up in N-Methyl-2-pyrrolidone (NMP) (10 ml) and treated with 1,1-dimethylethyl (3R)-3-(aminomethyl)-1-piperidinecarboxylate (1.179 g, 5.50 mmol) (Apollo Scientific Ltd) and diisopropylethylamine (1.310 ml, 7.50 mmol). The reaction was irradiated in a Biotage microwave at 130° C. for 30 min. The reaction was partitioned between EtOAc (100 ml) and water (100 ml). The organic layer was washed with brine (100 ml), dried using a hydrophobic frit and concentrated to give a black solid. This solid was purified on silica (50 g) and eluted with a 10-40% EtOAc/cyclohexane gradient. The appropriate fractions were combined and concentrated to give the title compound as a deep orange solid (1.542 g).

LCMS (Method B): Rt=1.28 min, MH⁺=377.92

### Intermediate 6: 1,1-dimethylethyl (3R)-3-[({7-[1-(phenylmethyl)-1H-pyrazol-4-yl]pyrido[3,4-b]pyrazin-5-yl}amino)methyl]-1-piperidinecarboxylate

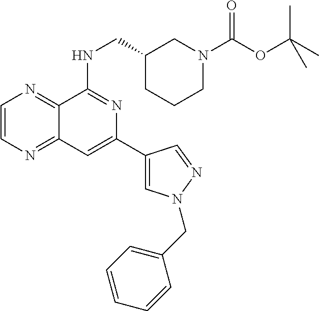 [SMILES: CC(C)(C)OC(=O)N1CCC[C@H](CNc2nc(-c3cnn(Cc4ccccc4)c3)cc3nccnc23)C1]

To a microwave vial under nitrogen was added cesium carbonate (2.59 g, 7.94 mmol) and 1-(phenylmethyl)-4-(4,4,5,5-tetramethyl-1,3,2-dioxaborolan-2-yl)-1H-pyrazole (0.827 g, 2.91 mmol) (Apollo Scientific Ltd). 1,1-dimethylethyl (3R)-3-{[(7-chloropyrido[3,4-b]pyrazin-5-yl)amino]methyl}-1-piperidinecarboxylate (1 g, 2.65 mmol) was dissolved in 1,4-Dioxane (11.5 ml) and water (2.3 ml) and added in one aliquot. Nitrogen was bubbled through the resultant suspension for ˜2 min. Tetrakis(triphenylphosphine)palladium(0) (0.306 g, 0.265 mmol) was then added in one portion and nitrogen bubbled through the yellow suspension for a further ˜1 min. The microwave vial was sealed and was heated at 150° C. in a microwave reactor for 1 h. The reaction was partitioned between water (30 ml) and ethyl acetate (30 ml). The aqueous layer was further extracted with ethyl acetate (2×30 ml) and the combined organics washed with brine (10 ml). The organics were dried (Na₂SO₄) and concentrated in vacuo. The residue was loaded on to a 100 g silica column and purified on SP4 eluting with a 0-100% ethyl acetate/cyclohexane gradient. Appropriate fractions were combined and concentrated in vacuo to give the title compound as yellow foam (1.27 g).

LCMS (Method B): Rt=1.31 min, MH⁺ 500

### Intermediate 7: 1-Cyclopentyl-4-(4,4,5,5-tetramethyl-1,3,2-dioxaborolan-2-yl)-1H-pyrazole

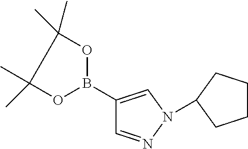 [SMILES: CC1(C)OB(c2cnn(C3CCCC3)c2)OC1(C)C]

4-(4,4,5,5-tetramethyl-1,3,2-dioxaborolan-2-yl)-1H-pyrazole (2 g, 10.31 mmol) (Aldrich) and cesium carbonate (5.04 g, 15.46 mmol) were suspended in acetonitrile (30 ml) and stirred at room temperature for 10 min. Bromocyclopentane (1.658 ml, 15.46 mmol) was added and the reaction stirred at 60° C. for 4 h. LCMS showed the reaction had not gone to completion. The reaction was stirred for 2 h. The reaction was allowed to cool, diluted with ether and filtered. The filtrate was concentrated, re-dissolved in ether and filtered again; the filtrate was again concentrated and dried to give the title compound (2.2 g).

LCMS (Method B): Rt=1.12 min, MH⁺=262.89

### Intermediate 8: 1,1-Dimethylethyl (3R)-3-({[7-(1-cyclopentyl-1H-pyrazol-4-yl)pyrido[3,4-b]pyrazin-5-yl]amino}methyl)-1-piperidinecarboxylate

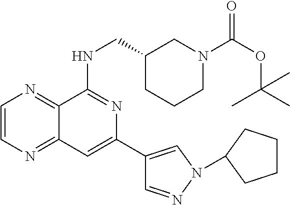 [SMILES: CC(C)(C)OC(=O)N1CCC[C@H](CNc2nc(-c3cnn(C4CCCC4)c3)cc3nccnc23)C1]

1,1-dimethylethyl (3R)-3-{[(7-chloropyrido[3,4-b]pyrazin-5-yl)amino]methyl}-1-piperidinecarboxylate (877 mg, 2.321 mmol) was dissolved in 1,4-Dioxane (8 ml) and water (2 ml). 1-cyclopentyl-4-(4,4,5,5-tetramethyl-1,3,2-dioxaborolan-2-yl)-1H-pyrazol (730 mg, 2.79 mmol), tetrakis(triphenylphosphine)palladium(0)(268 mg, 0.232 mmol) and cesium carbonate (2269 mg, 6.96 mmol) were added and sealed in a reaction vial which was heated at 130° C. for 1 hr in the microwave. LCMS showed starting material was still present. 1-cyclopentyl-4-(4,4,5,5-tetramethyl-1,3,2-dioxaborolan-2-yl)-1H-pyrazol (122 mg, 0.464 mmol) was added and the reaction was heated at 130° C. for 30 min. LCMS showed starting material was still present. Tetrakis(triphenylphosphine)palladium (0) (134 mg, 0.116 mmol) was added and the reaction was heated at 150° C. for 30 min. The reaction was partitioned between EtOAc and water. The organic layer was washed with water, dried using a hydrophobic frit and evaporated to give a brown oil (1.98 g). The oil was loaded in DCM on to a 100 g Silica cartridge and eluted using a 0-50% EtOAc/cyclohexane gradient. Appropriate fractions were combined and evaporated to give the title compound as a green oil (1.07 g).

LCMS (Method B): Rt=1.35 min, MH⁺=477.93

### Intermediate 9: Ethyl (3S)-3-fluoro-2-oxo-3-piperidinecarboxylate

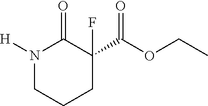

2,6-Lutidine (31.7 g, 296 mmol) was added drop wise over 30 min to a suspension of ethyl 2-oxo-3-piperidinecarboxylate (101.2 g, 591 mmol) (Aldrich), [(S)-(−)-2,2′-Bisphosphino)-1,1′-binaphthyl]palladium (II) dihydrate ditriflate (3.14 g, 2.96 mmol) (Sodeoka, M et al. Synlett 1997, 463-466; Fujii, A et al. J. Am. Chem. Soc. 1999, 121, 5450-5458) and N-fluorobenzenesulfonamide (242.0 g, 768 mmol) in ethanol (500 ml) at 0° C. in an ice bath. The temperature was maintained at approximately 10° C. during addition and then allowed to warm to room temperature overnight. Presence of solid around flask (3 L) neck suggests possible exotherm may have occurred overnight. The reaction was filtered and the solid was washed with ethanol, then DCM (200 ml). NMR confirmed no product in solid. The liquors were evaporated and re-dissolved in DCM (3500 ml). The organics were washed with saturated ammonium chloride solution (300 ml) and the aqueous was re-extracted with DCM (2×200 ml). The combined organics were evaporated and re-dissolved in DCM (300 ml), filtered through celite and washed with DCM (200 ml). The organic solution was left to stand overnight (sealed so no evaporation)—a fine precipitate appeared. The mixture was filtered through celite again and washed with DCM.

The combined organic layers were loaded onto a 1500 g silica column and purified on the companion XL eluting with 0-100% ethyl acetate in cyclohexane gradient. Appropriate fractions were combined and the solvent was removed to give the title compound as a yellow solid, which was dried under high vacuum for 1 hour (92.2 g).

LCMS (Method B): Rt=0.52 min, MH⁺ 190

Chiral analytical HPLC (25 cm Chiralpak IA, col.no.IAOOCE-MC024, 15% EtOH/C7, 1 ml/min, wavelength 215 nm, RT) showed enrichment of the fast eluter −44% ee.

The compound was purified further using preparative HPLC to improve the enantiomeric excess of the fast eluter to >99%.

| Column | Chiralpak AD, 330 × 50 mm, 20 μm | |
|---|---|---|
| Mobile Phase | A: Heptane | B: Ethanol |

| | |
|---|---|
| Gradient Profile | 15% B Isocratic |
| Run Time | 20 min |
| Flow Rate | 473 mL/min |
| Column Temperature | 20° C. |
| Wavelength | 220 nm |

### Intermediate 10: 1,1-Dimethylethyl (3S)-3-fluoro-3-(hydroxymethyl)-1-piperidinecarboxylate

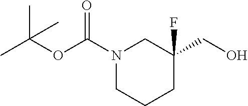 [SMILES: CC(C)(C)OC(=O)N1CCC[C@@](F)(CO)C1]

Ethyl (3S)-3-fluoro-2-oxo-3-piperidinecarboxylate (50 g, 264 mmol) was dissolved in THF (100 ml) and borane-THF complex (793 ml, 793 mmol, 1M solution) was added drop wise. The mixture was heated at reflux for 24 h, cooled to room temperature and the borane quenched by addition of methanol (150 ml).

2M HCl (200 ml) was added and the mixture heated to reflux for 20 min, then cooled and evaporated in vacuo. The residue was suspended in DCM (500 ml) and triethylamine (111 ml, 793 mmol) was added, followed by BOC anhydride (73.6 ml, 317 mmol). The mixture was stirred for 3 h, then washed with water (100 ml) and 0.5M HCl (100 ml), dried and evaporated to give 1,1-dimethylethyl (3S)-3-fluoro-3-(hydroxymethyl)-1-piperidinecarboxylate as pale yellow crystalline solid (52.85 g).

LCMS (Method B): Rt=0.80 min, MH⁺ 234

### Intermediate 11: 1,1-Dimethylethyl (3S)-3-fluoro-3-({[(trifluoromethyl)sulfonyl]oxy}methyl)-1-piperidinecarboxylate

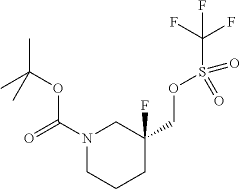 [SMILES: CC(C)(C)OC(=O)N1CCC[C@@](F)(COS(=O)(=O)C(F)(F)F)C1]

Triflic anhydride (24.1 ml, 142 mmol) was added to a solution of 1,1-dimethylethyl (3S)-3-fluoro-3-(hydroxymethyl)-1-piperidinecarboxylate (30.2 g, 129 mmol) and triethylamine (23.5 ml, 168 mmol) in DCM (100 ml) at −10° C. over 20 min. The mixture was stirred for 2 h, allowing to warm to 0° C., then washed with water and brine, dried and evaporated to give 1,1-dimethylethyl (3S)-3-fluoro-3-({[(trifluoromethyl)sulfonyl]oxy}methyl)-1-piperidinecarboxylate as a dark brown oil (50.2 g).

LCMS (Method B): Rt=1.23 min, MH⁺ 366

### Intermediate 12: 1,1-Dimethylethyl (3S)-3-(azidomethyl)-3-fluoro-1-piperidinecarboxylate

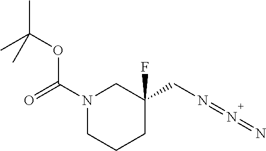

Sodium azide (9.79 g, 151 mmol) was added to a solution of 1,1-dimethylethyl (3S)-3-fluoro-3-({[(trifluoromethyl)sulfonyl]oxy}methyl)-1-piperidinecarboxylate (50 g, 137 mmol) in N,N-Dimethylformamide (DMF) (200 ml) and the mixture was heated to 80° C. for 1 h. A sample was taken and quenched with water, extracted with ether and the ether layer evaporated in vacuo. The residue was analysed by NMR showing complete consumption of starting material.

The mixture was cooled, diluted with water (1 L) and extracted with EtOAc (2×300 ml). The solvent was washed with water (2×300 ml), dried and evaporated to give 1,1-dimethylethyl (3S)-3-(azidomethyl)-3-fluoro-1-piperidinecarboxylate as an amber oil (36.7 g).

LCMS (Method B): Rt=1.12 min, MH⁺ 259

### Intermediate 13: 1,1-Dimethylethyl (3R)-3-(aminomethyl)-3-fluoro-1-piperidinecarboxylate

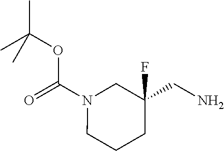 [SMILES: CC(C)(C)OC(=O)N1CCC[C@@](F)(CN)C1]

1,1-Dimethylethyl (3S)-3-(azidomethyl)-3-fluoro-1-piperidinecarboxylate (36 g, 139 mmol) was dissolved in ethanol (500 ml) and added under nitrogen to Pd/C (2.6 g, 1.222 mmol). The mixture was hydrogenated at atmospheric pressure overnight. The suspension was filtered and the filtrate evaporated in vacuo to give 1,1-dimethylethyl (3R)-3-(aminomethyl)-3-fluoro-1-piperidinecarboxylate as a pale yellow oil (32.7 g).

¹H NMR (CDC₃) 3.75-3.52 ppm (2H, 2×m, 2×CH); 3.30 ppm (1H, dd, CH); 3.20 ppm (1H, m, CH); 2.90-2.73 ppm (2H, m, CH₂); 1.96-1.72 ppm (2H, 2×m, CH₂); 1.70-1.58 ppm (1H, m, CH); 1.57-1.43 ppm (10H, m+s, CH+3×CH₃); 1.32 ppm (2H, br.s, NH₂).

### Intermediate 14: 4-(4,4,5,5-Tetramethyl-1,3,2-dioxaborolan-2-yl)-1-(2,2,2-trifluoroethyl)-1H-pyrazole

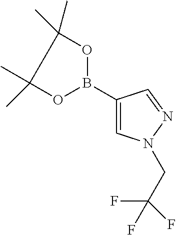 [SMILES: CC1(C)OB(c2cnn(CC(F)(F)F)c2)OC1(C)C]

2,2,2-Trifluoroethyl trifluoromethanesulfonate (28.7 g, 124 mmol) (Apollo Scientific) was added to a mixture of 4-(4,4,5,5-tetramethyl-1,3,2-dioxaborolan-2-yl)-1H-pyrazole (20 g, 103 mmol) (Aldrich) and cesium carbonate (67.2 g, 206 mmol) in N,N-Dimethylformamide (DMF) (150 ml) at 0° C. under nitrogen. The mixture was stirred for 30 min at 0° C. then allowed to warm to room temperature and stirred for a further 2 h. The mixture was quenched with water (200 ml) and extracted with EtOAc (200 ml). The organic layer was washed with water (200 ml), dried and evaporated to give a brown oil. This was dissolved in DCM (30 ml), the fine precipitate was filtered off and the filtrate loaded onto a 330 g silica column, then eluted with 0-50% EtOAc/cyclohexane. Appropriate fractions were combined and evaporated to give the title compound as a colourless oil (14.7 g)

¹H NMR (CDCl₃) 7.86 ppm (1H, s, CH); 7.82 ppm (1H, s, CH); 4.73 ppm (2H, q, CH₂); 1.34 ppm (12H, s, 4×CH₃).

### Intermediate 15: 1,1-Dimethylethyl (3R)-3-{[(7-chloropyrido[3,4-b]pyrazin-5-yl)amino]methyl}-3-fluoro-1-piperidinecarboxylate

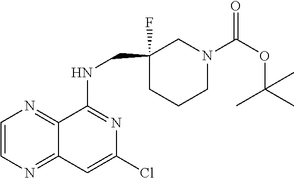 [SMILES: CC(C)(C)OC(=O)N1CCC[C@@](F)(CNc2nc(Cl)cc3nccnc23)C1]

To a solution of 1,1-dimethylethyl (3R)-3-(aminomethyl)-3-fluoro-1-piperidinecarboxylate (2.32 g, 10.00 mmol) in N-methyl-2-pyrrolidone (NMP) (5 ml) was added 5,7-dichloropyrido[3,4-b]pyrazine (2 g, 10.00 mmol) and diisopropylethylamine (3.49 ml, 20.00 mmol). This was heated at 130° C. in a Biotage Microwave for 1 h. The reaction had not gone to completion and so further amine (380 mg) was added and it was again heated to 110° C. for 30 min in a microwave. The reaction was partitioned between ethyl acetate and aqueous ammonium chloride. The layers were separated and the aqueous was re-extracted with ethyl acetate. The combined organics were washed with brine and passed through a hydrophobic frit, and concentrated in vacuo to yield a crude brown oil. This was dissolved in DCM and purified through silica (50 g) eluting with an ethyl acetate/DCM gradient. Appropriate fractions were combined and concentrated in vacuo to give the title compound as a yellow-orange gummy solid (3.13 g)

LCMS (Method B): Rt=1.24 min, MH⁺=395.8

### Intermediate 16: 1,1-dimethylethyl (3R)-3-[({7-[3,4-bis(methyloxy)phenyl]pyrido[3,4-b]pyrazin-5-yl}amino)methyl]-3-fluoro-1-piperidinecarboxylate

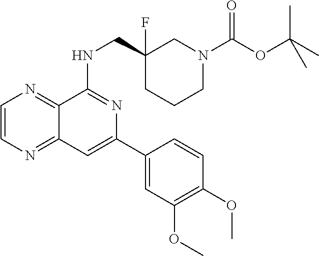 [SMILES: COc1ccc(-c2cc3nccnc3c(NC[C@]3(F)CCCN(C(=O)OC(C)(C)C)C3)n2)cc1OC]

To a 5 mL microwave vial under nitrogen was added cesium carbonate (449 mg, 1.379 mmol) and [3,4-bis(methyloxy)phenyl]boronic acid (109 mg, 0.598 mmol) (Aldrich). 1,1-dimethylethyl (3R)-3-{[(7-chloropyrido[3,4-b]pyrazin-5-yl)amino]methyl}-3-fluoro-1-piperidinecarboxylate (182 mg, 0.460 mmol) was dissolved in 1,4-dioxane (2 ml) and water (0.400 ml) and added in one aliquot. Nitrogen was bubbled through the resultant suspension for ˜2 min. Tetrakis(triphenylphosphine)palladium (0) (53.1 mg, 0.046 mmol) was then added in one portion and nitrogen bubbled through the yellow suspension for a further ˜1 min. The microwave vial was sealed and was heated at 150° C. in a microwave reactor for 1 h. The reaction was partitioned between water (20 ml) and ethyl acetate (20 ml). The aqueous layer was further extracted with ethyl acetate (2×20 ml). The combined organics were washed with brine (10 ml), dried (Na₂SO₄) and concentrated in vacuo. The resulting residue was purified on silica (25 g) using a 0-100% ethyl acetate/cyclohexane gradient. Appropriate fractions were combined and concentrated in vacuo to give the title compound as a colourless oil (171 mg).

LCMS (Method B): Rt=1.26 min, MH⁺ 498

### Intermediate 17: 1,1-Dimethylethyl (3R)-3-fluoro-3-({[7-(1-methyl-1H-pyrazol-4-yl)pyrido[3,4-b]pyrazin-5-yl]amino}methyl)-1-piperidinecarboxylate

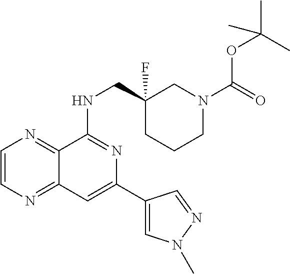 [SMILES: Cn1cc(-c2cc3nccnc3c(NC[C@]3(F)CCCN(C(=O)OC(C)(C)C)C3)n2)cn1]

To 1,1-dimethylethyl (3R)-3-{[(7-chloropyrido[3,4-b]pyrazin-5-yl)amino]methyl}-3-fluoro-1-piperidinecarboxylate (945 mg, 2.387 mmol) in 1,2-dimethoxyethane (DME) (5 ml), water (2.5 ml), ethanol (5 ml) was added 1-methyl-4-(4,4,5,5-tetramethyl-1,3,2-dioxaborolan-2-yl)-1H-pyrazole (596 mg, 2.86 mmol), potassium hydroxide (5.73 ml, 5.73 mmol, 1M aqueous solution) and [1,3-bis[2,6-bis(1-methylethyl)phenyl]-1,3-dihydro-2H-imidazol-2-ylidene](3-chloropyridinyl)dichloropalladium PEPPSI (162 mg, 0.239 mmol). The reaction was heated under reflux at 100° C. overnight under nitrogen. The reaction was filtered through celite (10 g) and washed with DCM. The solvent was removed and the resulting residue was dissolved in DCM. This was loaded onto a silica column (25 g) and purified on the SP4 using a 50-100% ethyl acetate in cyclohexane gradient. Appropriate fractions were combined and the solvent removed. The residue was dried under high vacuum for 2 h to give the title compound as a brown oil (739 mg).

LCMS (Method B): Rt=1.06 min, MH⁺ 442

### Intermediate 18: 1,1-Dimethylethyl (3S)-3-{[(7-chloropyrido[3,4-b]pyrazin-5-yl)oxy]methyl}-1-piperidinecarboxylate

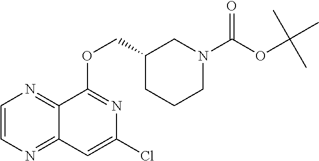 [SMILES: CC(C)(C)OC(=O)N1CCC[C@H](COc2nc(Cl)cc3nccnc23)C1]

1,1-Dimethylethyl (3S)-3-(hydroxymethyl)-1-piperidinecarboxylate (129 mg, 0.600 mmol) (Apollo Scientific Limited) was taken up in N,N-dimethylformamide (DMF) (3 ml), treated with sodium hydride (23.99 mg, 0.600 mmol) and allowed to stir at room temperature for 20 min, a yellow solution resulted. 5,7-dichloropyrido[3,4-b]pyrazine (100 mg, 0.500 mmol) was added and the reaction was allowed to stir at room temperature for a further 1 h. The reaction was partitioned between EtOAc (50 ml) and NH₄Cl (50 ml). The organic layer was dried using a hydrophobic frit and concentrated to give a brown oil. This oil was purified on silica (25 g) using a 0-40% EtOAc/cyclohexane gradient. The appropriate fractions were summed and concentrated to give the title compound as a yellow gum (91 mg).

LCMS (Method B): Rt=1.26 min, MH⁺=378.88

### Intermediate 19: 1,1-Dimethylethyl (3S)-3-[({7-[1-(2,2,2-trifluoroethyl)-1H-pyrazol-4-yl]pyrido[3,4-b]pyrazin-5-yl}oxy)methyl]-1-piperidinecarboxylate

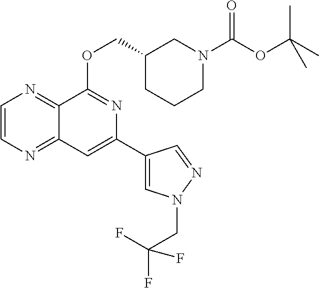 [SMILES: CC(C)(C)OC(=O)N1CCC[C@H](COc2nc(-c3cnn(CC(F)(F)F)c3)cc3nccnc23)C1]

4-(4,4,5,5-tetramethyl-1,3,2-dioxaborolan-2-yl)-1-(2,2,2-trifluoroethyl)-1H-pyrazole (1312 mg, 4.75 mmol), 1,1-dimethylethyl (3S)-3-{[(7-chloropyrido[3,4-b]pyrazin-5-yl)oxy]methyl}-1-piperidinecarboxylate (600 mg, 1.584 mmol), lithium hydroxide.monohydrate (198 mg, 4.75 mmol) and tetrakis(triphenylphosphine)palladium (0) (183 mg, 0.158 mmol) were combined and dissolved in 1,4-dioxane (3 ml) and water (2 ml). The reaction was heated in the microwave at 140° C. for 3 h. The reaction was partitioned between ethyl acetate (100 ml) and water (100 ml). The organic layer was washed with brine (100 ml) and the solvent was evaporated. The residue was dissolved in DCM and loaded on to a 50 g silica column and purified on the SP4 eluting with 10-90% ethyl acetate/cyclohexane gradient. Appropriate fractions were combined and evaporated to give the title compound as a brown oil which was dried under high vacuum overnight (174.6 mg).

LCMS (Method B): Rt=1.2 min, MH⁺ 493

### Intermediate 20: 1,1-Dimethylethyl (2S)-2-{[(7-chloropyrido[3,4-b]pyrazin-5-yl)oxy]methyl}-4-morpholinecarboxylate

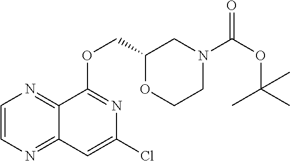 [SMILES: CC(C)(C)OC(=O)N1CCO[C@H](COc2nc(Cl)cc3nccnc23)C1]

1,1-Dimethylethyl (2S)-2-(hydroxymethyl)-4-morpholinecarboxylate (Preparation reference: WO 2009/071658) (586 mg, 2.70 mmol) was dissolved in N,N-Dimethylformamide (7 mL) and cooled in an ice bath to 5° C. under a nitrogen atmosphere. Sodium hydride 60% in mineral oil (162 mg, 4.05 mmol) was added portionwise over 15 min. 5,7-dichloropyrido[3,4-b]pyrazine (647 mg, 3.24 mmol) was then added portionwise and the mixture stirred at 5° C. for 35 min and quenched by addition of saturated aqueous ammonium chloride solution (20 mL). The solution was partitioned between ethyl acetate and water. The aqueous was re-extracted with ethyl acetate and the combined organic layers were washed with water, separated using a phase separation cartridge and the solvent removed to give a brown solid. The crude residue was dissolved in DCM and purified by silica chromatography eluting with a 12-62% ethyl acetate in petroleum ether gradient. The appropriate fractions were combined and the solvent was evaporated to give the title compound as a brown solid (917 mg).

LCMS (Method B): Rt=1.12 min, MH⁺=380.9

### Intermediate 21: 1,1-Dimethylethyl (2S)-2-({[7-(1,5-dimethyl-1H-pyrazol-4-yl)pyrido[3,4-b]pyrazin-5-yl]oxy}methyl)-4-morpholinecarboxylate

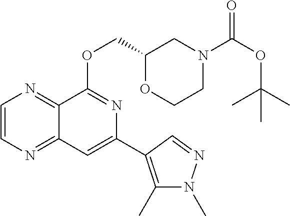 [SMILES: Cc1c(-c2cc3nccnc3c(OC[C@@H]3CN(C(=O)OC(C)(C)C)CCO3)n2)cnn1C]

To 1,1-dimethylethyl (2S)-2-{[(7-chloropyrido[3,4-b]pyrazin-5-yl)oxy]methyl}-4-morpholinecarboxylate (100 mg) in 1,4-dioxane (1.5 mL) and water (0.15 mL) was added cesium carbonate (257 mg), 1,5-dimethyl-4-(4,4,5,5-tetramethyl-1,3,2-dioxaborolan-2-yl)-1H-pyrazole (commercially available, e.g. Boron Molecular) (44 mg) and tetrakis(triphenylphosphine)palladium (0) (30 mg) and the mixture heated in a microwave at 130° C. for 1 h. Additional caesium carbonate (257 mg) and tetrakis(triphenylphosphine)palladium (0) (30 mg) added and the mixture heated in a microwave at 130° C. for 0.5 h. The mixture was partitioned between ethyl acetate and water. The aqueous was re-extracted with ethyl acetate and the combined organic layers were washed with water, separated using a phase separation cartridge and then concentrated in vacuo. The crude residue was dissolved in DCM and purified by silica chromatography eluting with a 2-13% 2M methanolic ammonia in DCM gradient. The appropriate fractions were combined and the solvent was evaporated to give the title compound as a yellow oil (104 mg).

LCMS (Method B): Rt=0.96 min, MH⁺=441.0

### Intermediate 22: 1,1-Dimethylethyl (2R)-2-{[(7-chloropyrido[3,4-b]pyrazin-5-yl)amino]methyl}-4-morpholinecarboxylate

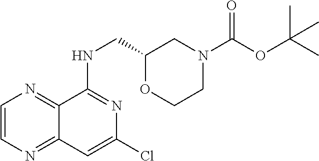 [SMILES: CC(C)(C)OC(=O)N1CCO[C@H](CNc2nc(Cl)cc3nccnc23)C1]

1,1-dimethylethyl (2R)-2-(aminomethyl)-4-morpholinecarboxylate (for preparation see: J. Medicinal Chemistry, 2009, 52 (15), 4810-4819) (6 g, 27.7 mmol) was dissolved in N-methyl-2-pyrrolidinone (NMP) (60 mL) and to this was added DIPEA (7.27 mL, 41.6 mmol) and 5,7-dichloropyrido[3,4-b]pyrazine (5.55 g, 27.7 mmol). This was split between 4 large microwave vials and each was heated at 130° C. for 30 min. They were monitored by LCMS and were given a further 10 min at 130° C. The reaction mixtures were partitioned between ethyl acetate (700 ml) and diluted aqueous ammonium chloride (1 litre). The aqueous was reextracted with ethyl acetate (300 ml) and the combined organics were washed with aqueous ammonium chloride (500 ml), dried over sodium sulfate and concentrated in vacuo to yield a crude brown oil. It was dissolved in DCM and passed through silica (70 g) eluting with DCM (6×40 ml) then 5% ethyl acetate in DCM (2×40 ml), 10% ethyl acetate in DCM (5×40 ml) then 15% ethyl acetate in DCM (2×40 ml) then 20% ethyl acetate in DCM (2×40 ml). Appropriate fractions were combined and concentrated in vacuo to yield: N8231-100-2, orange-yellow slightly gummy solid, 7.7 g

LCMS (Method B): Rt=1.17 min, MH⁺ 380

### Intermediate 23: 1,1-Dimethylethyl (2R)-2-({[7-(1-methyl-1H-pyrazol-4-yl)pyrido[3,4-b]pyrazin-5-yl]amino}methyl)-4-morpholinecarboxylate

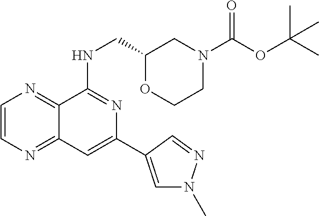 [SMILES: Cn1cc(-c2cc3nccnc3c(NC[C@@H]3CN(C(=O)OC(C)(C)C)CCO3)n2)cn1]

A mixture of 1,1-dimethylethyl (2R)-2-{[(7-chloropyrido[3,4-b]pyrazin-5-yl)amino]methyl}-4-morpholinecarboxylate (0.4 g, 1.053 mmol), 1-methyl-4-(4,4,5,5-tetramethyl-1,3,2-dioxaborolan-2-yl)-1H-pyrazole (0.263 g, 1.264 mmol) and cesium carbonate (0.858 g, 2.63 mmol) in 1,4-dioxane (15 mL) and Water (4 mL) was degassed with nitrogen. Tetrakis(triphenylphosphine)palladium (0) (0.049 g, 0.042 mmol) was added and this was heated under reflux for 8 h. The reaction had gone to completion, and the crude mixture was cooled.

A scale-up was carried out in which a mixture of 1,1-dimethylethyl (2R)-2-{[(7-chloropyrido[3,4-b]pyrazin-5-yl)amino]methyl}-4-morpholinecarboxylate (6.3 g, 16.59 mmol), 1-methyl-4-(4,4,5,5-tetramethyl-1,3,2-dioxaborolan-2-yl)-1H-pyrazole (3.80 g, 18.24 mmol) and caesium carbonate (11.89 g, 36.5 mmol) in 1,4-dioxane (150 mL) and Water (35 mL) was degassed with nitrogen and to this was added tetrakis(triphenylphosphine)palladium (0) (0.383 g, 0.332 mmol). This was degassed with nitrogen and heated under reflux for 16 h. The reaction had gone to completion and so was cooled.

These two reaction mixtures were combined and worked up as follows:

The combined crude material was partitioned between ethyl acetate and aqueous ammonium chloride. The aqueous was reextracted with ethyl acetate and the combined organics were washed with brine, dried over sodium sulfate and concentrated in vacuo to yield a crude product. It was dissolved in DCM and purified through silica (70 g) eluting with X % ethyl acetate in DCM where X=0, 5, 10, 15, 20, 25, 30, 35, 40, 45, 50, 60, 70, 80, 90 (1×40 ml of each), followed by 25×40 ml of ethyl acetate. Appropriate fractions were combined and concentrated in vacuo to yield the title compound, greyish-gold foam/solid, 7.8 g.

LCMS (Method C): rt=0.83 min, MH⁺=426

### Intermediate 24: 1,1-Dimethylethyl (3S)-3-(aminocarbonyl)-4-methyl-1-piperazinecarboxylate

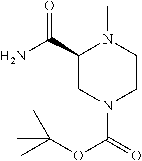 [SMILES: CN1CCN(C(=O)OC(C)(C)C)C[C@H]1C(N)=O]

1,1-Dimethylethyl (3S)-3-(aminocarbonyl)-1-piperazinecarboxylate (1.06 g, 4.62 mmol) (Arch corporation) was dissolved in ethanol (10 ml) and sodium bicarbonate (0.78 g, 9.28 mmol) was added, followed by the addition of methyl iodide (0.318 ml, 5.09 mmol). The reaction was stirred at room temperature overnight. TLC (after mini-work-up) (10% MeOH/DCM, visualised by KMnO₄) showed some starting material remaining. The reaction was refluxed for 3 h. After cooling, the solvent was evaporated in vacuo and the residue was partitioned between DCM and water. The aqueous was extracted with DCM. The combined organics were washed with brine, dried using a hydrophobic frit and evaporated to give the title compound as a white solid (919 mg).

¹H-NMR (CDC₃): 6.52 ppm (1H, br.s, NH); 5.64 ppm (1H, br.s, NH); 4.17 ppm (1H, m, CH); 4.01 ppm (1H, m, CH); 3.00-2.77 ppm (3H, br.m, 3×CH); 2.61 ppm (1H, dd, CH); 2.3 ppm (3H, s, CH₃); 2.19 ppm (1H, dt, CH); 1.45 ppm (9H, s, 3×CH₃).

### Intermediate 25: 1,1-Dimethylethyl (3R)-3-{[(7-chloropyrido[3,4-b]pyrazin-5-yl)amino]methyl}-4-methyl-1-piperazinecarboxylate

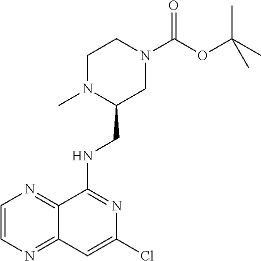 [SMILES: CN1CCN(C(=O)OC(C)(C)C)C[C@H]1CNc1nc(Cl)cc2nccnc12]

1,1-Dimethylethyl (3S)-3-(aminocarbonyl)-4-methyl-1-piperazinecarboxylate (0.5 g, 2.055 mmol) was dissolved in dry tetrahydrofuran (THF) (10 ml) and borane-tetrahydrofuran complex (8 ml, 8.00 mmol) was added. The reaction was refluxed under nitrogen overnight. A further portion of borane-tetrahydrofuran complex (8 ml, 8.00 mmol) was added and the reaction was refluxed under nitrogen for a further 24 h. After cooling, the reaction was cooled further in an ice bath and quenched by the addition of methanol (25 ml) and 1M HCl (5 ml), stirred for 90 min and left standing at room temperature for 2 h. Ethyl acetate (25 ml) was added and the layers were separated. The aqueous was extracted with ethyl acetate. The combined organics were dried using a hydrophobic frit and evaporated in vacuo to give a white solid (270 mg). TLC (10% MeOH/DCM, KMnO₄) looked like starting material. The aqueous layer was neutralised with 2M NaOH and extracted with DCM (×3). The combined organics were washed with brine, dried using a hydrophobic frit and evaporated in vacuo to give 1,1-dimethylethyl (3R)-3-(aminomethyl)-4-methyl-1-piperazinecarboxylate as a crude colourless oil (313 mg). 1,1-dimethylethyl (3R)-3-(aminomethyl)-4-methyl-1-piperazinecarboxylate (143 mg, 0.624 mmol) and diisopropylethylamine (0.131 ml, 0.750 mmol) were added to a solution of 5,7-dichloropyrido[3,4-b]pyrazine (100 mg, 0.500 mmol) in dry N-methyl-2-pyrrolidone (NMP) (2 ml). The reaction was heated at 130° C. in the microwave for 30 min. After cooling, the reaction was partitioned between ethyl acetate and water. The aqueous layer was extracted with ethyl acetate (×2). The combined organics were washed with brine, dried using a hydrophobic frit and evaporated to give an orange oil. The residue was loaded in dichloromethane and purified on silica (25 g) using a 0-100% ethyl acetate/cyclohexane gradient. Appropriate fractions were combined and evaporated to give the title compound as yellow oil (162 mg).

LCMS (Method A): Rt=1.2 min, MH⁺=393/395

### Intermediate 26: Ethyl 4,4-difluoro-1-(phenylmethyl)-3-piperidinecarboxylate

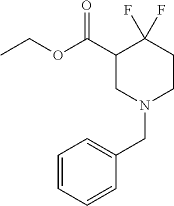 [SMILES: CCOC(=O)C1CN(Cc2ccccc2)CCC1(F)F]

Ethyl 4-oxo-1-(phenylmethyl)-3-piperidinecarboxylate (110 g, 0.421 mol) was slowly and cautiously added in small portions to a cold (˜−40° C.) stirring solution of AHF (anhydrous hydrogen fluoride) (50 ml) contained in a plastic conical flask and the resulting dark solution was transferred to a 1.0 L stainless steel autoclave. The autoclave was sealed, cooled (−196° C.) and evacuated before sulphur tetrafluoride (SF₄) (91 g, 0.842 mol) was condensed in. The vessel was allowed to warm to room temp overnight before the volatiles were vented through conc. KOH solution, the autoclave opened and the dark brown solution transferred to a plastic bucket. Ice was cautiously added and the contents of the bucket were slowly poured into a solution of potassium bicarbonate with stirring. The basic solution was extracted with DCM (3×250 ml), the organics were dried (Na₂SO₄), filtered and concentrated under reduced pressure. The product was distilled at 102° C. at 0.2 mmHg to yield the title compound as a colourless liquid that solidified on standing (85 g).

G.C 9.17 min

### Intermediate 27: [4,4-Difluoro-1-(phenylmethyl)-3-piperidinyl]methanol

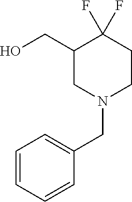 [SMILES: OCC1CN(Cc2ccccc2)CCC1(F)F]

Ethyl 4,4-difluoro-1-(phenylmethyl)-3-piperidinecarboxylate (65.0 g, 0.230 mol) was dissolved in THF (900 ml). The solution was cooled to 5° C., and lithium aluminium hydride (8.7 g, 0.230 mol, 1.0 eq) (Alfa) added in portions over 1 h, with the temperature kept below 5° C. The mixture was removed from the cooling, and stirred for a further 90 min. Once ¹H NMR confirmed the absence of starting material, the reaction mixture was cooled to below 5° C., and ethyl acetate (325 ml) added (slightly exothermic), followed by saturated sodium potassium tartrate solution (solid NaK tartrate from Aldrich, 1 L) (exothermic, bubbles a lot). The quenched mixture was allowed to reach room temperature and dichloromethane (1.5 L) added. The mixture was stirred overnight then transferred to a separating funnel, and the layers separated. The aqueous layer was extracted with dichloromethane (1.0 L), and the combined organic layers dried (MgSO₄), and the solvent evaporated to give the product as a pale yellow oil (58.1 g).

GC 8.46 min.

### Intermediate 28: 1,1-Dimethylethyl 4,4-difluoro-3-(hydroxymethyl)-1-piperidinecarboxylate

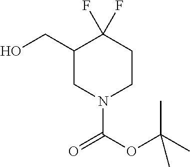 [SMILES: CC(C)(C)OC(=O)N1CCC(F)(F)C(CO)C1]

[4,4-Difluoro-1-(phenylmethyl)-3-piperidinyl]methanol (58.1 g) was dissolved in ethanol (1 L). 10% Pd/C (5.8 g) (Alfa) was added as a slurry in water, and the mixture placed under hydrogen (balloon). After 24 h, no starting material could be detected by ¹H NMR. The reaction mixture was placed under nitrogen then filtered through celite, washed (EtOH) and the solvent evaporated to give the product as an oil which solidified on standing to give an off-white solid (34.7 g). This was dissolved in DCM (500 ml) and triethylamine (38.4 ml, 0.276 mol) (Alfa) was added. Di-tert-butyl dicarbonate (55.2 g, 0.253 mol) (Alfa) in dichloromethane (500 ml) was added dropwise over 20 min. After stirring overnight, the mixture was transferred to a separating funnel, and the mixture washed with saturated ammonium chloride (1 L), saturated sodium bicarbonate (1 L) and water (1 L). The organic layer was dried (Na₂SO₄), and the solvent evaporated to give the crude product as a pale yellow oil, which solidified on standing to give a cream-coloured solid (65.7 g). The crude solid was purified by column chromatography (SiO₂, gradient elution, 0-20% EtOAc/petrol (40-60), product comes off the column in 20% EtOAc). Evaporation of the solvent gave the product as a white solid (48.6 g).

GC 6.67 min.

### Intermediate 29: 1,1-Dimethylethyl 3-{[(7-chloropyrido[3,4-b]pyrazin-5-yl)oxy]methyl}-4,4-difluoro-1-piperidinecarboxylate

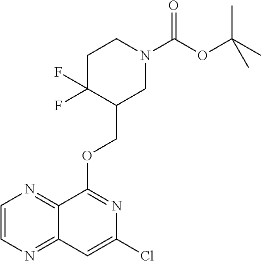 [SMILES: CC(C)(C)OC(=O)N1CCC(F)(F)C(COc2nc(Cl)cc3nccnc23)C1]

1,1-Dimethylethyl 4,4-difluoro-3-(hydroxymethyl)-1-piperidinecarboxylate (207 mg, 0.825 mmol) was dissolved in dry N,N-dimethylformamide (DMF) (5 ml) and sodium hydride (45.0 mg, 1.125 mmol) was added under nitrogen at room temperature. After 60 min, 5,7-dichloropyrido[3,4-b]pyrazine (150 mg, 0.750 mmol) was added and the reaction was stirred at room temperature under nitrogen for 2 h. The reaction was quenched by the addition of sat. ammonium chloride solution and extracted with ethyl acetate. The aqueous layer was extracted further with ethyl acetate. The combined organics were washed with water, dried using a hydrophobic frit and evaporated in vacuo to give an orange oil (369 mg). The residue was loaded in dichloromethane and purified on silica (50 g) column using a 0-100% ethyl acetate/cyclohexane gradient. Appropriate fractions were combined and evaporated to give the title compound as an orange oil (163 mg).

LCMS (Method B): Rt=1.25 min, MH⁺=415

### Intermediate 30: 1,1-Dimethylethyl 3-ethenyl-1-piperidinecarboxylate

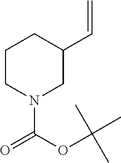

Potassium bis(trimethylsilyl)amide (51.6 mL, 25.8 mmol, 0.5M in toluene) (Aldrich) was added to a suspension of methyl(triphenyl)phosphonium bromide (9.21 g, 25.8 mmol) (Sigma-Aldrich) in tetrahydrofuran (THF) (100 mL) at 0° C. The mixture was stirred for 30 min, then 1,1-dimethylethyl 3-formyl-1-piperidinecarboxylate (5 g, 23.44 mmol) (Pharmacore, Inc) was added and the solution stirred for 3 h and allowed to warm to room temperature. The mixture was diluted with EtOAc (200 ml) and washed with water (2×200 ml) and brine (200 ml), dried and evaporated. The residue was triturated with ether and filtered to give an oil. NMR showed product plus triphenylphospine oxide. The mixture was loaded onto a silica column (100 g) and eluted with a 0-30% EtOAc/cyclohexane gradient. Appropriate fractions were combined and evaporated to give the title compound as a colourless oil (3.8 g).

¹H NMR (CDCl₃): 5.71 ppm (1H, m, CH); 5.06 ppm (1H, dt, CH); 5.02 ppm (1H, dt, CH); 4.20-3.82 ppm (2H, v. br. s+br. d, 2×CH); 2.73 ppm (1H, m, CH); 2.56 ppm (1H, v. br. s, CH); 2.14 ppm (1H, m, CH); 1.85 ppm (1H, m, CH); 1.66 ppm (1H, m, CH [+water]); 1.46 ppm (10H, s+m, 3×CH₃+CH); 1.26 ppm (1H, m, CH).

### Intermediate 31: 1,1-Dimethylethyl 3-[2-(7-chloropyrido[3,4-b]pyrazin-5-yl)ethyl]-1-piperidinecarboxylate

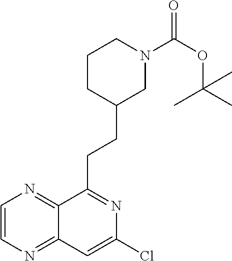 [SMILES: CC(C)(C)OC(=O)N1CCCC(CCc2nc(Cl)cc3nccnc23)C1]

9-Borabicyclo[3.3.1]nonane solution (9-BBN) (9.47 mL, 4.73 mmol, 0.5M in THF) (Aldrich) was added to 1,1-dimethylethyl 3-ethenyl-1-piperidinecarboxylate (1 g, 4.73 mmol) in THF (30 ml) and the mixture was heated at reflux under nitrogen for 2 h. The reaction was cooled and 5,7-dichloropyrido[3,4-b]pyrazine (0.947 g, 4.73 mmol), 1,1′-bis(diphenylphosphino)ferrocenedichloro palladium (II) (0.104 g, 0.142 mmol), potassium carbonate (1.308 g, 9.47 mmol), N,N-dimethylformamide (DMF) (30 ml) and water (4 ml) were added and the solution was heated at 80° C. for 3 h. The mixture was evaporated in vacuo, diluted with water (50 ml) and extracted with ether (2×50 ml). The combined organics were washed with water (50 ml), dried and evaporated to give a brown oil. The impure product was purified by chromatography (330 g silica column) eluting with a 0-80% EtOAc/cyclohexane gradient. Appropriate fractions were combined and evaporated to give the title compound as a colourless gum (1.31 g).

LCMS (Method B): Rt=1.35 min, MH⁺ 377

### Intermediate 32: 1,1-Dimethylethyl {4-[(7-chloropyrido[3,4-b]pyrazin-5-yl)amino]butyl}carbamate

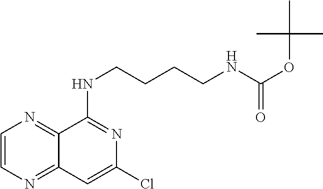 [SMILES: CC(C)(C)OC(=O)NCCCCNc1nc(Cl)cc2nccnc12]

To 5,7-dichloropyrido[3,4-b]pyrazine (650 mg, 3.25 mmol) was added 1,1-dimethylethyl (4-aminobutyl)carbamate (0.622 ml, 3.25 mmol) (Fluka) and diisopropylethylamine (0.851 ml, 4.87 mmol). To the mixture was added N-methyl-2-pyrrolidone (NMP) (10 ml). The microwave vial was sealed and heated to 130° C. for 30 min. The reaction mixture was partitioned between water (70 ml) and ethyl acetate (70 ml) and then separated. The aqueous layer was extracted with ethyl acetate (2×50 ml). The combined organics were passed through a phase separation cartridge and reduced in vacuo. The residue was dissolved in DCM and loaded onto a silica cartridge (50 g) and purified via SP4 using a 15-75% EtOAc in cyclohexane gradient. The appropriate fractions were combined and concentrated to give the title compound as a yellow film (1.01 g).

LCMS (Method C): Rt=1.11 min, MH⁺=352.0

### Intermediate 33: 1,1-Dimethylethyl-2-{[(7-chloropyrido[3,4-b]pyrazin-5-yl)amino]methyl}-4-morpholinecarboxylate

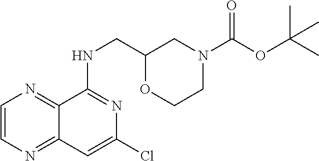 [SMILES: CC(C)(C)OC(=O)N1CCOC(CNc2nc(Cl)cc3nccnc23)C1]

1,1-Dimethylethyl-2-(aminomethyl)-4-morpholinecarboxylate (60 mg, 0.28 mmol) was dissolved in N-methyl-2-pyrrolidinone (NMP) (1 mL) and to this was added DIPEA (0.07 mL, 0.38 mmol) and 5,7-dichloropyrido[3,4-b]pyrazine (50 mg, 0.25 mmol). This was heated at 130° C. for 30 min. The reaction mixtures were partitioned between ethyl acetate (50 ml) and water (50 mL) and the organic layer washed with water (50 mL), dried over a hydrophobic frit and concentrated in vacuo to yield an orange gum. It was dissolved in DCM and passed through silica (10 g) eluting with a 10-40% ethyl acetate in cyclohexane gradient. Appropriate fractions were combined and concentrated in vacuo to yield the title compound as a yellow solid, 91 mg.

LCMS (Method B): Rt=1.17 min, MH⁺ 380

### Intermediate 34: 1,1-dimethylethyl-2-[({7-[6-(dimethylamino)-3-pyridinyl]pyrido[3,4-b]pyrazin-5-yl}amino)methyl]-4-morpholinecarboxylate (Isomer 1)

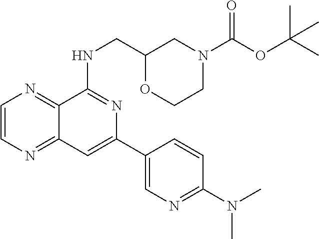 [SMILES: CN(C)c1ccc(-c2cc3nccnc3c(NCC3CN(C(=O)OC(C)(C)C)CCO3)n2)cn1]

A mixture of 1,1-dimethylethyl-2-{[(7-chloropyrido[3,4-b]pyrazin-5-yl)amino]methyl}-4-morpholinecarboxylate (89 mg, 0.23 mmol), [6-(dimethylamino)-3-pyridinyl]boronic acid hydrate (52 mg, 0.28 mmol), tetrakis(triphenylphosphine)palladium (0) (27 mg, 0.02 mmol) and caesium carbonate (229 mg, 0.70 mmol) in 1,4-dioxane (1 mL) and water (0.1 mL) was irradiated in the microwave at 130° C. for 30 min. The crude mixture was cooled and partitioned between ethyl acetate and water, dried over a hydrophobic frit and concentrated in vacuo to yield a crude product. It was dissolved in DCM and purified through silica (10 g) eluting with a 0-20% 2M methanolic ammonia in DCM gradient. Appropriate fractions were combined and concentrated in vacuo. The residue was dissolved in methanol and loaded onto a 2 g SCX SPE cartridge, washed with methanol and eluted with 2M methanolic ammonia. The solvent was removed to give a yellow gum.

Chiral separation was achieved (Prep Method: Approx 50 mg dissolved in 0.5 ml of DMF and 2 ml EtOH with heat (approx 45° C.) then 1 ml heptane was added. Before injection the sample was spun down in a centrifuge and the supernatent injected onto the column. Injection; 3.5 ml of the above sample solution was injected onto the column. 15% EtOH/heptane, f=75 ml/min, wavelength 300 nm, Column 5 cm×20 cm Chiralpak AD (20 um) self packed) to yield the title compound (first eluting peak from the chiral column) as a yellow solid (13 mg).

LCMS (Method B): Rt=0.83 min, MH⁺=466

The following intermediate was obtained as the second eluting peak from the chiral separation above:

### Intermediate 35: 1,1-dimethylethyl-2-[({7-[6-(dimethylamino)-3-pyridinyl]pyrido[3,4-b]pyrazin-5-yl}amino)methyl]-4-morpholinecarboxylate (Isomer 2)

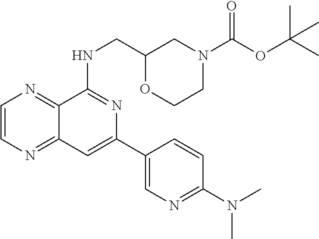 [SMILES: CN(C)c1ccc(-c2cc3nccnc3c(NCC3CN(C(=O)OC(C)(C)C)CCO3)n2)cn1]

LCMS (Method B): Rt=0.83 min, MH⁺=466

### Intermediate 36: 1,1-dimethylethyl 3-(aminomethyl)-4,4-difluoro-1-piperidinecarboxylate

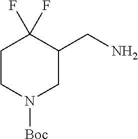 [SMILES: CC(C)(C)OC(=O)N1CCC(F)(F)C(CN)C1]

### Step 1—Mesylation

1,1-dimethylethyl 4,4-difluoro-3-(hydroxymethyl)-1-piperidinecarboxylate (5.0 g, 0.0199 mol, 1.0 eq) was dissolved in dichloromethane (50 ml), triethylamine (3.6 ml, 0.0259 mol, 1.3 eq) was then added, and the mixture cooled to below 5.0° C. Methanesulphonyl chloride (1.9 ml, 0.0239 mol, 1.2 eq) was then added dropwise over 20 min with the internal temperature kept below 5.0° C. The mixture was then removed from cooling and stirred for 30 min. Once the absence of starting material had been confirmed by NMR (¹H and ¹⁹F), dichloromethane (100 ml) was added, and the mixture washed with saturated NH₄Cl (150 ml) and saturated brine (150 ml), then dried (Na₂SO₄), and the solvent evaporated to give the product as a pale yellow oil (7.6 g—contains some solvent), which partially solidified on standing overnight. This was used in the next step without further purification.

### Step 2—Azide Formation

The mesylate from the previous step (7.6 g, 0.0231 mmol, 1.0 eq) and sodium azide (4.7 g, 0.0723 mol, 3.1 eq) were added to DMF (35 ml), and heated to 90° C. overnight. Once the absence of starting material was confirmed by ¹H and ¹⁹F NMR, the mixture was cooled. 10% sodium thiosulphate solution (70 ml) was added followed by ethyl acetate (70 ml). The layers were separated, and the organic layer washed with 10% sodium thiosulphate (70 ml) and water (2×70 ml), then dried (MgSO₄) and the solvent evaporated to give the product as a pale yellow oil (5.9 g) which was used in the next step without further purification.

### Step 3—Hydrogenation

The azide from the previous step (5.9 g, 0.0214 mol) was dissolved in ethanol (120 ml), and placed under nitrogen. 10% Pd/C (0.6 g) was added as a slurry in water, and the mixture placed under hydrogen (balloon). After stirring overnight, the absence of starting material was confirmed by TLC (50:50 EtOAc:petrol (40-60), visualised with 10% phosphomolybdic acid in EtOH), and the reaction mixture filtered through celite to give the crude product as a pale yellow oil.

The crude product was combined with that from a 3.7 g hydrogenation and purified by column chromatography on SiO₂ (gradient elution: 50:50 EtOAc:petrol (40-60) (1 L), EtOAc (1 L), 5% 2M NH₃ in MeOH/CH₂Cl₂, 10% 2M NH₃ in MeOH/CH₂Cl₂) to give the title compound as a pale yellow oil (5.51 g)

GC: 6.73 min.

¹H NMR (CDCl₃) 3.85-3.65 ppm (2H, m, CH₂); 3.45-3.35 ppm (1H, m, CH₂); 3.35-3.20 ppm (1H, m, CH₂); 3.05 ppm (1H, dd, CH₂); 2.65 ppm (1H, dd, CH₂); 2.10-1.80 ppm (3H, m, 1×CH, 2×CH₂); 1.50 ppm (9H, s, 3×CH₃).

### Intermediate 37: 1,1-dimethylethyl 3-{[(7-chloropyrido[3,4-b]pyrazin-5-yl)amino]methyl}-4,4-difluoro-1-piperidinecarboxylate (Isomer 2)

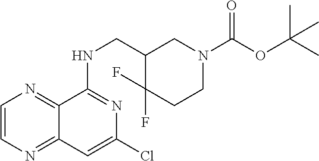 [SMILES: CC(C)(C)OC(=O)N1CCC(F)(F)C(CNc2nc(Cl)cc3nccnc23)C1]

5,7-Dichloropyrido[3,4-b]pyrazine (620 mg, 3.10 mmol) was dissolved in N-methyl-2-pyrrolidinone (NMP) (5 mL) and to this was added DIPEA (0.601 mL, 4.65 mmol) and 1,1-dimethylethyl 3-(aminomethyl)-4,4-difluoro-1-piperidinecarboxylate (776 mg, 3.10 mmol). This was heated in a microwave at 130° C. for 30 min. The reaction mixture was partitioned between ethyl acetate and water. The aqueous was re-extracted twice with ethyl acetate and the combined organic layers washed with brine, dried over a hydrophobic frit and concentrated in vacuo to yield a brown oil. It was dissolved in DCM and passed through silica (100 g) eluting with a 10-50% ethyl acetate in cyclohexane gradient. Appropriate fractions were combined and concentrated in vacuo to yield the title compound as a yellow solid, 1.0 g.

Chiral separation was achieved (Sample preparation: Sample dissolved in ethanol (30 ml) sonicating and heating with air gun as required. 4-5 ml injections were then pumped onto a preparative scale Whelk-O(S,S) column (2 inch). Details as follows: Column—Whelk-O(S,S) (50×250 mm, 10 micron); Detection—UV DAD—300 nm (bandwidth 180 nm, reference 550 nm (bandwidth 100 nm)); Flow Rate—70 ml/min; Mobile Phase A: Heptane; Mobile Phase B: IPA; Isocratic method (premixed) 5% B; Runtime—60 min; Number of runs—8) to yield the title compound (second eluting peak from the chiral column) as a yellow solid (441 mg).

LCMS (Method B): Rt=1.27 min, MH⁺=414

The following intermediate was obtained as the first eluting peak from the chiral separation above:

### Intermediate 38: 1,1-dimethylethyl 3-{[(7-chloropyrido[3,4-b]pyrazin-5-yl)amino]methyl}-4,4-difluoro-1-piperidinecarboxylate (Isomer 1)

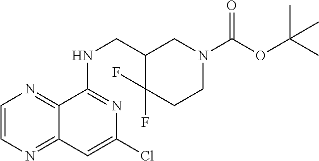 [SMILES: CC(C)(C)OC(=O)N1CCC(F)(F)C(CNc2nc(Cl)cc3nccnc23)C1]

LCMS (Method B): Rt=1.27 min, MH⁺=414

The following intermediate was prepared similarly:

### Intermediate 39: 1,1-dimethylethyl 5-{[(7-chloropyrido[3,4-b]pyrazin-5-yl)amino]methyl}-3,3-difluoro-1-piperidinecarboxylate

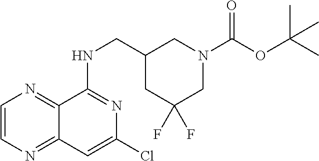 [SMILES: CC(C)(C)OC(=O)N1CC(CNc2nc(Cl)cc3nccnc23)CC(F)(F)C1]

LCMS (Method B): Rt=1.24 min, MH⁺=414

### Intermediate 40: 1,1-dimethylethyl (3R)-3-({[7-(3-oxo-1-piperazinyl)pyrido[3,4-b]pyrazin-5-yl]amino}methyl)-1-piperidinecarboxylate

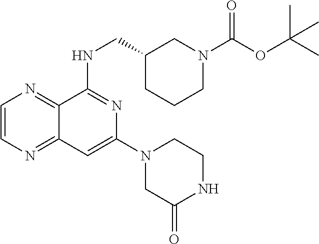 [SMILES: CC(C)(C)OC(=O)N1CCC[C@H](CNc2nc(N3CCNC(=O)C3)cc3nccnc23)C1]

1,1-Dimethylethyl (3R)-3-{[(7-chloropyrido[3,4-b]pyrazin-5-yl)amino]methyl}-1-piperidinecarboxylate (200 mg, 0.529 mmol), 2-piperazinone (265 mg, 2.65 mmol), DIPEA (0.185 mL, 1.059 mmol) and N-methyl-2-pyrrolidone (NMP) (1.5 mL) were all added to a microwave vial. The reaction mixture was heated for 10 h at 120° C. then for 4 h at 130° C. then 9 h at 140° C. The reaction mixture was partitioned between ethyl acetate and aqueous sodium bicarbonate. The organics were washed twice with aqueous sodium bicarbonate followed by brine. The organics were dried over magnesium sulfate, filtered and concentrated in vacuo to give a crude product. This was purified on silica (SP4) and a gradient was run of 1 column volume (CV) of neat DCM, 0-15% of 2M ammonia in methanol, in DCM, over 15 column volumes (CV). The fractions containing the product were combined and concentrated in vacuo to give the title compound (89.6 mg)

LCMS (Method A): Rt=1.02 min, MH⁺=442.3

The following intermediate was prepared similarly:

### Intermediate 41: 1,1-dimethylethyl (3R)-3-({[7-(1-piperazinyl)pyrido[3,4-b]pyrazin-5-yl]amino}methyl)-1-piperidinecarboxylate

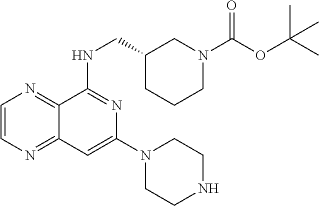 [SMILES: CC(C)(C)OC(=O)N1CCC[C@H](CNc2nc(N3CCNCC3)cc3nccnc23)C1]

LCMS (Method C): Rt=0.78 min, MH⁺=428

### Intermediate 42: 1,1-dimethylethyl (3R)-3-{[(7-chloropyrido[3,4-b]pyrazin-5-yl)oxy]methyl}-1-piperidinecarboxylate

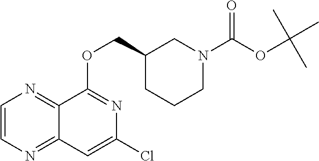 [SMILES: CC(C)(C)OC(=O)N1CCC[C@@H](COc2nc(Cl)cc3nccnc23)C1]

This was prepared similarly to intermediate 18 using 1,1-dimethylethyl (3R)-3-(hydroxymethyl)-1-piperidinecarboxylate as the alcohol.

LCMS (Method C): Rt=1.25 min, MH⁺=379.0

### Intermediate 43: [(2S)-1-methyl-2-piperazinyl]methanol

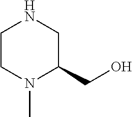

(2S)-1-{[(1,1-dimethylethyl)oxy]carbonyl}-2-piperazinecarboxylic acid (Commercial from ACESYS) (1.03 g, 4.47 mmol) was dissolved in dry Tetrahydrofuran (THF) (25 ml) and cooled to 0° C. under nitrogen. Lithium aluminium hydride (11 ml, 11.00 mmol) was added dropwise and the reaction was stirred at 0° C. for 15 mins and allowed to warm to room temperature. The solution was stirred for ˜1 hour at room temperature and then heated at reflux overnight. TLC (20% MeOH/DCM+few drops ammonia; visualised by KMnO4) showed the reaction had gone to completion. After cooling, the reaction was cooled to 0° C. and quenched by the dropwise sequential addition of water (0.5 ml), 2M NaOH (0.5 ml) and water (1 ml). The resulting slurry was filtered and washed with THF. The filtrate was evaporated in vacuo and the resulting oil was azeotroped with methanol (×2) to give the title compound as colourless oil (374 mg)

LCMS (Method B): Rt=0.18 min, MH⁺=131

### Intermediate 44: 1,1-dimethylethyl (3S)-3-(hydroxymethyl)-4-methyl-1-piperazinecarboxylate

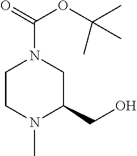 [SMILES: CN1CCN(C(=O)OC(C)(C)C)C[C@H]1CO]

[(2S)-1-methyl-2-piperazinyl]methanol (367 mg, 2.82 mmol) was dissolved in Dichloromethane (DCM) (10 ml) and triethylamine (0.432 ml, 3.10 mmol) and Boc-anhydride (677 mg, 3.10 mmol) were added. The reaction was stirred at room temperature under nitrogen overnight. TLC (20% MeOH/DCM+few drops ammonia; visualised by KMnO4) showed some starting material remained. A further portion of Boc-anhydride (323 mg, 1.480 mmol) was added and the reaction was stirred at room temperature for 4 hours. TLC still showed starting material. A further portion of Boc-anhydride (308 mg, 1.409 mmol) was added and the reaction was stirred at room temperature overnight. TLC showed no starting material remained. The reaction mixture was washed with water (×2). The aqueous was extracted with DCM (×2). The combined organics were washed with brine, dried using a hydrophobic frit and evaporated in vacuo to give a colourless oil. The residue was loaded in dichloromethane and purified on the Biotage SP4 silica (Si) 40+S column using a 0-20% 20% 2M ammonia methanol in DCM/DCM gradient over 27 CV's (collect all setting, visualised by TLC and KMnO₄ dip). Appropriate fractions were combined and evaporated in vacuo to give the title compound as a colourless oil (268 mg)

LCMS (Method B): Rt=0.45 min, MH⁺=231

### Intermediate 45: 1,1-dimethylethyl (3S)-4-ethyl-3-(hydroxymethyl)-1-piperazinecarboxylate

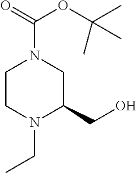 [SMILES: CCN1CCN(C(=O)OC(C)(C)C)C[C@H]1CO]

1,1-dimethylethyl (3S)-3-(hydroxymethyl)-1-piperazinecarboxylate (Commercial: e.g. Activate Scientific) (0.5 g, 2.312 mmol) and acetaldehyde (0.209 ml, 3.70 mmol) were dissolved in Methanol (10 ml) with molecular sieves and stirred at room temperature under nitrogen for 4 hours. Sodium borohydride (0.140 g, 3.70 mmol) was added and the reaction was stirred at room temperature for 18 hours. The reaction was quenched with 2M NaOH and the reaction was filtered through a celite column. The filtrate was extracted with ethyl acetate (×3). The combined organics were washed with water, dried using a hydrophobic frit and evaporated in vacuo to give the title compound as a colourless oil (0.546 g)

LCMS (Method B): Rt=0.45 min, MH⁺=245

### Intermediate 46: 1,1-dimethylethyl (3S)-3-(hydroxymethyl)-4-(trifluoroacetyl)-1-piperazinecarboxylate

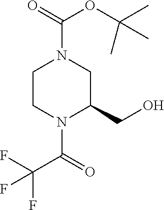 [SMILES: CC(C)(C)OC(=O)N1CCN(C(=O)C(F)(F)F)[C@H](CO)C1]

1,1-dimethylethyl (3S)-3-(hydroxymethyl)-1-piperazinecarboxylate (Commercial) (350 mg, 1.618 mmol) was dissolved in Dichloromethane (DCM) (10 ml) and cooled in an ice bath under nitrogen. Triethylamine (0.564 ml, 4.05 mmol) was added followed by the careful addition of trifluoroacetic anhydride (0.571 ml, 4.05 mmol). After ˜10 mins, the ice bath was removed and the reaction was allowed to warm to room temperature and stirred for 3 hours.

The reaction was washed with water. The aqueous was extracted with DCM. The combined organics were washed with water, dried using a hydrophobic frit and evaporated in vacuo to give a pale yellow oil (0.69 g)

N11516-44-1 was loaded in methanol and purified by SPE on sulphonic acid (SCX) 20 g using methanol. The fractions were combined and evaporated in vacuo to give the title compound as a pale yellow oil (0.6 g)

LCMS (Method B): Rt=0.45 min, MH⁺=245

### Intermediate 47: 1,1-dimethylethyl (3S)-3-(hydroxymethyl)-4-(2,2,2-trifluoroethyl)-1-piperazinecarboxylate

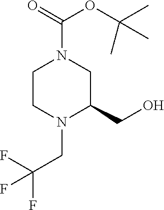 [SMILES: CC(C)(C)OC(=O)N1CCN(CC(F)(F)F)[C@H](CO)C1]

1,1-dimethylethyl (3S)-3-(hydroxymethyl)-4-(trifluoroacetyl)-1-piperazinecarboxylate (600 mg, 1.633 mmol) was dissolved in dry tetrahydrofuran (THF) (10 ml) and borane-tetrahydrofuran complex (8.17 ml, 8.17 mmol) was added slowly. The reaction was refluxed under nitrogen for 24 hours. After cooling, the reaction was cooled further in an ice bath and quenched by the addition of methanol (10 ml) and 1M HCl (5 ml) and stirred for 1 hour at room temperature. Ethyl acetate (25 ml) and water (25 ml) were added and the layers were separated. The ethyl acetate layer was dried using a hydrophobic frit and evaporated in vacuo to give the title compound as a colourless oil (251 mg)

LCMS (Method B): Rt=0.95 min, MH⁺=295

### Intermediate 48: 1,1-dimethylethyl (2S,3S)-3-(hydroxymethyl)-2-methyl-1-piperidinecarboxylate

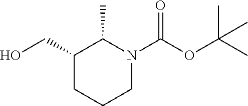 [SMILES: C[C@H]1[C@@H](CO)CCCN1C(=O)OC(C)(C)C]

(2S,3S)-1-{[(1,1-dimethylethyl)oxy]carbonyl}-2-methyl-3-piperidinecarboxylic acid (Commercial: eg ASW-MedChem, Inc.) (409 mg, 1.681 mmol) was dissolved in Tetrahydrofuran (THF) (6 mL) and at −10° C. stirring under nitrogen was added N-methylmorpholine (0.185 mL, 1.681 mmol) followed by ethyl chloroformate (0.161 mL, 1.681 mmol). After stirring for 25 min sodium borohydride (191 mg, 5.04 mmol) was added in one portion and over the next 15 min was added Methanol (16 mL) dropwise and bubbling was observed. To the mixture was added 1M HCl (2.5 ml) and then the mixture was reduced in vacuo. The mixture was partitioned between water (20 ml) and ethyl acetate (50 ml). The aqueous was reextracted with ethyl acetate (2×50 ml). The organics were washed with water (100 ml) and then passed through a phase separation cartridge and reduced in vacuo. to give an oil. The oil was dissolved in ethyl acetate (40 ml) and then washed with NaHCO₃ (25 ml) and water (25 ml). The organics were reduced in vacuo to give the title compound as an oil (230 mg)

LCMS (Method B): Rt=0.88 min, MH⁺=230.1

### Intermediate 49: 1-(1,1-dimethylethyl) 3-methyl 5-fluoro-1,3-piperidinedicarboxylate

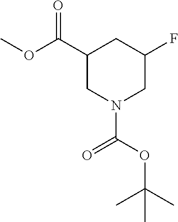 [SMILES: COC(=O)C1CC(F)CN(C(=O)OC(C)(C)C)C1]

To 1-(1,1-dimethylethyl)3-methyl 5-hydroxy-1,3-piperidinedicarboxylate (Commercial e.g. Activate Scientific) (0.915 g) in DCM (50 ml) was allowed to stir at −78° C. for 5 mins DAST (0.559 mL, 4.23 mmol) was then added dropwise over 5 mins maintaining the temp below −60° C. the reaction was then allowed to stir around −78° C. for 2 h and then to warm to room temperature over 3 h. The reaction was washed with NaHCO3 (2×100 ml) dried using a hydrophobic frit and concentrated to a brown gum. This gum was purified using a SP4 SNAP 50 column, eluting with 0-25% EtOAc/Cyclohexane (15CV). Appropriate fractions were summed and concentrated to give the title compound (284 mg)

LCMS (Method B): Rt=0.96 min, MH⁺=262 (weak)

### Intermediate 50: 1,1-dimethylethyl 3-fluoro-5-(hydroxymethyl)-1-piperidinecarboxylate

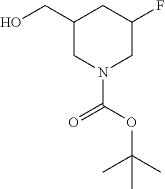 [SMILES: CC(C)(C)OC(=O)N1CC(F)CC(CO)C1]

1-(1,1-dimethylethyl) 3-methyl 5-fluoro-1,3-piperidinedicarboxylate (289 mg, 1.106 mmol) was taken up in Tetrahydrofuran (THF) (5 mL) and allowed to stir at 0° C. for 10 mins. Lithium borohydride (1.106 mL, 2.212 mmol) was then added and the reaction allowed to warm to rt over 3 h. The reaction was quenched with dropwise addition of water (effervesence) followed by aqueous ammonium chloride (50 ml). The product was extracted with DCM (50 ml), dried using a hydrophobic frit and concentrated to a yellow oil, 220 mg

LCMS (Method B): Rt=0.79 min, MH⁺=234 (weak)

### Intermediate 51: 1,1-dimethylethyl 5-(aminocarbonyl)-2-methyl-1-piperidinecarboxylate

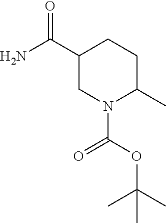 [SMILES: CC1CCC(C(N)=O)CN1C(=O)OC(C)(C)C]

6-methyl-3-piperidinecarboxamide (Commercial e.g. Enamine Building Blocks) (995 mg, 7.00 mmol) was suspended in N,N-Dimethylformamide (DMF) (3 mL) and to this was added triethylamine (1.463 mL, 10.50 mmol) and stirred under nitrogen. bis(1,1-dimethylethyl)dicarbonate (1527 mg, 7.00 mmol) was added and the solid quickly dissolved—the reaction was left stirring overnight. The mixture was reduced in vacuo and placed under high vacuum overnight to give a colourless gum. The gum was partitioned between ethyl acetate (50 ml) and water (50 ml). The layers were separated and the aqueous reextracted with ethyl acetate (2×50 ml). The combined organics were passed through a phase separation cartridge and reduced in vacuo to give a colourless gum. This was diluted with DCM (50 ml) and water (50 ml). The layers were separated and the aqueous reextracted with DCM (2×50 ml). The combined organics were washed with water (2×100 ml) and then with brine (2×100 ml). The organics were then passed through a phase separation cartridge and reduced in vacuo. The mixture was placed under high vacuum overnight to give the title compound as a colourless solid (1.391 g).

LCMS (Method B): Rt=0.84 min, MH⁺=243.3

### Intermediate 52: 1,1-dimethylethyl 5-(aminomethyl)-2-methyl-1-piperidinecarboxylate

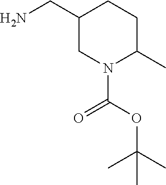 [SMILES: CC1CCC(CN)CN1C(=O)OC(C)(C)C]

1,1-dimethylethyl 5-(aminocarbonyl)-2-methyl-1-piperidinecarboxylate (1.22 g, 5.03 mmol) was dissolved in Tetrahydrofuran (THF) (45 mL) and 1M borane-tetrahydrofuran complex in THF (25.2 mL, 25.2 mmol) was added slowly and left to stir overnight. Further 1M borane-tetrahydrofuran complex in THF (15 mL, 15.00 mmol) was added and stirred at reflux for a further 4 days. The reaction was cooled to 0° C. and quenched with addition of methanol (50 ml) and stirred for 30 min and then 1M HCl and stirred for 1 h. The mixture was reduced in vacuo to give a white solid. The solid was partitioned between ethyl acetate (50 ml) and water (50 ml) and separated. The aqueous was washed with ethyl acetate (50 ml)

The aqueous was neutralised to pH7 using 2M sodium hydroxide and then extracted with DCM (3×50 ml) and then with ethyl acetate (4×50 ml). The extractions were combined and reduced in vacuo to give the title compound, 821 mg

LCMS (Method B): Rt=0.66 min, MH⁺=229.2

### Intermediate 53: [(2S)-6,6-dimethyl-4-(phenylmethyl)-2-morpholinyl]methanol

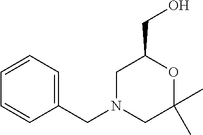 [SMILES: CC1(C)CN(Cc2ccccc2)C[C@@H](CO)O1]

To a solution of 2-methyl-1-[(phenylmethyl)amino]-2-propanol (Commercial e.g. American Custom Chemicals Corp.) (2.5 g, 13.95 mmol) in toluene (80 mL) was added (2R)-2-(chloromethyl)oxirane (Commercial e.g. Aldrich) (1.422 mL, 18.13 mmol) and lithium perchlorate (1.484 g, 13.95 mmol). This was stirred at ambient temperature for 3 days after which time the most of the starting material had disappeared. Sodium methoxide in methanol (7.97 mL, 34.9 mmol) was added and this was stirred at ambient temperature for 18 h. The reaction mixture was quenched with aqueous ammonium chloride and extracted twice with ethyl acetate. The combined organics were passed through a hydrophobic frit and concentrated in vacuo to yield a crude product. This was dissolved in DCM and purified through silica (50 g) eluting with a 0-60% gradient of ethyl acetate in cyclohexane. Appropriate fractions were combined and concentrated in vacuo to yield the title compound as a clear oil, 1.5 g

LCMS (Method C): Rt=0.50 min, MH⁺=236

### Intermediate 54: [(2S)-6,6-dimethyl-2-morpholinyl]methanol

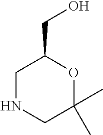

A solution of [(2S)-6,6-dimethyl-4-(phenylmethyl)-2-morpholinyl]methanol (1.4 g, 5.95 mmol) in Ethanol (30 mL) and hydrochloric acid (0.545 mL, 6.54 mmol) was hydrogenated over palladium on carbon (0.253 g, 0.238 mmol) under an atmosphere of hydrogen for 28 h. It was then filtered through celite (10 g) and washed with 4×40 ml of ethanol. The fractions with no UV absorbance which stained with permanganate were combined and concentrated in vacuo to yield a crude product as the hydrochloride salt. This was dissolved in methanol and loaded onto an aminopropyl cartridge (20 g). It was eluted with methanol (3×30 ml). The filtrate was concentrated in vacuo to yield the title compound as a clear gum, 0.822 g

¹H NMR (DMSO-d6) δ: 4.49 (br. s., 1H), 3.54 (dddd, J=10.5, 6.0, 5.0, 2.5 Hz, 1H), 3.23-3.34 (m, 1H), 3.10-3.22 (m, 1H), 2.81 (dd, J=12.0, 2.0 Hz, 1H), 2.49-2.53 (m, 1H), 2.33 (d, J=12.0 Hz, 1H), 2.13 (dd, J=12.0, 10.5 Hz, 1H), 1.20 (s, 3H), 1.02 (s, 3H)

### Intermediate 55: 1,1-dimethylethyl (6S)-6-(hydroxymethyl)-2,2-dimethyl-4-morpholinecarboxylate

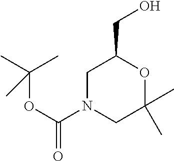 [SMILES: CC(C)(C)OC(=O)N1C[C@@H](CO)OC(C)(C)C1]

To an ice cooled solution of [(2S)-6,6-dimethyl-2-morpholinyl]methanol (0.82 g, 5.65 mmol) in Dichloromethane (DCM) (30 mL) and triethylamine (1.574 mL, 11.29 mmol) was added Boc-anhydride (1.377 mL, 5.93 mmol). This was warmed to ambient temperature and stirred for 18 h. Further triethylamine (0.8 ml), BOC anhydride (0.35 g) and also some DMAP (0.03 g) were added and stirring was continued for 22 h. 1M aqueous sodium hydroxide (10 ml, 10 mmol) was added and to the vigorously stirred biphasic mixture was added further BOC anhydride (1 m. eq.) and vigorous stirring was continued for 4 h. It was diluted with water and DCM. The layers were separated and the aqueous was reextracted with DCM. The combined organics were passed through a hydrophobic frit and concentrated in vacuo to yield a crude product. This was dissolved in DCM and purified through silica (50 g) eluting with a 0-100% gradient of ethyl acetate in DCM. Appropriate fractions were combined and concentrated in vacuo to yield the title compound as a clear oil, 562 mg

¹H NMR (DMSO-d6) δ: 4.70 (t, J=5.6 Hz, 1H), 3.82-4.02 (m, 1H), 3.51-3.74 (m, 2H), 3.33-3.41 (m, 1H), 3.18-3.29 (m, 1H), 2.35-2.73 (m, 2H), 1.40 (s, 9H), 1.11 (s, 6H)

### Example 1

### 7-[1-(Phenylmethyl)-1H-pyrazol-4-yl]-N-[(3S)-3-piperidinylmethyl]pyrido[3,4-b]pyrazin-5-amine

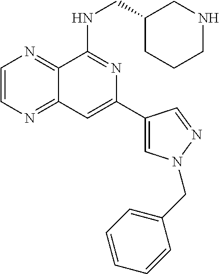 [SMILES: c1ccc(Cn2cc(-c3cc4nccnc4c(NC[C@H]4CCCNC4)n3)cn2)cc1]

To a solution of 1,1-dimethylethyl (3R)-3-[({7-[1-(phenylmethyl)-1H-pyrazol-4-yl]pyrido[3,4-b]pyrazin-5-yl}amino)methyl]-1-piperidinecarboxylate (1.27 g, 2.54 mmol) in dichloromethane (DCM) (7.5 ml) was added trifluoroacetic acid (4.50 ml, 58.5 mmol) and this was stirred at ambient temperature for 2 h. After this time the reaction had gone to completion and so was concentrated in vacuo to yield the crude product. This was dissolved in methanol and loaded onto an SCX cartridge (50 g). It was washed with methanol (3 column volumes) and product eluted as free base with 2M ammonia in methanol. The filtrate from the ammonia fractions was concentrated in vacuo to yield the title compound as yellow oil (1 g).

¹H NMR (CDCl₃): 8.75 ppm (1H, d, CH); 8.43 ppm (1H, d, CH); 8.05 ppm (1H, s, CH); 7.92 ppm (1H, s, CH); 7.36-7.22 ppm (5H, m, 5×CH); 7.13 ppm (1H, s, CH); 6.76 ppm (1H, br.t, NH); 5.33 ppm (2H, s, CH₂); 3.52 ppm (1H, m, CH); 3.15 ppm (1H, br.dm, CH); 2.98 ppm (1H, br.dt, CH); 2.56 ppm (1H, dt, CH); 2.43 ppm (1H, dd, CH); 1.89 ppm (1H, br.m, CH); 1.68 ppm (1H, br.m, CH); 1.45 ppm (1H, br.m, CH); 1.21 ppm (1H, br.m, CH).

LCMS (Method A): Rt=0.95 min, MH⁺=400

### Example 2

### 7-(1-Cyclopentyl-1H-pyrazol-4-yl)-N-[(3S)-3-piperidinylmethyl]pyrido[3,4-b]pyrazin-5-amine

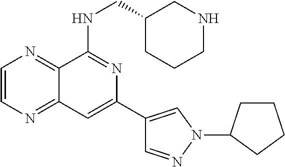 [SMILES: c1cnc2c(NC[C@H]3CCCNC3)nc(-c3cnn(C4CCCC4)c3)cc2n1]

1,1-dimethylethyl (3R)-3-({[7-(1-cyclopentyl-1H-pyrazol-4-yl)pyrido[3,4-b]pyrazin-5-yl]amino}methyl)-1-piperidinecarboxylate (1.07 g, 2.240 mmol) was dissolved in dichloromethane (DCM) (6 ml) and trifluoroacetic acid (2.416 ml, 31.4 mmol) was added. The reaction was stirred at r.t. for 30 min. The solvent was evaporated to give a red oil (2.2 g). The oil was loaded in methanol and purified by SPE on a sulphonic acid SCX column (10 g) using sequential solvents methanol, 2M ammonia/methanol. The NH₃/MeOH fractions were tested for UV activity by TLC, then appropriate fractions were combined and evaporated to give the title compound as a yellow oil (640 mg).

LCMS (Method B): Rt=0.71 min, MH⁺=377.91

¹H-NMR (DMSO-d6): 8.91 ppm (1H, br.s, CH); 8.63 ppm (1H, br.s, CH); 8.35 ppm (1H, s, CH); 8.06 ppm (1H, s, CH); 7.89 ppm (1H, br.t, NH); 7.22 ppm (1H, s, CH); 4.77 ppm (1H, m, CH); 3.46 ppm (2H, m, CH₂); 2.95 ppm (1H, br.d, ½CH₂); 2.80 ppm (1H, br.d, ½CH₂); 2.43 ppm (1H, br.t, ½CH₂); 2.29 ppm (1H, br.t, ½CH₂); 2.18-1.10 ppm (13H, 7×m, CH+6×CH₂).

### Example 3

### N-{[(3S)-3-fluoro-3-piperidinyl]methyl}-7-[1-(2,2,2-trifluoroethyl)-1H-pyrazol-4-yl]pyrido[3,4-b]pyrazin-5-amine

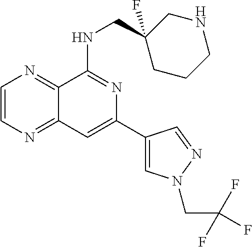 [SMILES: FC(F)(F)Cn1cc(-c2cc3nccnc3c(NC[C@]3(F)CCCNC3)n2)cn1]

To a solution of 1,1-dimethylethyl (3R)-3-{[(7-chloropyrido[3,4-b]pyrazin-5-yl)amino]methyl}-3-fluoro-1-piperidinecarboxylate (120 mg, 0.303 mmol) in 1,4-Dioxane (2 ml) was added 4-(4,4,5,5-tetramethyl-1,3,2-dioxaborolan-2-yl)-1-(2,2,2-trifluoroethyl)-1H-pyrazole (84 mg, 0.303 mmol), caesium carbonate (198 mg, 0.606 mmol) and water. Nitrogen was bubbled through for 1 min before adding tetrakis(triphenylphosphine)palladium (0) (10.51 mg, 9.09 μmol). This was heated in a microwave at 130° C. for 1.5 h. The reaction had gone to completion and so was partitioned between ethyl acetate and aqueous ammonium chloride. The aqueous was re-extracted with ethyl acetate and the combined organics were washed with brine, passed through a hydrophobic frit and concentrated in vacuo to give the crude product as a bright yellow gum (140 mg). This was dissolved in DCM (2 ml) and to it was added trifluoroacetic acid (2 ml) and the reaction was stirred at room temperature for 1 h. The mixture was concentrated in vacuo and the residue was dissolved in methanol and purified through an SCX cartridge (10 g) washing with methanol (3 column volumes). The product was eluted as the free base with 2M ammonia in methanol. This was concentrated in vacuo to give the title compound as a bright yellow gum (99 mg).

LCMS (Method C): Rt=0.67 min, MH⁺=409.8

### Example 4

### 7-[3,4-bis(Methyloxy)phenyl]-N-{[(3S)-3-fluoro-3-piperidinyl]methyl}pyrido[3,4-b]pyrazin-5-amine hydrochloride

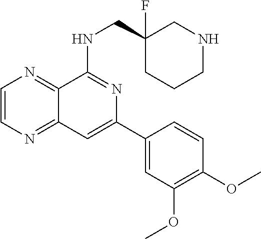 [SMILES: COc1ccc(-c2cc3nccnc3c(NC[C@]3(F)CCCNC3)n2)cc1OC]

To a solution of 1,1-dimethylethyl (3R)-3-[({7-[3,4-bis(methyloxy)phenyl]pyrido[3,4-b]pyrazin-5-yl}amino)methyl]-3-fluoro-1-piperidinecarboxylate (171 mg, 0.344 mmol) in dichloromethane (DCM) (1.2 ml) was added trifluoroacetic acid (0.609 ml, 7.90 mmol) and this was stirred at ambient temperature for 2 h. After this time the reaction had gone to completion and so was concentrated in vacuo to yield the crude product. This was dissolved in methanol and loaded onto an SCX cartridge (10 g) and washed with methanol and eluted with 2M ammonia in methanol. The filtrate from the ammonia fractions was concentrated in vacuo to yield a yellow oil. This free base was dissolved in dichloromethane (DCM) (1.5 ml) and to this was added HCl (1.0M in Et₂O) (0.344 ml, 0.344 mmol). A yellow solid immediately precipitated. The residual solvents were blown off and the resultant yellow solid dried in vacuo to give the title compound as a yellow solid (148 mg).

LCMS (Method B): Rt=0.69 min, MH⁺=398

### Example 5

### N-{[(3S)-3-Fluoro-3-piperidinyl]methyl}-7-(1-methyl-1H-pyrazol-4-yl)pyrido[3,4-b]pyrazin-5-amine hydrochloride

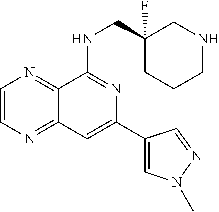 [SMILES: Cn1cc(-c2cc3nccnc3c(NC[C@]3(F)CCCNC3)n2)cn1]

To 1,1-dimethylethyl (3R)-3-fluoro-3-({[7-(1-methyl-1H-pyrazol-4-yl)pyrido[3,4-b]pyrazin-5-yl]amino}methyl)-1-piperidinecarboxylate (650 mg, 1.472 mmol) in dichloromethane (DCM) (5 ml) was added trifluoroacetic acid (2 ml, 26.0 mmol) and the reaction was left standing at room temperature for 30 min. LCMS showed half reaction. Additional TFA (1 ml) was added and the reaction was left standing for 3 h. LCMS showed complete conversion to product. The solvent was removed and the residue was dissolved in methanol and loaded onto a 10 g SCX cartridge. The column was washed with methanol and eluted with 2M methanolic ammonia. The solvent was removed and the residue was dried under high vacuum overnight to give the free base (452 mg). The free base was dissolved in DCM and ethereal HCl (1.32 ml, 1.0M) was added. The solvent was removed and the residue was dried under high vacuum overnight to give the title compound as a dark red solid (511 mg).

LCMS (Method B): Rt=0.53 min, MH⁺=342

### Example 6

### 5-{[(3S)-3-piperidinylmethyl]oxy}-7-[1-(2,2,2-trifluoroethyl)-1H-pyrazol-4-yl]pyrido[3,4-b]pyrazine hydrochloride

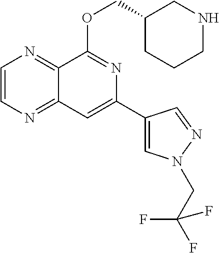 [SMILES: FC(F)(F)Cn1cc(-c2cc3nccnc3c(OC[C@H]3CCCNC3)n2)cn1]

1,1-Dimethylethyl (3S)-3-[({7-[1-(2,2,2-trifluoroethyl)-1H-pyrazol-4-yl]pyrido[3,4-b]pyrazin-5-yl}oxy)methyl]-1-piperidinecarboxylate (174.6 mg, 0.355 mmol) was dissolved in dichloromethane (5 ml) and trifluoroacetic acid (0.55 ml, 7.09 mmol) was added. The reaction was stirred for 30 min at 20° C. The solvent was evaporated. The mixture was loaded on to a 10 g SCX cartridge and washed with methanol and 2M methanolic ammonia. The basic fractions were combined, evaporated and dried under high vacuum overnight to give a yellow oil (104.4 mg). This was loaded on to a 10 g silica column and purified on the SP4 eluting with 5-40% ammonia in methanol/DCM gradient. Appropriate fractions were combined and evaporated to give a yellow oil which was dried under high vacuum overnight (17.8 mg). This was dissolved in DCM and HCl in diethyl ether (0.048 ml, 0.048 mmol) was added. The solvent was blown down to give the title compound as the hydrochloride salt as a yellow solid (21.8 mg).

LCMS (Method B): Rt=0.64 min, MH⁺=393

### Example 7

### 7-(1,5-Dimethyl-1H-pyrazol-4-yl)-5-{[(3S)-3-piperidinylmethyl]oxy}pyrido[3,4-b]pyrazine, hydrochloride

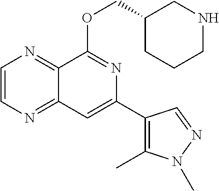 [SMILES: Cc1c(-c2cc3nccnc3c(OC[C@H]3CCCNC3)n2)cnn1C]

To 1,1-dimethylethyl (3S)-3-{[(7-chloropyrido[3,4-b]pyrazin-5-yl)oxy]methyl}-1-piperidinecarboxylate (150 mg, 0.396 mmol) was added tetrakis(triphenylphosphine)palladium(0) (45.8 mg, 0.040 mmol), caesium carbonate (0.594 mL, 1.188 mmol, 2M aqueous solution) and 1,5-dimethyl-4-(4,4,5,5-tetramethyl-1,3,2-dioxaborolan-2-yl)-1H-pyrazole (106 mg, 0.475 mmol) (Boron Molecular Pty Ltd). To the mixture was added 1,4-dioxane (1.5 ml). The reaction vessel was sealed and heated in the microwave at 130° C. for 60 min. The reaction mixture was partitioned between water (25 ml) and ethyl acetate (30 ml) and then separated. The aqueous was extracted with ethyl acetate (2×20 ml). The combined organics were passed through a phase separation cartridge and reduced in vacuo. The resulting residue was dissolved in DCM and loaded onto a silica cartridge (25 g) and purified using a 0-6% methanol/DCM gradient. The appropriate fractions were combined and concentrated to give a yellow film. This was dissolved in DCM and trifluoroacetic acid (0.031 ml, 0.396 mmol) was added. The mixture was left to stir under nitrogen for 15 min. The solvent was removed in vacuo to give the product as the TFA salt. The sample was loaded onto a 5 g SCX cartridge, washed with MeOH (70 ml) and eluted with 10% 2M NH₃ in MeOH (70 ml). The ammonia/methanol fractions were concentrated to yield the free base of the product as a yellow film. This was purified further by MDAP (Method B). The solvent was evaporated in vacuo and the residue was dissolved in methanol and loaded onto an SCX cartridge, washed with methanol and eluted with 2M ammonia in methanol. The fractions were collected and concentrated. 2M HCl in Et₂O (0.5 ml) was added to the resulting residue and the solvent was evaporated to give the title compound as an orange solid (104 mg).

LCMS (Method C): Rt=0.6 min, MH⁺=339.1

### Example 8

### N,N-Dimethyl-5-(5-{[(3S)-3-piperidinylmethyl]oxy}pyrido[3,4-b]pyrazin-7-yl)-2-pyridinamine hydrochloride

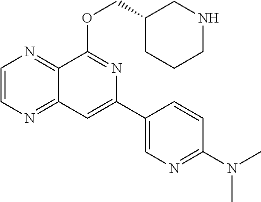 [SMILES: CN(C)c1ccc(-c2cc3nccnc3c(OC[C@H]3CCCNC3)n2)cn1]

1,1-Dimethylethyl (3S)-3-{[(7-chloropyrido[3,4-b]pyrazin-5-yl)oxy]methyl}-1-piperidinecarboxylate (2 g, 5.28 mmol) was taken up in 1,4-dioxane (40 ml) and water (5 ml) and treated with [6-(dimethylamino)-3-pyridinyl]boronic acid hydrate (1.263 g, 6.86 mmol) (Frontier Scientific Europe) and caesium carbonate (5.16 g, 15.84 mmol), N₂ (g) was bubbled through for 5 min before adding tetrakis(triphenylphosphine)palladium (0) (0.305 g, 0.264 mmol). The resulting suspension was allowed to stir at 80° C. for 1 h. The reaction was concentrated and partitioned between water (200 ml) and DCM (200 ml). The organic layer was washed with water (100 ml), dried using a hydrophobic frit and concentrated to give a black gum. This gum was purified on silica (100 g) using an SP4 and eluted with a 5-20% (20% 2M methanolic ammonia in DCM)/DCM gradient. Appropriate fractions were summed and concentrated to give an orange gum (1.810 g). The gum was taken up in TFA (5 ml, 64.9 mmol) and allowed to stand at r.t. for 15 min. The reaction was concentrated and eluted through an SCX SPE (20 g) using methanol and 2M ammonia in methanol. The ammonia fraction was concentrated to give a yellow solid. The mono HCl salt was made and was triturated with ether (50 ml) to give the title compound as a yellow solid (950 mg).

LCMS (Method C): Rt=0.49 min, MH⁺=365.02

### Example 9

### 7-(1-Methyl-1H-pyrazol-4-yl)-5-{[(3S)-3-piperidinylmethyl]oxy}pyrido[3,4-b]pyrazine hydrochloride

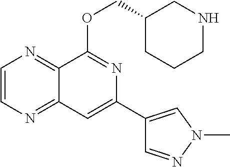 [SMILES: Cn1cc(-c2cc3nccnc3c(OC[C@H]3CCCNC3)n2)cn1]

1,1-Dimethylethyl (3S)-3-{[(7-chloropyrido[3,4-b]pyrazin-5-yl)oxy]methyl}-1-piperidinecarboxylate (200 mg, 0.528 mmol) was taken up in 1,4-dioxane (5 ml) and treated with 1-methyl-4-(4,4,5,5-tetramethyl-1,3,2-dioxaborolan-2-yl)-1H-pyrazole (154 mg, 0.739 mmol), caesium carbonate (516 mg, 1.584 mmol) and tetrakis(triphenylphosphine)palladium (0) (61.0 mg, 0.053 mmol). The resulting suspension was irradiated in a biotage microwave at 150° C. for 30 min. The reaction was concentrated and partitioned between water (100 ml) and DCM (100 ml). The organic layer was dried using a hydrophobic frit and concentrated to a brown oil. This oil was purified on silica (25 g) using a 1-4% 2M methanolic ammonia in DCM gradient. The appropriate fractions were summed and concentrated to give a green oil. This oil was taken up in 1.25M HCl/MeOH (10 ml) and allowed to stir at 50° C. for 1 h. The reaction was concentrated and eluted through a SCX SPE (5 g) using methanol (20 ml) and 2M NH₃/MeOH (20 ml). The ammonia fraction was concentrated to give a yellow gum (103 mg). This was further purified by MDAP (Method E). The appropriate fractions were concentrated, made the free base using an aminopropyl column and then made into the mono HCl salt to give the title compound as a yellow solid (51 mg).

LCMS (Method B): Rt=0.54 min, MH⁺=325.01

### Example 10

### 7-(1,5-Dimethyl-1H-pyrazol-4-yl)-5-{[(2S)-2-morpholinylmethyl]oxy}pyrido[3,4-b]pyrazine, hydrochloride

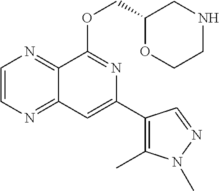 [SMILES: Cc1c(-c2cc3nccnc3c(OC[C@@H]3CNCCO3)n2)cnn1C]

To 1,1-dimethylethyl (2S)-2-({[7-(1,5-dimethyl-1H-pyrazol-4-yl)pyrido[3,4-b]pyrazin-5-yl]oxy}methyl)-4-morpholinecarboxylate (104 mg, 0.24 mmol) in DCM (2 mL) was added trifluoroacetic acid (0.4 mL, 5.19 mmol) and stirred at ambient temperature for 2 h. The solvent was removed in vacuo and the residue was loaded in methanol onto a SCX cartridge (1 g). It was washed with methanol and the product eluted as free base with 2M ammonia in methanol. The filtrate from the ammonia fractions was concentrated in vacuo to give a yellow solid. This was dissolved in DCM and 2M ethereal hydrogen chloride (0.15 mL, 0.29 mmol) and solvent removed. The sample was again dissolved in methanol and loaded onto a SCX cartridge (2 g). It was washed with methanol and the product eluted as free base with 2M ammonia in methanol. The filtrate from the ammonia fractions was concentrated in vacuo to give a yellow solid. This was dissolved in DCM and 2M ethereal hydrogen chloride (0.12 mL, 0.23 mmol) and solvent removed to give the title compound as an orange solid (39 mg).

¹H NMR (d6-DMSO): 9.67-9.47 ppm (2H, br m); 9.06 ppm (1H, s); 8.88 ppm (1H, s); 8.10 ppm (1H, s); 7.72 ppm (1H, s); 4.63 ppm (2H, br. m); 4.32 ppm (1H, br. m); 4.04 ppm (1H, br. d); 3.90-3.80 ppm (4H, m); 3.42 ppm (1H, br. d); 3.23 ppm (1H, br. d); 3.13-2.96 ppm (2H, br.m); 2.68 ppm (3H, s).

LCMS (Method A): Rt=0.67 min, MH⁺ 341.05

### Example 11

### 7-(1-Methyl-1H-pyrazol-4-yl)-N-[(2S)-2-morpholinylmethyl]pyrido[3,4-b]pyrazin-5-amine, hydrochloride

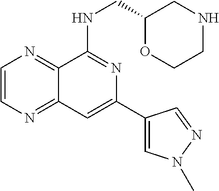 [SMILES: Cn1cc(-c2cc3nccnc3c(NC[C@@H]3CNCCO3)n2)cn1]

To an ice cooled solution of 1,1-dimethylethyl (2R)-2-({[7-(1-methyl-1H-pyrazol-4-yl)pyrido[3,4-b]pyrazin-5-yl]amino}methyl)-4-morpholinecarboxylate (6.5 g, 15.28 mmol) in Dichloromethane (DCM) (30 mL) was added trifluoroacetic acid (8 mL, 104 mmol). This was then warmed to ambient temperature and stirred. After 20 min there was no reaction and so further TFA (7 ml) was added and stirring was continued for a total of 20 h after which time the reaction had gone to completion. It was concentrated in vacuo, redissolved in DCM and passed through an aminopropyl cartridge (50 g) eluting with methanol. The combined filtrate was concentrated in vacuo. To ensure all of the TFA was removed, it was partitioned between DCM and aqueous sodium bicarbonate, stirring for 30 min. The layers were separated and the aqueous was reextracted with DCM. The combined organics were washed with brine, dried over sodium sulfate and concentrated and dried in vacuo to yield a greyish green solid, 5.00 g. To a portion of the solid (0.52 g) in DCM (10 ml) was added methanolic hydrogen chloride (1.25M, 1.2 ml, 1 m.eq.) and this was stirred and blown down under nitrogen to yield the title compound, orange-yellow solid, 520 mg

LCMS (Method C): rt=0.49 min, MH⁺=326

### Example 12

### N-{[(2S)-1-methyl-2-piperazinyl]methyl}-7-(1-methyl-1H-pyrazol-4-yl)pyrido[3,4-b]pyrazin-5-amine hydrochloride

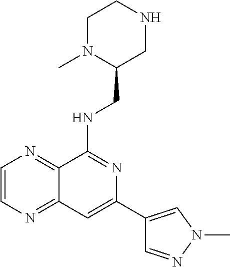 [SMILES: CN1CCNC[C@H]1CNc1nc(-c2cnn(C)c2)cc2nccnc12]

1,1-Dimethylethyl (3R)-3-{[(7-chloropyrido[3,4-b]pyrazin-5-yl)amino]methyl}-4-methyl-1-piperazinecarboxylate (79 mg, 0.201 mmol) was dissolved in 1,4-dioxane (2.5 ml) and caesium carbonate (197 mg, 0.603 mmol), tetrakis(triphenylphosphine)palladium (0) (23 mg, 0.020 mmol) and 1-methyl-4-(4,4,5,5-tetramethyl-1,3,2-dioxaborolan-2-yl)-1H-pyrazole (54 mg, 0.260 mmol) were added. The resulting suspension was heated in the biotage microwave at 130° C. for 30 min. LCMS showed reaction had gone ˜30%. The reaction was heated at 130° C. for 30 min. LCMS showed incomplete reaction. The reaction was heated at 130° C. for a further 30 min. LCMS still showed starting material. The reaction was heated at 130° C. for a further 2 h. LCMS still showed starting material, so the reaction was worked up. The reaction was concentrated and partitioned between DCM (50 ml) and water (50 ml). The organic layer was washed with water, dried using a hydrophobic frit and concentrated to give an orange gum. The residue was loaded in dichloromethane and purified on silica (10 g) using a 0-4% (2M ammonia methanol) in DCM gradient. Appropriate fractions were combined and evaporated to give an orange oil. The BOC protected compound was taken up in 1.25M HCl/MeOH and allowed to stir at 50° C. for 2 h. The reaction mixture was eluted through SCX SPE (5 g) using MeOH (15 ml) and 2M NH₃/MeOH (15 ml). The ammonia fraction was concentrated to give a yellow gum and made into the mono HCl salt to give the title compound as an orange solid (30 mg).

LCMS (Method A): Rt=0.65 min, MH⁺=339

### Example 13

### 5-{[(4,4-Difluoro-3-piperidinyl)methyl]oxy}-7-(1-methyl-1H-pyrazol-4-yl)pyrido[3,4-b]pyrazine

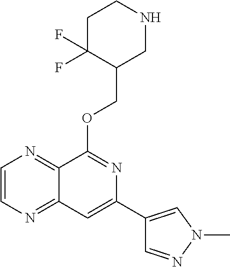 [SMILES: Cn1cc(-c2cc3nccnc3c(OCC3CNCCC3(F)F)n2)cn1]

1,1-Dimethylethyl 3-{[(7-chloropyrido[3,4-b]pyrazin-5-yl)oxy]methyl}-4,4-difluoro-1-piperidinecarboxylate (161 mg, 0.388 mmol) was dissolved in 1,4-dioxane (2 ml) and water (0.5 ml). 1-methyl-4-(4,4,5,5-tetramethyl-1,3,2-dioxaborolan-2-yl)-1H-pyrazole (105 mg, 0.505 mmol), caesium carbonate (379 mg, 1.164 mmol) and tetrakis(triphenylphosphine)palladium (0) (44.8 mg, 0.039 mmol) were added and the reaction was heated at 130° C. in the microwave for 1 h. The reaction was concentrated and partitioned between DCM (50 ml) and water (50 ml). The organic layer was washed with water, dried using a hydrophobic frit and concentrated to give a orange gum. The residue was loaded in dichloromethane and purified on silica (25 g) column using a 0-20% (20% 2M ammonia methanol) in DCM/dichloromethane gradient. Appropriate fractions were combined and evaporated in vacuo to give a yellow oil. This was dissolved in DCM (2 ml) and trifluoroacetic acid (1 ml, 12.98 mmol) was added and left standing at room temperature for 1 h. The solvent was evaporated in vacuo to give an orange oil. This was loaded in methanol and purified by SPE on sulphonic acid (SCX) 5 g using sequential solvents methanol, 2M ammonia/methanol. The NH₃/MeOH fraction was evaporated in vacuo to give a yellow oil (129 mg). The oil was purified further by MDAP (Method A). Appropriate fractions were combined and the solvent was evaporated in vacuo to give the title compound as a pale yellow solid (105 mg).

LCMS (Method A): Rt=0.79 min, MH⁺=361

### Example 14

### 7-(1-Methyl-H-pyrazol-4-yl)-5-[2-(3-piperidinyl)ethyl]pyrido[3,4-b]pyrazine hydrochloride

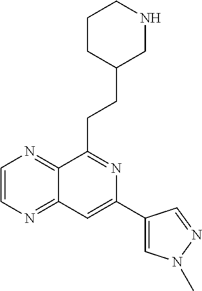 [SMILES: Cn1cc(-c2cc3nccnc3c(CCC3CCCNC3)n2)cn1]

1,1-Dimethylethyl 3-[2-(7-chloropyrido[3,4-b]pyrazin-5-yl)ethyl]-1-piperidinecarboxylate (500 mg, 1.327 mmol) was taken up in 1,4-dioxane (10 ml) and treated with 1-methyl-4-(4,4,5,5-tetramethyl-1,3,2-dioxaborolan-2-yl)-1H-pyrazole (331 mg, 1.592 mmol), tetrakis(triphenylphosphine)palladium (0) (153 mg, 0.133 mmol) and caesium carbonate (1297 mg, 3.98 mmol). The resulting suspension was irradiated in a biotage microwave at 130° C. for 30 min. The reaction was concentrated and partitioned between water (100 ml) and DCM (100 ml). The organic phase was washed with brine (100 ml), dried using a hydrophobic frit and concentrated to a black gum. This gum was treated with TFA (1 ml) and allowed to stir at room temperature for 2 h after which time conversion was complete. This was concentrated in vacuo to give a gum which was purified on silica (50 g) using a 1-5% (2M ammonia in methanol) in DCM gradient. The appropriate fractions were summed and concentrated to give a yellow gum. This gum was further purified by MDAP (Method B). Appropriate fractions were summed, concentrated and made into the mono HCl salt to give the title compound as a yellow solid (138 mg).

LCMS (Method A): Rt=0.70 min, MH⁺=323.26

### Example 15

### N-{7-[6-(Dimethylamino)-3-pyridinyl]pyrido[3,4-b]pyrazin-5-yl}-1,4-butanediamine hydrochloride

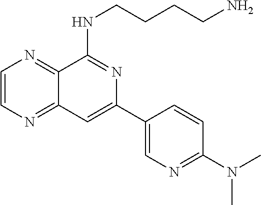 [SMILES: CN(C)c1ccc(-c2cc3nccnc3c(NCCCCN)n2)cn1]

To 1,1-dimethylethyl {4-[(7-chloropyrido[3,4-b]pyrazin-5-yl)amino]butyl}carbamate (250 mg, 0.711 mmol) was added tetrakis(triphenylphosphine)palladium (0) (82 mg, 0.071 mmol), aqueous caesium carbonate (2M, 1.066 ml, 2.132 mmol) and [6-(dimethylamino)-3-pyridinyl]boronic acid hydrate (177 mg, 0.853 mmol) (Boron Molecular). To the mixture was added 1,4-dioxane (2.5 ml). The reaction vessel was sealed and heated in the microwave at 130° C. for 60 min. The reaction mixture was partitioned between water (25 ml) and ethyl acetate (30 ml) and then separated. The aqueous layer was extracted with ethyl acetate (2×20 ml). The combined organics were passed through a phase separation cartridge and reduced in vacuo. The residue was dissolved in DCM and loaded onto a silica cartridge (25 g) and purified via SP4 using a 0-6% MeOH in DCM gradient. The appropriate fractions were combined and concentrated to give a yellow film. This was dissolved in DCM and trifluoroacetic acid (0.055 ml, 0.711 mmol) was added. The mixture was left to stir under nitrogen for 15 min. The solvent was removed in vacuo to to give the product as the TFA salt. The sample was loaded onto a 5 g SCX cartridge, washed with MeOH (70 ml) and eluted with 10% 2M NH₃ in MeOH (70 ml). The ammonia/methanol fractions were concentrated to yield the free base of the product as a yellow film. This was purified further by MDAP (Method B). The solvent was evaporated in vacuo and the residue was dissolved in methanol and washed through an SCX cartridge. The fractions were collected and concentrated. 2M HCl in Et₂O (0.5 ml) was added to the resulting residue and the solvent was evaporated to give the title compound as a yellow solid (134 mg).

LCMS (Method C): Rt=0.48 min, MH⁺=338.0

### Example 25

### 7-[6-(dimethylamino)-3-pyridinyl]-N-[(2)-2-morpholinylmethyl]pyrido[3,4-b]pyrazin-5-amine, hydrochloride (Isomer 1)

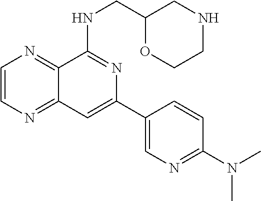 [SMILES: CN(C)c1ccc(-c2cc3nccnc3c(NCC3CNCCO3)n2)cn1]

To 1,1-dimethylethyl-2-[({7-[6-(dimethylamino)-3-pyridinyl]pyrido[3,4-b]pyrazin-5-yl}amino)methyl]-4-morpholinecarboxylate (Isomer 1) (13 mg, 0.03 mmol) was added 2M HCl in dioxan. After 2 h it was concentrated in vacuo, and loaded onto a 2 g SCX SPE cartridge, washed with methanol and eluted with 2M methanolic ammonia. The solvent was removed and the solid dissolved in DCM (1 ml). Ethereal hydrogen chloride (1.0M, 0.02 ml, 1 m.eq.) was added and this was evaporated to yield the title compound as an orange solid (8 mg).

LCMS (Method B): Rt=0.48 min, MH+=366

The following example was prepared similarly:

### Example 26

### 7-[6-(dimethylamino)-3-pyridinyl]-N-[2-morpholinylmethyl]pyrido[3,4-b]pyrazin-5-amine, hydrochloride (Isomer 2)

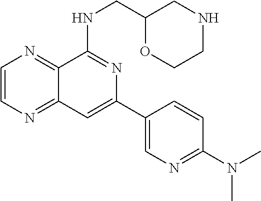 [SMILES: CN(C)c1ccc(-c2cc3nccnc3c(NCC3CNCCO3)n2)cn1]

LCMS (Method B): Rt=0.47 min, MH+=366

### Example 27

### N-[(4,4-difluoro-3-piperidinyl)methyl]-7-[6-(dimethylamino)-3-pyridinyl]pyrido[3,4-b]pyrazin-5-amine (Isomer 2)

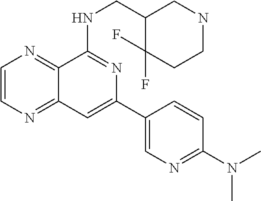 [SMILES: CN(C)c1ccc(-c2cc3nccnc3c(NCC3CNCCC3(F)F)n2)cn1]

A mixture of 1,1-dimethylethyl 3-{[(7-chloropyrido[3,4-b]pyrazin-5-yl)amino]methyl}-4,4-difluoro-1-piperidinecarboxylate (Isomer 2) (100 mg, 0.24 mmol), [6-(dimethylamino)-3-pyridinyl]boronic acid hydrate (48 mg, 0.29 mmol), tetrakis(triphenylphosphine)palladium (0) (28 mg, 0.02 mmol) and caesium carbonate (236 mg, 0.73 mmol) in 1,4-dioxane (2 mL) and water (0.5 mL) was irradiated in the microwave at 130° C. for 30 min. The crude mixture was cooled and partitioned between DCM and water×2 and the combined organic layers washed with brine and dried over a hydrophobic frit and concentrated in vacuo to yield a yellow/brown solid. This was purified through silica (40 g) eluting with a 10-50% ethyl acetate in cyclohexane gradient. Appropriate fractions were combined and concentrated in vacuo. The residue was dissolved in DCM (6 mL) and TFA (1 mL, 13.0 mmol) added and left to stand room temperature for 30 min. The solvent was removed and the residue dissolved in methanol and loaded onto a 5 g SCX SPE cartridge, washing with methanol and eluting with 2M methanolic ammonia. The basic layer was evaporated to give a yellow/orange solid, 99 mg.

LCMS (Method A): Rt=1.00 min, MH+=400

The following example was prepared similarly:

### Example 28

### N-[(4,4-difluoro-3-piperidinyl)methyl]-7-[6-(dimethylamino)-3-pyridinyl]pyrido[3,4-b]pyrazin-5-amine (Isomer 1)

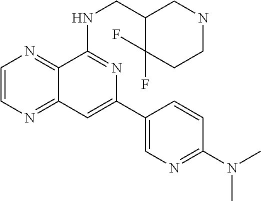 [SMILES: CN(C)c1ccc(-c2cc3nccnc3c(NCC3CNCCC3(F)F)n2)cn1]

LCMS (Method A): Rt=1.02 min, MH+=400

The following example was prepared similarly:

### Example 29

### N-((5,5-difluoropiperidin-3-yl)methyl)-7-(6-(dimethylamino)pyridin-3-yl)pyrido[3,4-b]pyrazin-5-amine

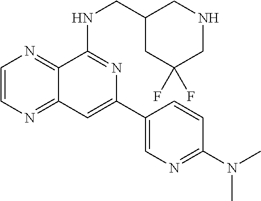 [SMILES: CN(C)c1ccc(-c2cc3nccnc3c(NCC3CNCC(F)(F)C3)n2)cn1]

LCMS (Method A): Rt=1.01 min, MH+=400

### Example 30

### 4-(5-{[(3S)-3-piperidinylmethyl]amino}pyrido[3,4-b]pyrazin-7-yl)-2-piperazinone, hydrochloride

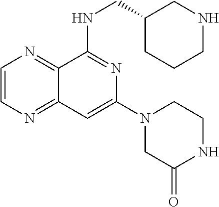 [SMILES: O=C1CN(c2cc3nccnc3c(NC[C@H]3CCCNC3)n2)CCN1]

1,1-Dimethylethyl (3R)-3-({[7-(3-oxo-1-piperazinyl)pyrido[3,4-b]pyrazin-5-yl]amino}methyl)-1-piperidinecarboxylate (91.2 mg, 0.207 mmol) and 5M HCl in propan-2-ol (3 ml, 99 mmol) were added together and the solution was left stirring, under N₂, at r. t., for 40 min. The reaction mixture was concentrated in vacuo and the oil formed was desalted using an aminopropyl cartridge (preconditioned, loaded and eluted with methanol). The eluted product was concentrated in vacuo and the monohydrochloride salt of the product made, yielding the title compound (82 mg).

LCMS (Method C): Rt=0.44 min, MH+=341.9

The following example was prepared similarly:

### Example 31

### 7-(1-piperazinyl)-N-[(3S)-3-piperidinylmethyl]pyrido[3,4-b]pyrazin-5-amine, hydrochloride

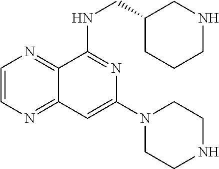 [SMILES: c1cnc2c(NC[C@H]3CCCNC3)nc(N3CCNCC3)cc2n1]

LCMS (Method A): Rt=0.67 min, MH+=328.3

### Example 32

### N,N-dimethyl-5-(5-{[(3R)-3-piperidinylmethyl]oxy}pyrido[3,4-b]pyrazin-7-yl)-2-pyridinamine

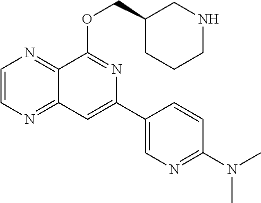 [SMILES: CN(C)c1ccc(-c2cc3nccnc3c(OC[C@@H]3CCCNC3)n2)cn1]

[6-(Dimethylamino)-3-pyridinyl]boronic acid) (87 mg, 0.475 mmol), 1,1-dimethylethyl (3R)-3-{[(7-chloropyrido[3,4-b]pyrazin-5-yl)oxy]methyl}-1-piperidinecarboxylate (150 mg, 0.396 mmol), sodium carbonate (126 mg, 1.188 mmol), and bis(triphenylphosphine)palladium (II) dichloride (27.8 mg, 0.040 mmol) were added to 1,2-dimethoxyethane (DME) (1.5 mL) and water (0.5 mL). The reaction mixture was heated in a microwave for 60 min at 130° C. The reaction was worked up with the addition of 40 ml of ethyl acetate. This was washed with water (3×30 ml) and brine (20 ml). The organics were passed through a hydrophobic frit and volatiles were removed under vacuum. The crude was dissolved in minimum DCM and loaded onto silica. A gradient was run of 1 CV of DCM then 0-4% 2M ammonia in methanol in DCM. The relevant fractions were combined and volatiles were removed under vacuum. TFA (2 ml) was added and the solution was left stirring for 20 min. The TFA was removed under vacuum and the product was desalted using an SCX cartridge (preconditioned, loaded and washed (2 CV) with methanol and eluted with 2M ammonia in methanol). The eluted product fractions were combined and volatiles were removed under vacuum to give a crude product that was purified by MDAP. Appropriate fractions were combined and concentrated in vacuo to yield the title compound (43 mg)

LCMS (Method C): Rt=0.51 min, MH⁺=365.1

Further Example compounds that were similarly prepared to Example 15, as the free base or a hydrochloride salt include:

| Example | Structure | Name | LCMS |
|---|---|---|---|
| 17 | 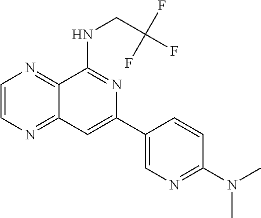 [SMILES: CN(C)c1ccc(-c2cc3nccnc3c(NCC(F)(F)F)n2)cn1] | 7-[6-(dimethylamino)- 3-pyridinyl]-N-(2,2,2- trifluoroethyl)pyrido[3, 4-b)]pyrazin-5-amine | LCMS (Method C): Rt = 0.72 min, [MH⁺] = 349 |
| 18 | 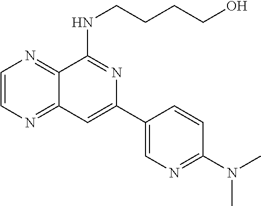 [SMILES: CN(C)c1ccc(-c2cc3nccnc3c(NCCCCO)n2)cn1] | 4-({7-[6- (dimethylamino)-3- pyridinyl]pyrido[3,4- 6]pyrazin-5-yl}amino)- 1-butanol | LCMS (Method B): Rt = 0.57 min, [MH⁺] = 339 |
| 19 | 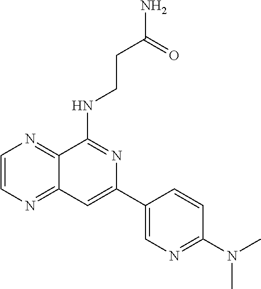 [SMILES: CN(C)c1ccc(-c2cc3nccnc3c(NCCC(N)=O)n2)cn1] | N³-{7-[6- (dimethylamino)-3- pyridinyl]pyrido[3,4- b]pyrazin-5-yl}-(β- alaninamide | LCMS (Method A): Rt = 0.81 min, [MH⁺] = 338.3 |
| 20 | 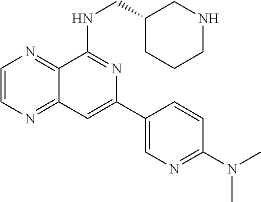 [SMILES: CN(C)c1ccc(-c2cc3nccnc3c(NC[C@H]3CCCNC3)n2)cn1] | 7-[6-(dimethylamino)- 3-pyridinyl]-N-[(3S)-3- piperidinylmethyl]pyrido [3,4-b]pyrazin-5- amine | LCMS (Method A): Rt = 0.89 min, [MH⁺] = 364.3 |
| 21 | 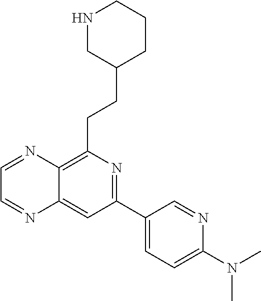 [SMILES: CN(C)c1ccc(-c2cc3nccnc3c(CCC3CCCNC3)n2)cn1] | N,N-dimethyl-5-{5-[2- (3- piperidinyl)ethyl]pyrido [3,4-6]pyrazin-7-yl}-2- pyridinamine | LCMS (Method B): Rt = 0.45 min, [MH⁺] = 363 |
| 22 | 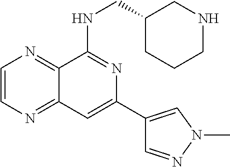 [SMILES: Cn1cc(-c2cc3nccnc3c(NC[C@H]3CCCNC3)n2)cn1] | 7-(1-methyl-1H- pyrazol-4-yl)-N- [(3S)-3- piperidinylmethyl] pyrido[3,4-b]pyrazin- 5-amine | LCMS (Method A): Rt = 0.69 min, [MH⁺] = 324.3 |
| 23 | 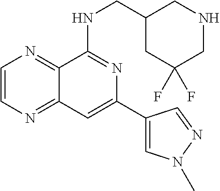 [SMILES: Cn1cc(-c2cc3nccnc3c(NCC3CNCC(F)(F)C3)n2)cn1] | N-[(5,5-difluoro-3- piperidinyl)methyl]-7- (1-methyl-1H-pyrazol- 4-yl)pyrido[3,4-b] pyrazin-5-amine | LCMS (Method A): Rt = 0.81 min, [MH⁺] = 360.3 |
| 24 | 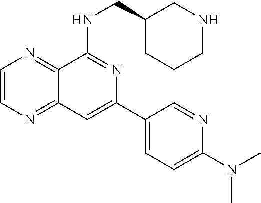 [SMILES: CN(C)c1ccc(-c2cc3nccnc3c(NC[C@@H]3CCCNC3)n2)cn1] | 7-[6-(dimethylamino)- 3-pyridinyl]-N-[(3R)- 3-piperidinylmethyl] pyrido[3,4-b] pyrazin-5-amine | LCMS (Method C): Rt = 0.51 min, [MH⁺] = 365 |
| 33 | 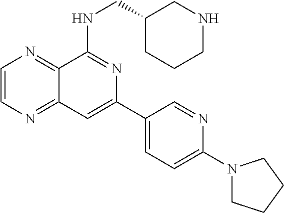 [SMILES: c1cnc2c(NC[C@H]3CCCNC3)nc(-c3ccc(N4CCCC4)nc3)cc2n1] | N-[(3S)-3- piperidinylmethyl]-7- [6-(1-pyrrolidinyl)-3- pyridinyl]pyrido[3,4- b]pyrazin-5-amine | LCMS (Method A): Rt = 0.98 min, [MH⁺] = 390.2 |
| 34 | 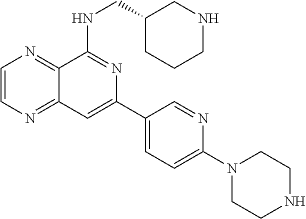 [SMILES: c1cnc2c(NC[C@H]3CCCNC3)nc(-c3ccc(N4CCNCC4)nc3)cc2n1] | 7-[6-(1-piperazinyl)- 3-pyridinyl]-N-[(3S)- 3-piperidinylmethyl] pyrido[3,4-b] pyrazin-5-amine | LCMS (Method B): Rt = 0.44 min, [2M + HCO2H] = 853.6 |
| 35 | 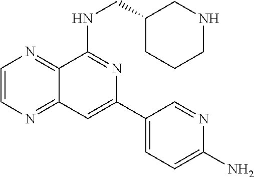 [SMILES: Nc1ccc(-c2cc3nccnc3c(NC[C@H]3CCCNC3)n2)cn1] | 7-(6-amino-3- pyridinyl)-N-[(3S)- 3-piperidinylmethyl] pyrido[3,4-b] pyrazin-5-amine | LCMS (Method B): Rt = 0.49 min, [MH⁺] = 335.9 |
| 36 | 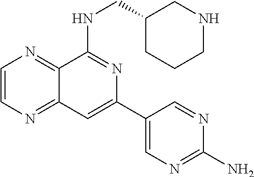 [SMILES: Nc1ncc(-c2cc3nccnc3c(NC[C@H]3CCCNC3)n2)cn1] | 7-(2-amino-5- pyrimidinyl)-N-[(3S)- 3-piperidinylmethyl] pyrido[3,4-b] pyrazin-5-amine | LCMS (Method B): Rt = 0.51 min, [MH⁺] = 337 |
| 37 | 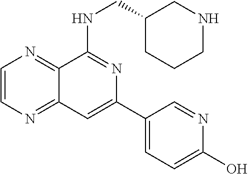 [SMILES: Oc1ccc(-c2cc3nccnc3c(NC[C@H]3CCCNC3)n2)cn1] | 5-(5-{[(3S)-3- piperidinylmethyl] amino}pyrido[3,4-b] pyrazin-7-yl)-2(1H)- pyridinone | LCMS (Method D): Rt = 1.19 min, [MH⁺] = 337 |
| 38 | 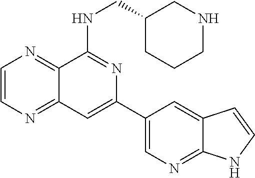 [SMILES: c1cnc2c(NC[C@H]3CCCNC3)nc(-c3cnc4[nH]ccc4c3)cc2n1] | N-[(3S)-3- piperidinylmethyl]- 7-(1H-pyrrolo [2,3-b]pyridin-5- yl)pyrido[3,4-b] pyrazin-5-amine | LCMS (Method B): Rt = 0.55 min, [MH⁺] = 360.2 |
| 39 | 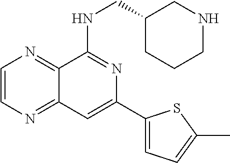 [SMILES: Cc1ccc(-c2cc3nccnc3c(NC[C@H]3CCCNC3)n2)s1] | 7-(5-methyl-2-thienyl)- N-[(3S)-3- piperidinylmethyl] pyrido[3,4-b]pyrazin- 5-amine | LCMS (Method B): Rt = 0.72 min, [MH⁺] = 340.1 |
| 40 | 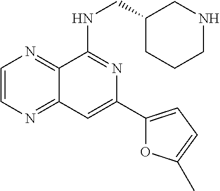 [SMILES: Cc1ccc(-c2cc3nccnc3c(NC[C@H]3CCCNC3)n2)o1] | 7-(5-methyl-2-furanyl)- N-[(3S)-3- piperidinylmethyl] pyrido[3,4-b]pyrazin- 5-amine | LCMS (Method B): Rt = 0.76 min, [MH⁺] = 324 |
| 41 | 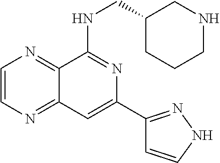 [SMILES: c1cnc2c(NC[C@H]3CCCNC3)nc(-c3cc[nH]n3)cc2n1] | N-[(3S)-3- piperidinylmethyl]-7- (1H-pyrazol-3- yl)pyrido[3,4-b] pyrazin-5-amine | LCMS (Method B): Rt = 0.53 min, [MH⁺] = 310 |
| 42 | 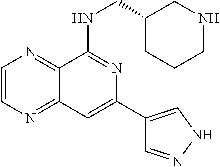 [SMILES: c1cnc2c(NC[C@H]3CCCNC3)nc(-c3cn[nH]c3)cc2n1] | N-[(3S)-3- piperidinylmethyl]- 7-(1H-pyrazol-4- yl)pyrido[3,4-b] pyrazin-5-amine | LCMS (Method B): Rt = 0.45 min, [MH⁺] = 310.2 |

The following example compound may be similarly prepared to Example 15, as the free base or a hydrochloride salt:

| Example | Structure | Name | LCMS |
|---|---|---|---|
| 16 | 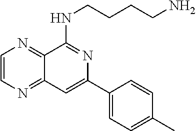 [SMILES: Cc1ccc(-c2cc3nccnc3c(NCCCCN)n2)cc1] | N-[7-(4- methylphenyl) pyrido[3,4-b] pyrazin-5-yl]- 1,4- butanediamine | LCMS (Method B): Rt = 1.58 min, [MH⁺] = 307.9 |

The following examples were prepared by similar methods:

| Example | Structure | Name (IUPAC) | LCMS |
|---|---|---|---|
| 43 | 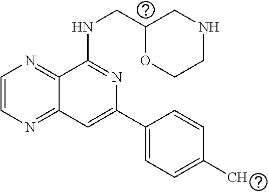 [SMILES: Cc1ccc(-c2cc3nccnc3c(NCC3CNCCO3)n2)cc1] | 7-(4-methylphenyl)-N- (morpholin-2-ylmethyl) pyrido[3,4-b]pyrazin-5- amine hydrochloride, single unknown enantiomer | LCMS (Method B): Rt = 0.75 min, MH⁺ 335.95 |
| 44 | 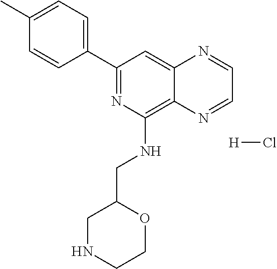 | 7-(4-methylphenyl)-N- (morpholin-2-ylmethyl) pyrido[3,4-b]pyrazin-5- amine hydrochloride, single unknown enantiomer | LCMS (Method B): Rt = 0.74 min, MH⁺ = 335.97 |
| 45 | 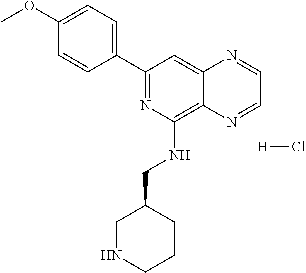 | 7-(4-methoxyphenyl)-N- [(3S)-piperidin-3- ylmethyl]pyrido[3,4-b] pyrazin-5-amine hydrochloride | LCMS (Method A): Rt = 2.46 min, MH⁺ = 350 |
| 46 | 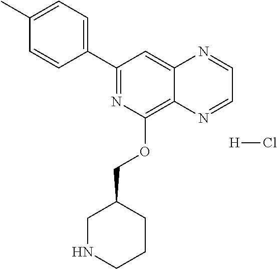 | (3S)-3-({[7-(4- methylphenyl)pyrido [3,4-b]pyrazin-5- yl]oxy}methyl) piperidine hydrochloride | LCMS (Method A): Rt = 2.64 min, MH⁺ = 334.95 |
| 47 | 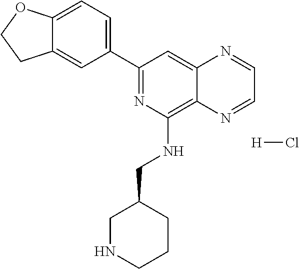 | 7-(2,3-dihydro-1-benzofuran- 5-yl)-N-[(3S)-piperidin-3- ylmethyl]pyrido[3,4-b]pyrazin- 5-amine hydrochloride | LCMS (Method B): Rt = 0.66 min, MH⁺ = 362.2 |
| 48 | 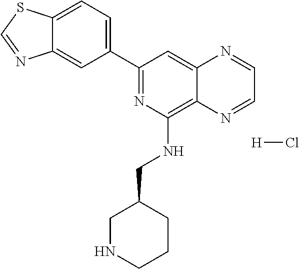 | 7-(1,3-benzothiazol-5-yl)-N- [(3S)-piperidin-3- ylmethyl]pyrido[3,4-b] pyrazin-5-amine hydrochloride | LCMS (Method B): Rt = 0.63 min, MH⁺ = 377.1 |
| 51 | 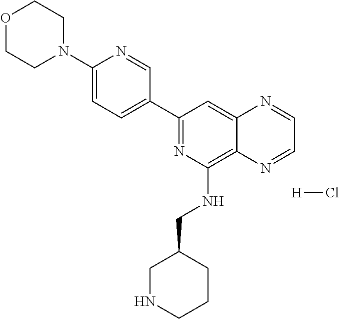 | 7-[6-(morpholin-4-yl)pyridin- 3-yl]-N-[(3S)-piperidin-3- ylmethyl]pyrido[3,4-b] pyrazin-5-amine hydrochloride | LCMS (Method B): Rt = 0.62 min, MH⁺ = 406 |
| 52 | 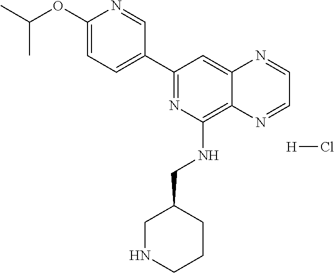 | N-[(3S)-piperidin-3- ylmethyl]-7-[6-(propan- 2-yloxy)pyridin-3- yl]pyrido[3,4-b]pyrazin- 5-amine hydrochloride | LCMS (Method B): Rt = 0.83 min, MH⁺ = 378.9 |
| 53 | 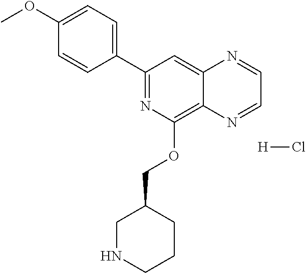 | (3S)-3-({[7-(4- methoxyphenyl)pyrido [3,4-b]pyrazin-5- yl]oxy}methyl)piperidine hydrochloride | LCMS (Method B): Rt = 0.74 min, MH⁺ = 351 |
| 54 | 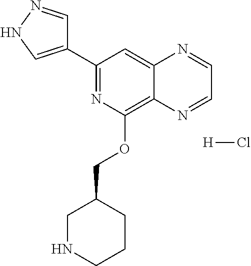 | (3S)-3-({[7-(1H-pyrazol-4- yl)pyrido[3,4-b]pyrazin-5- yl]oxy}methyl)piperidine hydrochloride | LCMS (Method B): Rt = 0.48 min, MH⁺ = 311 |
| 55 | 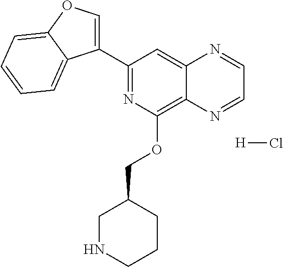 | (3S)-3-({[7-(1-benzofuran-3- yl)pyrido[3,4-b]pyrazin-5- yl]oxy}methyl)piperidine hydrochloride | LCMS (Method C): Rt = 0.85 min, MH⁺ = 360.9 |
| 56 | 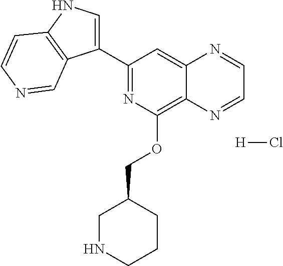 | (3S)-3-{[(7-{1H-pyrrolo [3,2-c]pyridin-3-yl}pyrido [3,4-b]pyrazin-5- yl)oxy]methyl}piperidine hydrochloride | LCMS (Method C): Rt = 0.47 min, MH⁺ = 361 |
| 57 | 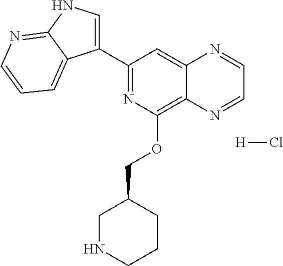 | (3S)-3-{[(7-{1H-pyrrolo [2,3-b]pyridin-3-yl}pyrido [3,4-b]pyrazin-5-yl)oxy] methyl}piperidine hydrochloride | LCMS (Method C): Rt = 0.52 min, MH⁺ = 360.9 |
| 58 | 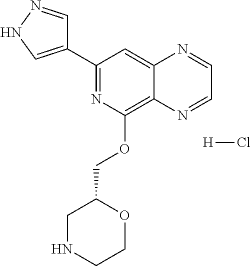 | (2R)-2-({[7-(1H-pyrazol-4- yl)pyrido[3,4-b]pyrazin-5- yl]oxy}methyl)morpholine hydrochloride | LCMS (Method B): Rt = 0.44 min, MH⁺ = 312.9 |
| 59 | 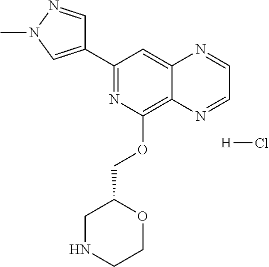 | (2R)-2-({[7-(1-methyl- 1H-pyrazol-4-yl) pyrido[3,4-b]pyrazin-5- yl]oxy}methyl)morpholine hydrochloride | LCMS (Method E): Rt = 1.64 min, MH⁺ = 326.9 |
| 60 | 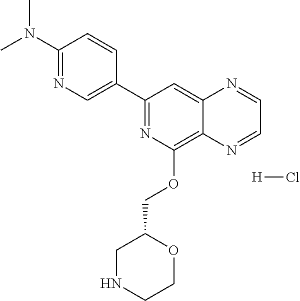 | N,N-dimethyl-5-{5-[(2R)- morpholin-2-ylmethoxy] pyrido[3,4-b]pyrazin- 7-yl}pyridin-2-amine hydrochloride | LCMS (Method E): Rt = 2.04 min, MH⁺ = 367 |
| 61 | 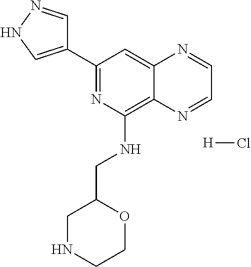 | N-(morpholin-2-ylmethyl)- 7-(1H-pyrazol-4-yl)pyrido [3,4-b]pyrazin-5-amine hydrochloride, single unknown enantiomer | LCMS (Method E): Rt = 1.61 min, MH⁺ = 312 |
| 62 | 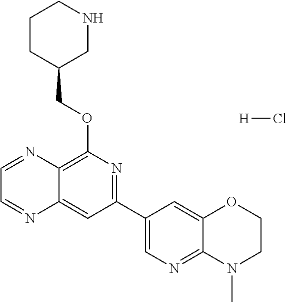 [SMILES: CN1CCOc2cc(-c3cc4nccnc4c(OC[C@H]4CCCNC4)n3)cnc21 ; Cl] | (3S)-3-{[(7-{4-methyl- 2H,3H,4H-pyrido[3,2-b] [1,4]oxazin-7-yl}pyrido [3,4-b]pyrazin-5-yl)oxy] methyl}piperidine hydrochloride | LCMS (Method B): Rt = 0.56 min, MH⁺ = 393.1 |
| 63 | 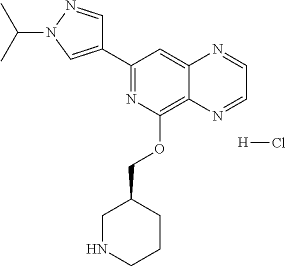 | (3S)-3-[({7-[1-(propan-2- yl)-1H-pyrazol-4-yl]pyrido [3,4-b]pyrazin- 5-yl}oxy)methyl]piperidine hydrochloride | LCMS (Method B): Rt = 0.62 min, MH⁺ = 353 |
| 64 | 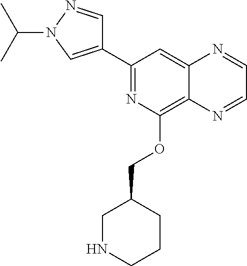 [SMILES: CC(C)n1cc(-c2cc3nccnc3c(OC[C@H]3CCCNC3)n2)cn1] | (3S)-3-[({7-[1-(propan-2- yl)-1H-pyrazol-4-yl]pyrido [3,4-b]pyrazin-5-yl}oxy) methyl]piperidine | LCMS (Method B): Rt = 0.63 min, MH⁺ = 353 |
| 65 | 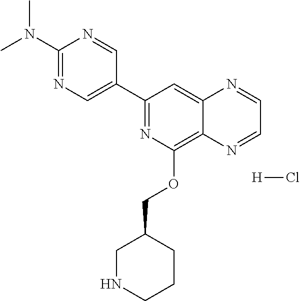 | N,N-dimethyl-5-{5-[(3S)- piperidin-3-ylmethoxy] pyrido[3,4-b]pyrazin-7-yl} pyrimidin-2-amine hydrochloride | LCMS (Method C): Rt = 0.61 min, MH⁺ = 366 |
| 66 | 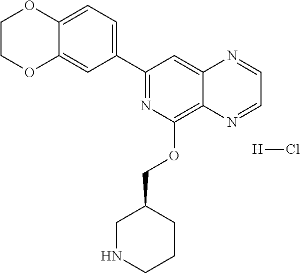 | (3S)-3-({[7-(2,3-dihydro- 1,4-benzodioxin-6-yl) pyrido[3,4-b]pyrazin-5- yl]oxy}methyl)piperidine hydrochloride | LCMS (Method C): Rt = 0.74 min, MH⁺ = 379 |
| 67 | 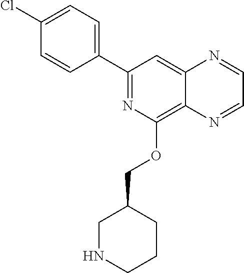 [SMILES: Clc1ccc(-c2cc3nccnc3c(OC[C@H]3CCCNC3)n2)cc1] | (3S)-3-({[7-(4- chlorophenyl)pyrido [3,4-b]pyrazin-5- yl]oxy}methyl) piperidine | LCMS (Method A): Rt = 1.12 min, MH⁺ = 355.2 |
| 68 | 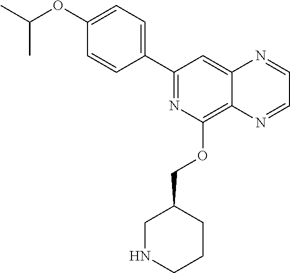 [SMILES: CC(C)Oc1ccc(-c2cc3nccnc3c(OC[C@H]3CCCNC3)n2)cc1] | (3S)-3-[({7-[4-(propan- 2-yloxy)phenyl]pyrido [3,4-b]pyrazin-5-yl}oxy) methyl]piperidine | LCMS (Method E): Rt = 0.76 min, MH⁺ = 379.1 |
| 69 | 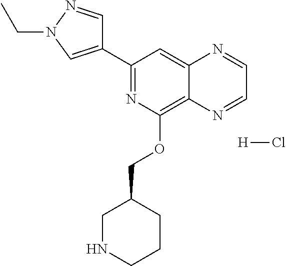 | (3S)-3-({[7-(1-ethyl-1H- pyrazol-4-yl)pyrido[3,4-b] pyrazin-5-yl]oxy}methyl) piperidine hydrochloride | LCMS (Method C): Rt = 0.59 min, MH⁺ = 339 |
| 70 | 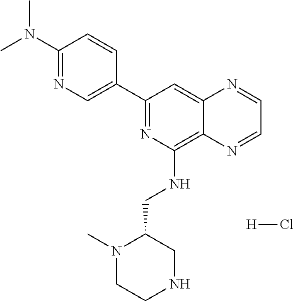 [SMILES: CN(C)c1ccc(-c2cc3nccnc3c(NC[C@@H]3CNCCN3C)n2)cn1 ; Cl] | N,N-dimethyl-5-[5-({[(2S)-1- methylpiperazin-2-yl]methyl} amino)pyrido[3,4-b]pyrazin- 7-yl]pyridin-2-amine hydrochloride | LCMS (Method A): Rt = 0.84 min, MH⁺ = 379.3 |
| 71 | 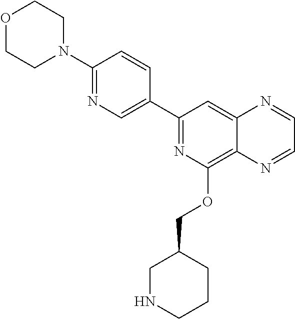 [SMILES: c1cnc2c(OC[C@H]3CCCNC3)nc(-c3ccc(N4CCOCC4)nc3)cc2n1] | 4-(5-{5-[(3S)-piperidin-3- ylmethoxy]pyrido[3,4-b] pyrazin-7-yl}pyridin-2-yl) morpholine | LCMS (Method B): Rt = 0.53 min, MH⁺ = 407 |
| 72 | 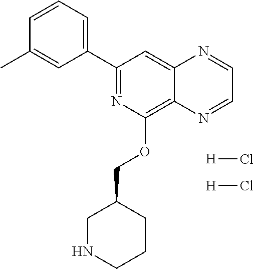 | (3S)-3-({[7-(3- methylphenyl)pyrido [3,4-b]pyrazin-5-yl] oxy}methyl)piperidine dihydrochloride | LCMS (Method A): Rt = 1.04 min, MH⁺ = 335.2 |
| 73 | 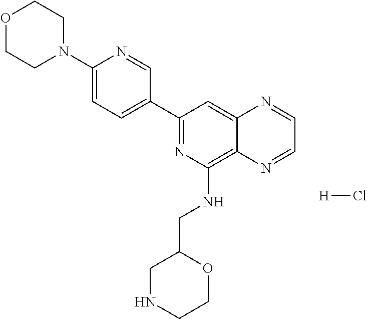 [SMILES: c1cnc2c(NCC3CNCCO3)nc(-c3ccc(N4CCOCC4)nc3)cc2n1 ; Cl] | N-(morpholin-2-ylmethyl)- 7-[6-(morpholin-4-yl) pyridin-3-yl]pyrido[3,4-b] pyrazin-5-amine hydrochloride, single unknown enantiomer | LCMS (Method C): Rt = 0.51 min, MH⁺ = 408 |
| 74 | 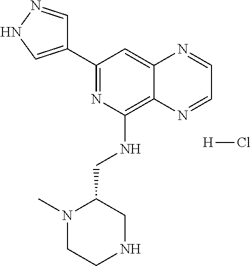 | N-{[(2S)-1-methylpiperazin- 2-yl]methyl}-7-(1H-pyrazol- 4-yl)pyrido[3,4-b]pyrazin-5- amine hydrochloride | LCMS (Method A): Rt = 0.62 min, MH⁺ = 325.3 |
| 75 | 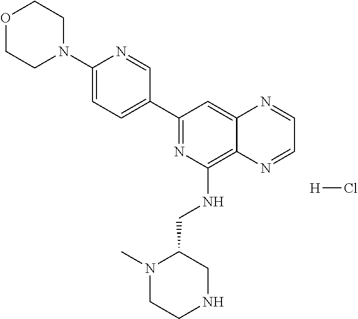 | N-{[(2S)-1-methylpiperazin- 2-yl]methyl}-7-[6-(morpholin- 4-yl)pyridin-3-yl]pyrido[3,4-b] pyrazin-5-amine hydrochloride | LCMS (Method A): Rt = 0.80 min, MH⁺ = 421.3 |
| 76 | 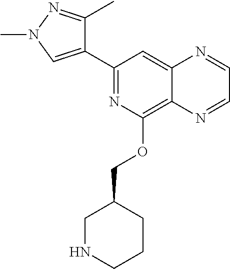 [SMILES: Cc1nn(C)cc1-c1cc2nccnc2c(OC[C@H]2CCCNC2)n1] | (3S)-3-({[7-(1,3-dimethyl- 1H-pyrazol-4-yl)pyrido[3,4-b] pyrazin-5-yl]oxy}methyl) piperidine | LCMS (Method A): Rt = 0.71 min, MH⁺ = 339.2 |
| 77 | 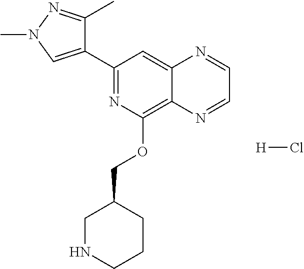 [SMILES: Cc1nn(C)cc1-c1cc2nccnc2c(OC[C@H]2CCCNC2)n1 ; Cl] | (3S)-3-({[7-(1,3-dimethyl- 1H-pyrazol-4-yl)pyrido[3,4-b] pyrazin-5-yl]oxy}methyl) piperidine hydrochloride | LCMS (Method B): Rt = 0.57 min, MH⁺ = 339.3 |
| 78 | 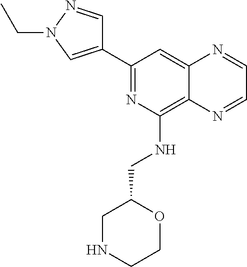 [SMILES: CCn1cc(-c2cc3nccnc3c(NC[C@H]3CNCCO3)n2)cn1] | 7-(1-ethyl-1H-pyrazol-4-yl)- N-[(2R)-morpholin-2- ylmethyl]pyrido[3,4-b] pyrazin-5-amine | LCMS (Method A): Rt = 0.74 min, MH⁺ = 340.2 |
| 79 | 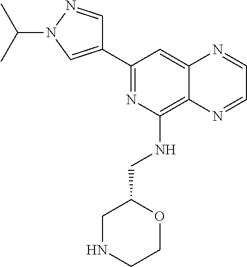 [SMILES: CC(C)n1cc(-c2cc3nccnc3c(NC[C@H]3CNCCO3)n2)cn1] | N-[(2R)-morpholin-2- ylmethyl]-7-[1-(propan- 2-yl)-1H-pyrazol-4-yl] pyrido[3,4-b]pyrazin- 5-amine | LCMS (Method A): Rt = 0.80 min, MH⁺ = 354.2 |
| 80 | 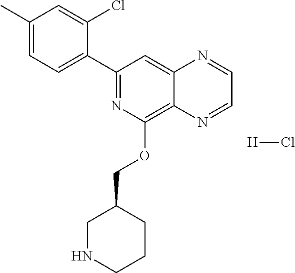 | (3S)-3-({[7-(2-chloro-4- methylphenyl)pyrido[3,4-b] pyrazin-5-yl]oxy}methyl) piperidine hydrochloride | LCMS (Method A): Rt = 1.09 min, MH⁺ = 369.2 |
| 81 | 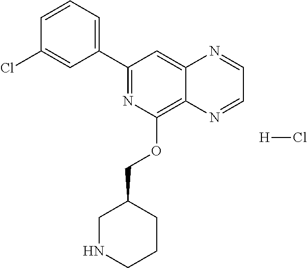 | (3S)-3-({[7-(3- chlorophenyl)pyrido[3,4-b] pyrazin-5-yl]oxy}methyl) piperidine hydrochloride | LCMS (Method A): Rt = 1.06 min, MH⁺ = 355 |
| 82 | 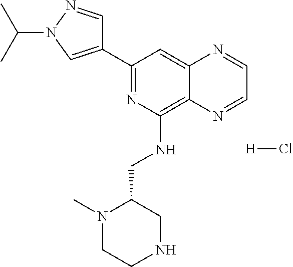 | N-{[(2S)-1- methylpiperazin-2- yl]methyl}-7-[1-(propan- 2-yl)-1H-pyrazol-4-yl] pyrido[3,4-b]pyrazin- 5-amine hydrochloride | LCMS (Method A): Rt = 0.77 min, MH⁺ = 367 |
| 83 | 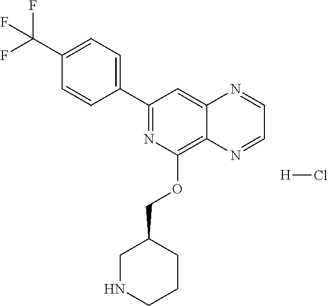 | (3S)-3-[({7-[4- (trifluoromethyl)phenyl] pyrido[3,4-b]pyrazin-5- yl}oxy)methyl]piperidine hydrochloride | LCMS (Method A): Rt = 1.11 min, MH⁺ = 389.2 |
| 84 | 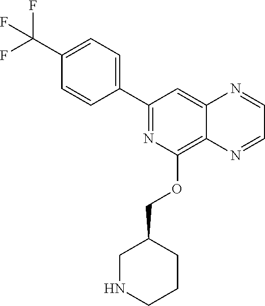 [SMILES: FC(F)(F)c1ccc(-c2cc3nccnc3c(OC[C@H]3CCCNC3)n2)cc1] | (3S)-3-[({7-[4- (trifluoromethyl)phenyl] pyrido[3,4-b]pyrazin-5- yl}oxy)methyl]piperidine | LCMS (Method A): Rt = 1.16 min, MH⁺ = 389.2 |
| 85 | 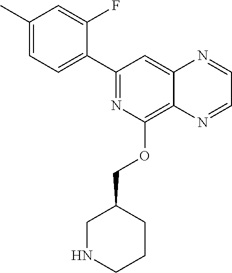 [SMILES: Cc1ccc(-c2cc3nccnc3c(OC[C@H]3CCCNC3)n2)c(F)c1] | (3S)-3-({[7-(2-fluoro- 4-methylphenyl)pyrido [3,4-b]pyrazin-5-yl]oxy} methyl)piperidine | LCMS (Method A): Rt = 1.07 min, MH⁺ = 353.2 |
| 86 | 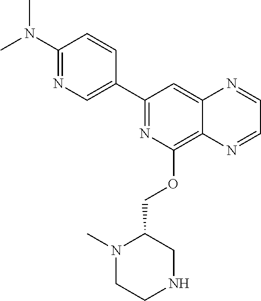 [SMILES: CN(C)c1ccc(-c2cc3nccnc3c(OC[C@@H]3CNCCN3C)n2)cn1] | N,N-dimethyl-5-(5-{[(2S)- 1-methylpiperazin-2- yl]methoxy}pyrido [3,4-b]pyrazin-7-yl) pyridin-2-amine | LCMS (Method A): Rt = 0.81 min, MH⁺ = 380.3 |
| 87 | 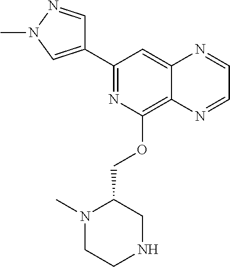 [SMILES: CN1CCNC[C@H]1COc1nc(-c2cnn(C)c2)cc2nccnc12] | (2S)-1-methyl-2-({[7- (1-methyl-1H-pyrazol- 4-yl)pyrido[3,4-b] pyrazin-5-yl]oxy}methyl) piperazine | LCMS (Method A): Rt = 0.64 min, MH⁺ = 340.3 |
| 88 | 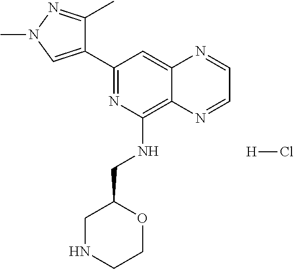 [SMILES: Cc1nn(C)cc1-c1cc2nccnc2c(NC[C@@H]2CNCCO2)n1 ; Cl] | 7-(1,3-dimethyl-1H- pyrazol-4-yl)-N-[(2S)- morpholin-2-ylmethyl] pyrido[3,4-b]pyrazin-5- amine hydrochloride | LCMS (Method A): Rt = 0.70 min, MH⁺ = 340.3 |
| 89 | 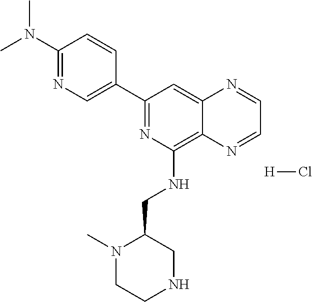 | N,N-dimethyl-5-[5- ({[(2R)-1-methylpiperazin- 2-yl]methyl}amino)pyrido [3,4-b]pyrazin-7-yl]pyridin- 2-amine hydrochloride | LCMS (Method A): Rt = 0.84 min, MH⁺ = 379.2 |
| 90 | 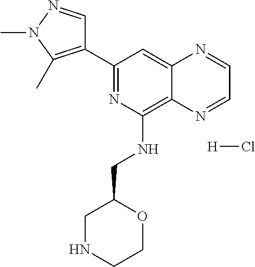 | 7-(1,5-dimethyl-1H- pyrazol-4-yl)-N-[(2S)- morpholin-2-ylmethyl] pyrido[3,4-b]pyrazin-5- amine hydrochloride | LCMS (Method C): Rt = 0.48 min, MH⁺ = 340 |
| 91 | 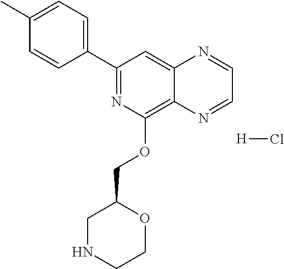 | (2S)-2-({[7-(4- methylphenyl)pyrido [3,4-b]pyrazin-5-yl]oxy} methyl)morpholine hydrochloride | LCMS (Method A): Rt = 0.99 min, MH⁺ = 337.2 |
| 92 | 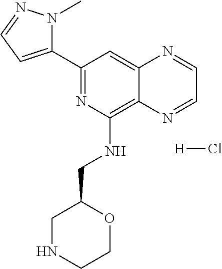 | 7-(1-methyl-1H-pyrazol- 5-yl)-N-[(2S)-morpholin- 2-ylmethyl]pyrido[3,4-b] pyrazin-5-amine hydrochloride | LCMS (Method C): Rt = 0.55 min, MH⁺ = 326 |
| 93 | 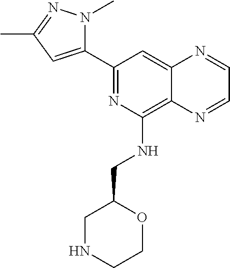 [SMILES: Cc1cc(-c2cc3nccnc3c(NC[C@@H]3CNCCO3)n2)n(C)n1] | 7-(1,3-dimethyl-1H-pyrazol- 5-yl)-N-[(2S)-morpholin-2- ylmethyl]pyrido[3,4-b]pyrazin- 5-amine | LCMS (Method C): Rt = 0.57 min, MH⁺ = 340 |
| 94 | 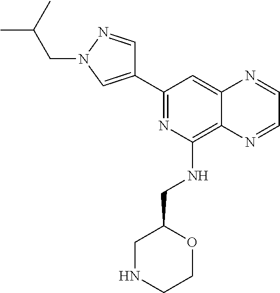 [SMILES: CC(C)Cn1cc(-c2cc3nccnc3c(NC[C@@H]3CNCCO3)n2)cn1] | 7-[1-(2-methylpropyl)-1H- pyrazol-4-yl]-N-[(2S)- morpholin-2-ylmethyl] pyrido[3,4-b]pyrazin-5-amine | LCMS (Method C): Rt = 0.63 min, MH⁺ = 368 |
| 95 | 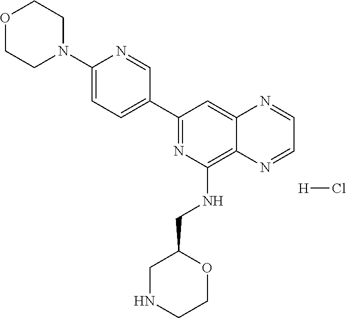 | N-[(2S)-morpholin-2- ylmethyl]-7-[6-(morpholin- 4-yl)pyridin-3-yl]pyrido [3,4-b]pyrazin-5-amine hydrochloride | LCMS (Method C): Rt = 0.49 min, MH⁺ = 408 |
| 96 | 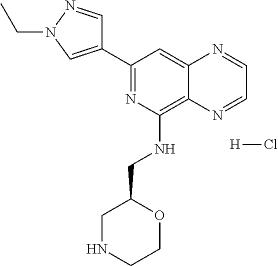 | 7-(1-ethyl-1H-pyrazol- 4-yl)-N-[(2S)-morpholin- 2-ylmethyl]pyrido[3,4-b] pyrazin-5-amine hydrochloride | LCMS (Method C): Rt = 0.52 min, MH⁺ = 340 |
| 97 | 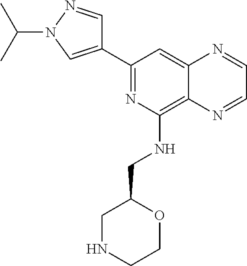 [SMILES: CC(C)n1cc(-c2cc3nccnc3c(NC[C@@H]3CNCCO3)n2)cn1] | N-[(2S)-morpholin-2- ylmethyl]-7-[1-(propan- 2-yl)-1H-pyrazol-4-yl] pyrido[3,4-b]pyrazin- 5-amine | LCMS (Method C): Rt = 0.56 min, MH⁺ = 354 |
| 98 | 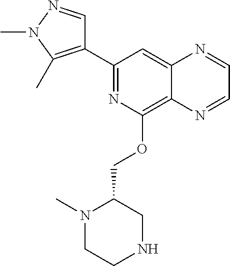 [SMILES: Cc1c(-c2cc3nccnc3c(OC[C@@H]3CNCCN3C)n2)cnn1C] | (2S)-2-({[7-(1,5-dimethyl- 1H-pyrazol-4-yl)pyrido [3,4-b]pyrazin-5-yl]oxy} methyl)-1-methylpiperazine | LCMS (Method A): Rt = 0.66 min, MH⁺ = 354.4 |
| 99 | 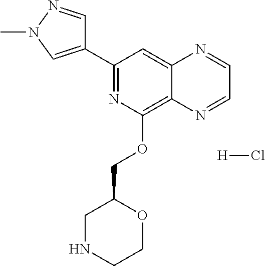 | (2S)-2-({[7-(1-methyl- 1H-pyrazol-4-yl)pyrido [3,4-b]pyrazin-5-yl]oxy} methyl)morpholine hydrochloride | LCMS (Method A): Rt = 0.63 min, MH⁺ = 327.2 |
| 100 | 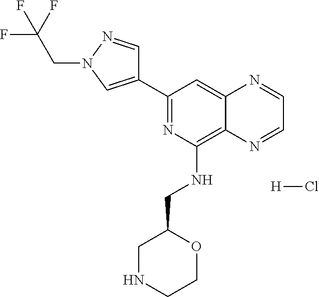 | N-[(2S)-morpholin-2- ylmethyl]-7-[1-(2,2,2- trifluoroethyl)-1H- pyrazol-4-yl]pyrido [3,4-b]pyrazin-5-amine hydrochloride | LCMS (Method A): Rt = 0.81 min, MH⁺ = 394.3 |
| 101 | 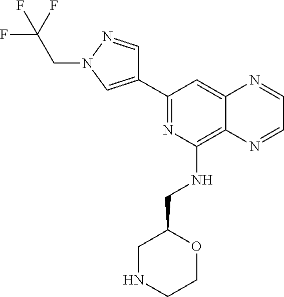 [SMILES: FC(F)(F)Cn1cc(-c2cc3nccnc3c(NC[C@@H]3CNCCO3)n2)cn1] | N-[(2S)-morpholin-2- ylmethyl]-7-[1-(2,2,2- trifluoroethyl)-1H- pyrazol-4-yl]pyrido [3,4-b]pyrazin-5-amine | LCMS (Method C): Rt = 0.61 min, MH⁺ = 394 |
| 102 | 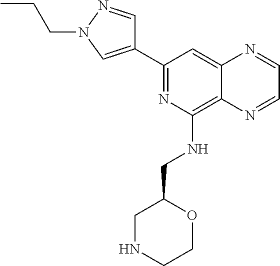 [SMILES: CCCn1cc(-c2cc3nccnc3c(NC[C@@H]3CNCCO3)n2)cn1] | N-[(2S)-morpholin-2- ylmethyl]-7-(1-propyl- 1H-pyrazol-4-yl)pyrido [3,4-b]pyrazin-5-amine | LCMS (Method C): Rt = 0.57 min, MH⁺ = 354 |
| 103 | 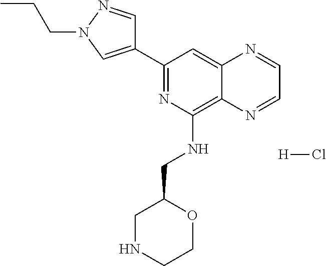 [SMILES: CCCn1cc(-c2cc3nccnc3c(NC[C@@H]3CNCCO3)n2)cn1 ; Cl] | N-[(2S)-morpholin-2- ylmethyl]-7-(1-propyl- 1H-pyrazol-4-yl)pyrido [3,4-b]pyrazin-5-amine hydrochloride | LCMS (Method C): Rt = 0.57 min, MH⁺ = 354 |
| 104 | 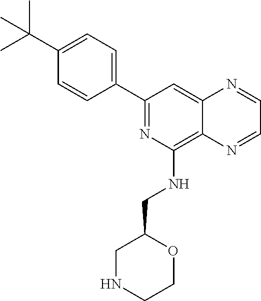 [SMILES: CC(C)(C)c1ccc(-c2cc3nccnc3c(NC[C@@H]3CNCCO3)n2)cc1] | 7-(4-tert-butylphenyl)- N-[(2S)-morpholin-2- ylmethyl]pyrido[3,4-b] pyrazin-5-amine | LCMS (Method B): Rt = 0.90 min, MH⁺ = 378.1 |
| 105 | 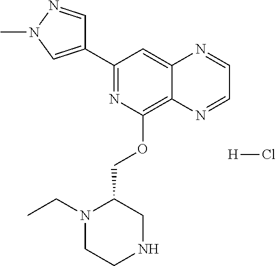 [SMILES: CCN1CCNC[C@H]1COc1nc(-c2cnn(C)c2)cc2nccnc12 ; Cl] | (2S)-1-ethyl-2-({[7- (1-methyl-1H-pyrazol- 4-yl)pyrido[3,4-b]pyrazin- 5-yl]oxy}methyl)piperazine hydrochloride | LCMS (Method A): Rt = 0.67 min, MH⁺ = 354.4 |
| 106 | 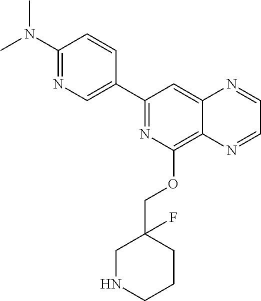 [SMILES: CN(C)c1ccc(-c2cc3nccnc3c(OCC3(F)CCCNC3)n2)cn1] | 5-{5-[(3-fluoropiperidin-3- yl)methoxy]pyrido[3,4-b] pyrazin-7-yl}-N,N- dimethylpyridin-2-amine | LCMS (Method A): Rt = 0.94 min, MH⁺ = 383.3 |
| 107 | 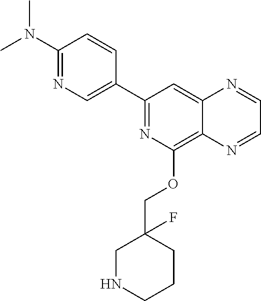 [SMILES: CN(C)c1ccc(-c2cc3nccnc3c(OCC3(F)CCCNC3)n2)cn1] | 5-{5-[(3-fluoropiperidin-3- yl)methoxy]pyrido[3,4-b] pyrazin-7-yl}-N,N- dimethylpyridin-2- amine, single unknown enantiomer | LCMS (Method A): Rt = 0.94 min, MH⁺ = 383.3 |
| 108 | 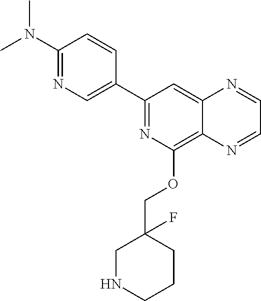 [SMILES: CN(C)c1ccc(-c2cc3nccnc3c(OCC3(F)CCCNC3)n2)cn1] | 5-{5-[(3-fluoropiperidin-3- yl)methoxy]pyrido[3,4-b] pyrazin-7-yl}-N,N- dimethylpyridin-2- amine, single unknown enantiomer | LCMS (Method A): Rt = 0.94 min, MH⁺ = 383.3 |
| 109 | 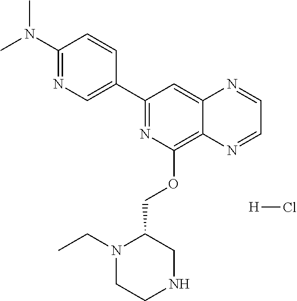 [SMILES: CCN1CCNC[C@H]1COc1nc(-c2ccc(N(C)C)nc2)cc2nccnc12 ; Cl] | 5-(5-{[(2S)-1-ethylpiperazin- 2-yl]methoxy}pyrido [3,4-b]pyrazin-7-yl)-N,N- dimethylpyridin-2- amine hydrochloride | LCMS (Method A): Rt = 0.87 min, MH⁺ = 394.4 |
| 110 | 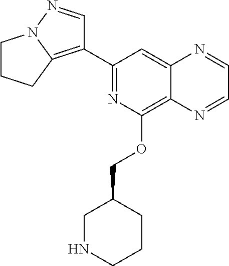 [SMILES: c1cnc2c(OC[C@H]3CCCNC3)nc(-c3cnn4c3CCC4)cc2n1] | (3S)-3-{[(7-{4H,5H,6H- pyrrolo[1,2-b]pyrazol-3- yl}pyrido[3,4-b]pyrazin-5- yl)oxy]methyl}piperidine | LCMS (Method C): Rt = 0.61 min, MH⁺ = 351.1 |
| 111 | 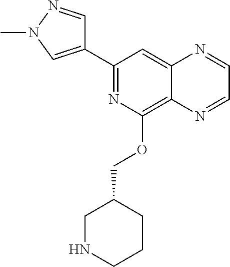 [SMILES: Cn1cc(-c2cc3nccnc3c(OC[C@@H]3CCCNC3)n2)cn1] | (3R)-3-({[7-(1-methyl- 1H-pyrazol-4-yl)pyrido [3,4-b]pyrazin-5- yl]oxy}methyl)piperidine | LCMS (Method C): Rt = 0.58 min, MH⁺ = 325 |
| 112 | 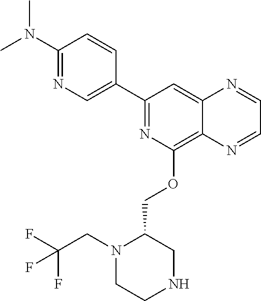 [SMILES: CN(C)c1ccc(-c2cc3nccnc3c(OC[C@@H]3CNCCN3CC(F)(F)F)n2)cn1] | N,N-dimethyl-5-(5-{[(2S)- 1-(2,2,2-trifluoroethyl) piperazin-2-yl]methoxy} pyrido[3,4-b]pyrazin- 7-yl)pyridin-2-amine | LCMS (Method A): Rt = 1.02 min, MH⁺ = 448.3 |
| 113 | 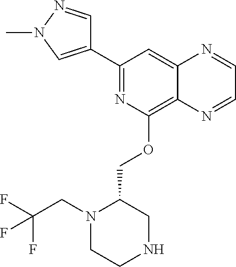 [SMILES: Cn1cc(-c2cc3nccnc3c(OC[C@@H]3CNCCN3CC(F)(F)F)n2)cn1] | (2S)-2-({[7-(1-methyl- 1H-pyrazol-4-yl)pyrido [3,4-b]pyrazin-5-yl]oxy} methyl)-1-(2,2,2- trifluoroethyl)piperazine | LCMS (Method A): Rt = 0.81 min, MH⁺ = 408 |
| 114 | 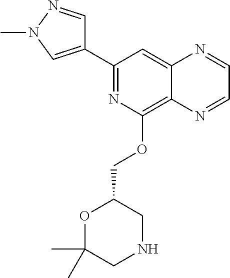 [SMILES: Cn1cc(-c2cc3nccnc3c(OC[C@@H]3CNCC(C)(C)O3)n2)cn1] | (6S)-2,2-dimethyl-6- ({[7-(1-methyl-1H- pyrazol-4-yl)pyrido [3,4-b]pyrazin-5- yl]oxy}methyl)morpholine | LCMS (Method B): Rt = 0.57 min, MH⁺ = 355 |
| 115 | 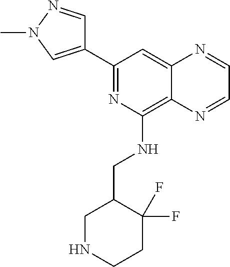 [SMILES: Cn1cc(-c2cc3nccnc3c(NCC3CNCCC3(F)F)n2)cn1] | N-[(4,4-difluoropiperidin-3- yl)methyl]-7-(1-methyl-1H- pyrazol-4-yl)pyrido[3,4-b] pyrazin-5-amine | LCMS (Method A): Rt = 0.83 min, MH⁺ = 360 |
| 116 | 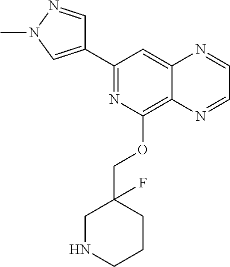 [SMILES: Cn1cc(-c2cc3nccnc3c(OCC3(F)CCCNC3)n2)cn1] | 3-fluoro-3-({[7-(1-methyl-1H- pyrazol-4-yl)pyrido[3,4-b] pyrazin-5-yl]oxy}methyl) piperidine, single unknown enantiomer | LCMS (Method B): Rt = 0.51 min, MH⁺ = 343 |
| 117 | 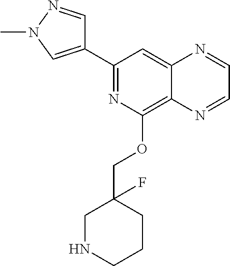 [SMILES: Cn1cc(-c2cc3nccnc3c(OCC3(F)CCCNC3)n2)cn1] | 3-fluoro-3-({[7-(1-methyl-1H- pyrazol-4-yl)pyrido[3,4-b] pyrazin-5-yl]oxy}methyl) piperidine, single unknown enantiomer | LCMS (Method A): Rt = 0.73 min, MH⁺ = 343 |
| 118 | 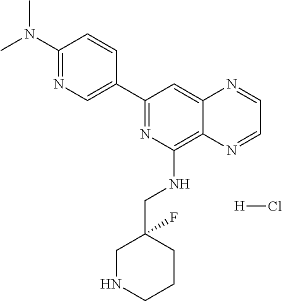 [SMILES: CN(C)c1ccc(-c2cc3nccnc3c(NC[C@@]3(F)CCCNC3)n2)cn1 ; Cl] | 5-[5-({[(3R)-3-fluoropiperidin- 3-yl]methyl}amino)pyrido [3,4-b]pyrazin-7-yl]-N,N- dimethylpyridin-2-amine hydrochloride | LCMS (Method B): Rt = 0.46 min, MH⁺ = 382.3 |
| 119 | 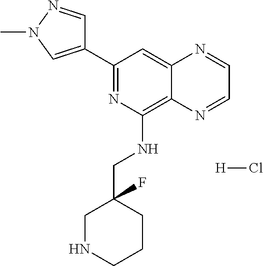 [SMILES: Cn1cc(-c2cc3nccnc3c(NC[C@]3(F)CCCNC3)n2)cn1 ; Cl] | N-{[(3S)-3-fluoropiperidin-3- yl]methyl}-7-(1-methyl-1H- pyrazol-4-yl)pyrido[3,4-b] pyrazin-5-amine hydrochloride | LCMS (Method B): Rt = 0.53 min, MH⁺ = 341.9 |
| 120 | 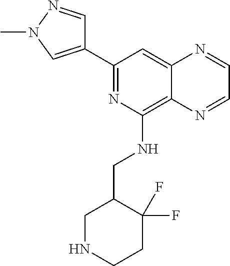 [SMILES: Cn1cc(-c2cc3nccnc3c(NCC3CNCCC3(F)F)n2)cn1] | N-[(4,4-difluoropiperidin-3- yl)methyl]-7-(1-methyl-1H- pyrazol-4-yl)pyrido[3,4-b] pyrazin-5-amine, single unknown enantiomer | LCMS (Method A): Rt = 0.84 min, MH⁺ = 360.3 |
| 121 | 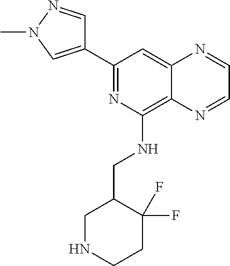 [SMILES: Cn1cc(-c2cc3nccnc3c(NCC3CNCCC3(F)F)n2)cn1] | N-[(4,4-difluoropiperidin-3- yl)methyl]-7-(1-methyl-1H- pyrazol-4-yl)pyrido[3,4-b] pyrazin-5-amine, single unknown enantiomer | LCMS (Method A): Rt = 0.84 min, MH⁺ = 360.3 |
| 122 | 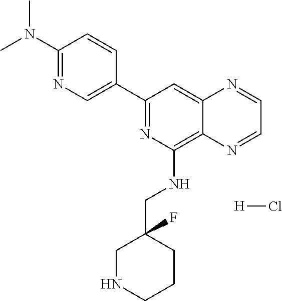 [SMILES: CN(C)c1ccc(-c2cc3nccnc3c(NC[C@]3(F)CCCNC3)n2)cn1 ; Cl] | 5-[5-({[(3S)-3-fluoropiperidin- 3-yl]methyl}amino)pyrido [3,4-b]pyrazin-7-yl]-N,N- dimethylpyridin-2-amine hydrochloride | LCMS (Method B): Rt = 0.44 min, MH⁺ = 382.1 |
| 123 | 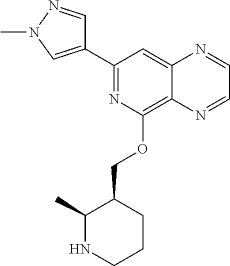 [SMILES: C[C@@H]1NCCC[C@@H]1COc1nc(-c2cnn(C)c2)cc2nccnc12] | (2S,3S)-2-methyl-3-({[7-(1- methyl-1H-pyrazol-4- yl)pyrido[3,4-b]pyrazin-5- yl]oxy}methyl)piperidine | LCMS (Method A): Rt = 0.70 min, MH⁺ = 339.3 |
| 124 | 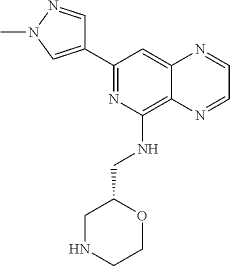 [SMILES: Cn1cc(-c2cc3nccnc3c(NC[C@H]3CNCCO3)n2)cn1] | 7-(1-methyl-1H-pyrazol-4-yl)- N-[(2R)-morpholin-2- ylmethyl]pyrido[3,4-b]pyrazin- 5-amine | LCMS (Method C): Rt = 0.49 min, MH⁺ = 325.9 |
| 125 | 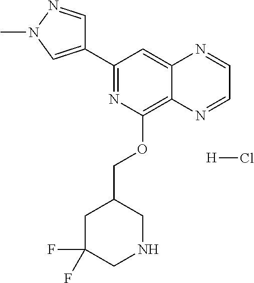 | 3,3-difluoro-5-({[7-(1- methyl-1H-pyrazol-4-yl) pyrido[3,4-b]pyrazin- 5-yl]oxy}methyl)piperidine hydrochloride | LCMS (Method B): Rt = 0.54 min, MH⁺ = 361 |
| 126 | 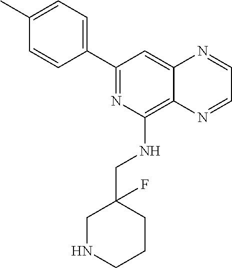 [SMILES: Cc1ccc(-c2cc3nccnc3c(NCC3(F)CCCNC3)n2)cc1] | N-[(3-fluoropiperidin- 3-yl)methyl]-7-(4- methylphenyl)pyrido[3,4-b] pyrazin-5-amine | LCMS (Method B): Rt = 0.85 min, MH⁺ = 351.9 |
| 127 | 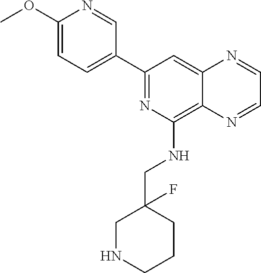 [SMILES: COc1ccc(-c2cc3nccnc3c(NCC3(F)CCCNC3)n2)cn1] | N-[(3-fluoropiperidin-3-yl) methyl]-7-(6-methoxypyridin- 3-yl)pyrido[3,4-b]pyrazin- 5-amine | LCMS (Method A): Rt = 0.97 min, MH⁺ = 369.3 |
| 128 | 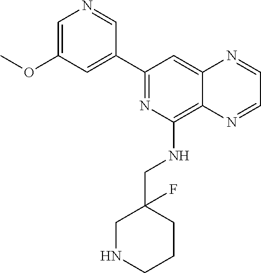 [SMILES: COc1cncc(-c2cc3nccnc3c(NCC3(F)CCCNC3)n2)c1] | N-[(3-fluoropiperidin- 3-yl)methyl]-7-(5- methoxypyridin-3- yl)pyrido[3,4-b]pyrazin- 5-amine | LCMS (Method A): Rt = 0.87 min, MH⁺ = 369.3 |
| 129 | 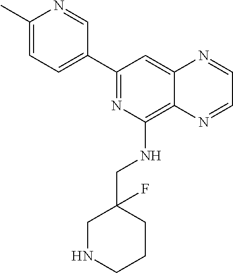 [SMILES: Cc1ccc(-c2cc3nccnc3c(NCC3(F)CCCNC3)n2)cn1] | N-[(3-fluoropiperidin- 3-yl)methyl]-7-(6- methylpyridin-3-yl) pyrido[3,4-b]pyrazin- 5-amine | LCMS (Method A): Rt = 0.88 min, MH⁺ = 353.3 |
| 130 | 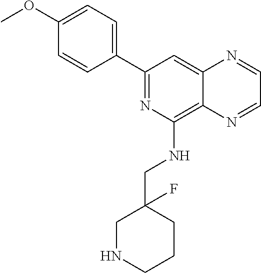 [SMILES: COc1ccc(-c2cc3nccnc3c(NCC3(F)CCCNC3)n2)cc1] | N-[(3-fluoropiperidin-3- yl)methyl]-7-(4- methoxyphenyl)pyrido [3,4-b]pyrazin-5-amine | LCMS (Method A): Rt = 1.05 min, MH⁺ = 368.3 |
| 131 | 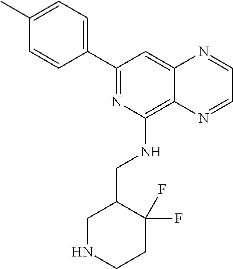 [SMILES: Cc1ccc(-c2cc3nccnc3c(NCC3CNCCC3(F)F)n2)cc1] | N-[(4,4-difluoropiperidin-3- yl)methyl]-7-(4- methylphenyl)pyrido[3,4-b] pyrazin-5-amine | LCMS (Method A): Rt = 1.18 min, MH⁺ = 370.3 |
| 132 | 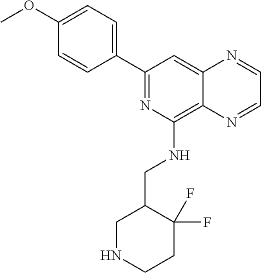 [SMILES: COc1ccc(-c2cc3nccnc3c(NCC3CNCCC3(F)F)n2)cc1] | N-[(4,4-difluoropiperidin-3- yl)methyl]-7-(4- methoxyphenyl)pyrido[3,4-b] pyrazin-5-amine | LCMS (Method A): Rt = 1.08 min, MH⁺ = 386.3 |
| 133 | 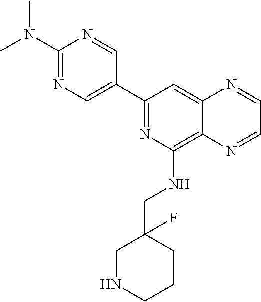 [SMILES: CN(C)c1ncc(-c2cc3nccnc3c(NCC3(F)CCCNC3)n2)cn1] | 5-(5-{[(3-fluoropiperidin-3- yl)methyl]amino}pyrido [3,4-b]pyrazin-7-yl)-N,N- dimethylpyrimidin-2-amine | LCMS (Method A): Rt = 0.94 min, MH⁺ = 383.3 |
| 134 | 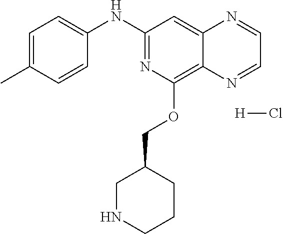 | N-(4-methylphenyl)-5- [(3S)-piperidin-3- ylmethoxy]pyrido[3,4-b] pyrazin-7-amine hydrochloride | LCMS (Method A): Rt = 0.97 min, MH⁺ = 350.3 |
| 135 | 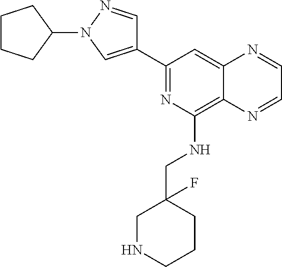 [SMILES: FC1(CNc2nc(-c3cnn(C4CCCC4)c3)cc3nccnc23)CCCNC1] | 7-(1-cyclopentyl-1H-pyrazol- 4-yl)-N-[(3-fluoropiperidin-3- yl)methyl]pyrido[3,4-b]pyrazin- 5-amine hydrochloride | LCMS (Method C): Rt = 0.74 min, MH⁺ = 396 |
| 136 | 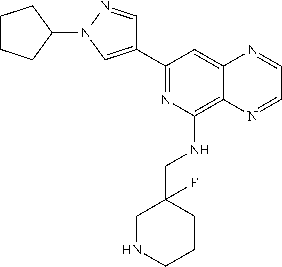 [SMILES: FC1(CNc2nc(-c3cnn(C4CCCC4)c3)cc3nccnc23)CCCNC1] | 7-(1-cyclopentyl-1H-pyrazol- 4-yl)-N-[(3-fluoropiperidin-3- yl)methyl]pyrido[3,4-b] pyrazin-5-amine | LCMS (Method B): Rt = 0.68 min, MH⁺ = 396.2 |
| 137 | 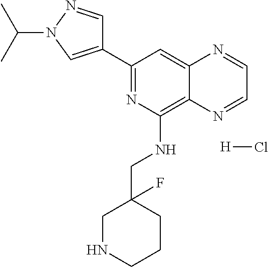 | N-[(3-fluoropiperidin-3-yl) methyl]-7-[1-(propan-2-yl)- 1H-pyrazol-4-yl]pyrido [3,4-b]pyrazin-5-amine hydrochloride | LCMS (Method C): Rt = 0.65 min, MH⁺ = 370 |
| 138 | 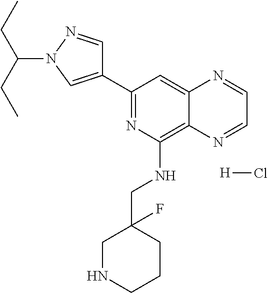 | N-[(3-fluoropiperidin-3-yl) methyl]-7-[1-(pentan-3-yl)- 1H-pyrazol-4- yl]pyrido[3,4-b]pyrazin- 5-amine hydrochloride | LCMS (Method C): Rt = 0.75 min, MH⁺ = 398.1 |
| 139 | 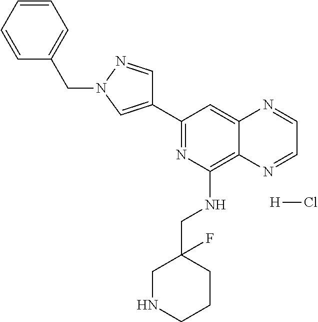 | 7-(1-benzyl-1H-pyrazol-4- yl)-N-[(3-fluoropiperidin-3- yl)methyl]pyrido[3,4-b] pyrazin-5-amine hydrochloride | LCMS (Method C): Rt = 0.75 min, MH⁺ = 418 |
| 140 | 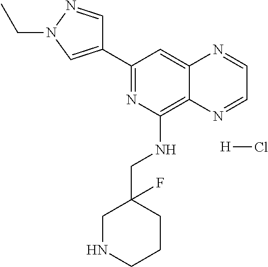 | 7-(1-ethyl-1H-pyrazol-4-yl)- N-[(3-fluoropiperidin-3- yl)methyl]pyrido[3,4-b] pyrazin-5-amine hydrochloride | LCMS (Method B): Rt = 0.62 min, MH⁺ = 356 |
| 141 | 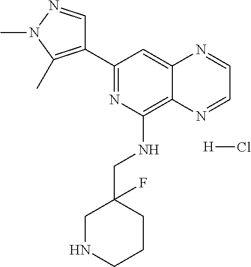 | 7-(1,5-dimethyl-1H-pyrazol- 4-yl)-N-[(3-fluoropiperidin-3- yl)methyl]pyrido[3,4-b] pyrazin-5-amine hydrochloride | LCMS (Method B): Rt = 0.56 min, MH⁺ = 356 |
| 142 | 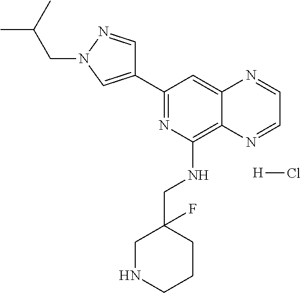 | N-[(3-fluoropiperidin-3-yl) methyl]-7-[1-(2-methylpropyl)- 1H-pyrazol-4-yl]pyrido [3,4-b]pyrazin-5-amine hydrochloride | LCMS (Method B): Rt = 0.70 min, MH⁺ = 384 |
| 143 | 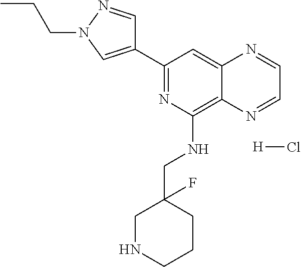 | N-[(3-fluoropiperidin-3-yl) methyl]-7-(1-propyl-1H- pyrazol-4-yl)pyrido[3,4-b] pyrazin-5-amine hydrochloride | LCMS (Method B): Rt = 0.63 min, MH⁺ = 370 |
| 144 | 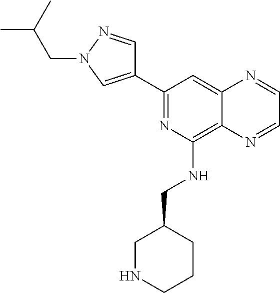 [SMILES: CC(C)Cn1cc(-c2cc3nccnc3c(NC[C@H]3CCCNC3)n2)cn1] | 7-[1-(2-methylpropyl)-1H- pyrazol-4-yl]-N-[(3S)- piperidin-3-ylmethyl]pyrido [3,4-b]pyrazin-5-amine | LCMS (Method B): Rt = 0.70 min, MH⁺ = 366.1 |
| 145 | 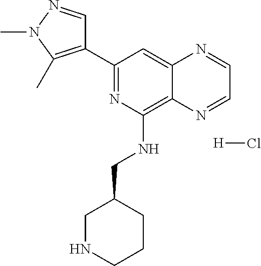 | 7-(1,5-dimethyl-1H-pyrazol- 4-yl)-N-[(3S)-piperidin-3- ylmethyl]pyrido[3,4-b] pyrazin-5-amine hydrochloride | LCMS (Method B): Rt = 0.56 min, MH⁺ = 338 |
| 146 | 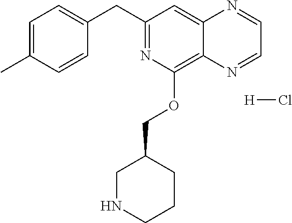 | (3S)-3-[({7-[(4- methylphenyl)methyl] pyrido[3,4-b]pyrazin-5- yl}oxy)methyl]piperidine hydrochloride | LCMS (Method B): Rt = 0.82 min, MH⁺ = 349 |
| 147 | 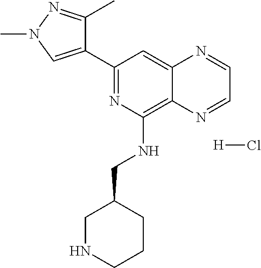 | 7-(1,3-dimethyl-1H-pyrazol- 4-yl)-N-[(3S)-piperidin-3- ylmethyl]pyrido[3,4-b] pyrazin-5-amine hydrochloride | LCMS (Method C): Rt = 0.52 min, MH⁺ = 338 |
| 148 | 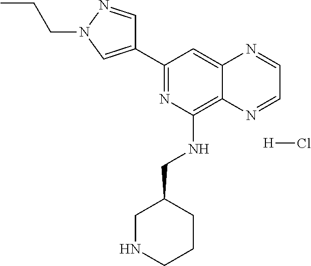 | N-[(3S)-piperidin-3- ylmethyl]-7-(1-propyl- 1H-pyrazol-4-yl)pyrido [3,4-b]pyrazin-5-amine hydrochloride | LCMS (Method B): Rt = 0.64 min, MH⁺ = 352 |
| 149 | 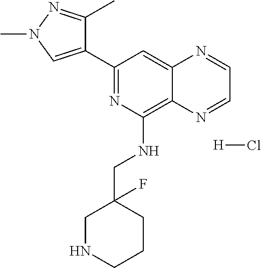 | 7-(1,3-dimethyl-1H-pyrazol- 4-yl)-N-[(3-fluoropiperidin- 3-yl)methyl]pyrido [3,4-b]pyrazin-5- amine hydrochloride | LCMS (Method C): Rt = 0.56 min, MH⁺ = 356 |
| 150 | 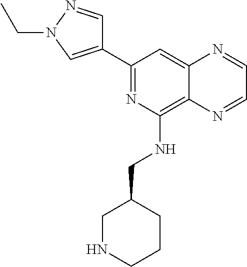 [SMILES: CCn1cc(-c2cc3nccnc3c(NC[C@H]3CCCNC3)n2)cn1] | 7-(1-ethyl-1H-pyrazol-4- yl)-N-[(3S)-piperidin-3- ylmethyl]pyrido[3,4-b] pyrazin-5-amine | LCMS (Method C): Rt = 0.56 min, MH⁺ = 338 |
| 151 | 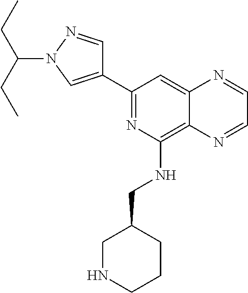 [SMILES: CCC(CC)n1cc(-c2cc3nccnc3c(NC[C@H]3CCCNC3)n2)cn1] | 7-[1-(pentan-3-yl)-1H- pyrazol-4-yl]-N-[(3S)- piperidin-3- ylmethyl]pyrido [3,4-b]pyrazin- 5-amine | LCMS (Method B): Rt = 0.76 min, MH⁺ = 380 |
| 152 | 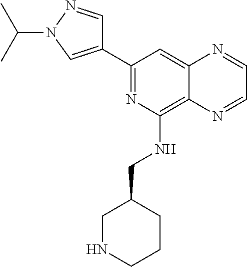 [SMILES: CC(C)n1cc(-c2cc3nccnc3c(NC[C@H]3CCCNC3)n2)cn1] | N-[(3S)-piperidin-3- ylmethyl]-7-[1-(propan- 2-yl)-1H-pyrazol-4- yl]pyrido[3,4-b] pyrazin-5-amine | LCMS (Method B): Rt = 0.65 min, MH⁺ = 352.1 |
| 153 | 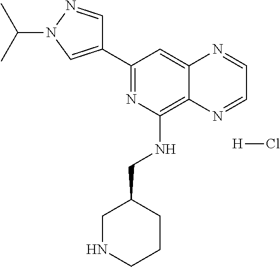 | N-[(3S)-piperidin-3- ylmethyl]-7-[1-(propan- 2-yl)-1H-pyrazol-4- yl]pyrido[3,4-b] pyrazin-5-amine hydrochloride | LCMS (Method B): Rt = 0.65 min, MH⁺ = 352 |
| 154 | 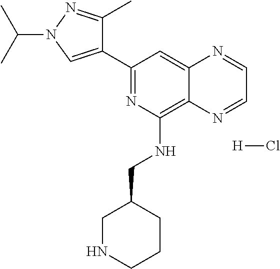 | 7-[3-methyl-1-(propan- 2-yl)-1H-pyrazol-4-yl]- N-[(3S)-piperidin-3- ylmethyl]pyrido[3,4-b] pyrazin-5-amine hydrochloride | LCMS (Method B): Rt = 0.65 min, MH⁺ = 366 |
| 155 | 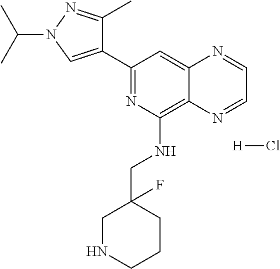 | N-[(3-fluoropiperidin-3-yl) methyl]-7-[3-methyl- 1-(propan-2-yl)-1H- pyrazol-4-yl]pyrido[3,4-b] pyrazin-5-amine hydrochloride | LCMS (Method B): Rt = 0.66 min, MH⁺ = 384 |
| 156 | 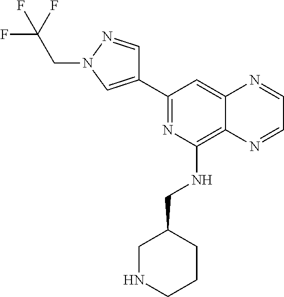 [SMILES: FC(F)(F)Cn1cc(-c2cc3nccnc3c(NC[C@H]3CCCNC3)n2)cn1] | N-[(3S)-piperidin-3- ylmethyl]-7- [1-(2,2,2-trifluoroethyl)- 1H-pyrazol-4-yl]pyrido [3,4-b]pyrazin- 5-amine | LCMS (Method C): Rt = 0.66 min, MH⁺ = 392 |
| 157 | 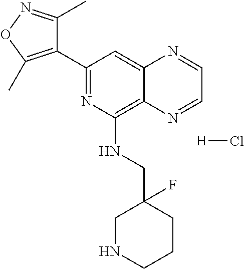 | 7-(dimethyl-1,2-oxazol-4-yl)- N-[(3-fluoropiperidin-3- yl)methyl]pyrido [3,4-b]pyrazin- 5-amine hydrochloride | LCMS (Method A): Rt = 0.91 min, MH⁺ = 357.2 |
| 158 | 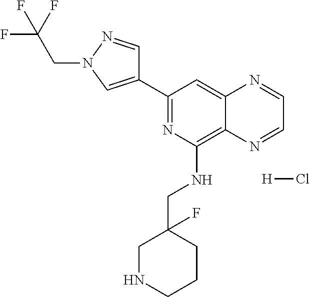 | N-[(3-fluoropiperidin-3-yl)methyl]- 7-[1-(2,2,2-trifluoroethyl)-1H- pyrazol-4-yl]pyrido[3,4-b]pyrazin- 5-amine hydrochloride | LCMS (Method A): Rt = 0.93 min, MH⁺ = 410.1 |
| 159 | 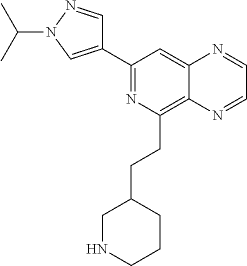 [SMILES: CC(C)n1cc(-c2cc3nccnc3c(CCC3CCCNC3)n2)cn1] | 3-(2-{7-[1-(propan-2-yl)-1H- pyrazol-4-yl]pyrido[3,4-b]pyrazin- 5-yl}ethyl)piperidine | LCMS (Method B): Rt = 0.64 min, MH⁺ = 351 |
| 160 | 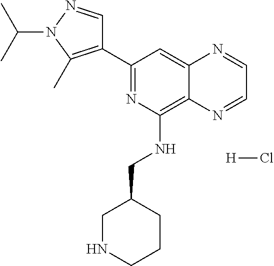 | 7-[5-methyl-1-(propan-2-yl)-1H- pyrazol-4-yl]-N-[(3S)-piperidin-3- ylmethyl]pyrido[3,4-b]pyrazin-5- amine hydrochloride | LCMS (Method B): Rt = 0.67 min, MH⁺ = 366 |
| 161 | 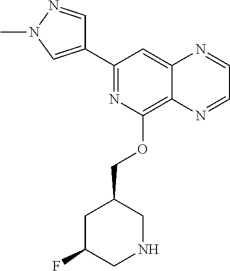 [SMILES: Cn1cc(-c2cc3nccnc3c(OC[C@H]3CNC[C@@H](F)C3)n2)cn1] | (+/−)(3S,5R)-3-fluoro-5- ({[7-(1-methyl-1H-pyrazol-4- yl)pyrido[3,4-b]pyrazin-5- yl]oxy}methyl)piperidine | LCMS (Method A): Rt = 0.74 min, MH⁺ = 343.2 |
| 162 | 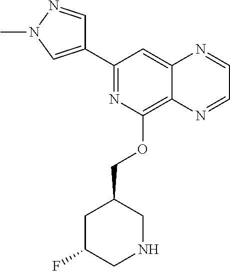 [SMILES: Cn1cc(-c2cc3nccnc3c(OC[C@H]3CNC[C@H](F)C3)n2)cn1] | (+/−)(3R,5R)-3-fluoro-5- ({[7-(1-methyl-1H-pyrazol-4- yl)pyrido[3,4-b]pyrazin-5- yl]oxy}methyl)piperidine | LCMS (Method A): Rt = 0.70 min, MH⁺ = 343.3 |
| 163 | 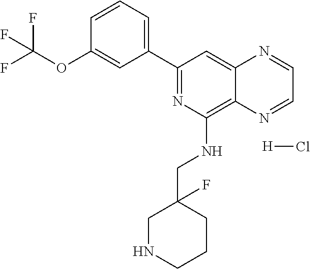 | N-[(3-fluoropiperidin-3-yl) methyl]-7-[3- (trifluoromethoxy)phenyl] pyrido[3,4-b]pyrazin-5- amine hydrochloride | LCMS (Method A): Rt = 1.24 min, MH⁺ = 422.2 |
| 164 | 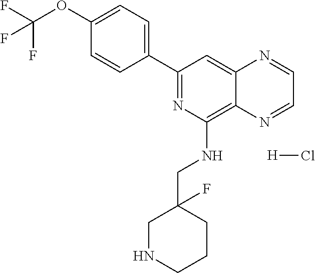 | N-[(3-fluoropiperidin-3-yl) methyl]-7-[4- (trifluoromethoxy)phenyl] pyrido[3,4-b]pyrazin-5- amine hydrochloride | LCMS (Method A): Rt = 1.24 min, MH⁺ = 422.2 |
| 165 | 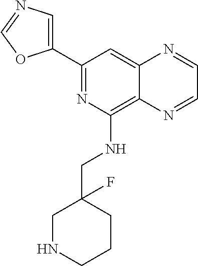 [SMILES: FC1(CNc2nc(-c3cnco3)cc3nccnc23)CCCNC1] | N-[(3-fluoropiperidin-3-yl) methyl]-7-(1,3-oxazol-5-yl) pyrido[3,4-b]pyrazin-5-amine | LCMS (Method A): Rt = 0.82 min, MH⁺ = 329.3 |
| 166 | 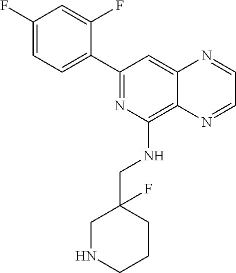 [SMILES: Fc1ccc(-c2cc3nccnc3c(NCC3(F)CCCNC3)n2)c(F)c1] | 7-(2,4-difluorophenyl)- N-[(3-fluoropiperidin-3- yl)methyl]pyrido[3,4-b] pyrazin-5-amine | LCMS (Method A): Rt = 1.12 min, MH⁺ = 374.2 |
| 167 | 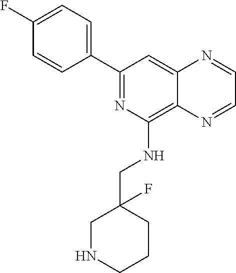 [SMILES: Fc1ccc(-c2cc3nccnc3c(NCC3(F)CCCNC3)n2)cc1] | 7-(4-fluorophenyl)-N-[(3- fluoropiperidin-3- yl)methyl]pyrido[3,4-b] pyrazin-5-amine | LCMS (Method A): Rt = 1.09 min, MH⁺ = 356.2 |
| 168 | 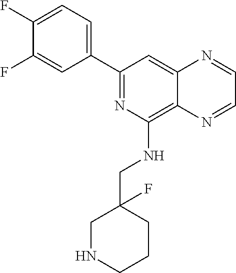 [SMILES: Fc1ccc(-c2cc3nccnc3c(NCC3(F)CCCNC3)n2)cc1F] | 7-(3,4-difluorophenyl)-N- [(3-fluoropiperidin-3- yl)methyl]pyrido[3,4-b] pyrazin-5-amine | LCMS (Method A): Rt = 1.14 min, MH⁺ = 374.2 |
| 169 | 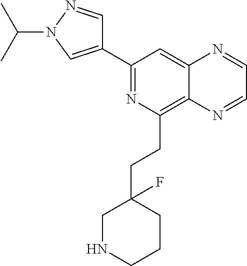 [SMILES: CC(C)n1cc(-c2cc3nccnc3c(CCC3(F)CCCNC3)n2)cn1] | 3-fluoro-3-(2-{7-[1-(propan- 2-yl)-1H-pyrazol-4-yl] pyrido[3,4-b]pyrazin-5-yl} ethyl)piperidine | LCMS (Method A): Rt = 0.95 min, MH⁺ = 369.3 |
| 170 | 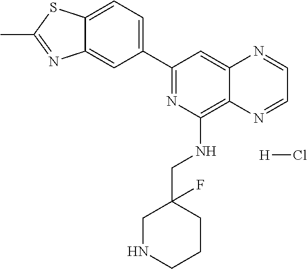 | N-[(3-fluoropiperidin-3- yl)methyl]-7-(2-methyl- 1,3-benzothiazol-5- yl)pyrido[3,4-b]pyrazin- 5-amine hydrochloride | LCMS (Method A): Rt = 1.06 min, MH⁺ = 409.2 |
| 171 | 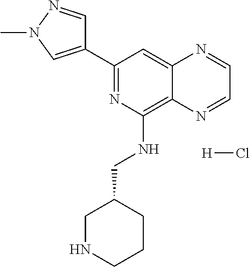 | 7-(1-methyl-1H-pyrazol- 4-yl)-N-[(3R)-piperidin-3- ylmethyl]pyrido[3,4-b] pyrazin-5-amine hydrochloride | LCMS (Method B): Rt = 0.54 min, MH⁺ = 324 |
| 172 | 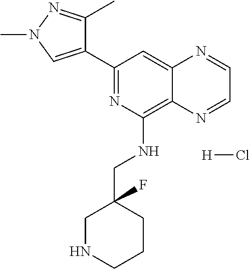 | 7-(1,3-dimethyl-1H-pyrazol- 4-yl)-N-{[(3S)-3- fluoropiperidin-3-yl]methyl} pyrido[3,4-b]pyrazin-5- amine hydrochloride | LCMS (Method A): Rt = 0.82 min, MH⁺ = 356.3 |
| 173 | 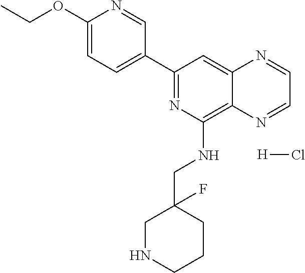 | 7-(6-ethoxypyridin-3-yl)- N-[(3-fluoropiperidin-3- yl)methyl]pyrido[3,4-b] pyrazin-5-amine hydrochloride | LCMS (Method B): Rt = 0.73 min, MH⁺ = 382.9 |
| 174 | 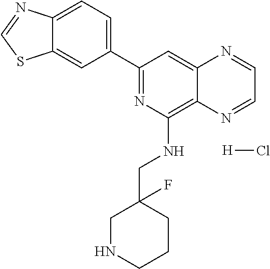 | 7-(1,3-benzothiazol-6-yl)- N-[(3-fluoropiperidin-3- yl)methyl]pyrido[3,4-b] pyrazin-5-amine hydrochloride | LCMS (Method A): Rt = 0.99 min, MH⁺ = 395.2 |
| 175 | 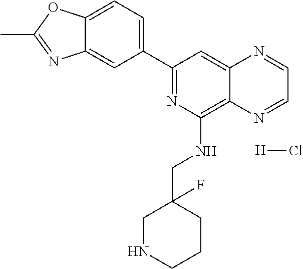 | N-[(3-fluoropiperidin-3-yl) methyl]-7-(2-methyl-1,3- benzoxazol-5-yl)pyrido [3,4-b]pyrazin-5-amine hydrochloride | LCMS (Method A): Rt = 0.99 min, MH⁺ = 393.3 |
| 176 | 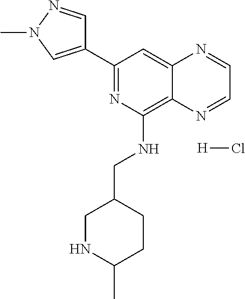 | 7-(1-methyl-1H-pyrazol- 4-yl)-N-[(6-methylpiperidin- 3-yl)methyl]pyrido[3,4-b] pyrazin-5-amine hydrochloride, single unknown enantiomer | LCMS (Method B): Rt = 0.54 min, MH⁺ = 338 |
| 177 | 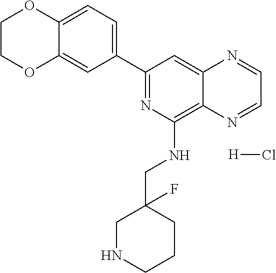 | 7-(2,3-dihydro-1,4- benzodioxin-6-yl)-N- [(3-fluoropiperidin-3- yl)methyl]pyrido[3,4-b] pyrazin-5-amine hydrochloride | LCMS (Method B): Rt = 0.72 min, MH⁺ = 396 |
| 178 | 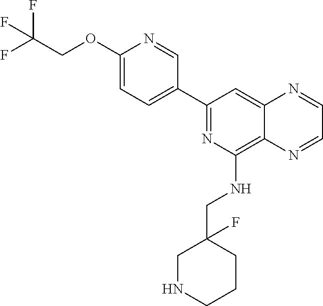 [SMILES: FC(F)(F)COc1ccc(-c2cc3nccnc3c(NCC3(F)CCCNC3)n2)cn1] | N-[(3-fluoropiperidin-3-yl) methyl]-7-[6-(2,2,2- trifluoroethoxy)pyridin-3- yl]pyrido[3,4-b]pyrazin-5- amine | LCMS (Method B): Rt = 0.86 min, MH⁺ = 437 |
| 179 | 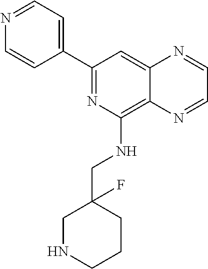 [SMILES: FC1(CNc2nc(-c3ccncc3)cc3nccnc23)CCCNC1] | N-[(3-fluoropiperidin-3-yl) methyl]-7-(pyridin-4-yl) pyrido[3,4-b]pyrazin-5- amine hydrochloride | LCMS (Method B): Rt = 0.41 min, MH⁺ = 338.9 |
| 180 | 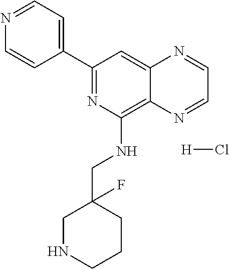 | 3-({[7-(1-ethyl-1H-pyrazol- 4-yl)pyrido[3,4-b]pyrazin-5- yl]oxy}methyl)-3- fluoropiperidine hydrochloride | LCMS (Method C): Rt = 0.60 min, MH⁺ = 357 |
| 181 | 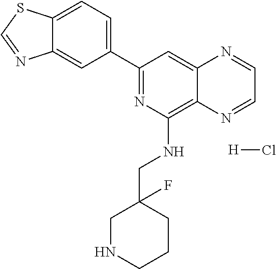 | 7-(1,3-benzothiazol-5- yl)-N-[(3-fluoropiperidin- 3-yl)methyl]pyrido[3,4-b] pyrazin-5-amine hydrochloride | LCMS (Method A): Rt = 0.95 min, MH⁺ = 395.1 |
| 182 | 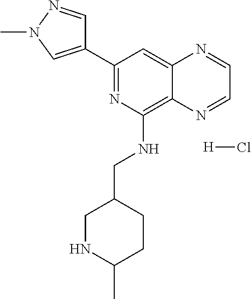 | 7-(1-methyl-1H-pyrazol-4-yl)- N-[(6-methylpiperidin-3- yl)methyl]pyrido[3,4-b] pyrazin-5-amine hydrochloride, single unknown enantiomer | LCMS (Method B): Rt = 0.54 min, MH⁺ = 338 |
| 183 | 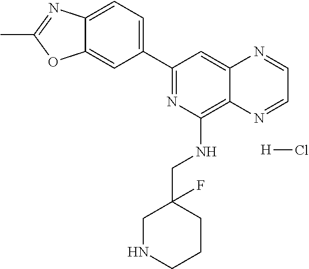 | N-[(3-fluoropiperidin-3-yl) methyl]-7-(2-methyl-1,3- benzoxazol-6-yl)pyrido [3,4-b]pyrazin-5-amine hydrochloride | LCMS (Method B): Rt = 0.68 min, MH⁺ = 393 |
| 184 | 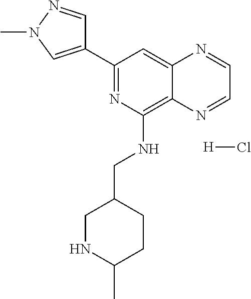 | 7-(1-methyl-1H-pyrazol-4- yl)-N-[(6-methylpiperidin-3- yl)methyl]pyrido[3,4-b] pyrazin-5-amine hydrochloride, single unknown enantiomer | LCMS (Method B): Rt = 0.55 min, MH⁺ = 338 |
| 185 | 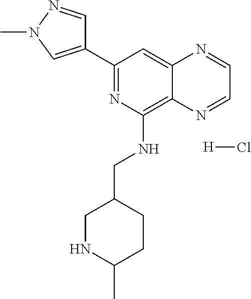 | 7-(1-methyl-1H-pyrazol-4- yl)-N-[(6-methylpiperidin- 3-yl)methyl]pyrido[3,4-b] pyrazin-5-amine hydrochloride, single unknown enantiomer | LCMS (Method B): Rt = 0.57 min, MH⁺ = 338 |
| 186 | 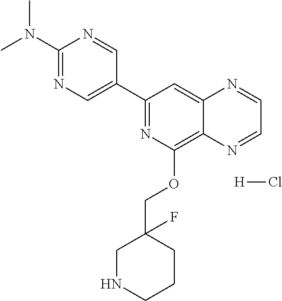 | 5-{5-[(3-fluoropiperidin-3- yl)methoxy]pyrido[3,4-b] pyrazin-7-yl}-N,N- dimethylpyrimidin-2- amine hydrochloride | LCMS (Method C): Rt = 0.62 min, MH⁺ = 383.9 |
| 187 | 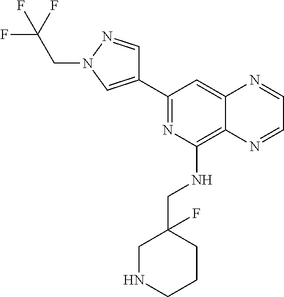 [SMILES: FC(F)(F)Cn1cc(-c2cc3nccnc3c(NCC3(F)CCCNC3)n2)cn1] | N-[(3-fluoropiperidin-3-yl) methyl]-7-[1-(2,2,2- trifluoroethyl)-1H-pyrazol- 4-yl]pyrido[3,4-b]pyrazin- 5-amine, single unknown enantiomer | LCMS (Method B): Rt = 0.63 min, MH⁺ = 409.9 |
| 188 | 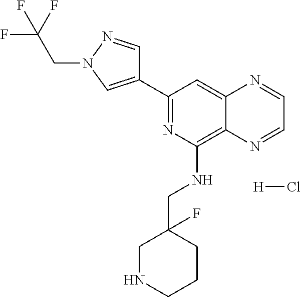 | N-[(3-fluoropiperidin-3- yl)methyl]-7-[1-(2,2,2- trifluoroethyl)-1H- pyrazol-4-yl]pyrido[3,4-b] pyrazin-5-amine hydrochloride, single unknown enantiomer | LCMS (Method C): Rt = 0.68 min, MH⁺ = 409.9 |
| 189 | 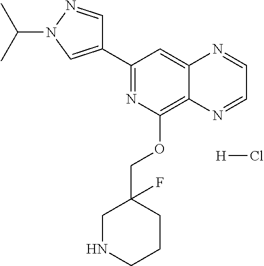 | 3-fluoro-3-[({7-[1-(propan- 2-yl)-1H-pyrazol-4-yl] pyrido[3,4-b]pyrazin-5- yl}oxy)methyl]piperidine hydrochloride | LCMS (Method B): Rt = 0.62 min, MH⁺ = 370.9 |
| 190 | 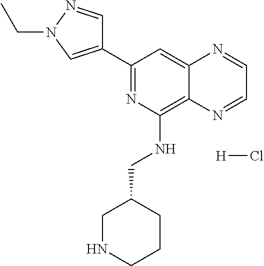 | 7-(1-ethyl-1H-pyrazol-4-yl)- N-[(3R)-piperidin-3- ylmethyl]pyrido[3,4-b]pyrazin- 5-amine hydrochloride | LCMS (Method A): Rt = 0.73 min, MH⁺ = 338.1 |
| 191 | 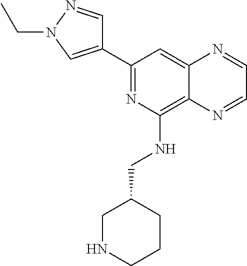 [SMILES: CCn1cc(-c2cc3nccnc3c(NC[C@@H]3CCCNC3)n2)cn1] | 7-(1-ethyl-1H-pyrazol-4-yl)- N-[(3R)-piperidin-3- ylmethyl]pyrido[3,4-b] pyrazin-5-amine | LCMS (Method B): Rt = 0.53 min, MH⁺ = 337.9 |
| 192 | 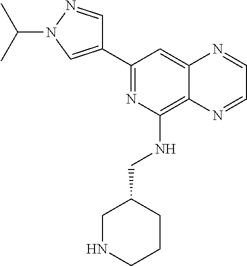 [SMILES: CC(C)n1cc(-c2cc3nccnc3c(NC[C@@H]3CCCNC3)n2)cn1] | N-[(3R)-piperidin-3-ylmethyl]- 7-[1-(propan-2-yl)-1H- pyrazol-4-yl]pyrido[3,4-b] pyrazin-5-amine | LCMS (Method B): Rt = 0.64 min, MH⁺ = 352 |
| 193 | 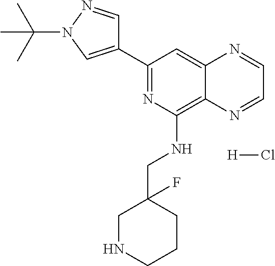 | 7-(1-tert-butyl-1H-pyrazol- 4-yl)-N-[(3-fluoropiperidin- 3-yl)methyl]pyrido[3,4-b] pyrazin-5-amine hydrochloride | LCMS (Method B): Rt = 0.70 min, MH⁺ = 383.9 |
| 194 | 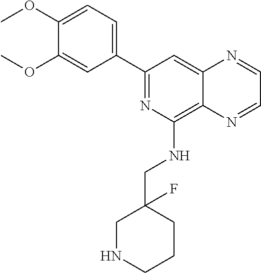 [SMILES: COc1ccc(-c2cc3nccnc3c(NCC3(F)CCCNC3)n2)cc1OC] | 7-(3,4-dimethoxyphenyl)- N-[(3-fluoropiperidin-3- yl)methyl]pyrido[3,4-b] pyrazin-5-amine | LCMS (Method C): Rt = 0.67 min, MH⁺ = 398 |
| 195 | 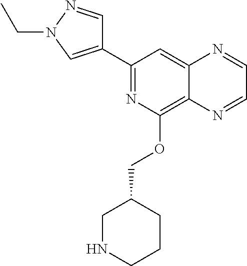 [SMILES: CCn1cc(-c2cc3nccnc3c(OC[C@@H]3CCCNC3)n2)cn1] | (3R)-3-({[7-(1-ethyl-1H- pyrazol-4-yl)pyrido[3,4-b] pyrazin-5-yl]oxy}methyl) piperidine | LCMS (Method B): Rt = 0.58 min, MH⁺ = 339 |
| 196 | 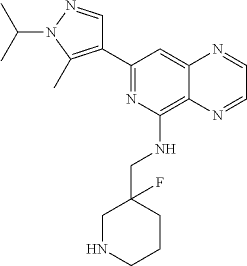 [SMILES: Cc1c(-c2cc3nccnc3c(NCC3(F)CCCNC3)n2)cnn1C(C)C] | N-[(3-fluoropiperidin-3-yl) methyl]-7-[5-methyl-1- (propan-2-yl)-1H-pyrazol- 4-yl]pyrido[3,4-b]pyrazin- 5-amine | LCMS (Method B): Rt = 0.66 min, MH⁺ = 384 |
| 197 | 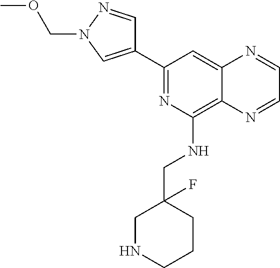 [SMILES: COCn1cc(-c2cc3nccnc3c(NCC3(F)CCCNC3)n2)cn1] | N-[(3-fluoropiperidin-3-yl) methyl]-7-[1-(methoxymethyl)- 1H-pyrazol-4-yl]pyrido [3,4-b]pyrazin-5-amine | LCMS (Method B): Rt = 0.55 min, MH⁺ = 371.9 |
| 198 | 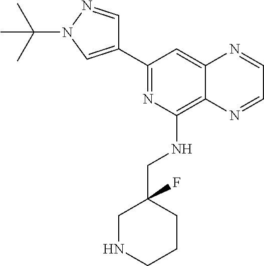 [SMILES: CC(C)(C)n1cc(-c2cc3nccnc3c(NC[C@]3(F)CCCNC3)n2)cn1] | 7-(1-tert-butyl-1H-pyrazol- 4-yl)-N-{[(3S)-3- fluoropiperidin-3-yl]methyl} pyrido[3,4-b]pyrazin-5- amine | LCMS (Method C): Rt = 0.69 min, MH⁺ = 383.9 |
| 199 | 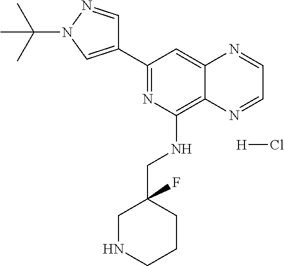 | 7-(1-tert-butyl-1H-pyrazol- 4-yl)-N-{[(3S)-3- fluoropiperidin-3-yl]methyl} pyrido[3,4-b]pyrazin-5- amine hydrochloride | LCMS (Method C): Rt = 0.69 min, MH⁺ = 383.9 |
| 200 | 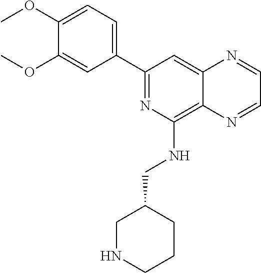 [SMILES: COc1ccc(-c2cc3nccnc3c(NC[C@@H]3CCCNC3)n2)cc1OC] | 7-(3,4-dimethoxyphenyl)-N- [(3R)-piperidin-3-ylmethyl] pyrido[3,4-b]pyrazin-5- amine | LCMS (Method C): Rt = 0.66 min, MH⁺ = 380.1 |
| 201 | 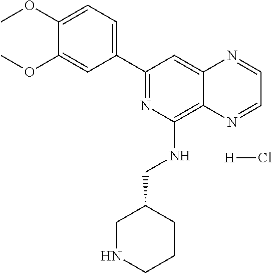 | 7-(3,4-dimethoxyphenyl)-N- [(3R)-piperidin-3-ylmethyl] pyrido[3,4-b]pyrazin-5- amine hydrochloride | LCMS (Method C): Rt = 0.67 min, MH⁺ = 380.1 |
| 202 | 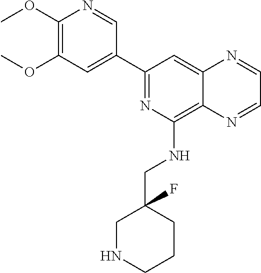 [SMILES: COc1cc(-c2cc3nccnc3c(NC[C@]3(F)CCCNC3)n2)cnc1OC] | 7-(5,6-dimethoxypyridin- 3-yl)-N-{[(3S)-3- fluoropiperidin-3-yl] methyl}pyrido[3,4-b] pyrazin-5-amine | LCMS (Method B): Rt = 0.67 min, MH⁺ = 399.2 |
| 203 | 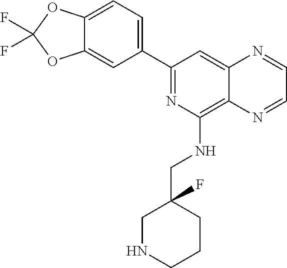 [SMILES: FC1(F)Oc2ccc(-c3cc4nccnc4c(NC[C@]4(F)CCCNC4)n3)cc2O1] | 7-(2,2-difluoro-2H-1,3- benzodioxol-5-yl)-N- {[(3S)-3-fluoropiperidin- 3-yl]methyl}pyrido[3,4-b] pyrazin-5-amine | LCMS (Method B): Rt = 0.85 min, MH⁺ = 418.2 |

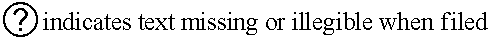

1. A compound of formula (I): 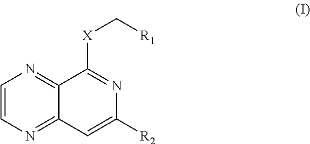
wherein:
X is O, CH₂ or NH;
R₁ is a 5- or 6-membered heterocyclyl or —(CH₂)ₙR₅;
wherein the heterocyclyl is optionally substituted by one or two groups each independently selected from fluoro, methyl, ethyl and trifluoroethyl;
R₂ is a 5- or 6-membered heteroaryl, heterocyclyl or phenyl, or a 9- or 10-membered fused heteroaryl;
wherein the heteroaryl, heterocyclyl, phenyl or fused heteroaryl is optionally substituted by one or two groups each independently selected from C₁₋₆alkyl, OH, C₁₋₆alkoxy, —NR₃R₄, C₁₋₆-fluoroalkyl, benzyl, C₃₋₆cycloalkyl, oxo (═O), OC₁₋₆-fluoroalkyl and halogen;
R₃ and R₄ are each independently selected from hydrogen and methyl, or R₃ and R₄ together with the nitrogen to which they are attached form a 5- or 6-membered heterocyclyl;
R₅ is —NH₂, —CF₃, —C(O)NH₂ or OH; and
n is an integer selected from 0, 1, 2 and 3; or
a pharmaceutically acceptable salt thereof.

2. A compound or a pharmaceutically acceptable salt thereof according to claim 1 wherein X is O or NH.

3. A compound or a pharmaceutically acceptable salt thereof according to claim 1 wherein R₁ is a 6-membered heterocyclyl.

4. A compound or a pharmaceutically acceptable salt thereof according to claim 3 wherein the 6-membered heterocyclyl is selected from piperidine, piperazine and morpholine.

5. A compound or a pharmaceutically acceptable salt thereof according to claim 1 wherein R₁ is —(CH₂)ₙR₅ and R₅ is —NH₂.

6. A compound or a pharmaceutically acceptable salt thereof according to claim 1 wherein R₂ is selected from pyrazole, pyridine and phenyl.

7. A compound or a pharmaceutically acceptable salt thereof according to claim 1 wherein R₃ and R₄ are both methyl.

8. A compound which is selected from the group consisting of:
7-[1-(Phenylmethyl)-1H-pyrazol-4-yl]-N-[(3S)-3-piperidinylmethyl]pyrido[3,4-b]pyrazin-5-amine;
7-(1-Cyclopentyl-1H-pyrazol-4-yl)-N-[(3S)-3-piperidinylmethyl]pyrido[3,4-b]pyrazin-5-amine;
N-{[(3S)-3-fluoro-3-piperidinyl]methyl}-7-[1-(2,2,2-trifluoroethyl)-1H-pyrazol-4-yl]pyrido[3,4-b]pyrazin-5-amine;
7-[3,4-bis(Methyloxy)phenyl]-N-{[(3S)-3-fluoro-3-piperidinyl]methyl}pyrido[3,4-b]pyrazin-5-amine hydrochloride;
N-{[(3S)-3-Fluoro-3-piperidinyl]methyl}-7-(1-methyl-1H-pyrazol-4-yl)pyrido[3,4-b]pyrazin-5-amine hydrochloride;
5-{[(3S)-3-piperidinylmethyl]oxy}-7-[1-(2,2,2-trifluoroethyl)-1H-pyrazol-4-yl]pyrido[3,4-b]pyrazine hydrochloride;
7-(1,5-Dimethyl-1H-pyrazol-4-yl)-5-{[(3S)-3-piperidinylmethyl]oxy}pyrido[3,4-b]pyrazine, hydrochloride;
N,N-Dimethyl-5-(5-{[(3S)-3-piperidinylmethyl]oxy}pyrido[3,4-b]pyrazin-7-yl)-2-pyridinamine hydrochloride;
7-(1-Methyl-1H-pyrazol-4-yl)-5-{[(3S)-3-piperidinylmethyl]oxy}pyrido[3,4-b]pyrazine hydrochloride;
7-(1,5-Dimethyl-1H-pyrazol-4-yl)-5{[(2S)-2-morpholinylmethyl]oxy}pyrido[3,4-b]pyrazine, hydrochloride;
7-(1-Methyl-1H-pyrazol-4-yl)-N-[(2S)-2-morpholinylmethyl]pyrido[3,4-b]pyrazin-5-amine, hydrochloride;
N-{[(2S)-1-methyl-2-piperazinyl]methyl}-7-(1-methyl-1H-pyrazol-4-yl)pyrido[3,4-b]pyrazin-5-amine hydrochloride;
5-{[(4,4-Difluoro-3-piperidinyl)methyl]oxy}-7-(1-methyl-1H-pyrazol-4-yl)pyrido[3,4-b]pyrazine;
7-(1-Methyl-1H-pyrazol-4-yl)-5-[2-(3-piperidinyl)ethyl]pyrido[3,4-b]pyrazine hydrochloride;
N-{7-[6-(Dimethylamino)-3-pyridinyl]pyrido[3,4-b]pyrazin-5-yl}-1,4-butanediamine hydrochloride;
7-[6-(dimethylamino)-3-pyridinyl]-N-[(2)-2-morpholinylmethyl]pyrido[3,4-b]pyrazin-5-amine, hydrochloride (Isomer 1);
7-[6-(dimethylamino)-3-pyridinyl]-N-[2-morpholinylmethyl]pyrido[3,4-b]pyrazin-5-amine, hydrochloride (Isomer 2);
N-[(4,4-difluoro-3-piperidinyl methyl]-7-[6-(dimethylamino)-3-pyridinyl]pyrido[3,4-b]pyrazin-5-amine (Isomer 2);
N-[(4,4-difluoro-3-piperidinyl methyl]-7-[6-(dimethylamino)-3-pyridinyl]pyrido[3,4-b]pyrazin-5-amine (Isomer 1);
N-((5,5-difluoropiperidin-3-yl)methyl)-7-(6-(dimethylamino)pyridin-3-yl)pyrido[3,4-b]pyrazin-5-amine;
4-(5-{[(3S)-3-piperidinyl methyl]amino}pyrido[3,4-b]pyrazin-7-yl)-2-piperazinone, hydrochloride;
7-(1-piperazinyl)-N-[(3S)-3-piperidinylmethyl]pyrido[3,4-b]pyrazin-5-amine, hydrochloride;
N,N-dimethyl-5-(5-{[(3R)-3-piperidinylmethyl]oxy}pyrido[3,4-b]pyrazin-7-yl)-2-pyridinamine;
7-[6-(dimethylamino)-3-pyridinyl]-N-(2,2,2-trifluoroethyl)pyrido[3,4-b]pyrazin-5-amine;
4-({7-[6-(dimethylamino)-3-pyridinyl]pyrido[3,4-b]pyrazin-5-yl}amino)-1-butanol;
N³-{7-[6-(dimethylamino)-3-pyridinyl]pyrido[3,4-b]pyrazin-5-yl}-β-alaninamide;
7-[6-(dimethylamino)-3-pyridinyl]-N-[(3S)-3-piperidinylmethyl]pyrido[3,4-b]pyrazin-5-amine;
N,N-di methyl-5-{5-[2-(3-piperidinyl ethyl]pyrido[3,4-b]pyrazin-7-yl}-2-pyridinamine;
7-(1-methyl-1H-pyrazol-4-yl)-N-[(3S)-3-piperidinylmethyl]pyrido[3,4-b]pyrazin-5-amine;
N-[(5,5-difluoro-3-piperidinyl)methyl]-7-(1-methyl-1H-pyrazol-4-yl)pyrido[3,4-b]pyrazin-5-amine;
7-[6-(dimethylamino)-3-pyridinyl]-N-[(3R)-3-piperidinylmethyl]pyrido[3,4-b]pyrazin-5-amine;
N-[(3S)-3-piperidinylmethyl]-7-[6-(1-pyrrolidinyl)-3-pyridinyl]pyrido[3,4-b]pyrazin-5-amine;
7-[6-(1-piperazinyl)-3-pyridinyl]-N-[(3S)-3-piperidinyl methyl]pyrido[3,4-b]pyrazin-5-amine;
7-(6-amino-3-pyridinyl)-N-[(3S)-3-piperidinylmethyl]pyrido[3,4-b]pyrazin-5-amine;
7-(2-amino-5-pyrimidinyl)-N-[(3S)-3-piperidinylmethyl]pyrido[3,4-b]pyrazin-5-amine;
5-(5-{[(3S)-3-piperidinyl methyl]amino}pyrido[3,4-b]pyrazin-7-yl)-2(1H)-pyridinone;
N-[(3S)-3-piperidinylmethyl]-7-(1H-pyrrolo[2,3-b]pyridin-5-yl)pyrido[3,4-b]pyrazin-5-amine;
7-(5-methyl-2-thienyl)-N-[(3S)-3-piperidinylmethyl]pyrido[3,4-b]pyrazin-5-amine;
7-(5-methyl-2-furanyl)-N-[(3S)-3-piperidinylmethyl]pyrido[3,4-b]pyrazin-5-amine;
N-[(3S)-3-piperidinylmethyl]-7-(1H-pyrazol-3-yl)pyrido[3,4-b]pyrazin-5-amine;
N-[(3S)-3-piperidinylmethyl]-7-(1H-pyrazol-3-yl)pyrido[3,4-b]pyrazin-5-amineN-[7-4-methylphenyl)pyrido[3,4-b]pyrazin-5-yl]-1,4-butanediamine;
7-(4-methylphenyl)-N-(morpholin-2-ylmethyl)pyrido[3,4-b]pyrazin-5-amine hydrochloride, single unknown enantiomer;
7-(4-methylphenyl)-N-(morpholin-2-ylmethyl)pyrido[3,4-b]pyrazin-5-amine hydrochloride, single unknown enantiomer;
7-(4-methoxyphenyl)-N-[(3S)-piperidin-3-ylmethyl]pyrido[3,4-b]pyrazin-5-amine hydrochloride;
(3S)-3-({[7-(4-methylphenyl)pyrido[3,4-b]pyrazin-5-yl]oxy}methyl)piperidine hydrochloride;
7-(2,3-dihydro-1-benzofuran-5-yl)-N-[(3S)-piperidin-3-ylmethyl]pyrido[3,4-b]pyrazin-5-amine hydrochloride;
7-(1,3-benzothiazol-5-yl)-N-[(3S)-piperidin-3-ylmethyl]pyrido[3,4-b]pyrazin-5-amine hydrochloride;
7-(1H-indol-5-yl)-N-[(3S)-piperidin-3-ylmethyl]pyrido[3,4-b]pyrazin-5-amine hydrochloride;
7-(2,3-dihydro-1,4-benzodioxin-6-yl)-N-[(3S)-piperidin-3-ylmethyl]pyrido[3,4-b]pyrazin-5-amine hydrochloride;
7-[6-(morpholin-4-yl)pyridin-3-yl]-N-[(3S)-piperidin-3-ylmethyl]pyrido[3,4-b]pyrazin-5-amine hydrochloride;
N-[(3S)-piperidin-3-ylmethyl]-7-[6-propan-2-yloxyl)pyridin-3-yl]pyrido[3,4-b]pyrazin-5-amine hydrochloride;
(3S)-3-({[7-(4-methoxyphenyl)pyrido[3,4-b]pyrazin-5-yl]oxy}methyl)piperidine hydrochloride;
(3S)-3-({[7-(1H-pyrazol-4-yl)pyrido[3,4-b]pyrazin-5-yl]oxy}methyl)piperidine hydrochloride;
(3S)-3-({[7-(1-benzofuran-3-yl)pyrido[3,4-b]pyrazin-5-yl]oxy}methyl)piperidine hydrochloride;
(3S)-3-{[(7-{1H-pyrrolo[3,2-c]pyridin-3-yl}pyrido[3,4-b]pyrazin-5-yl)oxy]methyl}piperidine hydrochloride;
(3S)-3-{[(7-{1H-pyrrolo[2,3-b]pyridin-3-yl}pyrido[3,4-b]pyrazin-5-yl)oxy]methyl}piperidine hydrochloride;
(2R)-2-({[7-(1H-pyrazol-4-yl)pyrido[3,4-b]pyrazin-5-yl]oxy}methyl)morpholine hydrochloride;
(2R)-2-({[7-(1-methyl-1H-pyrazol-4-yl)pyrido[3,4-b]pyrazin-5-yl]oxy}methyl)morpholine hydrochloride;
N,N-dimethyl-5-{5-[(2R)-morpholin-2-ylmethoxy]pyrido[3,4-b]pyrazin-7-yl}pyridin-2-amine hydrochloride;
N-(morpholin-2-ylmethyl)-7-(1H-pyrazol-4-yl)pyrido[3,4-b]pyrazin-5-amine hydrochloride, single unknown enantiomer;
(3S)-3-{[(7-{4-methyl-2H,3H,4H-pyrido[3,2-b][1,4]oxazin-7-yl}pyrido[3,4-b]pyrazin-5-yl)oxy]methyl}piperidine hydrochloride;
(3S)-3-[({7-[1-(propan-2-yl)-1H-pyrazol-4-yl]pyrido[3,4-b]pyrazin-5-yl}oxy)methyl]piperidine hydrochloride;
(3S)-3-[({7-[1-(propan-2-yl)-1H-pyrazol-4-yl]pyrido[3,4-b]pyrazin-5-yl}oxy)methyl]piperidine;
N,N-dimethyl-5-{5-[(3S)-piperidin-3-ylmethoxy]pyrido[3,4-b]pyrazin-7-yl}pyrimidin-2-amine hydrochloride;
(3S)-3-({[7-(2,3-dihydro-1,4-benzodioxin-6-yl)pyrido[3,4-b]pyrazin-5-yl]oxy}methyl)piperidine hydrochloride;
(3S)-3-({[7-(4-chlorophenyl)pyrido[3,4-b]pyrazin-5-yl]oxy}methyl)piperidine;
(3S)-[({7-[4-(propan-2-yloxy)phenyl]pyrido[3,4-b]pyrazin-5-yl}oxy)methyl]piperidine;
(3S)-3-({[7-(1-ethyl-1H-pyrazol-4-yl)pyrido[3,4-b]pyrazin-5-yl]oxy}methyl)piperidine hydrochloride;
N,N-dimethyl-5-[5-({[(2S)-1-methylpiperazin-2-yl]methyl}amino)pyrido[3,4-b]pyrazin-7-yl]pyridin-2-amine hydrochloride;
4-(5-{5-[(3S)-piperidin-3-ylmethoxy]pyrido[3,4-b]pyrazin-7-yl}pyridin-2-yl)morpholine
(3S)-3-({[7-(3-methylphenyl)pyrido[3,4-b]pyrazin-5-yl]oxy}methyl)piperidine dihydrochloride;
N-(morpholin-2-ylmethyl) 7-[6-(morpholin-4-yl)pyridin-3-yl]pyrido[3,4-b]pyrazin-5-amine hydrochloride; single unknown enantiomer;
N-{[(2S)-1-methylpiperazin-2-yl]methyl}-7-(1H-pyrazol-4-yl)pyrido[3,4-b]pyrazin-5-amine hydrochloride;
N-{[(2S)-1-methylpiperazin-2-yl]methyl}-7-[6-(morpholin-4-yl)pyridin-3-yl]pyrido[3,4-b]pyrazin-5-amine hydrochloride;
(3S)-3-({[7-(1,3-dimethyl-1H-pyrazol-4-yl)pyrido[3,4-b]pyrazin-5-yl]oxy}methyl)piperidine;
(3S)-3-({[7-(1,3-dimethyl-1H-pyrazol-4-yl)pyrido[3,4-b]pyrazin-5-yl]oxy}methyl)piperidine hydrochloride;
7-(1-ethyl-1H-pyrazol-4-yl)-N-[(2R)-morpholin-2-ylmethyl]pyrido[3,4-b]pyrazin-5-amine;
N-[(2R)-morpholin-2-ylmethyl]-7-[1-(propan-2-yl)-1H-pyrazol-4-yl]pyrido[3,4-b]pyrazin-5-amine;
(3S)-3-({[7-(2-chloro-4-methylphenyl)pyrido[3,4-b]pyrazin-5-yl]oxy}methyl)piperidine hydrochloride;
(3S)-3-({[7-(3-chloropheny)pyrido[3,4-b]pyrazin-5-yl]oxy}methyl)piperidine hydrochloride;
N-{[(2S)-1-methylpiperazin-2-yl]methyl}-7-[1-(propan-2-yl)-1H-pyrazol-4-yl]pyrido[3,4-b]pyrazin-5-amine hydrochloride;
(3S)-[({7-[4-(trifluoromethyl phenyl]pyrido[3,4-b]pyrazin-5-yl}oxy)methyl]piperidine hydrochloride;
(3S)-[({7-[4-(trifluoromethyl)phenyl]pyrido[3,4-b]pyrazin-5-yl}oxy)methyl]piperidine;
(3S)-3-({[7-(2-fluoro-4-methylphenyl)pyrido[3,4-b]pyrazin-5-yl]oxy}methyl)piperidine;
N,N-dimethyl-5-(5-{[(2S)-1-methylpiperazin-2-yl]methoxy}pyrido[3,4-b]pyrazin-7-yl)pyridin-2-amine;
(2S)-1-methyl-2-({[7-(1-methyl-1H-pyrazol-4-yl)pyrido[3,4-b]pyrazin-5-yl]oxy}methyl)piperazine
7-(1,3-dimethyl-1H-pyrazol-4-yl)-N-[(2S)-morpholin-2-ylmethyl]pyrido[3,4-b]pyrazin-5-amine hydrochloride;
N,N-dimethyl-5-[5-({[(2R)-1-methylpiperazin-2-yl]methyl}amino)pyrido[3,4-b]pyrazin-7-yl]pyridin-2-amine hydrochloride;
7-(1,5-dimethyl-1H-pyrazol-4-yl)-N-[(2S)-morpholin-2-ylmethyl]pyrido[3,4-b]pyrazin-5-amine hydrochloride;
(2S)-2-({[7-(4-methylphenyl)pyrido[3,4-b]pyrazin-5-yl]oxy}methyl)morpholine hydrochloride;
7-(1-methyl-1H-pyrazol-5-yl)-N-[(2S)-morpholin-2-ylmethyl]pyrido[3,4-b]pyrazin-5-amine hydrochloride;
7-(1,3-dimethyl-1H-pyrazol-5-yl)-N-[(2S)-morpholin-2-ylmethyl]pyrido[3,4-b]pyrazin-5-amine;
7-[1-(2-methylpropyl)-1H-pyrazol-4-yl]-N-[(2S)-morpholin-2-ylmethyl]pyrido[3,4-b]pyrazin-5-amine;
N-[(2S)-morpholin-2-ylmethyl]-7-[6-(morpholin-4-yl)pyridin-3-yl]pyrido[3,4-b]pyrazin-5-amine hydrochloride;
7-(1-ethyl-1H-pyrazol-4-yl)-N-[(2S)-morpholin-2-ylmethyl]pyrido[3,4-b]pyrazin-5-amine hydrochloride;
N-[(2S)-morpholin-2-ylmethyl]-7-[1-(propan-2-yl)-1H-pyrazol-4-yl]pyrido[3,4-b]pyrazin-5-amine;
(2S)-2-({[7-(1,5-dimethyl-1H-pyrazol-4-yl)pyrido[3,4-b]pyrazin-5-yl]oxy}methyl)-1-methylpiperazine
(2S)-2-({[7-(1-methyl-1H-pyrazol-4-yl)pyrido[3,4-b]pyrazin-5-yl]oxy}methyl)morpholine hydrochloride;
N-[(2S)-morpholin-2-ylmethyl]-7-[1-(2,2,2-trifluoroethyl)-1H-pyrazol-4-yl]pyrido[3,4-b]pyrazin-5-amine hydrochloride;
N-[(2S)-morpholin-2-ylmethyl]-7-[1-(2,2,2-trifluoroethyl)-1H-pyrazol-4-yl]pyrido[3,4-b]pyrazin-5-amine;
N-[(2S)-morpholin-2-ylmethyl]-7-(1-propyl-1H-pyrazol-4-yl)pyrido[3,4-b]pyrazin-5-amine;
N-[(2S)-morpholin-2-ylmethyl]-7-(1-propyl-1H-pyrazol-4-yl)pyrido[3,4-b]pyrazin-5-amine hydrochloride;
7-(4-tert-butylphenyl)-N-[(2S)-morpholin-2-ylmethyl]pyrido[3,4-b]pyrazin-5-amine;
(2S)-1-ethyl-2-({[7-(1-methyl-1H-pyrazol-4-yl)pyrido[3,4-b]pyrazin-5-yl]oxy}methyl)piperazine hydrochloride;
5-{5-[(3-fluoropiperidin-3-yl)methoxy]pyrido[3,4-b]pyrazin-7-yl}-N,N-dimethylpyridin-2-amine;
5-{5-[(3-fluoropiperidin-3-ylmethoxy]pyrido[3,4-b]pyrazin-7-yl}-N,N-dimethylpyridin-2-amine, single unknown enantiomer;
5-{5-[(3-fluoropiperidin-3-yl)methoxy]pyrido[3,4-b]pyrazin-7-yl}-N,N-dimethylpyridin-2-amine, single unknown enantiomer;
5-(5-{[(2S)-1-ethylpiperazin-2-yl]methoxy}pyrido[3,4-b]pyrazin-7-yl)-N,N-dimethylpyridin-2-amine hydrochloride;
(3S)-3-{[(7-{4H,5H,6H-pyrrolo[1,2-b]pyrazol-3-yl}pyrido[3,4-b]pyrazin-5-yl)oxy]methyl}piperidine;
(3R)-3-({[7-(1-methyl-1H-pyrazol-4-yl)pyrido[3,4-b]pyrazin-5-yl]oxy}methyl)piperidine;
N,N-dimethyl-5-(5-{[(2S)-1-(2,2,2-trifluoroethyl)piperazin-2-yl]-methoxy}pyrido[3,4-b]pyrazin-7-yl)pyridin-2-amine;
(2S)-2-({[7-(1-methyl-1H-pyrazol-4-yl)pyrido[3,4-b]pyrazin-5-yl]oxy}methyl)-1-(2,2,2-trifluoroethyl)piperazine
(6S)-2,2-dimethyl-6-({[7-(1-methyl-1H-pyrazol-4-yl)pyrido[3,4-b]pyrazin-5-yl]oxy}methyl)morpholine
N-[(4,4-difluoropiperidin-3-yl)methyl]-7-(1-methyl-1H-pyrazol-4-yl)pyrido[3,4-b]pyrazin-5-amine;
3-fluoro-3-({[7-(1-methyl-1H-pyrazol-4-yl)pyrido[3,4-b]pyrazin-5-yl]oxy}methyl)piperidine, single unknown enantiomer;
3-fluoro-3-({[7-(1-methyl-1H-pyrazol-4-yl)pyrido[3,4-b]pyrazin-5-yl]oxy}methyl)piperidine, single unknown enantiomer;
5-[5-({[(3R)-3-fluoropiperidin-3-yl]methyl}amino)pyrido[3,4-b]pyrazin-7-yl]-N,N-dimethylpyridin-2-amine hydrochloride;
N-{[(3S)-3-fluoropiperidin-3-yl]methyl}-7-(1-methyl-1H-pyrazol-4-yl)pyrido[3,4-b]pyrazin-5-amine hydrochloride;
N-[(4,4-difluoropiperidin-3-yl)methyl]-7-(1-methyl-1H-pyrazol-4-yl)pyrido[3,4-b]pyrazin-5-amine, single unknown enantiomer;
N-[(4,4-difluoropiperidin-3-yl)methyl]-7-(1-methyl-1H-pyrazol-4-yl)pyrido[3,4-b]pyrazin-5-amine, single unknown enantiomer;
5-[5-({[(3S)-3-fluoropiperidin-3-yl]-methyl}amino)pyrido[3,4-b]pyrazin-7-yl]-N,N-dimethylpyridin-2-amine hydrochloride;
(2S,3S)-2-methyl-3-({[7-(1-methyl-1H-pyrazol-4-yl)pyrido[3,4-b]pyrazin-5-yl]oxy}methyl)piperidine;
7-(1-methyl-1H-pyrazol-4-yl)-N-[(2R)-morpholin-2-ylmethyl]pyrido[3,4-b]pyrazin-5-amine;
3,3-difluoro-5-({[7-(1-methyl-1H-pyrazol-4-yl)pyrido[3,4-b]pyrazin-5-yl]oxy}methyl)piperidine hydrochloride;
N-[(3-fluoropiperidin-3-yl)methyl]-7-(4-methylphenyl)pyrido[3,4-b]pyrazin-5-amine;
N-[(3-fluoropiperidin-3-yl)methyl]-7-(6-methoxypyridin-3-yl)pyrido[3,4-b]pyrazin-5-amine;
N-[(3-fluoropiperidin-3-yl)methyl]-7-(5-methoxypyridin-3-yl)pyrido[3,4-b]pyrazin-5-amine;
N-[(3-fluoropiperidin-3-yl)methyl]-7-(6-methylpyridin-3-yl)pyrido[3,4-b]pyrazin-5-amine;
N-[(3-fluoropiperidin-3-yl)methyl]-7-(4-methoxyphenyl)pyrido[3,4-b]pyrazin-5-amine;
N-[(4,4-difluoropiperidin-3-yl)methyl]-7-(4-methyl phenyl)pyrido[3,4-b]pyrazin-5-amine;
N-[(4,4-difluoropiperidin-3-yl)methyl]-7-(4-methoxyphenyl)pyrido[3,4-b]pyrazin-5-amine;
5-(5-{[(3-fluoropiperidin-3-yl)methyl]amino}pyrido[3,4-b]pyrazin-7-yl)-N,N-dimethylpyrimidin-2-amine;
N-(4-methylphenyl)-5-[(3S)-piperidin-3-ylmethoxy]pyrido[3,4-b]pyrazin-7-amine hydrochloride;
7-(1-cyclopentyl-1H-pyrazol-4-yl)-N-[(3-fluoropiperidin-3-yl)methyl]pyrido[3,4-b]pyrazin-5-amine hydrochloride;
7-(1-cyclopentyl-1H-pyrazol-4-yl)-N-[(3-fluoropiperidin-3-yl)methyl]pyrido[3,4-b]pyrazin-5-amine;
N-[(3-fluoropiperidin-3-yl)methyl]-7-[1-(propan-2-yl)-1H-pyrazol-4-yl]pyrido[3,4-b]pyrazin-5-amine hydrochloride;
N-[(3-fluoropiperidin-3-yl)methyl]-7-[1-(pentan-3-yl)-1H-pyrazol-4-yl]pyrido[3,4-b]pyrazin-5-amine hydrochloride;
7-(1-benzyl-1H-pyrazol-4-yl)-N-[(3-fluoropiperidin-3-yl)methyl]pyrido[3,4-b]pyrazin-5-amine hydrochloride;
7-(1-ethyl-1H-pyrazol-4-yl)-N-[(3-fluoropiperidin-3-yl)methyl]pyrido[3,4-b]pyrazin-5-amine hydrochloride;
7-(1,5-dimethyl-1H-pyrazol-4-yl)-N-[(3-fluoropiperidin-3-yl)methyl]pyrido[3,4-b]pyrazin-5-amine hydrochloride;
N-[(3-fluoropieridin-3-yl)methyl]-7-[1-(2-methylpropyl)-1H-pyrazol-4-yl]pyrido[3,4-b]pyrazin-5-amine hydrochloride;
N-[(3-fluoropieridin-3-yl)methyl]-7-(1-propyl-1H-pyrazol-4-yl)pyrido[3,4-b]pyrazin-5-amine hydrochloride;
7-[1-(2-methylpropyl)-1H-pyrazol-4-yl]-N-[(3S)-piperidin-3-ylmethyl]pyrido[3,4-b]pyrazin-5-amine;
7-(1,5-dimethyl-1H-pyrazol-4-yl)-N-[(3S)-piperidin-3-ylmethyl]pyrido[3,4-b]pyrazin-5-amine hydrochloride;
(3S)-[({7-[(4-methylphenyl methyl]pyrido[3,4-b]pyrazin-5-yl}oxy)methyl]piperidine hydrochloride;
7-(1,3-dimethyl-1H-pyrazol-4-yl)-N-[(3S)-piperidin-3-ylmethyl]pyrido[3,4-b]pyrazin-5-amine hydrochloride;
N-[(3S)-piperidin-3-ylmethyl]-7-(1-propyl-1H-pyrazol-4-yl)pyrido[3,4-b]pyrazin-5-amine hydrochloride;
7-(1,3-dimethyl-1H-pyrazol-4-yl)-N-[(3-fluoropiperidin-3-yl)methyl]pyrido[3,4-b]pyrazin-5-amine hydrochloride;
7-(1-ethyl-1H-pyrazol-4-yl)-N-[(3S)-piperidin-3-ylmethyl]pyrido[3,4-b]pyrazin-5-amine;
7-[1-(pentan-3-yl)-1H-pyrazol-4-yl]-N-[(3S)-piperidin-3-ylmethyl]pyrido[3,4-b]pyrazin-5-amine;
N-[(3S)-piperidin-3-ylmethyl]-7-[1-(propan-2-yl)-1H-pyrazol-4-yl]pyrido[3,4-b]pyrazin-5-amine;
N-[(3S)-piperidin-3-ylmethyl]-7-[1-(propan-2-yl)-1H-pyrazol-4-yl]pyrido[3,4-b]pyrazin-5-amine hydrochloride;
7-(dimethyl-1,2-oxazol-4-yl)-N-[(3-fluoropiperidin-3-yl)methyl]pyrido[3,4-b]pyrazin-5-amine hydrochloride;
N-[(3-fluoropiperidin-3-yl)methyl]-7-[1-(2,2,2-trifluoroethyl)-1H-pyrazol-4-yl]pyrido[3,4-b]pyrazin-5-amine hydrochloride;
3-(2-{7-[1-(propan-2-yl)-1H-pyrazol-4-yl]pyrido[3,4-b]pyrazin-5-yl}ethyl)piperidine;
7-[5-methyl-1-(propan-2-yl)-1H-pyrazol-4-yl]-N-[(3S)-piperidin-3-ylmethyl]pyrido[3,4-b]pyrazin-5-amine hydrochloride;
(+/−)(3S,5R)-3-fluoro-5-({[7-(1-methyl-1H-pyrazol-4-yl)pyrido[3,4-b]pyrazin-5-yl]oxy}methyl)piperidine;
(+/−)(3R,5R)-3-fluoro-5-({[7-(1-methyl-1H-pyrazol-4-yl)pyrido[3,4-b]pyrazin-5-yl]oxy}methyl)piperidine;
N-[(3-fluoropiperidin-3-yl)methyl]-7-[3-(trifluoromethoxy)phenyl]pyrido[3,4-b]pyrazin-5-amine hydrochloride;
N-[(3-fluoropiperidin-3-yl)methyl]-7-[4-(trifluoromethoxy phenyl]pyrido[3,4-b]pyrazin-5-amine hydrochloride;
N-[(3-fluoropiperidin-3-yl)methyl]-7-(1,3-oxazol-5-yl)pyrido[3,4-b]pyrazin-5-amine;
7-(2,4-difluorophenyl)-N-[(3-fluoropiperidin-3-yl)methyl]pyrido[3,4-b]pyrazin-5-amine;
7-(4-fluorophenyl)-N-[(3-fluoropiperidin-3-yl)methyl]pyrido[3,4-b]pyrazin-5-amine;
7-(3,4-difluorophenyl)-N-[(3-fluoropiperidin-3-yl)methyl]pyrido[3,4-b]pyrazin-5-amine;
3-fluoro-3-(2-{7-[1-(propan-2-yl)-1H-pyrazol-4-yl]pyrido[3,4-b]pyrazin-5-yl}ethyl)piperidine;
N-[(3-fluoropiperidin-3-yl)methyl]-7-(2-methyl-1,3-benzothiazol-5-yl)pyrido[3,4-b]pyrazin-5-amine hydrochloride;
7-(1-methyl-1H-pyrazol-4-yl)-N-[(3R)-piperidin-3-ylmethyl]pyrido[3,4-b]pyrazin-5-amine hydrochloride;
7-(1,3-dimethyl-1H-pyrazol-4-yl)-N-{[(3S)-3-fluoropiperidin-3-yl]methyl}pyrido[3,4-b]pyrazin-5-amine hydrochloride;
7-(6-ethoxypyridin-3-yl)-N-[(3-fluoropiperidin-3-yl)methyl]pyrido[3,4-b]pyrazin-5-amine hydrochloride;
7-(1,3-benzothiazol-6-yl)-N-[(3-fluoropiperidin-3-yl)methyl]pyrido[3,4-b]pyrazin-5-amine hydrochloride;
N-[(3-fluoropiperidin-3-yl)methyl]-7-(2-methyl-1,3-benzoxazol-5-yl)pyrido[3,4-b]pyrazin-5-amine hydrochloride;
7-(1-methyl-1H-pyrazol-4-yl)-N-[(6-methylpiperidin-3-yl)methyl]pyrido[3,4-b]pyrazin-5-amine hydrochloride; single unknown enantiomer;
7-(2,3-dihydro-1,4-benzodioxin-6-yl)-N-[(3-fluoropiperidin-3-yl)methyl]pyrido[3,4-b]pyrazin-5-amine hydrochloride;
N-[(3-fluoropiperidin-3-yl)methyl]-7-[6-(2,2,2-trifluoroethoxy)pyridin-3-yl]pyrido[3,4-b]pyrazin-5-amine;
N-[(3-fluoropiperidin-3-yl)methyl]-7-(pyridin-4-yl)pyrido[3,4-b]pyrazin-5-amine hydrochloride;
3-({[7-(1-ethyl-1H-pyrazol-4-yl)pyrido[3,4-b]pyrazin-5-yl]oxy}methyl)-3-fluoropiperidine hydrochloride;
7-(1,3-benzothiazol-5-yl)-N-[(3-fluoropiperidin-3-yl)methyl]pyrido[3,4-b]pyrazin-5-amine hydrochloride;
7-(1-methyl-1H-pyrazol-4-yl)-N-[(6-methylpiperidin-3-yl)methyl]pyrido[3,4-b]pyrazin-5-amine hydrochloride, single unknown enantiomer;
N-[(3-fluoropiperidin-3-yl)methyl]-7-(2-methyl-1,3-benzoxazol-6-yl)pyrido[3,4-b]pyrazin-5-amine hydrochloride;
7-(1-methyl-1H-pyrazol-4-yl)-N-[(6-methylpiperidin-3-yl)methyl]pyrido[3,4-b]pyrazin-5-amine hydrochloride, single unknown enantiomer;
7-(1-methyl-1H-pyrazol-4-yl)-N-[(6-methylpiperidin-3-yl)methyl]pyrido[3,4-b]pyrazin-5-amine hydrochloride, single unknown enantiomer;
5-{5-[(3-fluoropiperidin-3-yl)methoxy]pyrido[3,4-b]pyrazin-7-yl}-N,N-dimethylpyrimidin-2-amine hydrochloride;
N-[(3-fluoropiperidin-3-yl)methyl]-7-[1-(2,2,2-trifluoroethyl)-1H-pyrazol-4-yl]pyrido[3,4-b]pyrazin-5-amine, single unknown enantiomer;
N-[(3-fluoropiperidin-3-yl)methyl]-7-[1-(2,2,2-trifluoroethyl)-1H-pyrazol-4-yl]pyrido[3,4-b]pyrazin-5-amine hydrochloride, single unknown enantiomer;
3-fluoro-3-[({7-[1-(propan-2-yl)-1H-pyrazol-4-yl]pyrido[3,4-b]pyrazin-5-yl}oxy)methyl]piperidine hydrochloride;
7-(1-ethyl-1H-pyrazol-4-yl)-N-[(3R)-piperidin-3-ylmethyl]pyrido[3,4-b]pyrazin-5-amine hydrochloride;
7-(1-ethyl-1H-pyrazol-4-yl)-N-[(3R)-piperidin-3-ylmethyl]pyrido[3,4-b]pyrazin-5-amine;
N-[(3R)-piperidin-3-ylmethyl]-7-[1-(propan-2-yl)-1H-pyrazol-4-yl]pyrido[3,4-b]pyrazin-5-amine;
7-(1-tert-butyl-1H-pyrazol-4-yl)-N-[(3-fluoropiperidin-3-yl)methyl]pyrido[3,4-b]pyrazin-5-amine hydrochloride;
7-(3,4-dimethoxyphenyl)-N-[(3-fluoropiperidin-3-yl)methyl]pyrido[3,4-b]pyrazin-5-amine;
(3R)-3-({[7-(1-ethyl-1H-pyrazol-4-yl)pyrido[3,4-b]pyrazin-5-yl]oxy}methyl)piperidine;
N-[(3-fluoropiperidin-3-yl)methyl]-7-[5-methyl-1-(propan-2-yl)-1H-pyrazol-4-yl]pyrido[3,4-b]pyrazin-5-amine;
N-[(3-fluoropiperidin-3-yl)methyl]-7-[1-(methoxymethyl)-1H-pyrazol-4-yl]pyrido[3,4-b]pyrazin-5-amine;
7-(1-tert-butyl-1H-pyrazol-4-yl)-N-{[(3S)-3-fluoropiperidin-3-yl]methyl}pyrido[3,4-b]pyrazin-5-amine;
7-(1-tert-butyl-1H-pyrazol-4-yl)-N-{[(3S)-3-fluoropiperidin-3-yl]methyl}pyrido[3,4-b]pyrazin-5-amine hydrochloride;
7-(3,4-dimethoxyphenyl)-N-[(3R)-piperidin-3-ylmethyl]pyrido[3,4-b]pyrazin-5-amine;
7-(3,4-dimethoxyphenyl)-N-[(3R)-piperidin-3-ylmethyl]pyrido[3,4-b]pyrazin-5-amine hydrochloride;
7-(5,6-dimethoxypyridin-3-yl)-N-{[(3S)-3-fluoropiperidin-3-yl]methyl}pyrido[3,4-b]pyrazin-5-amine;
7-(2,2-difluoro-2H-1,3-benzodioxol-5-yl)-N-{[(3S)-3-fluoropiperidin-3-yl]methyl}pyrido[3,4-b]pyrazin-5-amine;
and salts thereof.

9. A pharmaceutical composition which comprises a compound of formula (I) or a pharmaceutically acceptable salt thereof as defined in claim 1, and one or more pharmaceutically acceptable carriers, diluents or excipients.

10-17. (canceled)

18. A method of treating an autoimmune condition, which comprises administering in a subject in need thereof a therapeutically effective amount of compound of formula (I) or a pharmaceutically acceptable salt thereof, as defined in claim 1.

19. A method of treating an autoimmune condition according to claim 18, wherein the autoimmune condition is selected from systemic lupus erythematosus (SLE), discoid (cutaneous) lupus, Sjorgens syndrome, Wegners granulomatosis and other vasculitides, bullous pemphigoid and pemphigus, idiopathic thrombocytopenic purpura (ITP), giant cell arteriosis, chronic idiopathic urticaria with and without auto-antibody status, glomerulonephritis, chronic transplant rejection and rheumatoid arthritis.

20. A method of treating cancer which comprises administering to a patient in need thereof a therapeutically effective amount of compound of formula (I) or a pharmaceutically acceptable salt thereof, as defined in claim 1.

21. A method of treating an inflammatory disease and/or allergic disorder, which comprises administering to a subject in need thereof a therapeutically effective amount of a compound of formula (I) or a pharmaceutically acceptable salt thereof, as defined in claim 1.
